SEQUENCE LISTING (86 sequences)

SEQ ID NO: 1 - RNA, 11 nt, Artificial Sequence
  Root seed sequence
guucuuucuuu

SEQ ID NO: 2 - RNA, 11 nt, Artificial Sequence
  Root seed sequence
agcuucuucuu

SEQ ID NO: 3 - RNA, 11 nt, Artificial Sequence
  Root seed sequence
ggucuuauuug

SEQ ID NO: 4 - RNA, 11 nt, Artificial Sequence
  Root seed sequence
gguguucauuu

SEQ ID NO: 5 - RNA, 11 nt, Artificial Sequence
  Root seed sequence
uucuuucuuuu

SEQ ID NO: 6 - RNA, 11 nt, Artificial Sequence
  Root seed sequence
gcgcuucucuu

SEQ ID NO: 7 - RNA, 11 nt, Artificial Sequence
  Root seed sequence
uuuuuucucuu

SEQ ID NO: 8 - RNA, 11 nt, Artificial Sequence
  Root seed sequence
guuuucuuucu

SEQ ID NO: 9 - RNA, 11 nt, Artificial Sequence
  Root seed sequence
guucucuucuc

SEQ ID NO: 10 - RNA, 11 nt, Artificial Sequence
  Root seed sequence
cuuuuguucuc

SEQ ID NO: 11 - RNA, 11 nt, Artificial Sequence
  Root seed sequence
cuucuucuuuc

SEQ ID NO: 12 - RNA, 11 nt, Artificial Sequence
  Root seed sequence
guucuuguuuc

SEQ ID NO: 13 - RNA, 11 nt, Artificial Sequence
  Root seed sequence
cgguuucguuu

SEQ ID NO: 14 - RNA, 11 nt, Artificial Sequence
  Root seed sequence
gcgccuuucuu

SEQ ID NO: 15 - RNA, 11 nt, Artificial Sequence
  Root seed sequence
guccuugcuuc

SEQ ID NO: 16 - RNA, 11 nt, Artificial Sequence
  Root seed sequence
cuuccuucuuc

SEQ ID NO: 17 - RNA, 11 nt, Artificial Sequence
  Root seed sequence
gccucuuuucc

SEQ ID NO: 18 - RNA, 11 nt, Artificial Sequence
  Root seed sequence
gcguuccuucu

SEQ ID NO: 19 - RNA, 11 nt, Artificial Sequence
  Root seed sequence
gggucguuuuu

SEQ ID NO: 20 - RNA, 11 nt, Artificial Sequence
  Root seed sequence
gcgccgauuuu

SEQ ID NO: 21 - RNA, 11 nt, Artificial Sequence
  Root seed sequence
gcucccuuucu

SEQ ID NO: 22 - RNA, 11 nt, Artificial Sequence
  Root seed sequence
agucuuuucuu

SEQ ID NO: 23 - RNA, 11 nt, Artificial Sequence
  Root seed sequence
aggcuuucuuu

SEQ ID NO: 24 - RNA, 11 nt, Artificial Sequence
  Root seed sequence
gcgcucucuuc

SEQ ID NO: 25 - RNA, 11 nt, Artificial Sequence
  Root seed sequence
guuucguucga

SEQ ID NO: 26 - RNA, 11 nt, Artificial Sequence
  Root seed sequence
guugcguucua

SEQ ID NO: 27 - RNA, 11 nt, Artificial Sequence
  Root seed sequence
guucucgucuc

SEQ ID NO: 28 - RNA, 11 nt, Artificial Sequence
  Root seed sequence
ggccgaccuac

SEQ ID NO: 29 - RNA, 11 nt, Artificial Sequence
  Root seed sequence
gucccuucuuc

SEQ ID NO: 30 - RNA, 11 nt, Artificial Sequence
  Root seed sequence
gcccucccuga

SEQ ID NO: 31 - RNA, 22 nt, Homo sapiens
uggcagugucuuagcugguugu

SEQ ID NO: 32 - RNA, 12 nt, Artificial Sequence
  Synthetic Sequence
cgcuucucuuuu

SEQ ID NO: 33 - RNA, 21 nt, Artificial Sequence
  Synthetic Sequence
uggcagugucgcuucucuuuu

SEQ ID NO: 34 - RNA, 19 nt, Artificial Sequence
  Synthetic Sequence
uggcagugucgcuucucuu

SEQ ID NO: 35 - RNA, 22 nt, Artificial Sequence
  Synthetic Sequence
uggcagugguucuuucuuuuuu

SEQ ID NO: 36 - RNA, 22 nt, Artificial Sequence
  Synthetic Sequence
acaaccagcuaagacacugcca

SEQ ID NO: 37 - RNA, 22 nt, Artificial Sequence
  Synthetic Sequence
acaaccagcuaagacacugcuu

SEQ ID NO: 38 - RNA, 22 nt, Artificial Sequence
  Synthetic Sequence
accagcuaagacacugccaaaa

SEQ ID NO: 39 - RNA, 21 nt, Artificial Sequence
  Synthetic Sequence
ccagcuaagacacugccagaa

SEQ ID NO: 40 - RNA, 19 nt, Artificial Sequence
  Synthetic Sequence
uugucucugguccuuacuu

SEQ ID NO: 41 - RNA, 22 nt, Artificial Sequence
  Synthetic Sequence
uugucucugguccuuacuuuuu

SEQ ID NO: 42 - DNA, 19 bp, Artificial Sequence
  Synthetic Sequence
agatggccatggcgcggac

SEQ ID NO: 43 - RNA, 21 nt, Artificial Sequence
  Synthetic Sequence
agauggccauggcgcggacuu

SEQ ID NO: 44 - RNA, 21 nt, Artificial Sequence
  Synthetic Sequence
cgauggccauggcgcggacuu

SEQ ID NO: 45 - DNA, 18 bp, Artificial Sequence
  Synthetic Sequence
gatggccatggcgcggac

SEQ ID NO: 46 - RNA, 21 nt, Artificial Sequence
  Synthetic Sequence
gauggccauggcgcggacuuu

SEQ ID NO: 47 - DNA, 21 bp, Artificial Sequence
  Synthetic Sequence
tugucucugguccuuacuuaa

SEQ ID NO: 48 - RNA, 21 nt, Artificial Sequence
  Synthetic Sequence
uugucucugguccuuacuuaa

SEQ ID NO: 49 - DNA, 21 bp, Artificial Sequence
  Synthetic Sequence
tgaacauuggaauaguuucaa

SEQ ID NO: 50 - RNA, 21 nt, Artificial Sequence
  Synthetic Sequence
ugaacauuggaauaguuucaa

SEQ ID NO: 51 - DNA, 21 bp, Artificial Sequence
  Synthetic Sequence
tuaucuauaaugaucagguaa

SEQ ID NO: 52 - RNA, 21 nt, Artificial Sequence
  Synthetic Sequence
uuaucuauaaugaucagguaa

SEQ ID NO: 53 - DNA, 21 bp, Artificial Sequence
  Synthetic Sequence
tuaagacuugagaugauccaa

SEQ ID NO: 54 - RNA, 21 nt, Artificial Sequence
  Synthetic Sequence
uuaagacuugagaugauccaa

SEQ ID NO: 55 - DNA, 21 bp, Artificial Sequence
  Synthetic Sequence
tatgagaacatactttcccaa

SEQ ID NO: 56 - RNA, 21 nt, Artificial Sequence
  Synthetic Sequence
uaugagaacauacuuucccaa

SEQ ID NO: 57 - DNA, 21 bp, Artificial Sequence
  Synthetic Sequence
taugagaacauacuuucccaa

SEQ ID NO: 58 - RNA, 21 nt, Artificial Sequence
  Synthetic Sequence
acauuaaagucuguugucauu

SEQ ID NO: 59 - RNA, 21 nt, Artificial Sequence
  Synthetic Sequence
uuuuauuucaccaugauuuuu

SEQ ID NO: 60 - RNA, 21 nt, Artificial Sequence
  Synthetic Sequence
uugaucuaaaugcauuguguu

SEQ ID NO: 61 - RNA, 21 nt, Artificial Sequence
  Synthetic Sequence
uuaagagaagccuuacugguu

SEQ ID NO: 62 - RNA, 21 nt, Artificial Sequence
  Synthetic Sequence
auuucaggaauuguuaaaguu

SEQ ID NO: 63 - RNA, 21 nt, Artificial Sequence
  Synthetic Sequence
uugguauucagugugaugauu

SEQ ID NO: 64 - RNA, 22 nt, Artificial Sequence
  Synthetic Sequence
uaaggcacgcggugaaugccuu

SEQ ID NO: 65 - RNA, 21 nt, Artificial Sequence
  Synthetic Sequence
uaaggcacgcgcuucucuuuu

SEQ ID NO: 66 - RNA, 21 nt, Artificial Sequence
  Synthetic Sequence
uaaggcacggccgaccuacuu

SEQ ID NO: 67 - RNA, 21 nt, Artificial Sequence
  Synthetic Sequence
uaaggcacgaacugagcguuu

SEQ ID NO: 68 - RNA, 21 nt, Artificial Sequence
  Synthetic Sequence
uaaggcacgucccuucuucuu

SEQ ID NO: 69 - RNA, 21 nt, Artificial Sequence
  Synthetic Sequence
uaaggcacgcauucgccccuu

SEQ ID NO: 70 - DNA, 21 bp, Artificial Sequence
  Synthetic Sequence
taaggcacggccgaccuacuu

SEQ ID NO: 71 - DNA, 21 bp, Artificial Sequence
  Synthetic Sequence
taaggcacgaacugagcguuu

SEQ ID NO: 72 - DNA, 21 bp, Artificial Sequence
  Synthetic Sequence
taaggcacgucccuucuucuu

SEQ ID NO: 73 - DNA, 21 bp, Artificial Sequence
  Synthetic Sequence
taaggcacgcauucgccccuu

SEQ ID NO: 74 - DNA, 22 bp, Artificial Sequence
  Synthetic Sequence
taaggcacgcggugaaugccuu

SEQ ID NO: 75 - DNA, 21 bp, Artificial Sequence
  Synthetic Sequence
taaggcacgcgcuucucuuuu

SEQ ID NO: 76 - RNA, 21 nt, Artificial Sequence
  Synthetic Sequence
uggagugugacaaugguguuu

SEQ ID NO: 77 - RNA, 21 nt, Artificial Sequence
  Synthetic Sequence
uggagugugcgcuucucuuuu

SEQ ID NO: 78 - DNA, 21 bp, Artificial Sequence
  Synthetic Sequence
tggagugugacaaugguguuu

SEQ ID NO: 79 - DNA, 21 bp, Artificial Sequence
  Synthetic Sequence
tggagugugcgcuucucuuuu

SEQ ID NO: 80 - RNA, 21 nt, Artificial Sequence
  Synthetic Sequence
uggaggugacaaugguguuuu

SEQ ID NO: 81 - DNA, 21 bp, Artificial Sequence
  Synthetic Sequence
tggaggugacaaugguguuuu

SEQ ID NO: 82 - RNA, 14 nt, Artificial Sequence
  Synthetic Sequence
cgcuucucuunnnn

SEQ ID NO: 83 - RNA, 14 nt, Artificial Sequence
  Synthetic Sequence
gccgaccuacnnnn

SEQ ID NO: 84 - RNA, 14 nt, Artificial Sequence
  Synthetic Sequence
aacugagcgunnnn

SEQ ID NO: 85 - RNA, 14 nt, Artificial Sequence
  Synthetic Sequence
ucccuucuucnnnn

SEQ ID NO: 86 - RNA, 14 nt, Artificial Sequence
  Synthetic Sequence
cauucgccccnnnn

(19) United States
(12) Patent Application Publication
(10) Pub. No.: US 2015/0291957 A1
(43) Pub. Date: Oct. 15, 2015

(54) METHODS AND COMPOSITIONS TO PRODUCE ss-RNAi ACTIVITY WITH ENHANCED POTENCY

(71) Applicant: Larry J. SMITH, Omaha, NE (US)

(72) Inventor: Larry J. Smith, Omaha, NE (US)

(21) Appl. No.: 14/438,857

(22) PCT Filed: Oct. 28, 2013
(86) PCT No.: PCT/US13/67153

§ 371 Date: Apr. 27, 2015

Related U.S. Application Data
(60) Provisional application No. 61/719,325, filed on Oct. 26, 2012.

(51) Int. Cl. C12N 15/113

(52) U.S. Cl. CPC C12N 15/113; C12N 15/1137; C12N 2310/32; C12N 2310/14

(57) ABSTRACT

Compositions and methods for down modulating expression of target nucleic acids are disclosed. This invention relates to the fields of medicine, drug development and modulation of target nucleic acid expression. More specifically, the invention provides compositions and methods of use thereof that facilitate the modulation of target nucleic acid expression using novel oligonucleotide based drugs that act through an inhibitory RNA (RNAi) mechanism of action.

This application claims priority to U.S. Provisional Application No. 61/719,325 filed Oct. 26, 2012, the entire contents being incorporated herein by reference as though set forth in full.

### FIELD OF THE INVENTION

This invention relates to the fields of medicine, drug development and modulation of target nucleic acid expression. More specifically, the invention provides compositions and methods of use thereof that facilitate the modulation of target nucleic acid expression using novel oligonucleotide based drugs that act through an inhibitory RNA (RNAi) mechanism of action.

### BACKGROUND OF THE INVENTION

Numerous publications and patent documents, including both published applications and issued patents, are cited throughout the specification in order to describe the state of the art to which this invention pertains. Each of these citations is incorporated herein by reference as though set forth in full.

RNA interference (RNAi) refers to molecules and mechanisms whereby certain double stranded RNA (dsRNA) structures (RNAi triggers) cause sequence specific gene inhibition. Two main categories of RNAi triggers have been distinguished: small inhibitory RNA (siRNA) and microRNA (miRNA). In the case of naturally occurring siRNA the original source of the dsRNA is exogenous to the cell or it is derived from transposable elements within the cell. Cells may then process the dsRNA to produce siRNA that can specifically suppress the activity of the source of the dsRNA. The exogenous sources include certain viruses where the siRNA generated provides a defense mechanism against such invaders.

In contrast, naturally occurring miRNA is produced from precursor molecules that are generated from independent genes or from very short intron sequences found in some protein encoding genes. Unlike siRNA molecules, miRNA molecules broadly inhibit multiple different genes rather than being narrowly focused on a particular gene. Thus, naturally occurring siRNA characteristically performs more narrowly focused inhibitory actions than does miRNA.

These differences are reflected, in part, in the “targeting codes” that are associated with these two classes of RNAi. The targeting code can be briefly defined as the subset of the antisense strand sequence that is primarily or fully responsible for recognizing the target sequence by complementary base pairing. Ambros et al., provide a more detailed description of how naturally occurring siRNA and miRNA can be experimentally distinguished and annotated (RNA 9: 277-9, 2003.)

The general mechanisms that underlie the implementation of siRNA and miRNA-dependent activity are substantially overlapping, but the particulars of how siRNA and miRNA function to suppress gene expression are substantially different. At the heart of the general mechanisms applicable to both of these types of RNAi is the RNA-induced silencing complex (RISC). The double stranded siRNA or miRNA is loaded into RISC. Next the sense strand is discarded and the antisense strand is used to direct RISC to its target(s).

In the case of siRNA typically and for a subset of miRNAs, the RISC complex includes an enzyme called argonaute-2 (AGO-2) that cleaves a specific mRNA target. Other enzymes recognize the bifurcated mRNA as abnormal and further degrade it while a more complicated mechanism is engendered by miRNA. MRNA or miRNA cleavage by AGO-2 directed by seqsiRNA, seqIMiRs, ss-siRNA and ss-IMiRs as for the conventional compounds requires a high degree of sequence complementarity between the antisense strand and its target particularly with respect to the nucleosides adjacent to the AGO-2 cleavage side that are located at positions 10 and 11 counting from the 5′-end of the guide strand along with two nucleosides on either side of positions 10 and 11 for a total of 6 contiguous nucleosides. The nucleoside sequence found at this location (central region) is the targeting code in this context. Additional nucleosides out side of this targeting code also affect the efficiency of the ARO-2 dependent target cleavage by RISC but some mismatches and the universal bases provided herein can be tolerated in these flanking areas outside of the central region with three or less being preferred. It is also preferred that when there is more than one mismatch or universal base that they not be contiguous and that any mismatches in the seed sequence not involve positions 4 and 5 from the 5′-end. Mismatches in the terminal positions of a complementary strand are the least disruptive of binding affinity. One or two mismatches in the central region can still result in a repression of target mRNA translation but by a steric hindrance rather than by a catalytic mechanism. This approach can be used to distinguish between mRNA produced by mutant vs. normal alleles.

Genome wide identification of miRNA targets and computational predictions estimate that each mammalian miRNA on average inhibits the expression of hundreds of different mRNAs. Thus, miRNA can be involved in coordinating patterns of gene expression. The ability of particular miRNAs to produce a particular cellular phenotype, however, can be based on the modulation of the expression of as few genes as one. Most mammalian genes appear to be post-transcriptionally regulated by miRNAs. Abnormalities in the expression of particular miRNAs have pathogenic roles in a wide range of medical disorders.

The targeting code most commonly used by miRNA resides in a so called “seed sequence” that is made up of nucleosides 2-8 (or 2-7 in the case of some endogenous miRNAs) counting in from the 5′-end of the guide or antisense strand. This sequence is the major determinant of target recognition and is sufficient to trigger translational silencing. Target sequences usually are found in the 3′-untranslated region (3′UTR) of the mRNA targets. Infrequently, complementarity between nucleosides down-stream of the seed sequence and the target contribute to target recognition particularly when the seed sequence has a weak match with the target. These are called 3′-supplementary or 3′-compensatory sites.

Another category of miRNA utilizes a target code involving “centered sites” that consist of 11 or 12 consecutive nucleosides that begin at position 4 or 5 downstream from the 5′-end of the guide or antisense strand. To date no 3′-supplementary or 3′-compensatory sites have been uncovered that support target recognition by the targeting code.

MiRNA, other than the few with a siRNA-like inhibitory mechanism, can suppress the translation of specific sets of mRNA by interfering with the translation machinery without affecting mRNA levels and/or by causing the mRNA to be degraded by promoting the conditions necessary to activate the naturally occurring 5′-to-3′ mRNA decay pathway.

In addition to the common targeting of the 3′UTR of mRNA, some miRNAs have been found to target the 5′-UTR or to the coding region of some mRNAs. In some of these cases the miRNA/RISC complex inhibits the translation of the target mRNA and in others translation is promoted. Further, there are instances of certain miRNAs forming complexes with ribonucleoproteins and thus interfering with their RNA binding functions in a RISC-independent manner. Finally, there are also documented instances in which miRNAs can affect transcription of particular genes by binding to DNA.

Over the last dozen years, RNAi related mechanisms involving siRNA and miRNA have been substantially elucidated and found to occur widely in both plants and animals including in all human cell types. In turn, these advances have been applied to the design and use of RNAi based drugs for use as therapeutic candidates and as a tool for various research and drug development purposes. Tuschl's group first reported the administration of synthetic siRNA to cells more than 10 years ago (Elbashir et al., Nature 411: 494-498, 2001). Administration of conventional siRNA therapeutics has very recently reached the stage where significant RNAi activity can be achieved in the livers of primates as well as man. The best of these results to date are based on the use of second-generation lipid nanoparticles (LNPs) that envelop the siRNA and promote its delivery to hepatic cells. These data come from interim results from a phase I trial of a siRNA directed to PCSK9.

MiRNA is comparatively a fundamentally more complex area of RNAi than siRNA and consequently attempts to acquire miRNA-based drug candidates for therapeutic as well as use as a tool for various research and drug development purposes have lagged behind siRNA. Potential miRNA therapeutics includes miRNA inhibitors and miRNA mimics. Most advanced is the use of antisense oligonucleotides (oligos) with a steric hindrance mechanism to inhibit the function of certain miRNAs. One example is a mixed LNA/DNA nucleoside phosphorothioate oligo that inhibits miR-122 and which has completed phase II testing with promising results. Mir-122 is highly expressed by liver and is required for HCV production and increases the level of total cholesterol in plasma.

Least advanced is the delivery of miRNA mimics to tissues in vivo for therapeutic or research or drug development purposes. In part this is because the field is still in the early stages of elucidating the functions and identities of therapeutically relevant miRNAs. A relatively small number of miRNAs, however, have have been described in the literature and support key roles for such miRNAs in certain medical conditions. A number of these miRNAs function as anti-oncogenes for particular types of cancer where they are pathologically under expressed. Importantly replacement of the deficient miRNA often has a substantial anti-cancer activity, for example, miR-34 and let-7 family members.

It is well recognized in the art that the single most important barrier to the development of siRNA and miRNA mimics as drugs is the poor uptake of these compounds by tissues in the body (Aliabadi et al., Biomaterials 33: 2546, 2012; Kanasty et al., Mol Ther published online ahead of print Jan. 17, 2012). It is widely held that for general use complex carriers are needed that will envelop the siRNA or miRNA mimic and promote their delivery in to tissues in a bioavailable manner. To date the success of this approach is essentially limited to the delivery of such compounds to liver.

In contrast, steric hindrance antisense oligos being used to inhibit miRNAs are being successfully delivered tissues without the need for a carrier. Further, clinically important endpoints are being achieved. Such oligos, however, require high doses and perhaps most importantly very high affinity for their target miRNA (Elmen et al., Nature 452: 896, 2008; Lanford et al., Science 327: 198, 2010). Thus, miRNAs with relatively high G/C content should be most susceptible to this form of inhibition. It may not be possible to effectively target the majority of miRNAs using this approach and existing antisense oligo chemistries because of the high affinity requirement.

The miRNA sequences and nomenclature used herein are taken from the miRBase (www.mirbase.org) which has been described in Griffiths-Jones et al., Nucleic Acids Research 34: D140-D144, 2006. In brief, numbers that immediately follow the designation miR-, for example, miR-29, designate particular miRNAs. This designation is applied to the corresponding miRNAs across various species. Letters, for example in miR-34a and miR-34b, distinguish particular miRNAs differing in only one or two positions in the mature miRNA (antisense strand). Numbers following a second dash, for example in miR-24-1 and miR-24-2, distinguish distinct loci that give rise to identical mature miRNAs. These miRNAs can have different sense strands. Multiple miRNAs family members that differ in only one or two nucleoside positions from some other member(s) for the family in the mature miRNA and which also come from distinct hairpin loci have both letters and additional numbers following the letters, for example, miR-29b-1 and miR-29b-2 with the other family members being miR-29a and miR-29c. Finally, in some instances two different mature miRNA sequences are excised from the same hairpin precursor where one comes from the 5′ arm and the other from the 3′ arm. These are designated −5p and -3p respectively, for example, miR-17-5p and miR-17-3p

### SUMMARY OF THE INVENTION

In accordance with the present invention, methods and compositions that provide RNAi activity in tissues against targets of choice in vitro and in vivo are disclosed. The compositions of the present invention can be delivered to subjects as single strand oligos in a vehicle such as a physiological buffer, with out the requirement for a carrier or prodrug design while ultimately being capable of suppressing the intended target(s) in a wide variety of tissue types. The present inventor has provide designs for individual oligo strands with features that allow them survive administration in vivo, become bioavailable in a wide variety of tissues where they are capable of silencing the target in the absence of a partner strand.

The types of compositions of the present invention fall into three basic groups to include those that: (1) inhibit the expression of individual target nucleic acid or small numbers of target nucleic acids such as mRNA, endogenous antisense and long non coding RNA by an AGO-2 based cleavage mechanism; (2) inhibit the expression of particular miRNAs; and (3) provide miRNA-like functions through partially mimicking the actions of particular endogenous miRNAs or generating miRNA-like compounds with novel seed sequences.

Single stranded compounds where only the guide strand is administered to cultured cells or to subjects with these three types of activity are referred to as ss-siRNA, ss-IMiRs and ss-MiRs respectively. The ss-RNAi compounds of the present invention are designed to be much more efficiently engaged by the RNAi mechanism than ss-RNAi compounds known in the art. The result is a surprisingly higher potency compared to ss-RNAi not designed in this manner. This novel approach is called Accommodating Helical Design.

Thus, in accordance with the present invention, a composition for inhibiting expression of at least one target nucleic acid sequence of interest in a cell in vivo and in vitro is provided. An exemplary method comprises providing a modified single stranded oligoribonucleic acid in a pharmaceutically acceptable vehicle, said strand comprising one or more accommodating helical design (AHD) modifications and other chemical modifications effective to alter at least one parameter selected from the group consisting of enhanced resistance to 5′ and 3′ exonucleases and endonucleases in vivo; enhanced C3′-endo conformation in one or more flexible sugar moieties in said oligoribonucleotide strand comprising said AHD modifications; increased potency in vivo and in vitro; reduced steric hinderance of strand interaction with RISC machinery via omission of moieties which project into major or minor grooves of duplexed RNAi triggers while maintaining RNAi activity; reduced off-target effects; and enhanced activity of the RNAi mechanism within cells relative to RNA strands lacking said AHD modifications. The oligoribonucleotides present in the composition lacking a pro-drug design; being between 16 and 22 nucleotides in length, exclusive of any 3′-end overhang precursor and having a region of complementarity to the target ribonucleic acid that is at least 6 contiguous nucleosides in length, wherein the oligoribonucleotide comprises at least one of ribose, 2′-fluoro modification, 2′-O-methyl modification, an AHD modified sugar or sugar substitute, and an AHD modified base and optionally comprises a 5′ end modification, a 3′ end modification, a modification which increases resistance to endonucleases and a 3′-end overhang precursor between 1 and 4 units in length wherein said modified oligoribonucleotide strand exhibits increased inhibition of expression of said target ribonucleic acid within said cell relative to identical oligoribonucleotide strands lacking said AHD modifications.

In yet another aspect of the invention, a method of inhibiting expression of a target nucleic acid comprising contacting a cell expressing the target nucleic acid with an effective amount of the ss-RNAi, ss-MiR or ss-IMir compositions disclosed herein is provided, wherein the said composition is effective to degrade target RNA or inhibit translation of mRNA encoding a protein produced by said target nucleic acid or by inhibiting transcription.

Finally, in yet another embodiment, an in vitro method of improving an RNAi effect in vitro or in vivo against a target nucleic acid is provided. An exemplarily method comprises obtaining or providing an oligoribonucleotide sequence which specifically hybridizes to said target nucleic acid; introducing one or more accommodating helical design (AHD) modifications and other chemical modifications into said oligoribonucleotide, thereby producing a modified oligoribonucleotide, wherein said modifications are effective to modulate at least one parameter selected from the group consisting of enhanced resistance to 5′ and 3′ exonucleases and endonucleases in vivo; enhanced C3′-endo conformation in one or more flexible sugar moieties in said oligoribonucleotide strand comprising said AHD modifications; increased potency in vivo and in vitro; reduced steric hinderance of strand interaction with RISC machinery via omission of moieties which project into major or minor grooves of duplexed RNAi triggers while maintaining RNAi activity; reduced off-target effects; and enhanced activity of the RNAi mechanism within target tissue in vivo relative to RNA strands lacking said AHD modifications; and contacting a first population of cells expressing said target nucleic acid with the modified oligoribonucleotide of step ii) and a second population of identical cells expressing said target nucleic acid with an identical oligoribonucleotide strand lacking said modifications. After a suitable period of time, the effects of said contact on said parameter is determined, parameters being affected by those strands comprising said AHD modifications being identified as AHD modifications which improve RNAi effects in vitro and in vivo.

### BRIEF DESCRIPTION OF THE DRAWINGS

FIG. 1: Key to Strand Modifications.

FIG. 2: CENA

FIG. 3: HM

FIG. 4: UNA (Unlocked Nucleic Acid)

FIG. 5: ANA

FIG. 6: EA

FIG. 7: HNA

FIG. 8: AENA

FIG. 9: CRN (Conformationally Restricted Nucleoside)

FIG. 10: LNA (Locked Nucleic Acids)

FIG. 11: Examples of Abasic Nucleosides

FIG. 12: CeNA

FIG. 13: 5′ Carbon: Site for 5′-End Terminal Modifications

FIG. 14: 2′-O-Methoxyethyl (2′-MOE) Modification of a Ribonucleoside

FIG. 15: C10 (T_{C10}) and C16 (T_{C16}) Conjugation to a Nucleoside Illustrated with a Thymine Containing Nucleoside

FIG. 16: Structure for 5′-(E)-vinyl-phosphonate Conjugated to a Thymine Containing 2′-O-MOE Nucleoside (5′-VP-T)

FIG. 17:Human/Murine MiR-34a-5p ss-MiR

FIG. 18: Human/Murine MiR-34a-5p ss-IMiR

FIG. 19: Human/Murine PTEN ss-siRNA

FIG. 20: Human/Non-human primate/Murine p53 ss-siRNA

FIG. 21: Application of AHD to Human/Murine PTEN ss-siRNA

FIG. 22: Application of AHD to Murine Factor VII ss-siRNA

FIG. 23: Application of AHD to Murine Apo-CIII ss-siRNA

FIG. 24: Application of AHD to Murine SSB ss-siRNA

FIG. 25: Application of AHD to Murine Apo-B ss-siRNA

FIG. 26: Application of AHD to MiR-124 ss-MiR

FIG. 27: Application of AHD to MiR-122 ss-MiR

FIG. 28: Examples of Five Seed Vehicles Suitable for use with the Nine Terminal 5′-End Nucleosides of any MiRNA Guide Strand

### DETAILED DESCRIPTION OF THE INVENTION

### A. Overview of Prior Art

It is currently assumed in the art that the broad application of siRNA-based compounds and miRNA mimics as drugs will require the development of carriers that do not currently exist and that likely will involve different designs for different cell types. Existing carriers are designed for distribution to tissues from the general circulation and have shown limited but meaningful success in obtaining siRNA activity in the liver in vivo. There is also some evidence indicating that such carriers may be able to deliver conventional RNAi drugs to areas in the body where the capillary bed has become leaky due to certain pathological conditions such as cancer. For targeting to other tissue types, it is generally believed that the carriers required to establish conventional siRNA and miRNA mimics as drug platforms will be of a complex structure and will frequently entail approaches that envelop siRNA or miRNA duplexes.

Carriers are believed to be needed for multiple reasons based on what happens when naked siRNA is injected into subjects including: (1) poor uptake by cells; (2) destruction by nucleases; and (3) rapid clearance of intact duplexes from the body by the kidneys. Further, the carriers being developed for general drug use variably have a variety of associated problems including, but not limited to, toxicity, difficulties in formulation, a short shelf half-life, long development times and very restricted range of organ/tissue/cell types with which they will work.

One of the most consistent problems is the large size of the carriers in clinical use and those in development for systemic use. Most macromolecules and particles that can move out of the circulation appear to move into the tissues by passively diffusing through capillaries presumably through pores. SNALP carriers in clinical use are about 70 nM in diameter while the large majority of the pores hypothesized to exist in the capillaries are about 5-12 nM in width with relatively scarce pores of about 25 nanometers in width depending on the tissue. Data suggests the liver is an exception with apparent larger pore sizes of about 110 nM. Particles of up to 60 nM in size have been shown to pass from the circulation into the bone marrow by an active process, but there appear to be no publications that look at the ability of RNAi-based drug carriers to get into the bone marrow from the circulation. Thus, RNAi-based drug carriers are too large to pass from the circulation to the large majority of normal tissues in the body. In addition, the kidneys very rapidly remove nanoparticles in the circulation smaller than 20 nM so these particles are not practical for administration routes that involve the circulation. Some types of trauma can make capillaries more leaky and thus less likely to restrain the movement of large molecular structures such as the carriers under consideration. Solid cancers, for example, can disrupt and/or replace the normal vasculature at least the more superficial cancer cells in a tumor can be available to SNALPs. Thus, the notion of treating cancer with nanoparticles carrying RNAi-based drugs makes good sense but only up to a point. In general terms the vascularization of solid tumors is complex and highly variable. The variability depends on the type of cancer, the individual characteristics of particular cancers and on the stage of the disease. Areas of particular cancers can be sufficiently deficient in blood supply to hinder chemotherapeutic treatment. Further, once outside the vasculature the carrier must cross other barriers such as the extracellular matrix, basement membranes and the membrane around the target cell. In some situations, for example in solid cancers, the pressure gradient between the circulation and the interstitial fluid can be unfavorable for the passage of large molecules and particles into the cancer.

SNALPs and other types of RNAi-based drug carriers do not have to pass through vascular pores in order to be in contact with the cells that line the vasculature or the cells in the circulation. Further, the administration of RNAi-based drugs associated with carriers topically or to compartments in the body, (e.g., intraocular and intravesiclular spaces), where high concentrations can be maintained for a period of time, in contact with the cells to be treated, are also amenable to treatment using the existing conventional technology. In addition, the administration of carrier-free siRNA to the lungs by inhalation has been shown to have local activity. So in each of these situations where cells in the body are not protected by vascular pores the cells are potential targets for the delivery of RNAi-based drugs using the conventional technology. These situations, however, only apply to a relatively small number of cell types in the body.

Ss-RNAi is an attractive concept but until recently has received little attention. Ss-RNAi was proposed as a possibly viable approach to gene silencing in vitro and in vivo as early as 2002 (Martinez et al., Cell 110: 563, 2002; Schwarz et al., Mol Cell 10: 537, 2002; Holen et al., Nucleic Acids Res 31: 2401, 2003; US 2006/0166910; WO 2004/007718; WO 2004/063375). The antisense strands found in naturally occurring RNAi triggers, however, generally cannot efficiently function as a trigger without their partner sense strand although exceptional strands with modest activity have been occasionally described in the literature. Recently, the extensive use of 2′-fluoro modified nucleosides exclusively or with some ribose and/or 2′-O-methyl containing nucleosides have been shown to provide the means to obtain ss-RNAi compounds with significant silencing activity on a regular basis albeit with substantially less potency and for a shorter period of time than the corresponding double stranded RNAi triggers. This ss-RNAi activity was asserted or shown depending on the sequence and/or modications to require or to be boosted by the use of strands with a 5′-end ribose 5′ carbon that is either phosphorylated or has a particular phosphate bioisostere. (Lima et al., Cell 150: 883, 2012; Yu et al., Cell 150: 895, 2012; Haringsma et al., Nucleic Acids Res 40: 4125, 2012; Chorn et al., RNA 18: 1796, 2012; US 2009/002365; WO 2011/046983; WO 2011/139699; WO 2011/139702; WO 2012/145729; WO 2012/0272206).

The present invention provides a novel method for the creation of potent ss-RNAi compounds based on accommodating helical design (AHD). This approach involves generating ss-RNAi compounds that, in the presence of the RNAi molecular machinery, assume a conformation sufficiently like the one it would assume if it existed as part of a conventional double strand RNAi trigger. As a result the strand is surprisingly more active in producing the intended silencing activity than are the ss-RNAi compounds designed according to prior art methods.

### B. Definitions

The following definitions and terms are provided to facilitate an understanding of the invention.

“2′-fluoro” refers to a nucleoside modification where the fluorine has the same stereochemical orientation as the hydroxyl in ribose. In instances where the fluorine has the opposite orientation, the associated nucleoside will be referred to as FANA or 2′-deoxy-2′fluoro-arabinonucleic acid.

“3′-supplementary or 3′-compensatory sites” refers to sites in some miRNA antisense strands down-stream of the seed sequence that are complementary to the target sequence and contribute to target selection particularly when the seed sequence has a weak match with the target.

3′UTR is an abbreviation for the 3′ untranslated region of an mRNA.

“5′-to-3′ mRNA decay pathway” refers to a naturally occurring pathway for degrading mRNA that is initiated by the removal of the poly(A) tail by deadenylases. This is followed by removal of the 5′-cap and subsequent 5′ to 3′ degradation of the rest of the mRNA.

“Abasic nucleoside” as used herein refers to any of a number of structures that typically have a normal or modified nucleoside sugar or sugar analog, including those provided herein, and can be linked to other nucleosides using the linkages provided for herein. These abasic nucleosides, however, can radically depart from normal ribose structure to include novel five membered or six membered rings or no ring at all as shown in FIG. 11. Typically the position normally occupied by the base has some chemical moiety such as a methylene group that is not an isostere of any naturally occurring base found in nucleic acids and that has essentially no meaningful charge-charge or steric hindrance type interactions with any opposing nucleoside in a nucleic acid to which the strand containing the abasic nucleoside binds.

“Accommodating Helical Design” or “AHD” refers to the design of ss-RNAi strands as provided for herein such that they are thermodynamically preorganized to readily assume the same conformation they would have in an A-type helix (the conformation they would have if they were in a seqRNAi or conventional RNAi trigger duplex) when they contact the components of the RNAi machinery responsible for properly inserting an antisense strand into RISC including the contribution made by RISC itself. This preorganization involves the use of accommodating modifications that increase the probability that multiple nucleoside sugar or sugar substitutes in the ss-RNAi will be in the C3′-endo conformation.

“Accommodating modification” refers to a modification that is useful in realizing the main goal of generating ss-RNAi compounds that in the presence of molecular components of the RNAi machinery readily assume a conformation sufficiently similar to the conformation the strand would have if it were in a conventional RNAi trigger. The result of this modification(s) being the efficient and proper loading of the strand into RISC such that silencing of the intended target(s) can be achieved at the level(s) provided for herein.

“AENA” is an abbreviation for a 2′-deoxy-2′-N,4′-C-ethylene-LNA. It is shown in FIG. 8 where B is any of the bases provided for herein.

“Antisense oligos or strands” are oligos that are complementary to sense oligos, pre-mRNA, mRNA or to mature miRNA and which bind to such nucleic acids by means of complementary base pairing. The antisense oligo need not base pair with every nucleoside in the target. All that is necessary is that there be sufficient binding to provide for a Tm of greater than or equal to 40 ° C. under physiologic salt conditions at submicromolar oligo concentrations unless otherwise stated herein.

“Algorithms” refers to sets of rules used to design oligo strands for use in the generation of seqRNAi sets or pairs.

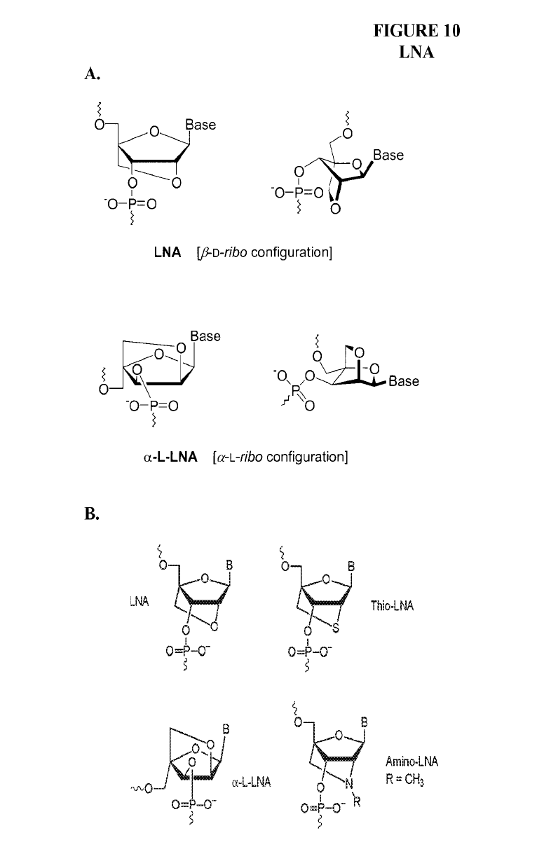

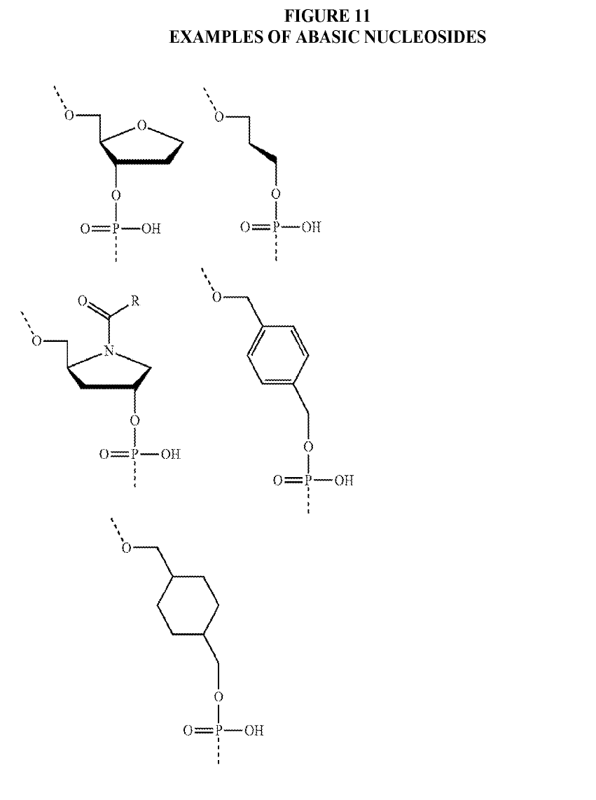

“ALN” is an acronym for alpha-L-LNA. It has an alpha-L-ribo configuration and is illustrated in FIG. 10 panels A and B along with one phosphodiester linkage and where B is any of the bases provided for herein.

“ANA” is an acronym for altritol nucleic acid. It is illustrated in FIG. 5 where B is any of the bases provided for herein.

“Antisense strand vehicle” is used to describe an antisense strand structure into which particular seed sequences can be inserted as a starting point for the design of ss-MiR compounds. These vehicles are designed and/or selected to minimize off target effects and to promote efficient RISC loading.

“Architecture” refers to one of the possible architectural configurations of the seqRNAi-based duplexes formed after a set of seqRNAi strands undergoes complementary base pairing or it refers to the group of such architectures.

“Asymmetry rule” refers to the naturally occurring mechanism whereby the likelihood of a particular strand in a siRNA, miRNA or seqRNAi-based duplex is selected by RISC as the antisense strand. It has been applied to the design of conventional siRNA compounds and it can apply to seqRNAi compounds. In brief, the relative Tm of the 4 terminal duplexed nucleosides at one end of the duplex compared to the corresponding nucleosides at the other terminus of the duplex plays a key role in determining the relative degree to which each strand will function as the antisense strand in RISC. The strand with its 5′-end involved in the duplexed terminus with the lower interstrand Tm more likely will be loaded into RISC as the antisense strand. The Tm effect, however, is not evenly distributed across the duplexed terminal nucleosides because the most terminal is the most important with the successive nucleosides being progressively less important with the terminal 4 duplexed nucleosides being the most significant.

“Backbone” refers to the alternating linker/sugar or sugar analog structure of oligos while the normal bases or their substitutes occur as appendages to the backbone.

“Base” refers to a component moiety of a nucleoside or nucleoside analog suitable for use as part of a seqRNAi passenger or guide strand or AHD ss-RNAi strand. Bases are distinguishable from the sugar or sugar analog component nucleoside or nucleoside analog and from the linkage joining the nucleoside or nucleoside analogs in the strand. A base directly interacts with a base in a complementary strand or within the same strand through charge/charge interactions that can either increase or decrease the affinity of the two strands or regions of the same strand for each other. The standard naturally occurring bases are cytosine, uracil, adenine, guanine and thymine. The term base also includes any of the other moieties provided for herein that are conjugated to a sugar or sugar analog and that have a direct interaction with a base in a complementary strand or the same strand when the strand containing the base forms a duplex with a complementary strand or with itself in the form of a hairpin.

“Bulge structures or bulge” refers to regions in a miRNA duplex or seqMiR-based duplex where multiple interior contiguous nucleosides in one strand fail to base pair with the partner strand in a manner that results in the formation of a bulge in the duplex composed of these nucleosides. Bulge structures include bulge loops that occur when the nucleosides that fail to base pair with the partner strand are only in one strand and interior loops that occur when opposing nucleosides in both strands cannot base pair.

“Carriers” are molecular entities distinguishable from the carried matter, said matter capable of altering the levels of particular molecules in cells. Carried matter includes without limitation, conventional siRNA, conventional miRNA, conventional ss-RNAi and conventional antisense oligonucleotides as well as seqRNAi guide and passenger strands and the ss-RNAi and ss-MiR of the present invention. Carriers can facilitate entry of the carried matter into cells, enhance associations with particular cells containing the molecular target to be manipulated. Carriers might be covalently attached to such carried matter or non-covalently associate with them. Carriers exclude physiological buffers such as phosphate buffered saline or matrices employed for extended release of the compounds disclosed herein.

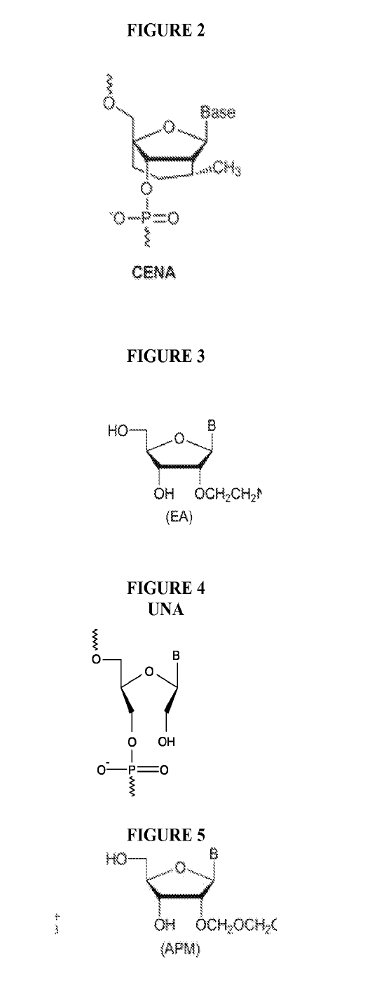

“CENA” is an acronym for 2′,4′-carbocyclic-ethylene-bridged or 2′,4′-carbocyclic-ENA-bridged locked nucleic acid. It is illustrated in FIG. 2 where “base” represents any of the bases provided for herein.

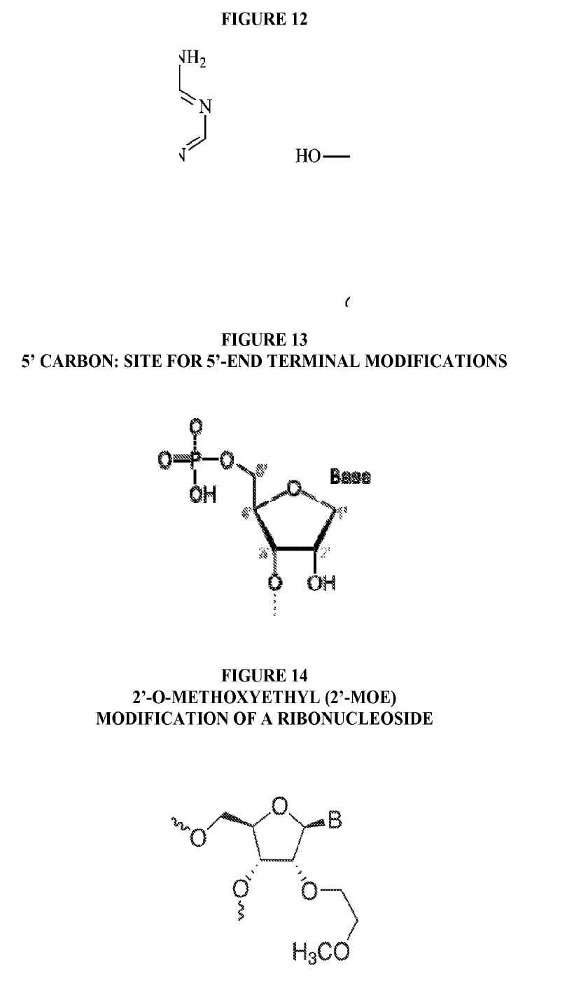

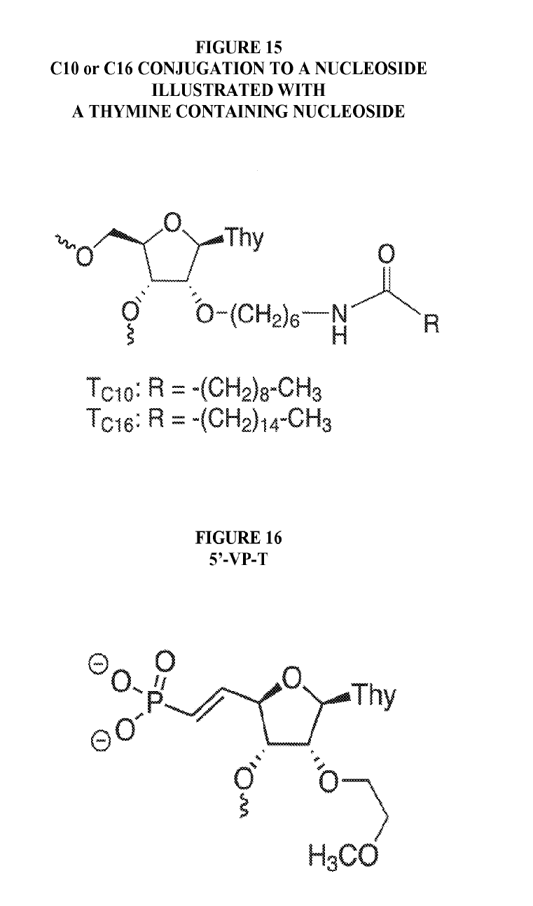

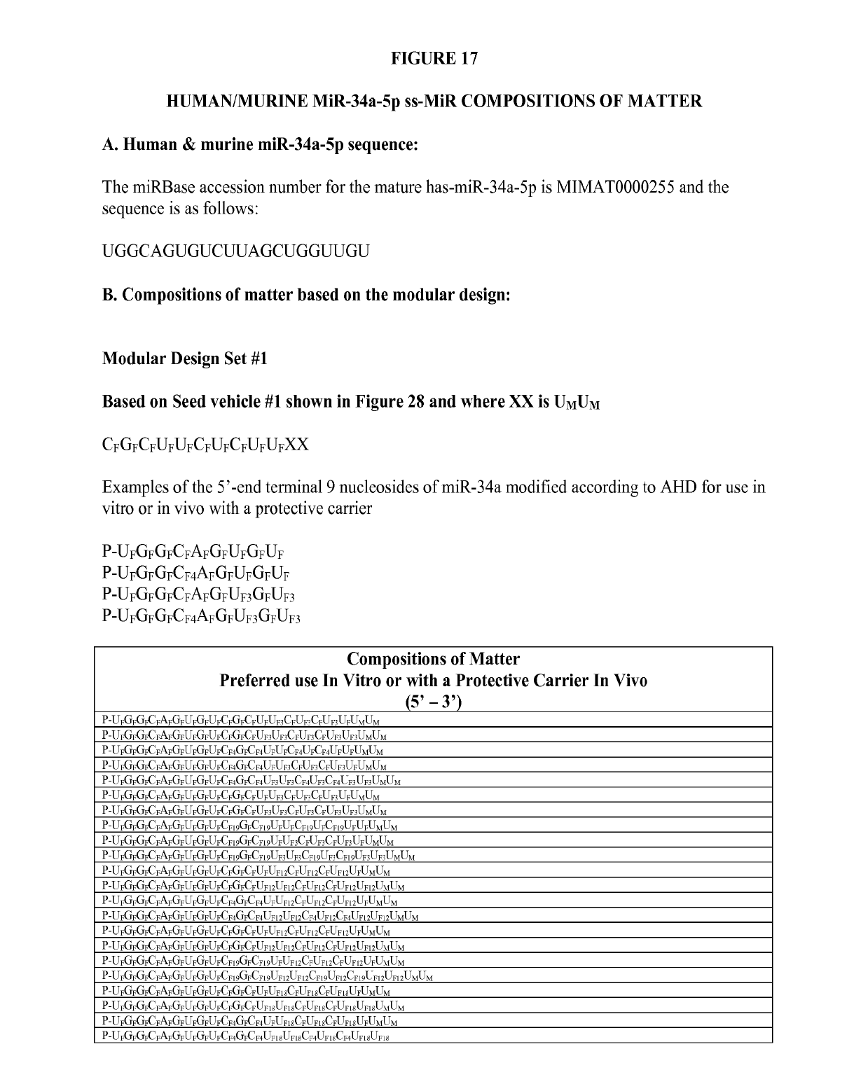

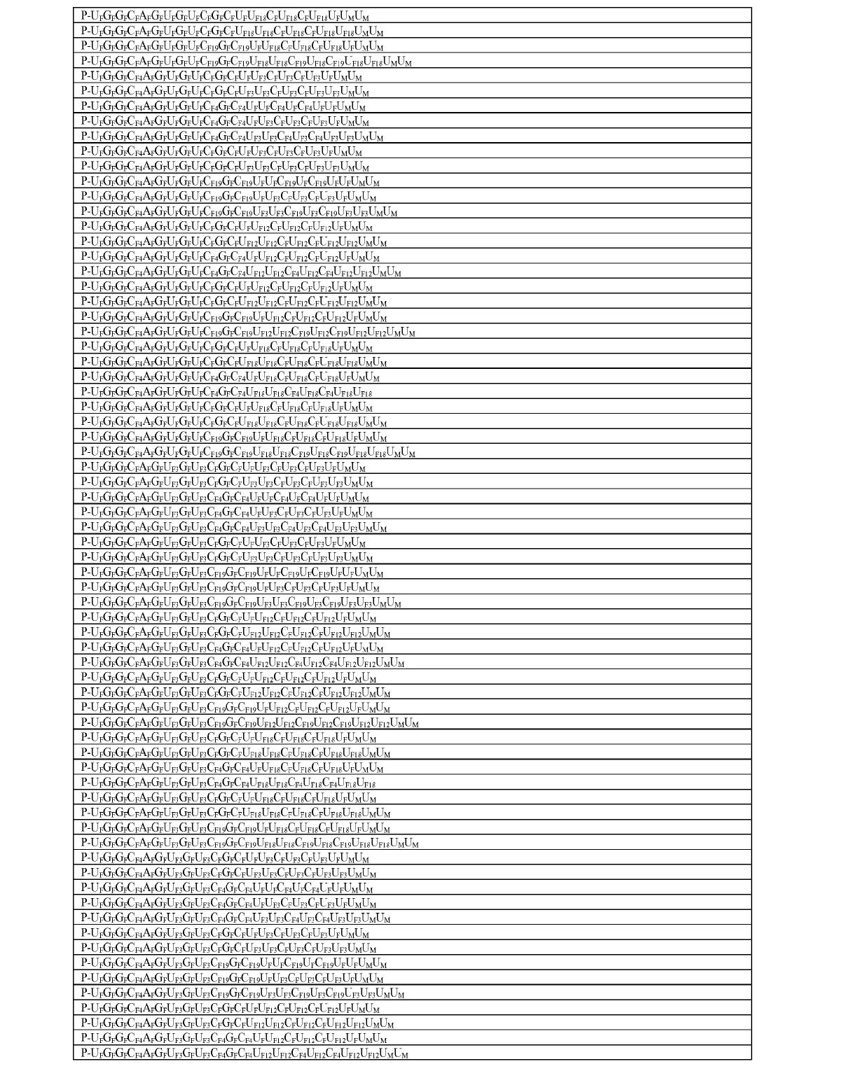

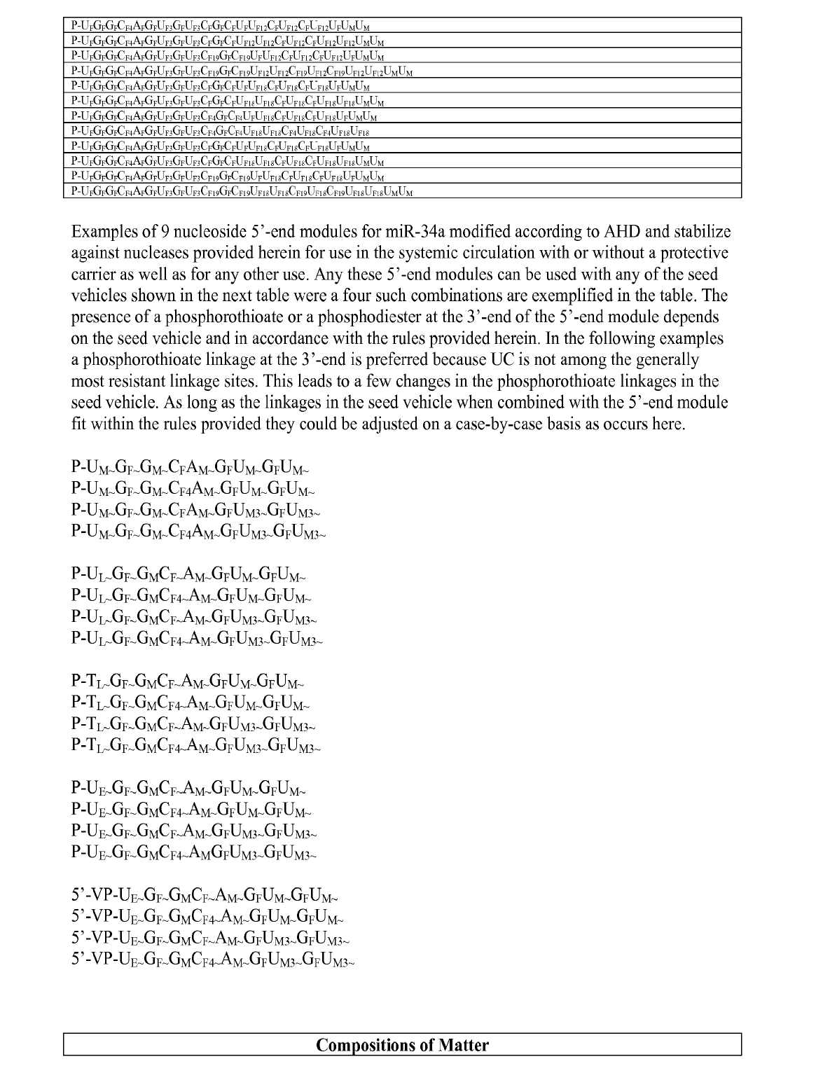

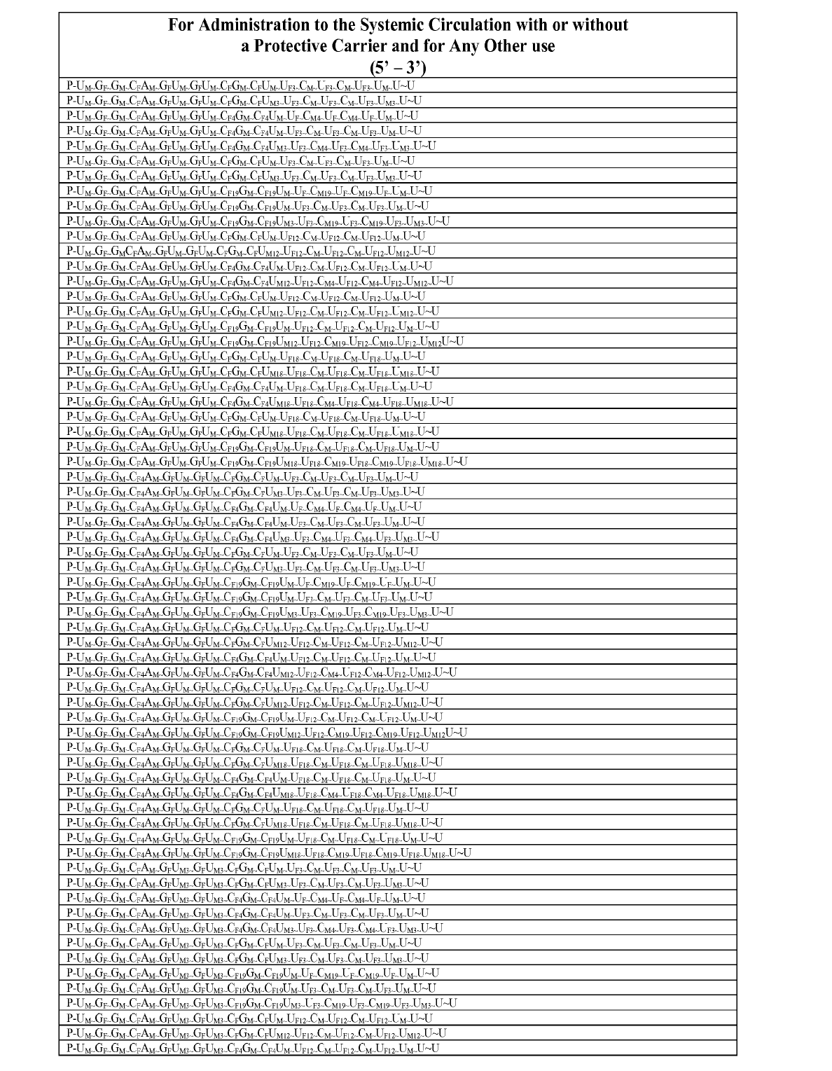

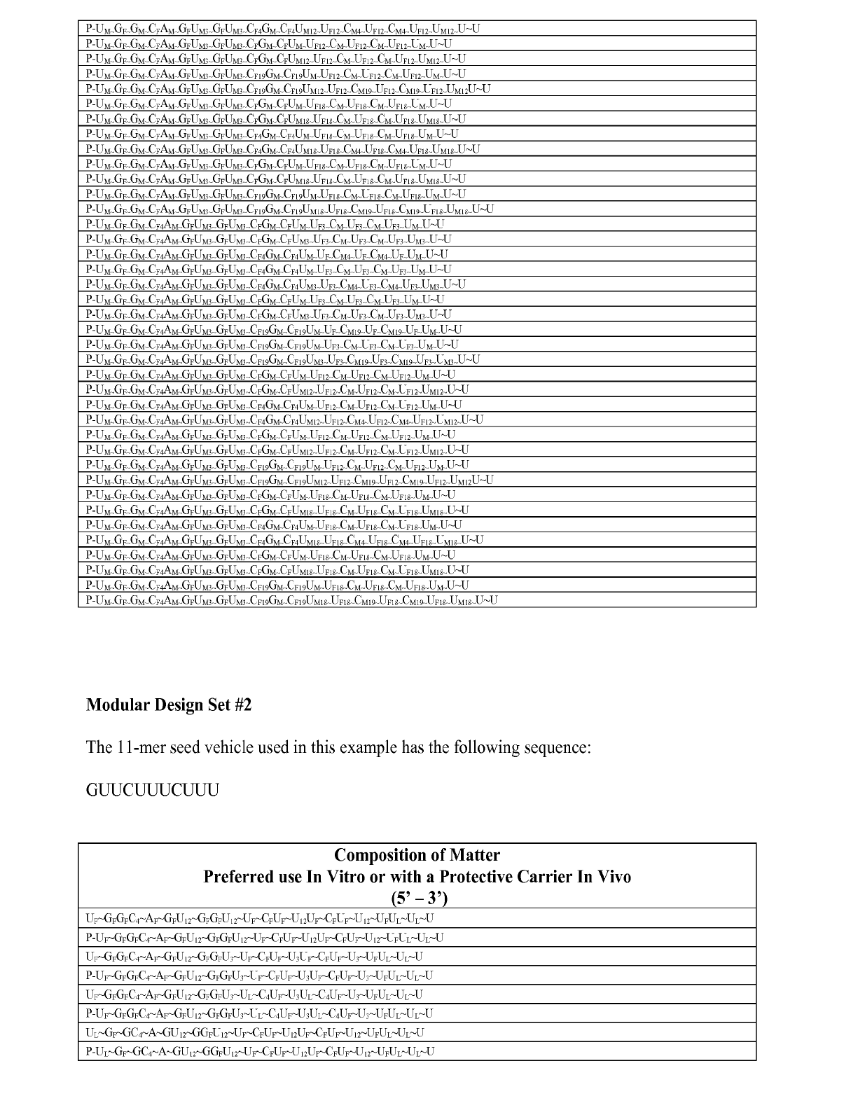

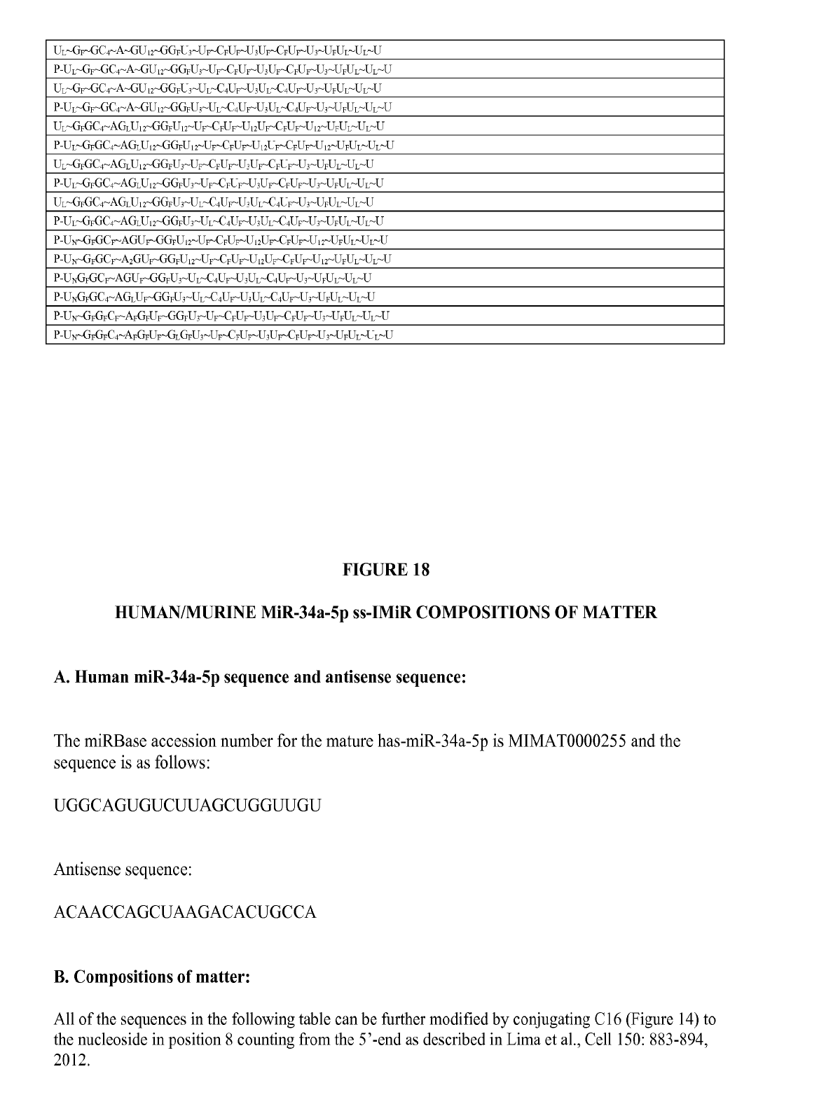

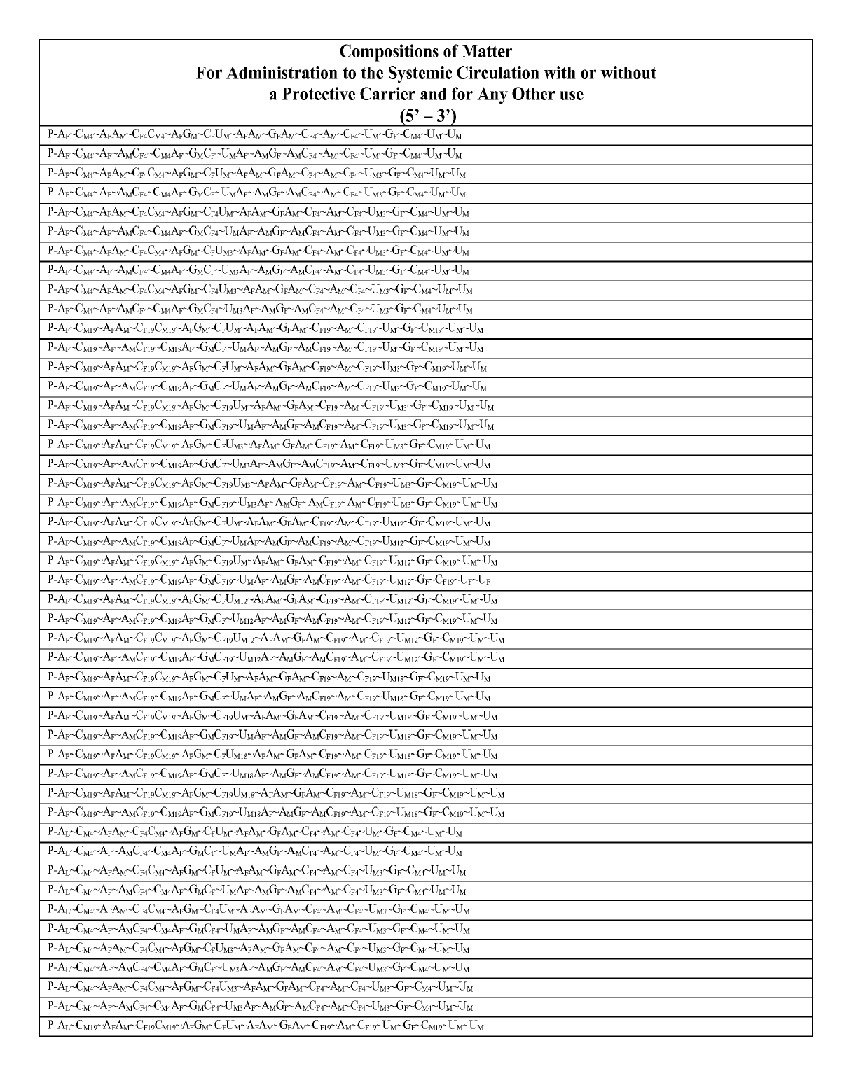

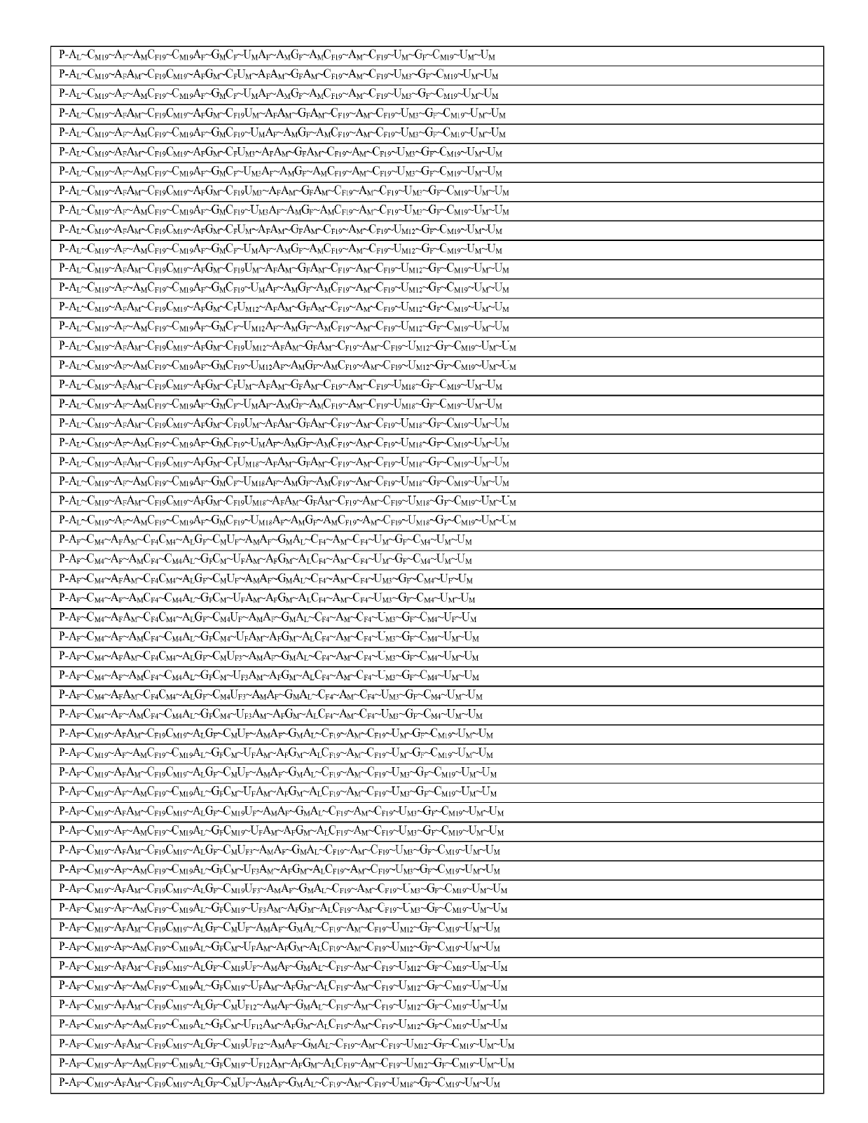

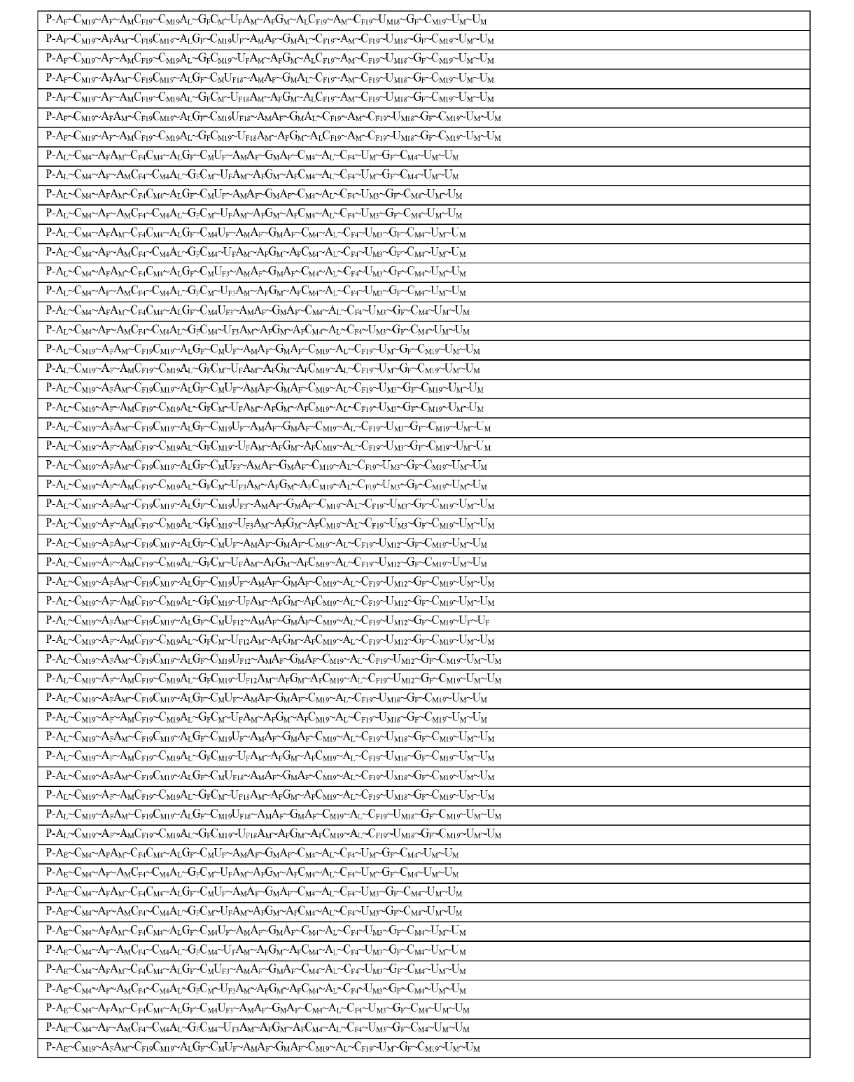

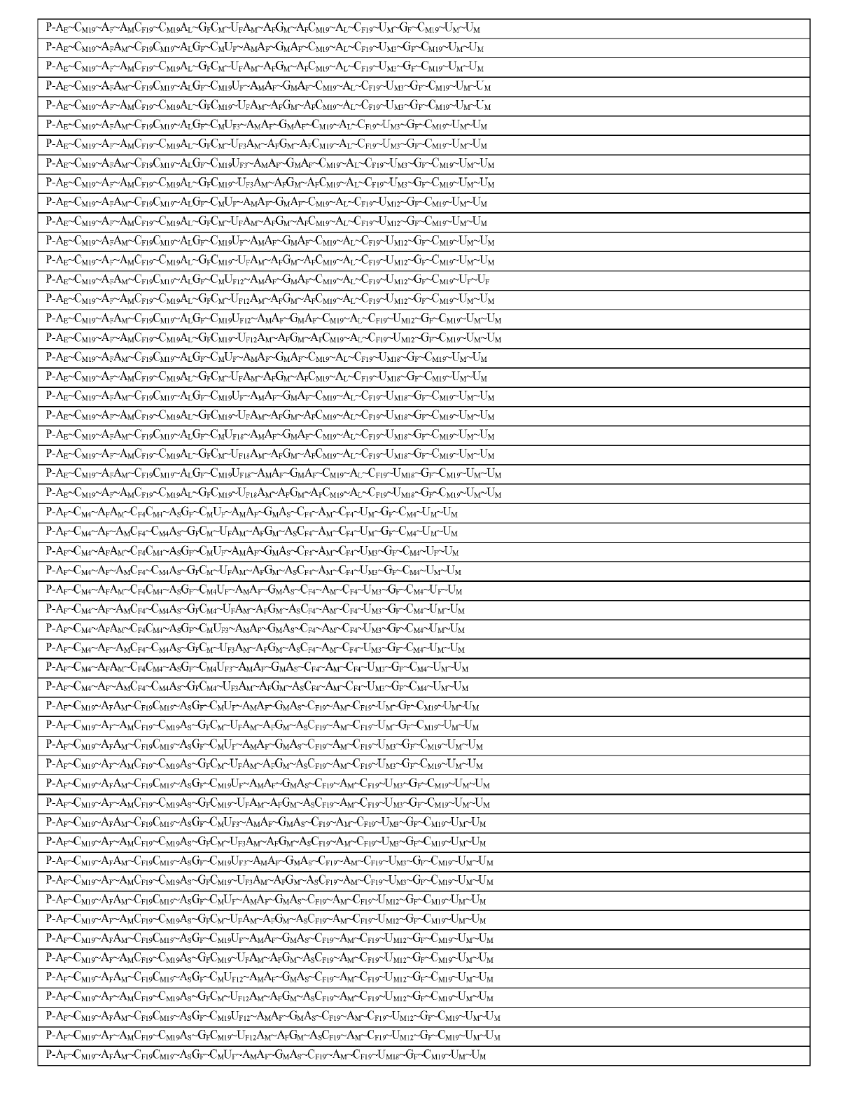

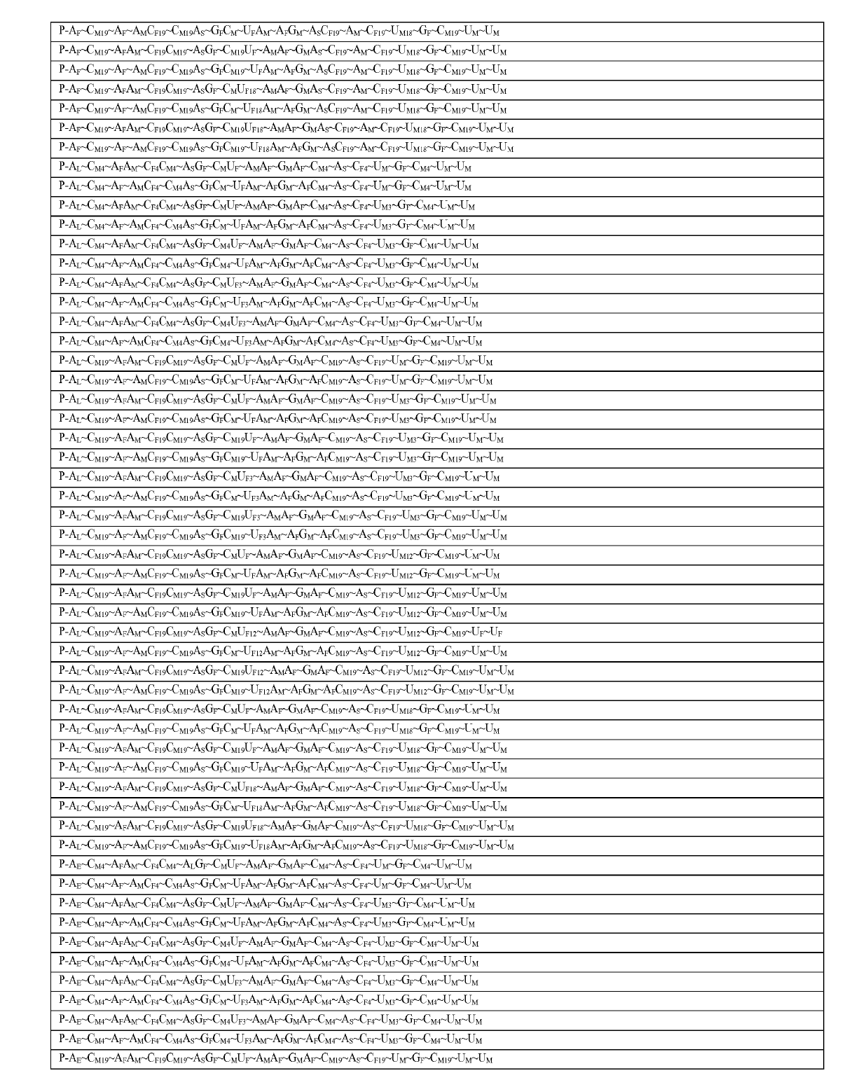

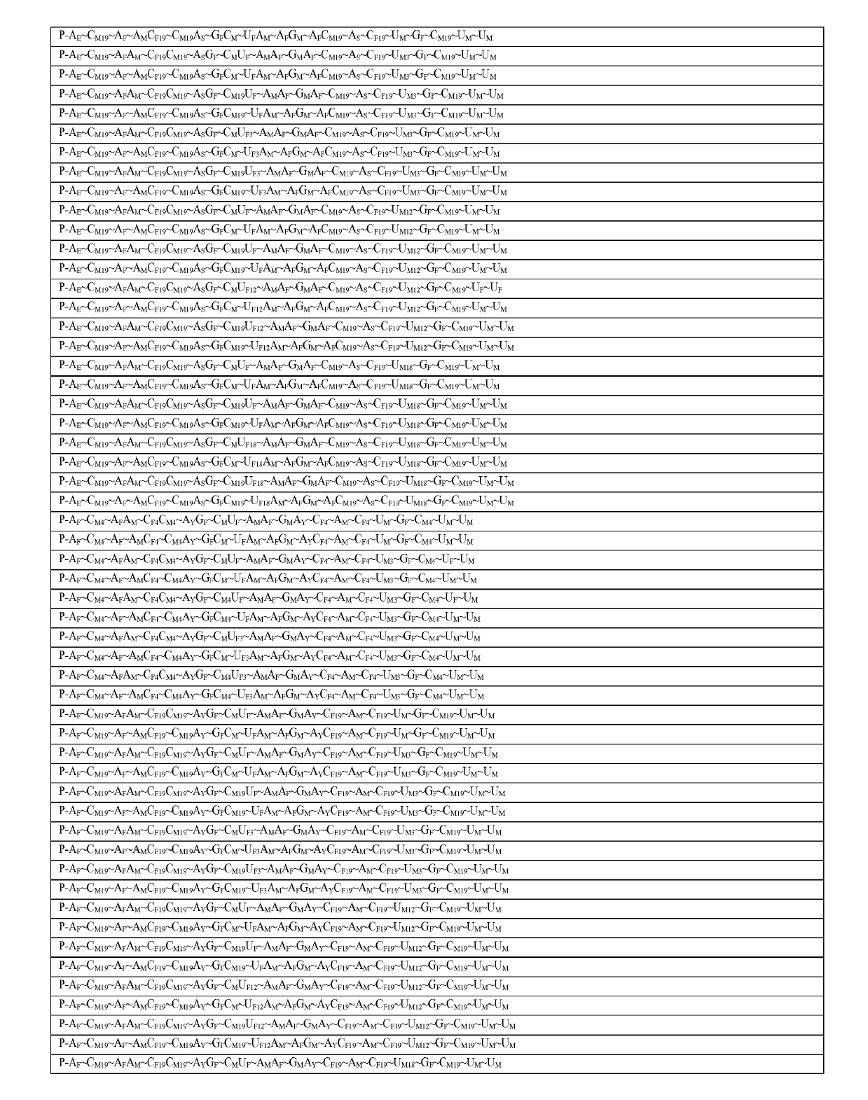

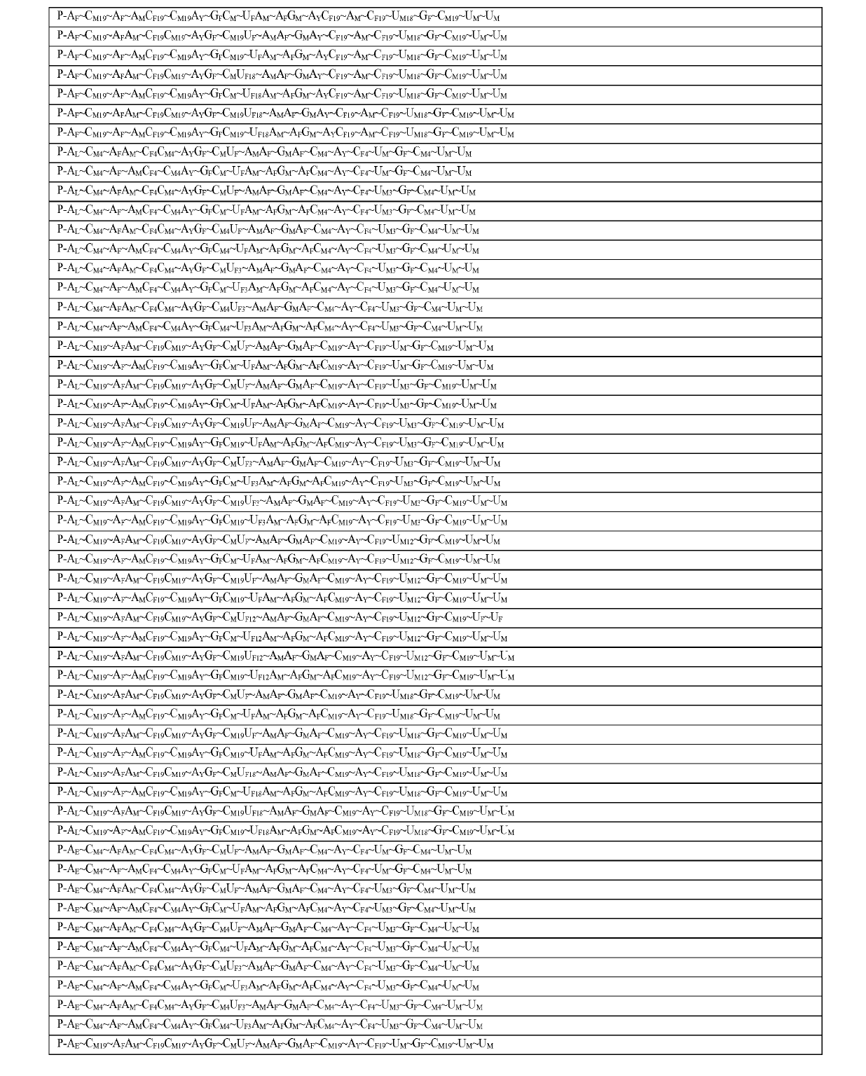

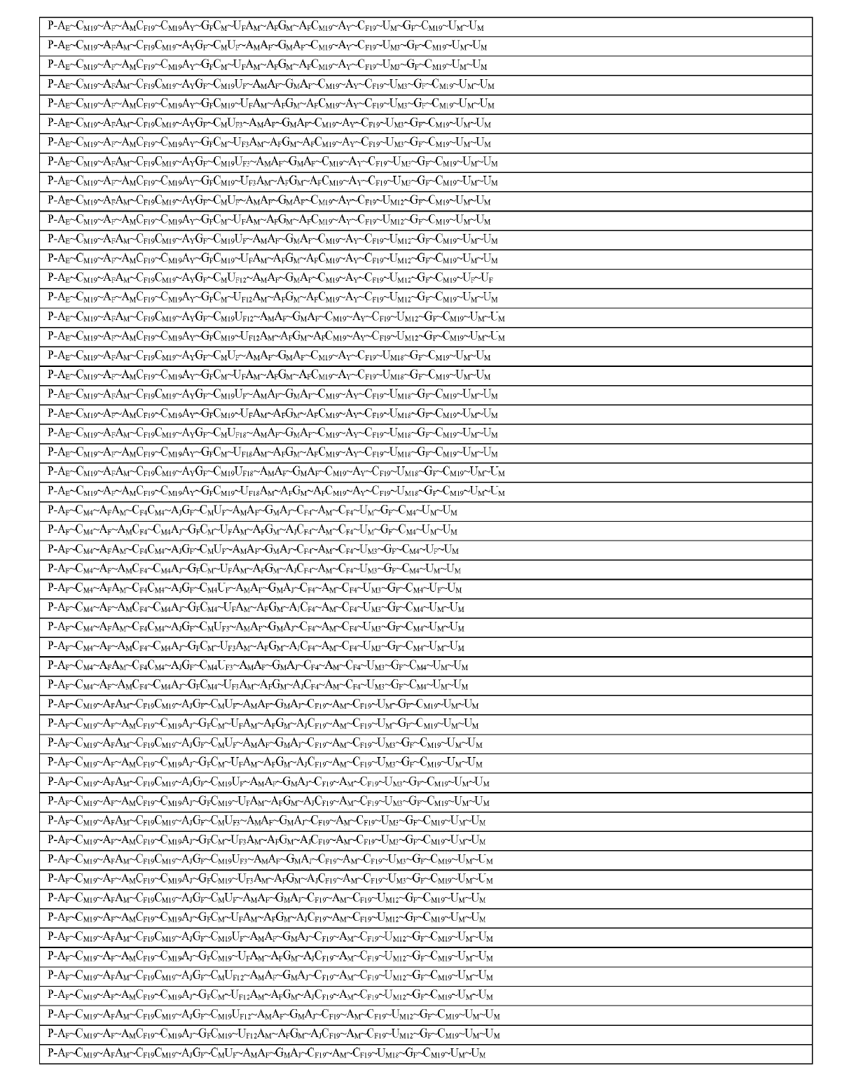

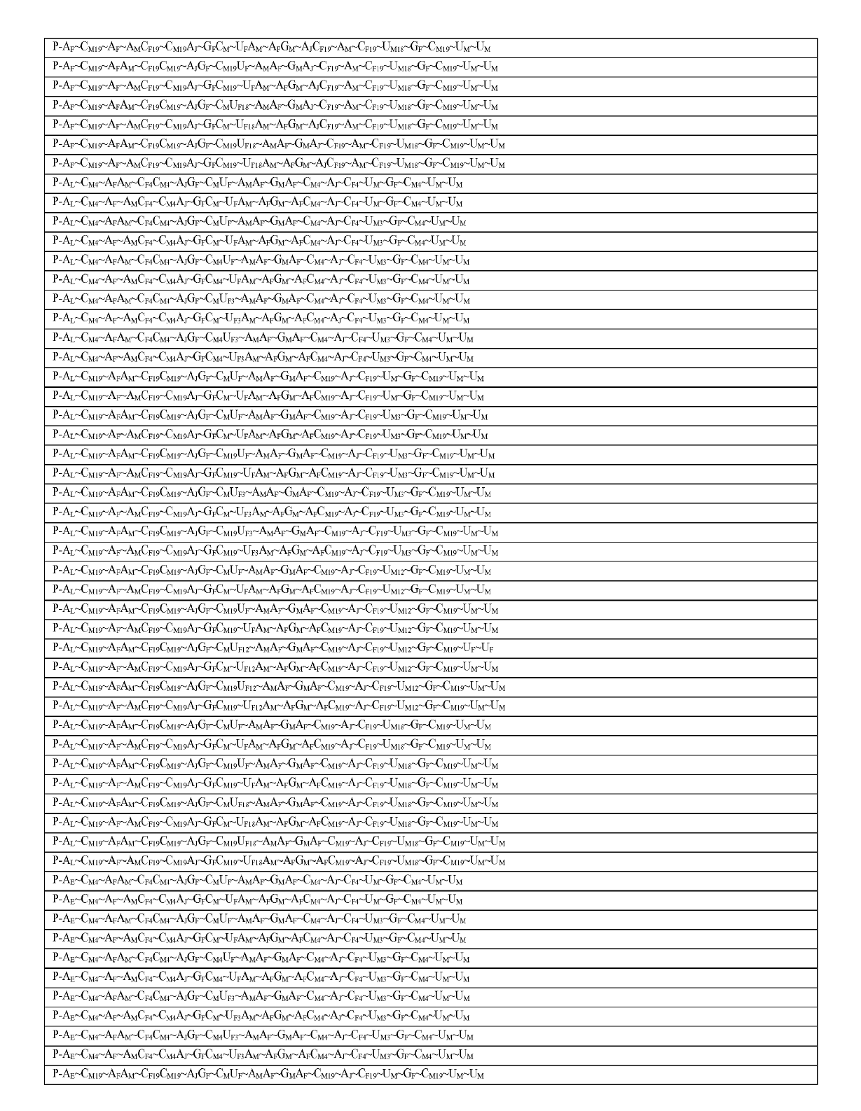

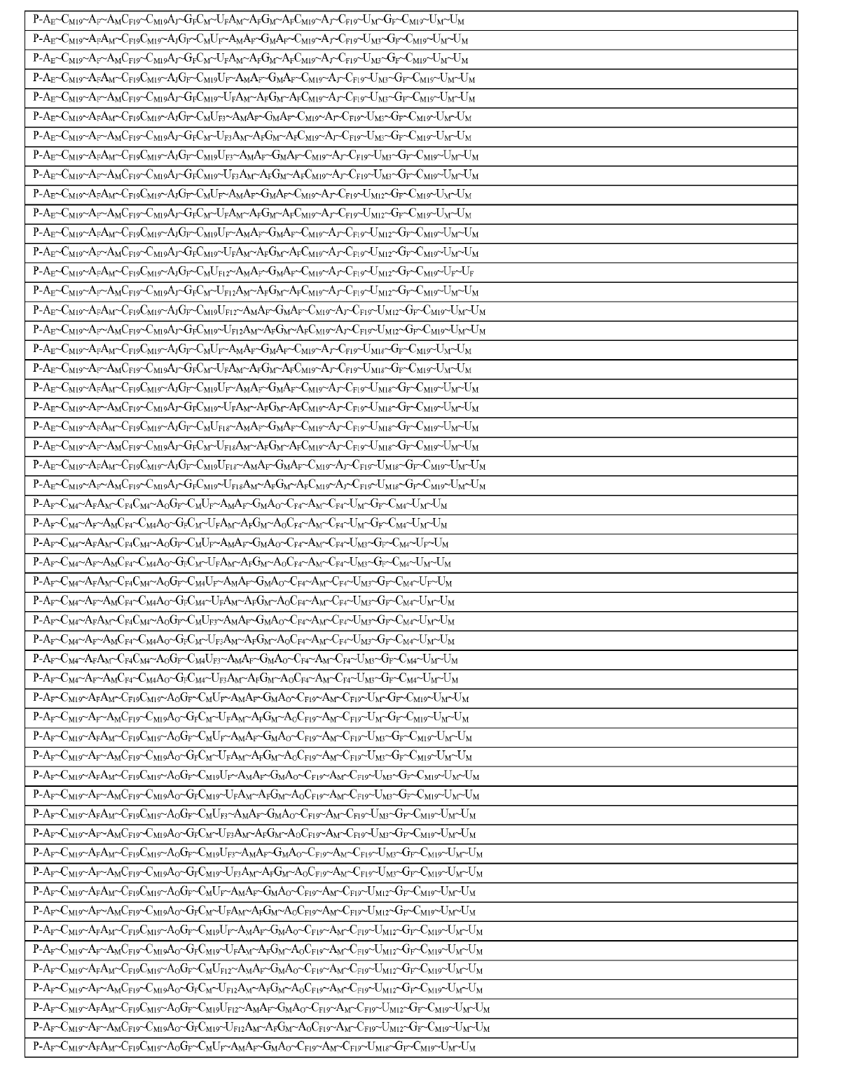

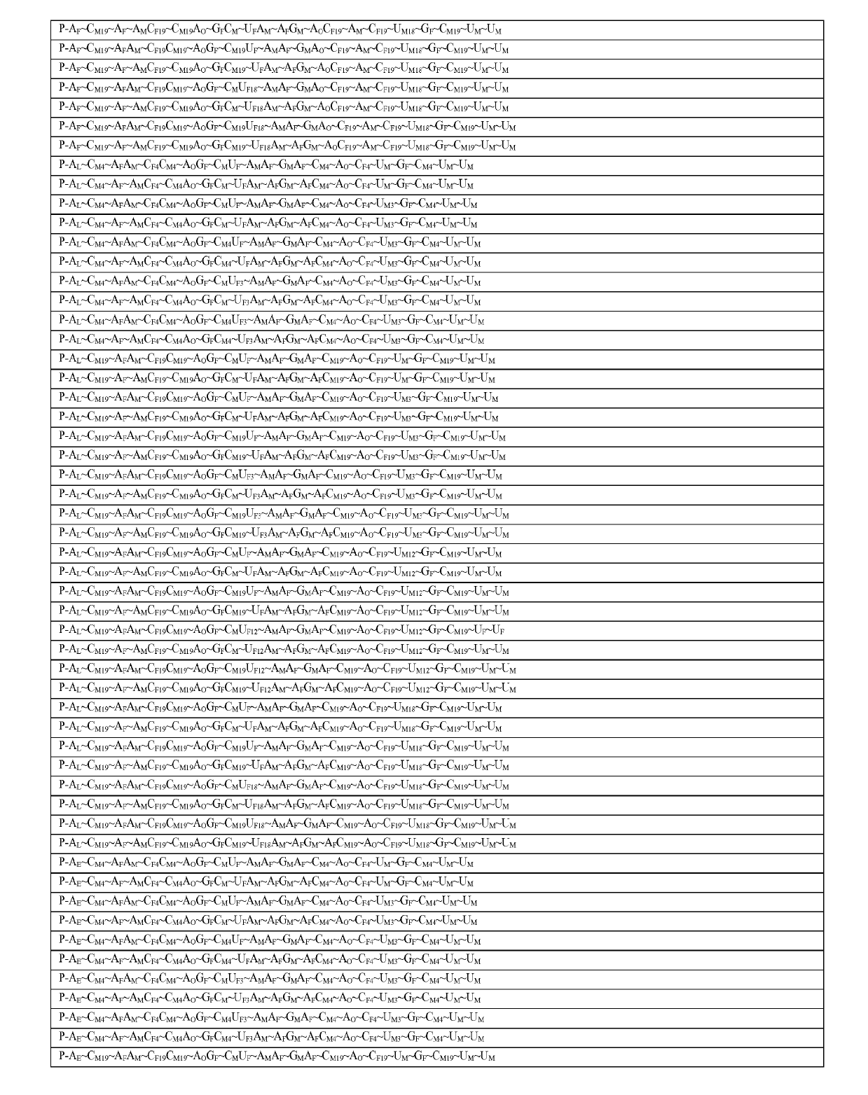

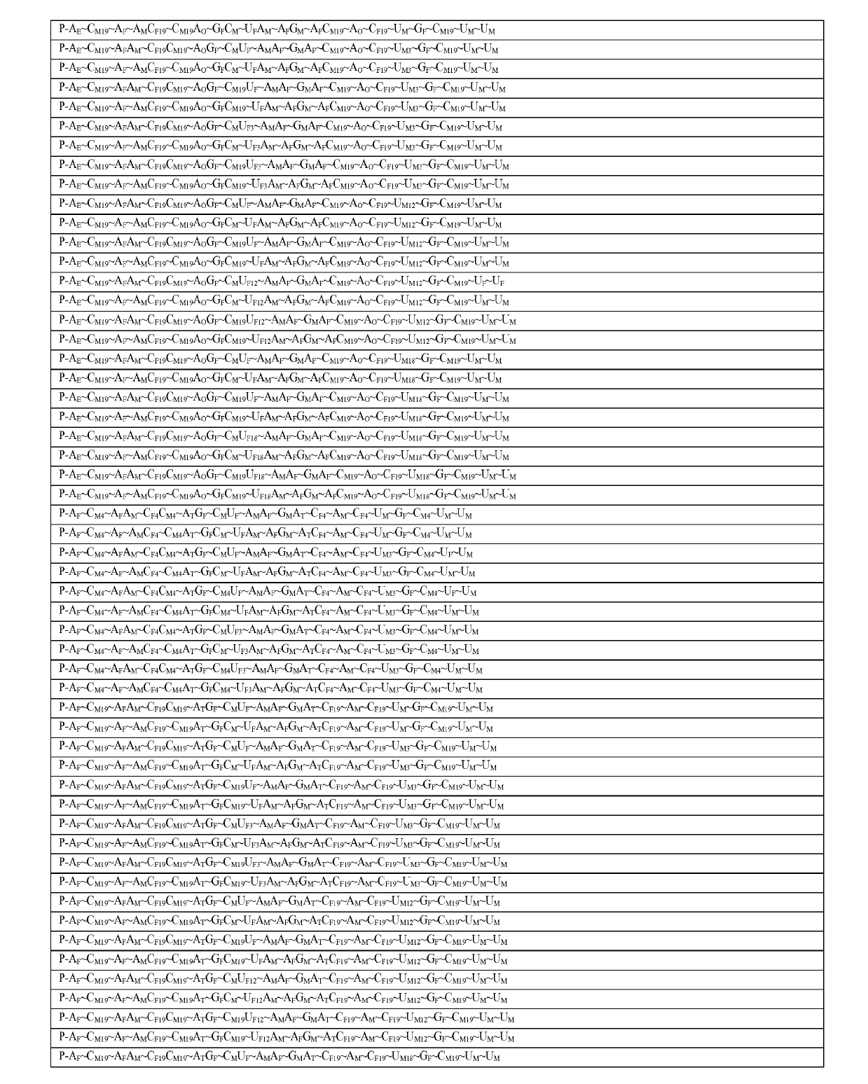

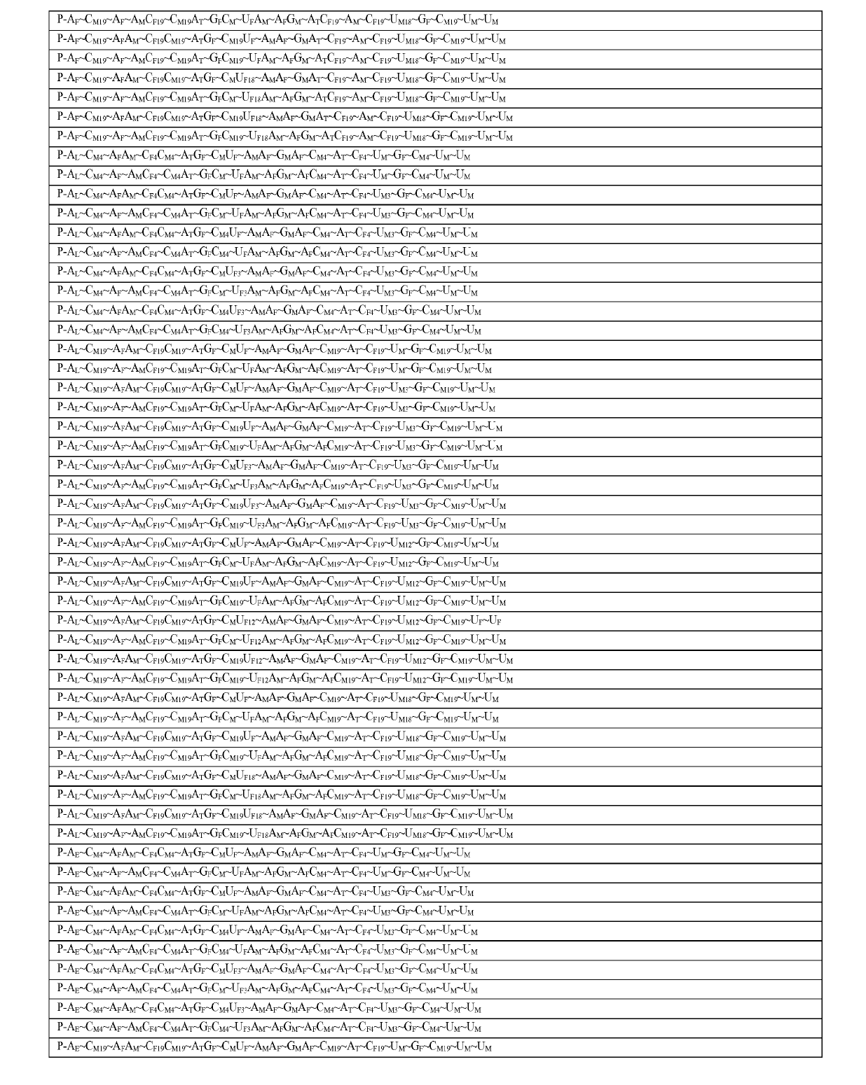

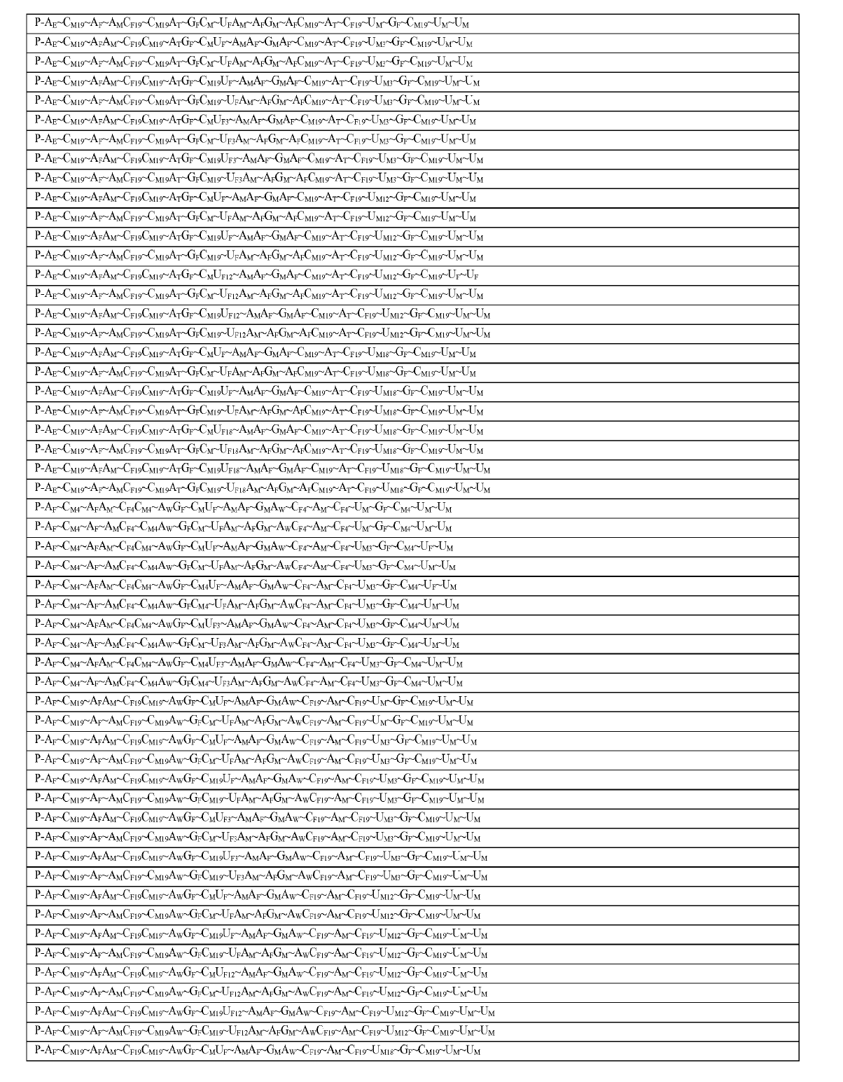

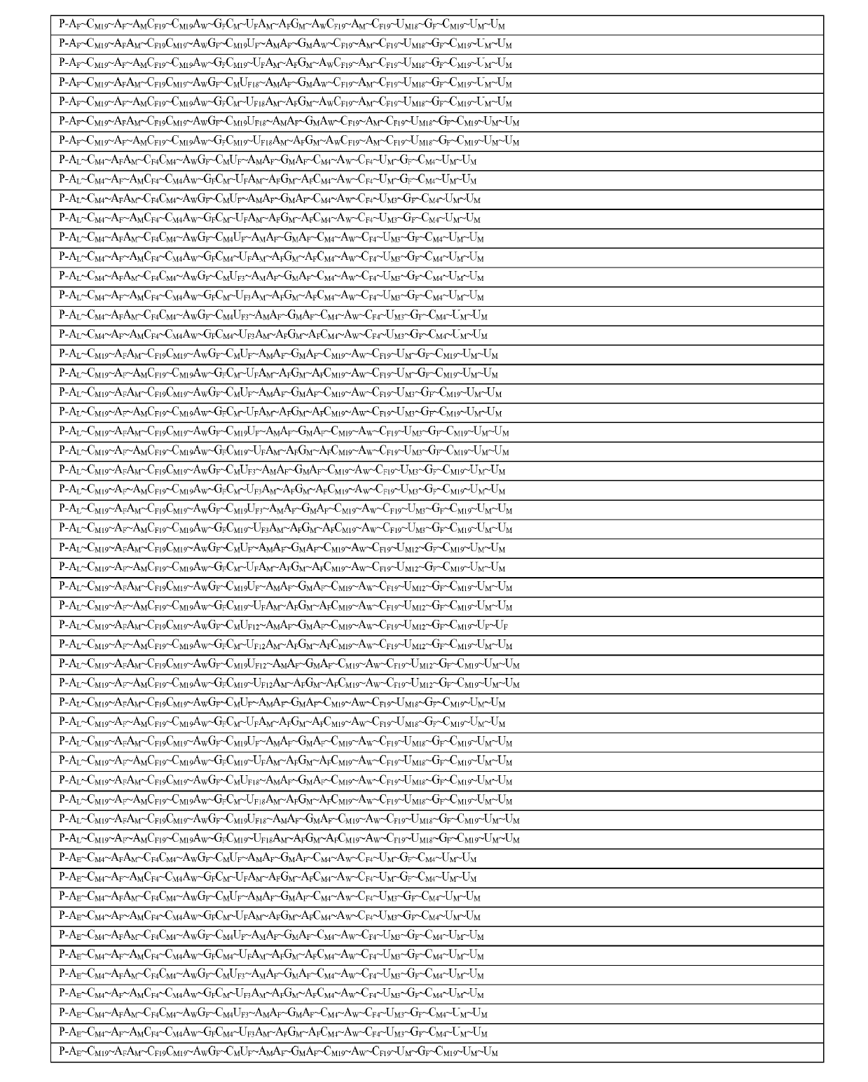

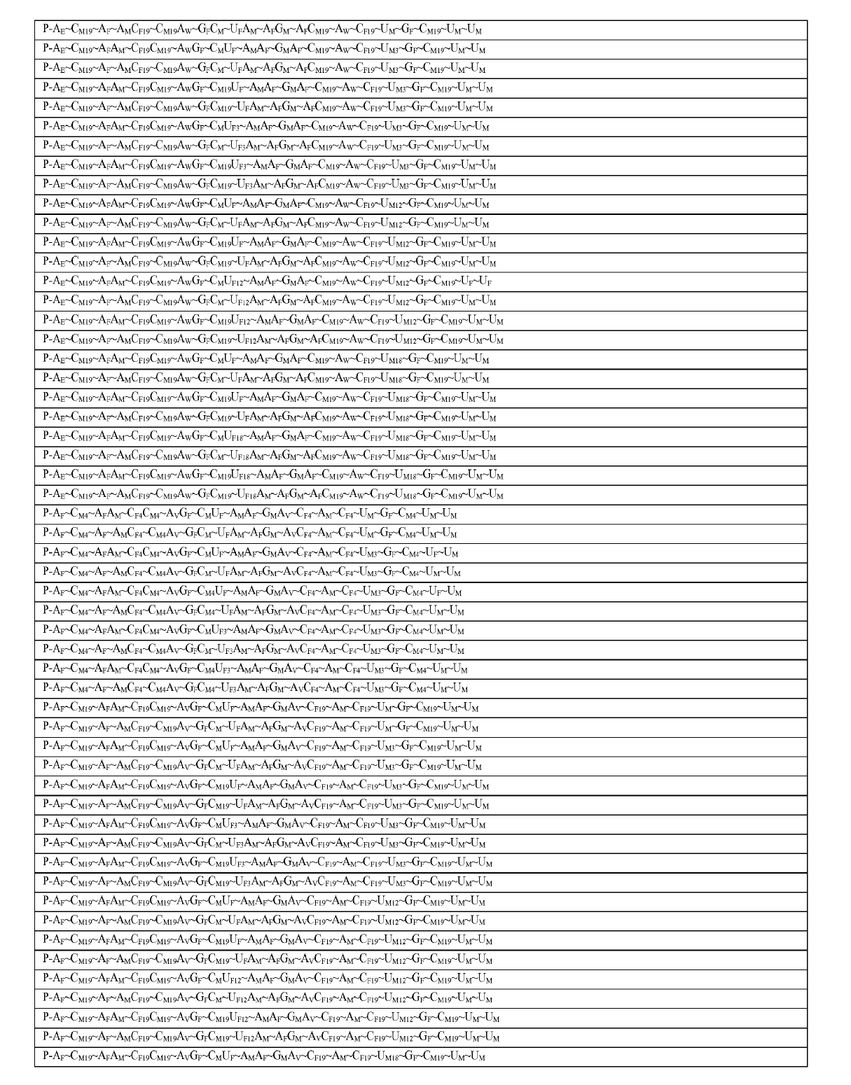

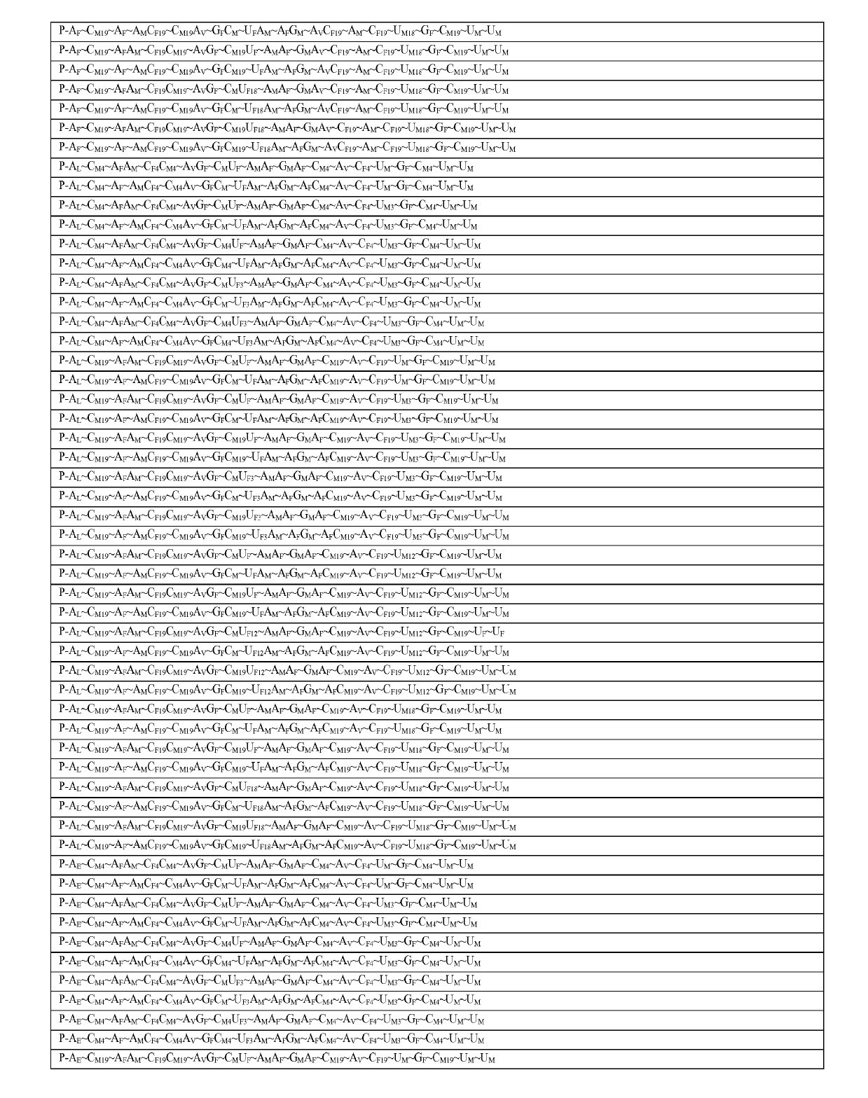

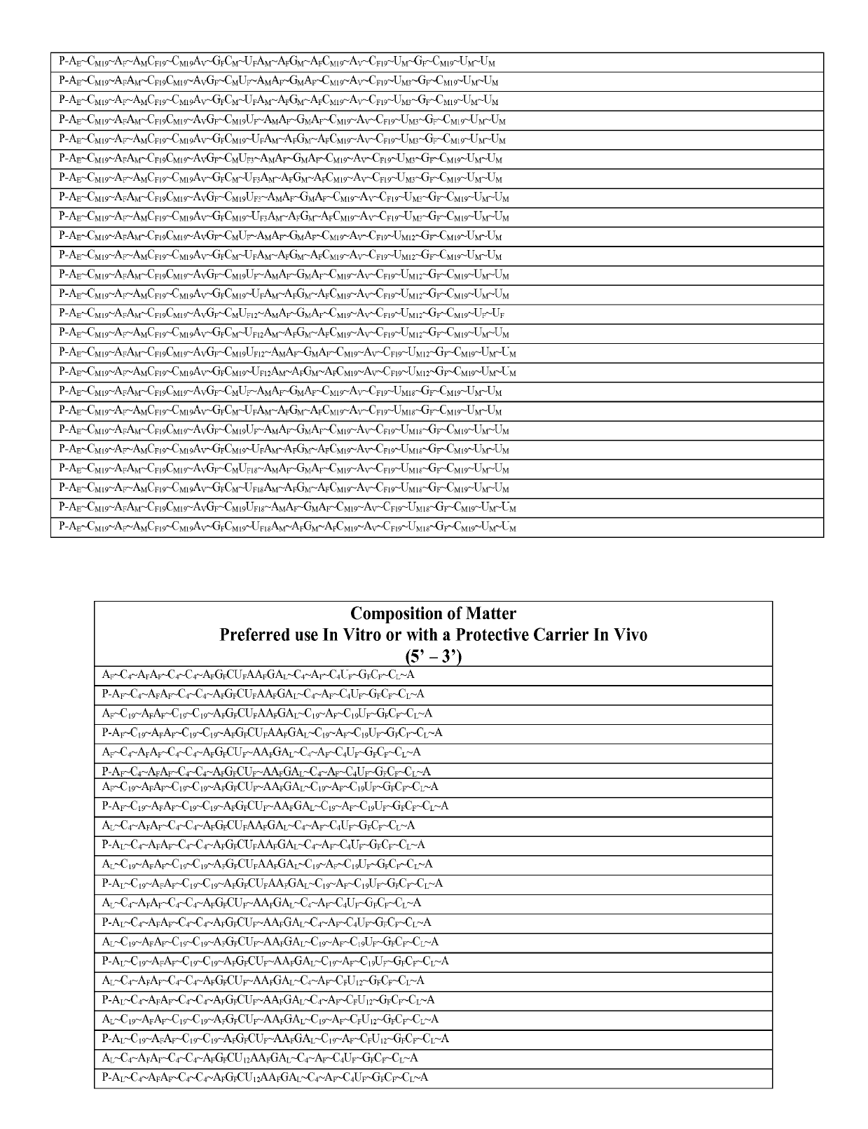

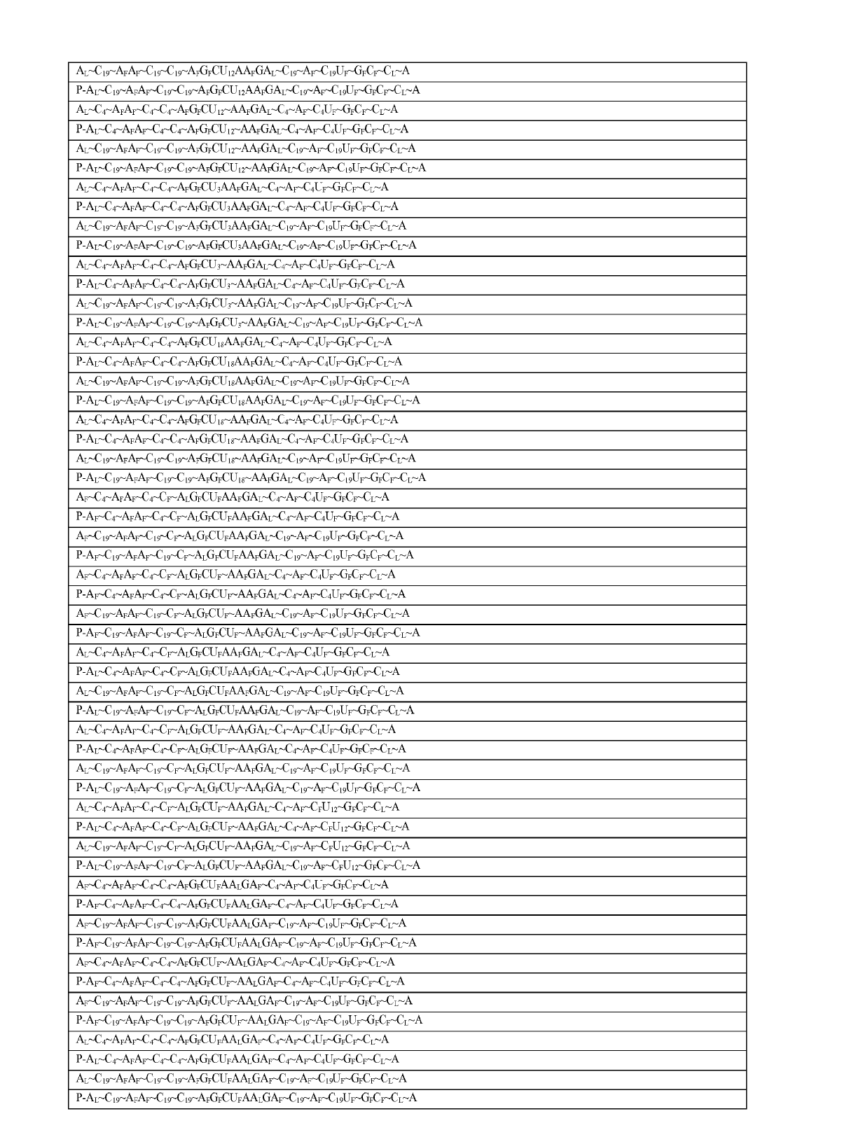

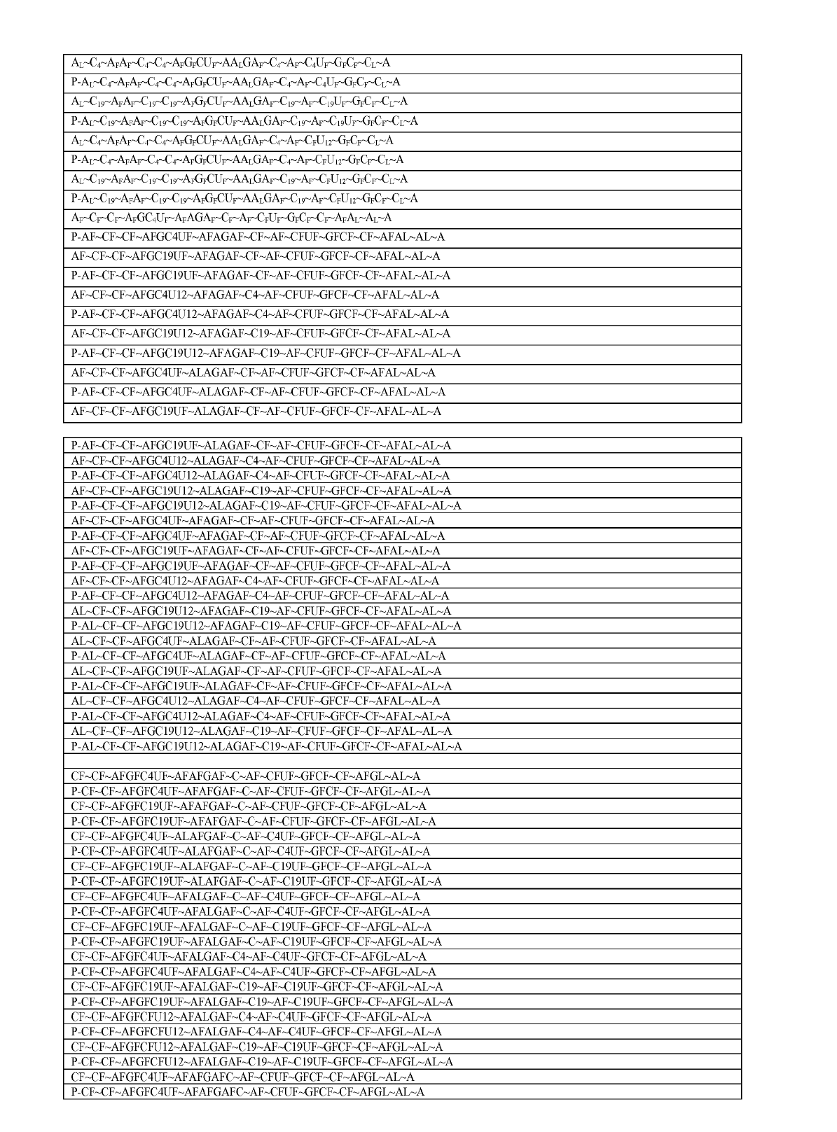

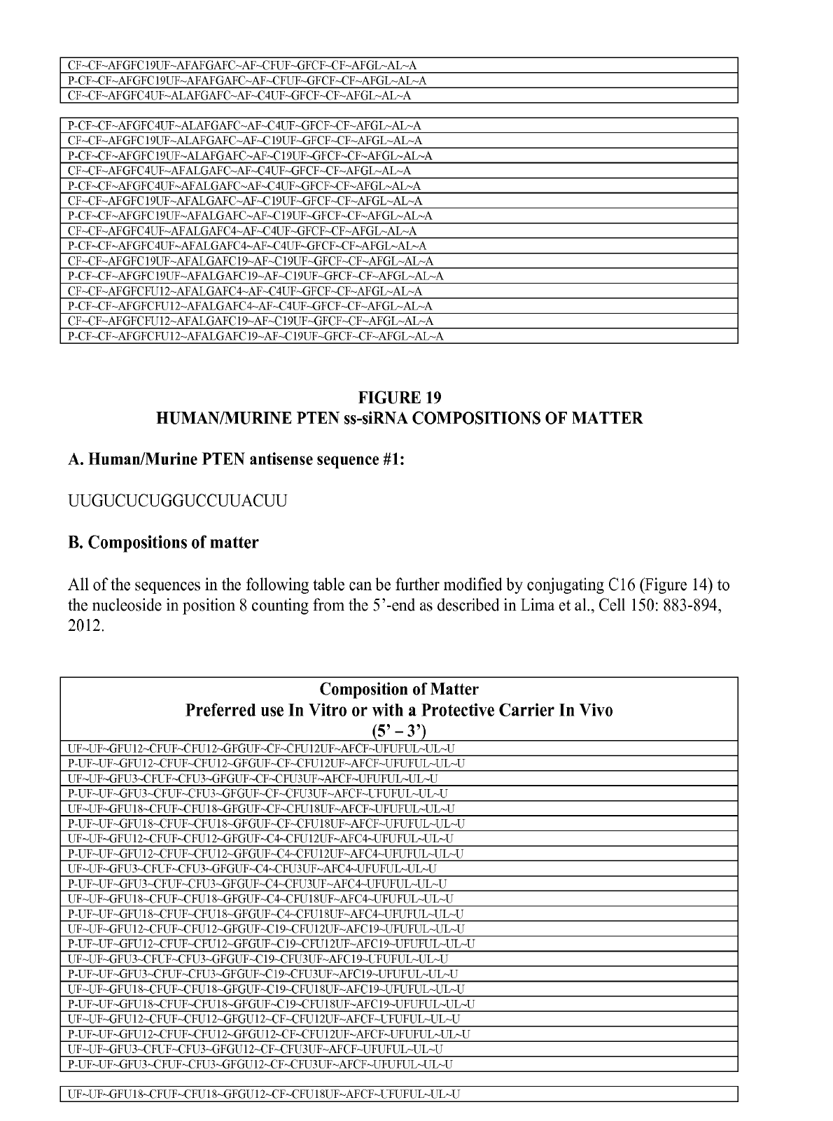

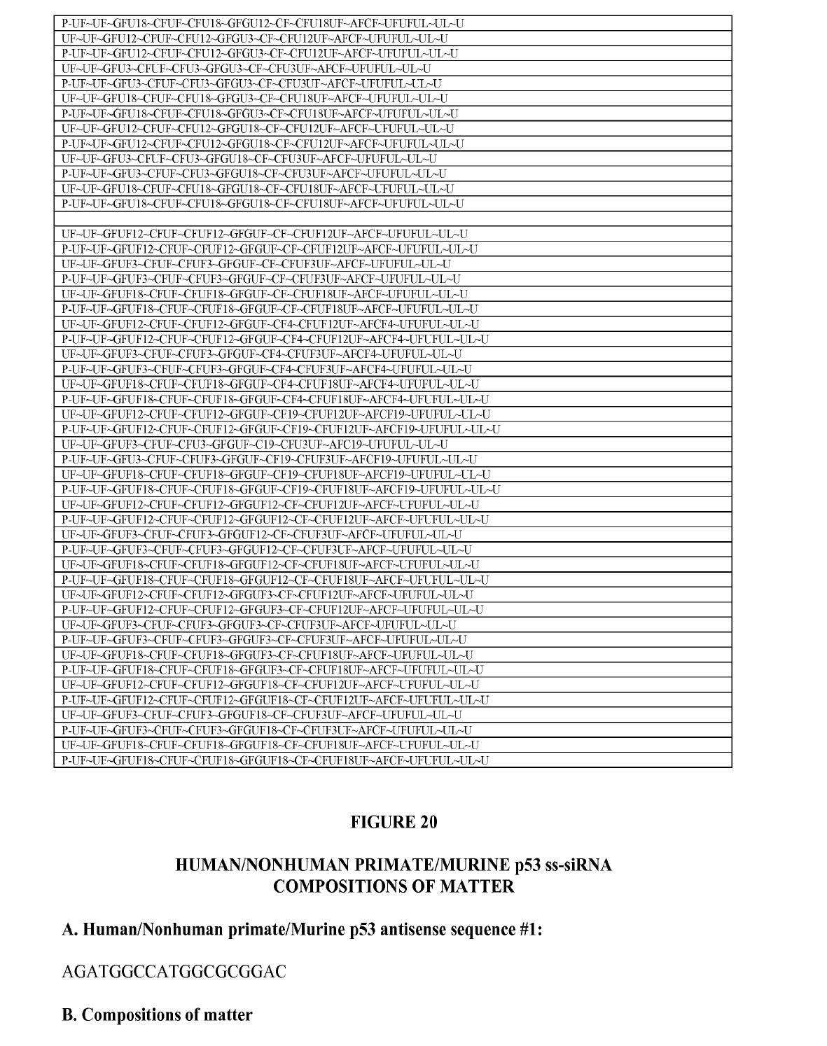

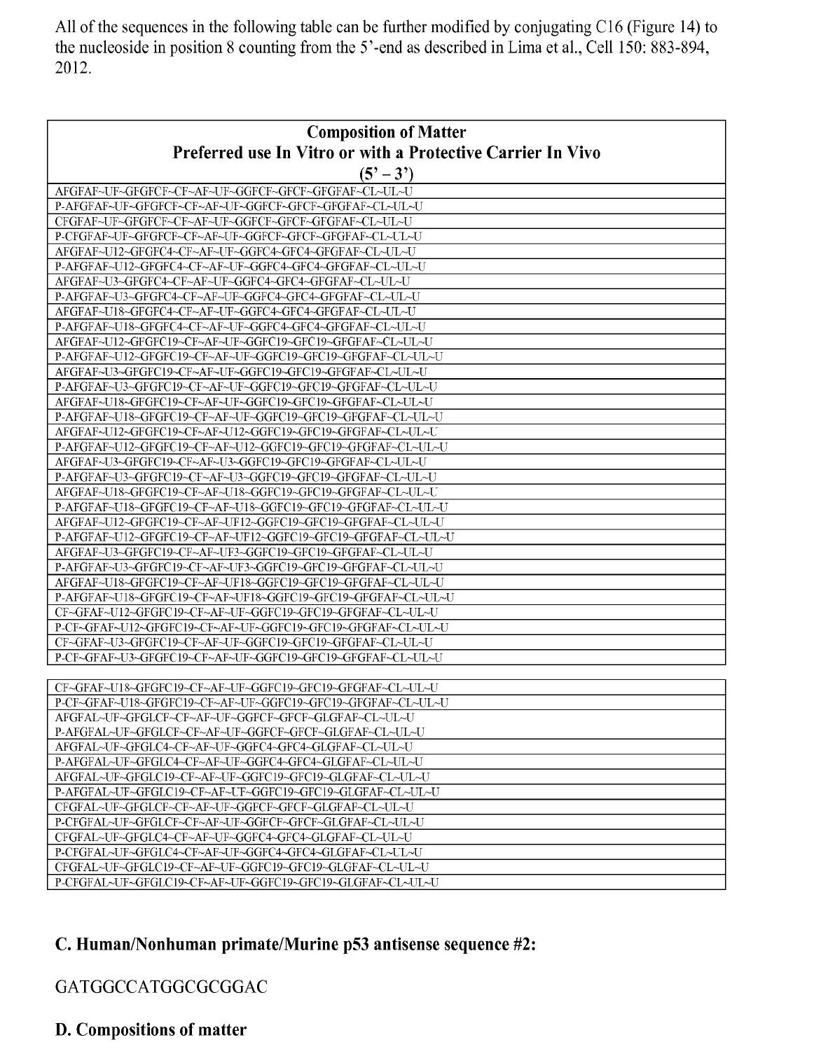

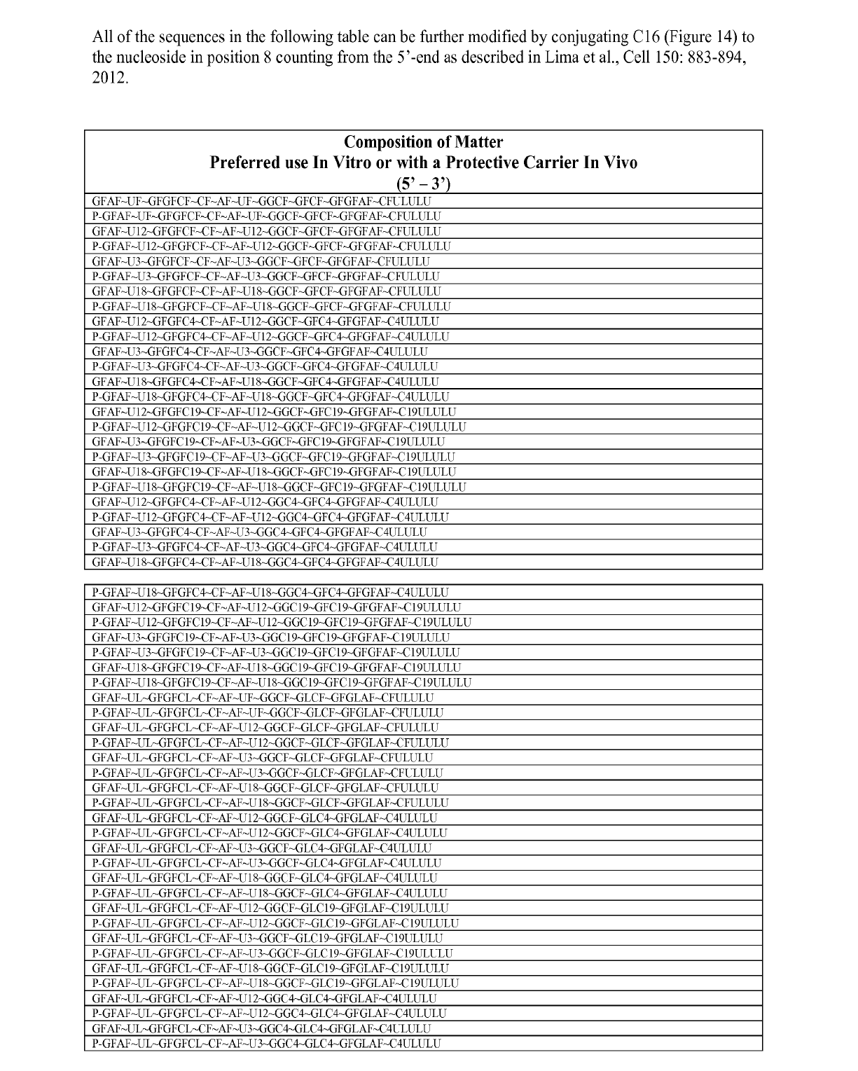

“CeNA” is an acronym for cyclohexenyl nucleic acid. It is illustrated in FIG. 12 where “base” represents any of the bases provided for herein. Further, CeNA nucleosides can be linked to adjacent nucleosides by any of the linkages provided for herein.

“Central region of the antisense stand” is defined as nucleosides 10 and 11 from the 5′end along with the adjacent two nucleosides on each side of these including all the intervening linkages, i.e., the regions comprised of the 8 contiguous nucleosides in positions 8-13 counting from the 5′-end. It is also referred to as the “targeting code” when it is a seqsiRNA or seqIMiR antisense strand.

“Chemically modified” or “chemical modification” is applied to oligos used as conventional antisense oligos, conventional siRNA, conventional miRNA or seqRNAi (seqsiRNA, seqMiRs, or seqIMiR) where the term refers to any chemical differences between what appears in such compounds and the corresponding standard natural components of native RNA and DNA (U, T, A, C and G bases, ribose or deoxyribose sugar and phosphodiester linkages). Chemical modifications such as 5′ modification and 3′ modification and other known modifications useful to inhibit nuclease activity in the serum are encompassed by this phrase. During manufacture chemical modifications of this type do not have to literally be made to native DNA or RNA components. Also included in this term are any nucleoside substitutes that can be used as units in overhang precursors.

“Chimeric oligonucleotides” are ones that contain ribonucleosides as well as 2′-deoxyribonucleosides.

“Compounds” refers to compositions of matter that include conventional siRNA, conventional miRNA, as well as the sense, antisense strands that make up particular seqRNAi sets in addition to the seqRNAi-based duplexes they can form by complementary base pairing with each other.

The phrase “consisting essentially of” when referring to a particular nucleotide or amino acid means a sequence having the properties of a given SEQ ID NO. For example, when used in reference to a ss-RNAi sequence, the phrase includes the sequence per se and those molecular modifications described herein which increase desirable biological properties, e.g., nuclease resistance, potency, tissue retention time, stability in circulation.

“Conventional antisense oligos” are single stranded oligos described in the prior art that inhibit the expression of the targeted gene by one of the following mechanisms: (1) Steric hindrance—e.g., the antisense oligo interferes with some step in the sequence of events involved in gene expression and/or production of the encoded protein by directly interfering with one of these steps. Such steps can include transcription of the gene, splicing of the pre-mRNA and translation of the mRNA; (2) Induction of enzymatic digestion of the RNA transcripts of the targeted gene by RNase H; (3) Induction of enzymatic digestion of the RNA transcripts of the targeted gene by RNase L; (4) Induction of enzymatic digestion of the RNA transcripts of the targeted gene by RNase P: (5) Induction of enzymatic digestion of the RNA transcripts of the targeted gene by double stranded RNase; and (6) Combined steric hindrance and induction of enzymatic digestion activity in the same antisense oligo. Conventional antisense oligos do not have an RNAi mechanism of action. The latter can be distinguished in several ways including the requirement for a recognized RNAi protein that combines with an RNAi antisense strand such that the antisense strand directs the RNAi protein to the intended target(s) and where said protein is required for silencing of said target(s). Such RNAi proteins include but are not limited to AGO-1, 2, 3 and 4.

“Conventional miRNA” are those compounds described in the prior art and administered to cells in vitro or in vivo as an oligo duplex and the term excludes those unusual cases where it is delivered as single stranded miRNA (ss-miRNA)—i.e., where the antisense stand is administered without a sense strand and produces a substantial RNAi silencing effect. Administration of conventional miRNA nearly always requires the use of a carrier (in vitro or in vivo) or other means such as hydrodynamic injection (in vivo) to get the compound into cells in an active form.

“Conventional siRNA” are those compounds described in the art and administered to cells in vitro or in vivo as an oligo duplex and the term excludes those unusual cases where it is delivered as single stranded siRNA (ss-siRNA)—i.e., where the antisense stand is administered without a sense strand and produces a substantial RNAi silencing effect. Administration of conventional siRNA nearly always requires the use of a carrier (in vitro or in vivo) or other means such as hydrodynamic injection (in vivo) to get the compound into cells in an active form.

“Conventional ss-RNAi” are those single strand RNAi compounds described in the art.

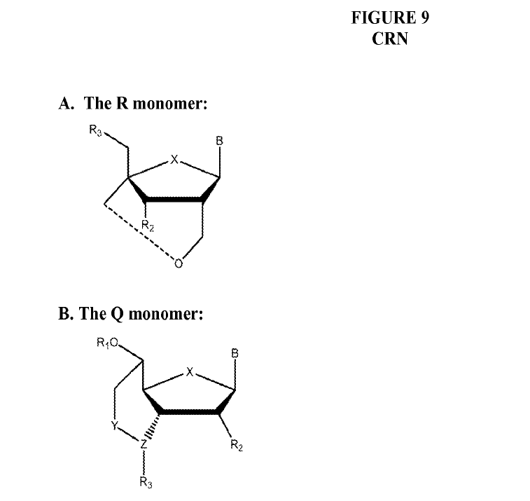

“CRN” is an acronym for conformationally restricted nucleoside or nucleomonomer as described in WO 2011/139710 except the linkages used to connect CRNs to other nucleosides are not necessarily phosphodiester. CRN nucleosides come in two basic forms illustrated in FIG. 9. In the R monomer, X can be independently selected for each occurrence from the group consisting of O, S, CH₂, C═O, C═S, C═CH₂, CHF or CF₂; R₂ and R₃ are the linkages and B is a nucleobase or nucleobase analog where the linkages, nucleobases or nucleobase analogs are independently selected from the group consisting of those provided for herein. In the Q monomer X and Y can be independently selected for each occurrence from O, S, CH₂, C′O, C═S, C═CH₂, CHF or CF₂; R₁ and R₃ are the linkages, R₂ is independently selected for each occurrence from the group consisting of H, F, OH, OCH₃, OCH₃OCH₃, OCH₂CH₃OCH₃, CH₂CH₃OCH₃, CH(OCH₃)CH₃, allyl; Z is independently selected for each occurrence from the group consisting of N or CH; and B is a nucleobase or nucleobase analog where the linkages, nucleobases or nucleobase analogs are independently selected from the group consisting of those provided for herein. When the Q monomer is used in the strands of the present invention R₂ is selected from the group consisting of H, F, OH, OCH₃ and OCH₃OCH₃ unless otherwise specified.

“Duplex vehicle” is used to describe a duplex comprised of a sense and an antisense strand into which particular seed sequences and their sense strand complement can be inserted as a starting point for the design of seqMiR compounds. These seed sequences can come from endogenous miRNA or be novel. Duplex vehicles are designed and/or selected to minimize off target effects and to promote efficient RISC loading and retention of the intended antisense strand.

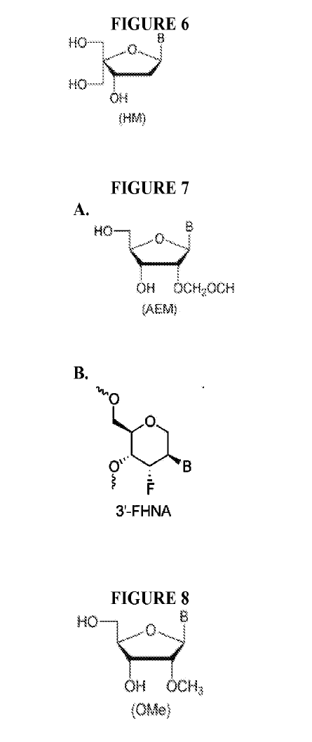

“EA” is an abbreviation for 2′-aminoethyl nucleoside. It is illustrated in FIG. 6 where B represents any of the bases provided for herein.

“Exosomes” are endosome-derived vesicles that transport molecular species such as miRNA and siRNA from one cell to another. They have a particular composition that reflects the cells of origin and typically this directs the payload to particular cells. Once these secondary cells take up the siRNA or miRNA they exert their RNAi functions.

“FANA” refers to a nucleoside modification where the fluorine has the opposite stereochemical orientation as the hydroxyl in ribose. It can also be referred to as 2′-deoxy-2′fluoro-arabinonucleic acid.

“F—CeNA” is an acronym for fluoro cyclohexenyl nucleic acid. The basic CeNA structure is illustrated in FIG. 12 where “base” represents any of the bases provided for herein. In the case of F—CeNA the fluorine appears in the 2′ position while the base is in the 1′ position. F—CeNA nucleosides can be linked to adjacent nucleosides by any of the linkages provided for herein.

“FHNA” is an abbreviation for 3′fluoro hexitol nucleic acid. The basic HNA nucleoside structure is shown in FIG. 7 where B is one of the bases provided for herein.

“Flexible sugar” is used as is or with modifiers that indicate degrees of flexibility in terms of the ease with which a sugar or sugar analog in a nucleoside changes in its conformation (pucker) in response to outside influences. Sugar and sugar analogs classified as most flexible include ribose, 2′-fluoro and 2′-0-methyl; those classified as flexible include HNA, FHNA, ANA, CeNA and F—CeNA; those classified as semi-flexible include CRN and CENA and one classified as rigid is LNA.

“General circulation” or “Systemic circulation” refers to the flow of blood that passes though the blood vessels that can be used to supply drugs, including ss-RNAi, to those areas of the body that are not protected by barriers that limit the passages of such drugs such as the central nervous system, testicles, placenta, fetus and the interior of the eye (aqueous and vitreous humour).

“Guide strand” is used interchangeably with antisense strand in the context of miRNA, siRNA or ss-RNAi compounds.

“HM” is an abbreviation for the 4′-C-hydroxymethyl-DNA nucleoside shown in FIG. 3 where B is one of the bases provided for herein.

“HNA” is an abbreviation for hexitol nucleic acid and includes the nucleoside shown in FIG. 7 where B is one of the bases provided for herein and it is conjugated to the 2′ position carbon of the ring.

“Identity” as used herein and as known in the art, is the relationship between two or more oligo sequences, and is determined by comparing the sequences. Identity also means the degree of sequence relatedness between oligo sequences, as determined by the match between strings of such sequences. Identity can be readily calculated (see, e.g., Computation Molecular Biology, Lesk, A. M., ed., Oxford University Press, New York (1998), and Biocomputing: Informatics and Genome Projects, Smith, D. W., ed., Academic Press, New York (1993), both of which are incorporated by reference herein). While a number of methods to measure identity between two polynucleotide sequences are available, the term is well known to skilled artisans (see, e.g., Sequence Analysis in Molecular Biology, von Heinje, G., Academic Press (1987); and Sequence Analysis Primer, Gribskovm, M. and Devereux, J., eds., M. Stockton Press, New York (1991)). Methods commonly employed to determine identity between oligo sequences include, for example, those disclosed in Carillo, H., and Lipman, D., Siam J. Applied Math. (1988) 48:1073.

“Inhibit expression” or “inhibition of expression” refers to the reduction or cessation of target nucleic acid expression as demonstrated by a reduction in mRNA levels, degradation of RNA, a reduction in protein synthesis or all of the above.

“Internal linkage sites” refers to linkage sites that are not at the 5′ or 3′-ends of an oligo strand. These sites are potentially subject to single strand endonuclease attack and to double strand endonuclease attack if they are part of a duplex with a partner strand. Such sites may also be simply referred to as linkage sites.

iPS cell or iPSC are abbreviations for induced pluripotent stem cells. They are created (induced) from somatic cells by experimental manipulation. Such manipulation has typically involved the use of expression vectors to cause altered (increased or decreased) expression of certain genes in the somatic cells. “Pluripotent” refers to the fact that such stem cells can produce daughter cells committed to one of several possible differentiation programs.

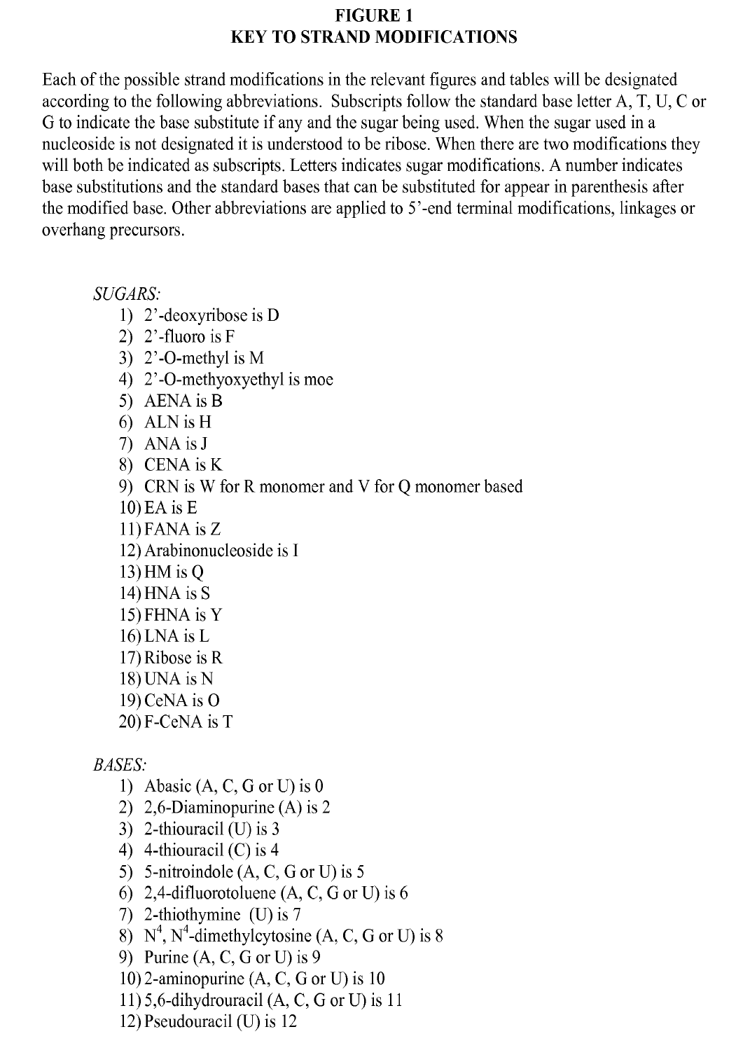

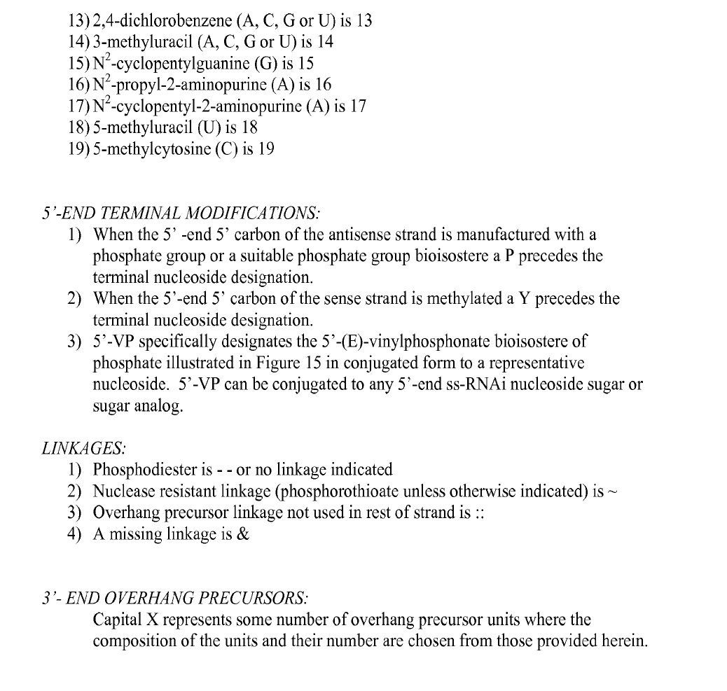

“Linkage site” refers to a particular linkage site or type of linkage site within an oligo that is defined by the nature of the linkage and the identities of the contiguous 5′ and 3′ nucleosides or nucleoside substitutes. Linkage sites generally can be designated by “X-Y” where X and Y each represent nucleosides with one of the normal bases (A, C, G, T or U) or other bases provided for herein or nucleoside substitutes and the dash indicates the linkage between them.

Where the description of the modifications are based on FIG. 1 the dash refers to a phosphodiester linkage and a squiggle (˜) to a nuclease resistant linkage typically phosphorothioate.

“LNA” is the acronym for locked nucleic acid. Standard LNA and three common variants are illustrated in FIG. 10 along with one phosphodiester linkage where “base” or B represents any of the bases provided for herein. Unless otherwise stated when LNA is referred to it should be interpreted as referring to one of the three version with the β-D-ribo configuration shown in panel B of FIG. 10, i.e., standard LNA, thio-LNA and amino-LNA but not alpha-L-LNA. The latter can be referred to as ALN. LNA nucleosides can be linked to adjacent nucleosides by any of the linkages provided for herein.

“Mismatch” refers to a nucleoside in an oligo that does not undergo complementary base pairing with a nucleoside in a second nucleic acid or with another nucleoside in the same oligo and where the effect is to antagonize interstrand or intrastrand duplex formation by setting up a repulsion of the opposing nucleoside base.

“MicroRNAs (miRNAs)” are a category of naturally occurring dsRNAs that typically trigger the post-transcriptional repression of protein encoding genes after one of the strands is loaded into RISC. Uncommonly, some miRNAs can also cause the increased expression of their RNA target. The miRNA antisense strand can be referred to as mature miRNA. It directs RISC to specific mRNA targets as recognized by the seed region of the mature miRNA. Most commonly the seed sequence recognizes completely matched sequences in the 3′UTR of mRNAs transcribed from multiple genes.

“MicroRNA mimics or miRNA mimics” are a category of manufactured compounds that when administered to cells utilize the cellular mechanisms involved in implementing the activity of naturally occurring miRNA in order to produce a modulation in the expression of a particular set of genes. MicroRNA mimics of the present invention can be designed to modulate some or all of the same genes modulated by a particular naturally occurring miRNA. When based on an endogenous miRNA, a mimic of the present invention will have the same seed sequence as the endogenous miRNA. The rest of the mimic may have all, some or none of the sequence found in the endogenous miRNA from which the seed sequence was taken. The miRNA mimics of the present invention also can be designed to modulate the expression of a set of genes by using a novel seed sequence. This novel seed sequence may replace the seed sequence of an endogenous miRNA or a siRNA designed to silence a particular target or be a duplex vehicle that is non-silencing in the absence of the novel seed sequence. The miRNA mimics of the present invention are referred to as seqMiRs or ss-MiRs depending on whether they involve one or two strands.

“Modulate”, “modulating” or “modulation” refer to changing the rate at which a particular process occurs, inhibiting a particular process, reversing a particular process, and/or preventing the initiation of a particular process , e.g., cellular signaling, protein transport, drug efflux, cell growth, morphological alterations, differentiation etc. If the particular process is tumor growth or metastasis for example, the term “modulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis.

“Moiety or moieties” is used as is understood in the art to refer to a part of a molecule that may include either whole functional groups or parts of functional groups as substructures. For example, an ester (RCOOR′) has an ester functional group (COOR) and is composed of an alkoxy moiety (—OR′) and an acyl moiety (RCO—), or, equivalently, it may be divided into carboxylate (RCOO—) and alkyl (—R′) moieties. This definition allows for a recursive nature, where moieties may contain functional groups which may contain moieties.

“Native RNA” is naturally occurring RNA (i.e., RNA with normal C, G, U and A bases, ribose sugar and phosphodiester linkages).

“Nucleoside” is to be interpreted to include the nucleoside analogs provided for herein. Such analogs can be modified either in the sugar or the base or both. These analogs can be substantially different than the normal counterparts, for example, certain six membered rings can replace ribose. Further, in particular embodiments, the nucleotides or nucleosides within an oligo sequence may be an abasic nucleoside. In overhang precursors and overhangs in RNAi triggers, each nucleoside and its 5′ linkage can be referred to as a unit.

“Nucleoside substitute” refers to structures with radically different chemistries, such as the aromatic structures that may appear in the 3′-end overhang precursors or overhangs of seqRNAi-based siRNA duplexes, but which play at least one role typically undertaken by a nucleosides. It is to be understood that the scope of the rules that apply to 3′-end overhang precursors are broader than the rules that apply to structures that occur in the regions of the seqRNAi strand that would form a duplex with its partner strand(s). In overhang precursors and overhangs each nucleoside substitute and its 5′ linkage can be referred to as a unit.

“Oligo(s)” is an abbreviation for oligonucleotide(s).

“Overhang” in the context of conventional siRNA and conventional miRNA refers to any portion of the sense and/or antisense strand that extends beyond the duplex formed by these strands and that is comprised of nucleoside or nucleoside substitute units.

“Overhang precursor” refers to that portion, if any, of a seqRNAi strand that would form an overhang when combined with a partner seqRNAi strand to form a seqRNAi-based duplex. The term also applies to ss-RNAi based on seqRNAi antisense designs where there are one or more units at the 3′-end of the strand that do not undergo complementary base pairing with the intended target and which would form an overhang if the strand were duplexed with a seqRNAi sense strand. The possible compositions that make up overhang precursors are provided for herein.

“Passenger strand” is used interchangeably with “sense strand” in the context of miRNA or siRNA compounds or their components. It forms a complex with its partner guide or antisense strand to form one of these compounds.

“Pharmacologically effective amount,” “therapeutically effective amount” or simply “effective amount” refers to that amount of an agent effective to produce a commercially viable pharmacological, therapeutic, preventive or other commercial result.

“Potency” refers to the amount of an ss-RNAi or other drug that must be administered in vivo or in vitro to get a particular level of activity against an intended target(s) in cells. In contrast, activity refers to the level of the effect a drug can have on its target. Thus, for example, two drugs directed to the same target can have the same level of activity, say both can suppress the expression of their target by 90% in a subject but they can very greatly in potency, say one produces the 90% suppression at 1 mg/kg while the other does so at 100 mg/kg.

“Prodrug” refers to a compound that is administered in a form that is inactive but becomes active in the body after undergoing chemical modifications typically through metabolic processes. In the context of RNAi-dependent compounds, prodrug designs have been proposed as a means of protecting such compounds from nucleases and/or promoting their uptake by cells. As for prodrugs generally any RNAi-dependent prodrugs have to undergo modification in the body to produce a compound capable of RISC loading and processing to induce silencing of the intended target(s). The administration of RNAi-dependent compounds without 5′-end phosphorylation of the antisense strand is not considered to constitute the administration of a prodrug.

“Protective carrier” refers to a type of carrier such as a lipid nanoparticle that provides nuclease protection to ss-RNAi, siRNA, miRNA or seqRNAi.

“Purine rich region” is defined as a region in an ss-RNAi strand with 4 contiguous nucleosides where at least 3 have a purine base.

“RNAi” is an abbreviation for RNA-mediated interference or RNA interference. It refers to the system of cellular mechanisms that produces RNAi triggers which are responsible for effecting silencing activity. Multiple types of RNAi activities are recognized with the two most prominent being siRNA and miRNA. Nearly always the RNAi triggers associated with these activities are double stranded RNA oligos most commonly in the 20-23-mer-size range. A common feature of the RNAi mechanism is the loading of one of these double stranded molecules into RISC following by the sense or passenger strand being discarded and the antisense or guide strand being retained and used to direct RISC to the target(s) to be silenced.

“RNAi antisense strand” is an antisense oligo that binds to an RNAi protein such that the antisense strand directs the RNAi protein to the intended target(s) and where said protein is required for silencing of said target(s).

“RNAi-dependent” refers to the use of an RNAi based mechanism as defined above to silence gene expression. Compounds using this mechanism include conventional siRNA, shRNA, dicer substrates, miRNA and the three types of seqRNAi (seqsiRNA, seqMiR and seqIMiR) as well as ss-siRNA, ss-IMiRs and ss-MiRs.

“RNAi mechanism” refers to the molecules and processes directly involved in engaging an RNAi trigger or ss-RNAi and using the guide strand to direct the mechanism to a target complementary to the targeting code where the end result is an inhibition of the function of the target either by steric hindrance or by causing the target to be degraded. RNAi machinery is an equivalent term.

“RNAi protein” is a protein that combines with an RNAi antisense strand such that the antisense strand directs the RNAi protein to the intended target(s) and where said protein is required for silencing of said target(s). Such RNAi proteins include but are not limited to AGO-1,2,3 and 4.

“RNAi trigger” refers to a double stranded RNA compound most commonly in the 20-23-mer size range that loads into RISC and provides the targeting entity (guide or antisense strand) used to direct RNAi activity. RNAi triggers can be siRNA or miRNA.

“Sd-MiR” refers to a single stranded miRNA mimic composed of an antisense strand designed according to the rules provided herein to be capable of self-dimer formation that is capable of being administered to a subject without a carrier or prodrug design.

“Seed sequence” or “seed region” the region comprised of the 6-7 contiguous nucleosides in positions 2-8 (or 2-7) counting in from the 5′-end of the antisense strand of conventional siRNA, miRNA or seqRNAi or ss-RNAi. In the case of a seqMiR it is also referred to as the “targeting code.”

“Seed vehicle” “Seed sequence vehicle” or “seed region vehicle” is used to describe a single strand into which particular seed sequences can be inserted as a starting point for the design of ss-MiR compounds. These seed sequences can come from endogenous miRNA or be novel. These vehicles are designed and/or selected to minimize off target effects and to promote efficient RISC loading and retention of the strand.

“Seed duplex” refers to the duplex formed between the seed sequence in a de facto antisense stand and its complement in an mRNA.

“Sense oligos or strands” are oligos that are complementary to antisense oligos or antisense strands of particular genes and which bind to such nucleic acids by means of complementary base pairing. When binding to an antisense oligo, the sense oligo need not base pair with every nucleoside in the antisense oligo. All that is necessary is that there be sufficient binding to provide for a Tm of greater than or equal to 40 ° C. under physiologic salt conditions at submicromolar oligo concentrations unless otherwise provided for herein.

“Silencing” refers to the inhibition of gene expression that occurs as a result of RNAi activity. In general, siRNA typically has a higher level of silencing activity (greater than 50% reduction) against its target at an optimal concentration while endogenous miRNA typically has a lower level of silencing activity (less than 50% reduction) against numerous targets.

“Ss-IMiR” refers to an antisense strand that is designed according to the rules provided herein and can be administered to a subject without a carrier or prodrug design, without the administration of a complementary sense strand and which suppresses the function of the targeted miRNA.

“Ss-MiR” refers to an antisense strand that is designed according to the rules provided herein and can be administered to a subject without a carrier or prodrug design, without the administration of a complementary sense strand and which suppresses the function of the targeted mRNA(s). The targeting code is primarily or exclusively provided by the seed sequence.

“Ss-miRNA” refers to a single stranded miRNA mimic composed of an antisense or guide strand that is capable of being loaded into RISC and subsequently directed to a set of targets for silencing of target gene expression, e.g., inhibition of a particular set of mRNAs containing the complementary binding sequences. The targeting code is primarily if not exclusively provided by the seed sequence.

“Ss-RNAi” refers to ss-siRNA and/or to ss-miRNA and/or to ss-MiR and/or to ss-IMiR compounds as well as to conventional ss-RNAi.

“Ss-siRNA” refers to an antisense strand that is designed according to the rules provided herein and can be administered to a subject without a carrier or prodrug design, without the administration of a complementary sense strand and which suppresses the function of the targeted mRNA. The principle targeting code is comprised of the central region of the strand.

“Stem cell” refers to a rare cell type in the body that exhibits a capacity for self-renewal. Specifically when a stem cell divides the resulting daughter cells are either committed to undergoing a particular differentiation program or they undergo self-renewal in which case they produce a replica of the parent stem cell. By undergoing self-renewal, stem cells function as the source material for the maintenance and/or expansion of a particular tissue or cell type.

“Subject” refers to a mammal including man.

“Substantially identical,” as used herein, means there is a very high degree of homology preferably >90% sequence identity between two nucleic acid sequences.

“Sugar or sugar analog” refers to a moiety component of a nucleoside or nucleoside analog consisting of a 5 or 6 membered ring that is suitable for use as part of a seqRNAi passenger or guide strand or AHD ss-RNAi strand as provided for herein. The sugar or sugar analog moiety is to be distinguished from the base moiety of a nucleoside or nucleoside analog. The sugar or sugar analog moiety is conjugated to one or two linkages that hold the nucleosides or nucleoside analogs in a strand in a particular order. The standard naturally occurring sugars in nucleosides are ribose and 2′-deoxyribose. The terms sugar or sugar analog also refers to any of the other moieties provided for herein that are components of nucleosides or nucleoside analogs and are conjugated to a base and to one or two linkages that incorporate it into a seqRNAi or AHD ss-RNAi strand. Sugar or sugar analogs can have a flexible or a ridged ring structure conformation that involves puckering of the ring. A rigid ring pucker conformation is forced upon a sugar analog by a bridge structure that covalently joins two atoms in its structure that are not contiguous. The pucker of the sugars and/or sugar analogs in a nucleotides strand determines the conformation of the duplex or region of the duplex that is formed when the seqRNAi or AHD ss-RNAi strand being considered binds to another strand or to itself through complementary base pairing. A sugar analog can also be referred to as a modified sugar or sugar substitute.

“Synthetic” means chemically manufactured by man.

“Targeting code” refers to a contiguous nucleoside sequence that is a subset of the guide or antisense strand sequence of a siRNA, miRNA or seqRNAi compound that is primarily or exclusively responsible for directing RISC to a specific target(s). Targeting codes typically can be distinguished on the basis of their particular positions within the guide or antisense strand relative to its 5′-end. Preferred codes contain for example, between 4 and 7, 5 and 8 continguous nucleosides, etc. For seqsiRNA, seqIMiRs, ss-siRNA and ss-IMiRs the targeting code is the central region if it is a seq-MiR or ss-MiR then it is the seed sequence.

“Target nucleic acid” refers to a target for the ss-RNAi of the present invention. Such targets include genes, mRNA, and regulatory non-coding RNA, for example, miRNA, endogenous antisense and long non-coding RNA (lncRNA).

“Tm” or melting temperature is the midpoint of the temperature range over which an oligo separates from a complementary nucleotide sequence. At this temperature, 50% helical (hybridized) and 50% coiled (unhybridized) forms are present. Tm is measured by using the UV spectrum to determine the formation and breakdown (melting) of hybridization using techniques that are well known in the art. There are also formulas available for estimating Tm on the basis of nearest neighbor considerations or in the case of very short duplexes in accordance with the relative G:C and U:A content. For the purposes of the present invention Tm measurements are based on physiological pH (about 7.4) and salt concentrations (about 150 mM).

“Treatment” refers to the application or administration of a single or double stranded oligo(s) or another drug to a subject or patient, or application or administration of an oligo or other drug to an isolated tissue or cell line from a subject or patient, who has a medical condition, e.g., a disease or disorder, a symptom of disease, or a predisposition toward a disease, with the purpose to inhibit the expression of one or more target genes for research and development purposes or to cure, heal, alleviate, relieve, alter, remedy, ameliorate, improve, or affect the disease, the symptoms of disease, or the predisposition toward disease. Tissues or cells or cell lines grown in vitro may also be “treated” by such compounds for these purposes.

“Unit” refers to the nucleoside or nucleoside substitutes that appear in overhang precursors and overhangs along with their 5′-end linkage. Nucleosides may appear in 5′-end or 3′-end overhangs but nucleoside substitutes can only appear in 3′-end overhang precursors and overhangs.

“UNA” is an acronym for unlocked nucleic acid or nucleosides. The ribose sugar ring in the nucleoside becomes acyclic by virtue of lacking the bond between the 2′ and 3′ carbon atoms as shown in FIG. 4 where B represents any of the bases provided for herein.

“Upstream” and “Downstream” respectively refer to moving along a nucleotide strand in a 3′ to 5′ direction or a 5′ to 3′ direction respectively.

“Vehicle” refers a substance of no therapeutic value that does not act as a carrier but is used to convey an active medicine or compound for administration to a subject in need thereof. An example of a vehicle suitable for use with the compounds of the present invention is buffered saline. The term vehicle also includes substances in which a drug can be suspended or desolved that provide for the release of the drug over time.

### C. The Embodiments

In one embodiment, rules and methods are provided for modifying guide strands so that they can induce the intended RNAi-dependent activity in vitro and in subjects in the absence of a partner passenger strand. Such ss-RNAi can be used therapeutically, or for research and development work such as target validation or for screening guide strands for activity that will then be used with partner passenger strands as needed to further boost activity. Ss-MiRs can have seed sequences taken from endogenous miRNAs or have novel seed sequences. Either of these types of seed sequence can be combined with a seed vehicle which as been designed to promote ss-MiR activity. Affinity increasing modifications can be made to either type of seed sequence in order to boost the desired silencing activity.

In yet another embodiment novel ss-RNAi designs, methods, compounds and compositions are provided. This technology provides ss-RNAi that has surprisingly greater potency and/or duration of effect than the ss-RNAi known in the art. The ss-RNAi of this invention can be administered to cells in tissue culture typically by means of a carrier as is the case for nucleic acid based drugs generally or in vivo where they can be administered with or without a carrier. A novel approach called Accommodating Helical Design renders the ss-RNAi of the invention much better able to be engaged by the RNAi mechanism by providing means that substantially compensate for the absence of a passenger strand.

### D. Comments on Terminology

The term “nucleoside” is to be interpreted to cover normal ribonucleosides and 2′-deoxyribonucleosides (also called 2′-deoxynucleosides) as well as the nucleoside analogs provided. It is to be understood that the stereochemical orientations of the compound referred to are subject to the same assumptions as are found in the literature generally when short hand terminology is used, for example, when ribose is referred to it is to be understood as being D-ribose or when arabinonucleic acids are referred to the are D-arabinonucleic acids. Capital C, G, A, T or U refers to cytosine, guanine, adenine, thymine or uracil bases. Ribose is not considered to be a modified sugar while all other sugars are including 2′-deoxyribose.

“Nucleoside substitute” refers to structures with chemistries radically different from nucleosides, but which play at least one role undertaken by a nucleoside in other situations. In addition, it is to be understood that the scope of the modifications that apply to 3′-end overhangs are broader than those applying to structures that occur in the regions of the seqRNAi strand that will form a duplex with its partner strand(s).

Statements such as “unless otherwise specified” or “unless otherwise provided for” refer to other specified modifications described herein that provide for a different modification(s) under certain circumstances. In these and in other instances where two or more rules specifying different modifications for the same entity, the more narrowly applicable rule (applies to fewer ss-RNAi strands) will dominate. For example, rules applicable to a particular architecture dominate architectural independent rules.

The terms “preferred” and “most preferred” are used to designate the optimal range of configurations for strands. In some instances, due to factors such as those arising from sequence specific differences, the optimal variant for a particular specification will not be what is generally preferred or most preferred. In such instances the selected variant still will fall within the more general range of variants provided for herein. Any such decision related to the use of variants that are not otherwise preferred or most preferred will be primarily based on balancing the desired level of silencing potency for the intended target along with the desired duration of this silencing vs. reductions in off-target effects. Off-target effects include minimizing the suppression of the expression of unintended targets and minimizing unintended modulation of innate immunity. These undesired effects are commonly associated with conventional siRNA duplexes and/or their component strands. They can be measured using methods well known in the art.

“Dose” is given as mg/kg of subject weight. When sequentially administered seqRNAi strands are being compared to the administration of a duplex RNAi drug or trigger then the combined dose of the sequentially administered strands will be the same as the dose of the duplex.

“AGO-2 based catalytic off-target silencing activity” or any similar statement refers to situations where the central region of a seqMiR antisense strand directs RISC to unintended target(s) that are then silenced. Various methods are provided herein to inhibit such siRNA-like off-target activity. These modifications can reverse the off-target inhibition of 1, 2, 3 or >3 genes by 20, 30, 40, 50, 60, 70, 80, 90 or >90% compared to compounds without the modification. The amount of expression for a particular gene being measured by the amount of corresponding RNA transcripts produced and/or amount of corresponding protein expressed.

“Off-target silencing due to the seed sequence” or any similar statement refers to situations where the ss-RNAi directs RISC to unintended targets that are then silenced. Various methods are provided herein to inhibit such seed sequence based off-target activity. The modification(s) can reduce the number of off-target genes being inhibited by 20, 30, 40, 50, 60, 70, 80, 90 or >90% compared to compounds without the modification(s) and/or reverse the off-target inhibition of 1, 2, 3, 4, or 5 genes by 20, 30, 40, 50, 60, 70, 80, 90 or >90% compared to compounds without the modification. The amount of expression for a particular gene being measured by the amount of corresponding RNA produced and/or amount of corresponding protein expressed. Off-target effects are problematic because they produce side-effects in subjects that are not justifiable given the benefits obtained by the intended effect and can confound otherwise reliable research and development data related to the silencing of the intended target(s).

“Unacceptably stimulates innate immune response” or any similar statement refers to situations where certain nucleosides in the ssRNAi strand, such as some of those where U, A or G are coupled with ribose or 2′-fluoro, activate the release of inflammatory cytokines (such as TNF-α, IL-1, IL-6, IL-12 and IL-16) from cells as a result of stimulating receptors involved in the innate immune response such as TLR 7 and 8. Various well-established assays are available to monitor the effects of oligoribonucleotides and other compounds on innate immunity. The induction of innate immunity becomes unacceptable when it produces side-effects in subjects (such as fever, chills and weight loss) that are not justifiable given the level of benefits obtained by the intended effect or when it confounds obtaining otherwise reliable research and development data related to the silencing of the intended target(s). Various methods are provided herein to inhibit such immune stimulation. The modification(s) can reduce levels of cytokine(s) induced by the innate immune system by 20, 30, 40, 50, 60, 70, 80, 90 or >90% compared to compounds without the modification(s)

“Silencing activity” or simply activity or any similar term in the context of a silencing effect refers to achieving a level of inhibition against an intended target(s) by a ss-RNAi agent used at a concentration producing a maximal silencing effect if not otherwise specified. In subjects the indicated silencing activities apply when the compounds in question are used without a carrier. The measurements can be made on any tissue that expresses the intended target(s) at levels suitable for analysis where the tissue is accessible to the compound(s). The central nervous system is an example of an area where tissues are relatively inaccessible when the compound(s) is administered systemically. Silencing activity can be measured either at the RNA (e.g., via hybridization assays such as Northern blots or amplification assays such as PCR) or at the protein level (e.g., via quantitative Western blotting, ELISA, immunohistochemistry or FACs analysis. RNA measurements generally are the simplest and are usually preferred for screening purposes but RNAi effects can involve functional inhibition without degradation of the RNA target so in such cases target protein analysis is required.

Unless otherwise specified it is to be understood that for simplicity certain linkage alternatives to the natural phosphodiester that are described herein (chirally specific phosphorothioate, boranophosphate) can substitute for one or more “phosphorothioate linkages” described in sections that refer to phosphorothioates generically.

E. Algorithms: Architectural Dependent Rules for ss-RNAi

### 1. Description:

A drug development platform based on ss-RNAi compounds must provide the means for ss-RNAi compounds to be routinely generated that have a number of essential properties. One of these involves providing sufficient in vivo nuclease stability to the ss-RNAi. Another is having the ability to be efficiently loaded into RISC in a manner that results in the intended silencing activity. This is a major unmet challenge because efficient guide strand loading has not been routinely achieved in the absence of a sense strand. This is evidenced by the low potency of the best existing ss-RNAi compounds known in the art. Further, these compounds are retained by RISC for a shorter period of time then are the same guide strands when they are delivered in duplex form with a sense strand.

Experimentally produced ss-RNAi compounds have been known in the art since around 2002. For the past 10 years there have been relatively few reports in the literature as no uniform approach for generating active compounds has yet been developed. In the earlier reports, compounds had minimal or no chemical modifications rendering them very susceptible to nuclease attack.

Recently it has been found that by making extensive use of the 2′-fluoro modification active ss-RNAi compounds can be generated with greater regularity. The 2′-fluoro modification by itself does not provide sufficient nuclease resistance for in vivo use, however, without a carrier or the addition of numerous nuclease resistant linkages. Further, it does not provide the means to generate ss-RNAi compounds with sufficient silencing activity for general therapeutic or R&D use. This is true even when the 5′-end 5′-carbon of the ss-RNAi is manufactured with a phosphate or phosphate bioisostere that can boost the activity of these compounds.

In vivo studies have shown that heavily 2′-fluoro modified ss-RNAi compounds used without a carrier and stabilized with phosphorothioate linkages and terminal nucleoside modifications that are more resistant to nuclease attack can silence the intended target in at least a few tissues in mice but it required conjugating C16 to the ss-RNAi to get substantial activity in tissues out side of liver (Lima et al., Cell 150: 883, 2012; Yu et al., Cell 150: 895, 2012). Doses of 100 mg/kg per treatment or substantially greater are required to achieve these effects in a very limited number of tissues, primarily liver, that are known to take up the largest amounts of single strand oligos along with the CNS following injection of the ss-RNAi into the brain side of the blood brain barrier. Thus the organ/tissue range for these ss-RNAi compounds is much smaller that it is for conventional antisense oligos. This dosing requirement for these ss-RNAi compounds is predictive of unacceptable adverse and other unacceptable off-target effects in humans and in non-human primates such as serious injection site reactions, sequelae associated with events such as complement activation, the stimulation of innate immunity and the inhibition of a variety of intracellular enzymes including those involved in the processing of nucleic acids and cell proliferation. Thus, a basic problem with the ss-RNAi compounds known in the art is that they cannot produce the intended silencing effect at doses and infusion rates suitable for therapeutic use in man due to adverse events or for R&D purposes where it is important to not have off-target effects that interfere with the interpretation of data dealing with the effects of silencing the intended target(s). These ss-RNAi compounds are also more expensive than conventional antisense oligos given the much larger doses required.

Originally it was assumed that the 2′-fluoro modification of a strand promotes a higher binding affinity between it and a complementary RNA or DNA strand as a result of a proposed 2′-fluoro-dependent conformational preorganization that favors the A-type helical conformation when a duplex is formed. Recent detailed conformational studies, however, have shown that there is little difference between unmodified single stranded RNA in this regard and single stranded 2′-fluoro RNA (Pallan et al., Nucleic Acids Res 39: 3482, 2011). Instead the effects of this modification on duplex stability have been found to be due to enthalpy. Duplexes involving 2′-fluoro RNA strand or strands are less hydrated than unmodified RNA duplexes.

Applicable standards for reasonable potency for a single stranded oligo catalytic inhibitor of a target RNA has been established in the art by the extensive in vivo work that has been done with antisense oligonucleotides (oligos) with an RNase H dependent mechanism of action. Single strand oligos with a preponderance of phosphorothioate linkages in their back bone such as ss-RNAi stabilized for in vivo use and RNase H dependent antisense oligos will share many properties including pharmacology and toxicology. From this work we know that there are unacceptable toxicities in patients and non-human primates related to high infusion rates and to the injection of high doses into tissues. The upper limits for infusion rates in to the circulation, for example, are on the order of 10 mg/kg/hr while a 20 mg/kg injection into a tissue is about the upper limit needed to get an optimal single dose effect while limiting adverse injection site reactions in humans. It is well know that certain other species, such as mice, can tolerate substantially higher infusion rates and tissue injection doses of antisense oligos. This tolerance is both due to an intrinsic greater resistance on the part of some species and to a lower standard of what is acceptable in terms of off-target effects in these animals, leaving aside the issue of drug toxicity. Further, it is well established that pharmacokinetics for antisense oligos extrapolate relatively well between species including humans and mice compared to other drug types such as small molecules. Thus, a need for substantially higher infusion rates and/or tissue injection doses in mice than are permissible in humans and non-human primates for a particular antisense oligo or type of antisense oligos is predictive of unsuitability for use in humans and non-human primates for either therapeutic and/or R&D purposes. These features exist for the ss-RNAi currently known in the art. Thus, there is a pressing need to substantially increase the potency of ss-RNAi compounds.

A unifying invention provided herein termed accommodating helical design (AHD) provides the means for routinely generating ss-RNAi compounds of three types (ss-siRNA, ss-IMiRs and ss-MiRs) that are more potent in vitro (using the same carrier) and in vivo (using the same carrier or not using any carrier). This increased potency is evidenced by a lower dose of the AHD ss-RNAi producing the same or higher level of RNA and/or protein target reduction, than the corresponding ss-RNAi compound without the AHD features and than an ss-RNAi compound with the same sequence modified according to methods currently present in the art (conventional ss-RNAi). There are many examples in the art of carriers suitable for use with single strand oligos, for example, carriers suitable for use in the present invention are found in WO 2011/139911.

AHD involves the use of certain nucleoside base and sugar or sugar substitute modifications (accommodating modifications). An accommodating modification significantly increases the probability that the nucleoside containing it and at least one of the two continguous nucleoside sugars or sugar substitutes to it in an oligo strand will be in the C3′-endo conformation. The accommodating modification can be either a nucleoside base or sugar or sugar substitute. When the accommodating modification is a base the nucleosides comprising said oligonucleotide being a mixture individually selected from the group consisting of ribose, 2′-fluoro and 2′-O-methyl. Other than any accommodating base the nucleosides comprising the oligos strand will have an essentially equal mix (as allowed by the chosen oligo length) of the standard A, U, C and G bases and will have no more than three purine containing bases in a row. When the accommodating modification is a sugar substitute then the other nucleoside sugars in the oligo will be a mixture selected from the group consisting of ribose, 2′-fluoro and 2′-O-methyl.

The other attribute of accommodating modifications is that when they are present in conventional RNAi triggers they do not project any part of their structure into the one of the two grooves in the A-type helix to a sufficient degree that the RNAi activity of the trigger is substantially interfered with. Substantial interference being equal to or greater than a 20, 30, 40, 50, 60, 70 or 80% reduction in the level of target suppression for siRNA or for the exression of 1, 2, 3, 4, or >5 targets for miRNA when either RNAi type is used at a dose giving optimal suppression. When accommodating modifications are incorporated into ss-RNA as provided for herein then the effect will be to increase the potency of the ss-RNAi relative to an ss-RNAi of the same sequence but without the accommodating modifications.

Without being limited by theory, RISC engages with guide strands according to the degree to which the sugars or sugar substitutes in their nucleosides (exclusive of any overhang) are in the C3′-endo conformation. As single strands the individual passenger and guide strand nucleoside sugars have at most a 50% probability of being in the C3′-endo conformation. When the guide and passenger strands of an RNAi trigger are in the double stranded configuration the majority of the nucleoside sugars are in the C3′-endo conformation and as a result the duplex is in an A-type helical conformation. It is well established in the art that the A-type helix is a necessary but not sufficient attribute of an effective conventional double strand RNAi trigger. This effect of the passenger strand on the guide strand is almost certainly the main reason double stranded RNAi triggers are more potent than ss-RNAi. The potency of the ss-RNAi can be increased by increasing the probability that multiple sugars in an ss-RNA will be in the C3′-endo conformation through the use of accommodating modifications.

Potency for ss-siRNA and ss-IMiRs of the present invention and for conventional ss-RNAi that cause their RNA target to be degraded can be measured by determining the ss-RNAi dose at which the RNA target is reduced by a particular percentage selected from the group equal to or greater than 40, 50, 60, 70, 80 and 90%. Ss-siRNA RNA targets can include mRNA as well as non-coding RNA such as LncRNA and endogenous antisense RNA.

Potency for ss-siRNA and ss-IMiRs of the present invention and for conventional ss-RNAi that do not cause their mRNA target to be degraded can be measured by determining the dose at which the protein encoded by the mRNA target is reduced by a particular percentage selected from the group equal to or greater than 40, 50, 60, 70, 80 and 90%.

Potency for ss-MiR of the present invention and for conventional ss-MiRs that cause their mRNA target(s) to be degraded can be measured by determining the dose at which 1, 2, 3, 4, 5 or >5 of the mRNA targets are reduced by a particular percentage selected from the group equal to or greater than 20, 30, 40, 50, 60 and 70%.

Potency for ss-MiR of the present invention and for conventional ss-RNAi that do not cause their mRNA target to be degraded can be measured by determining the dose at which 1, 2, 3, 4, 5 or >5 of the proteins encoded by the targeted mRNA are reduced by a particular percentage selected from the group equal to or greater than 20, 30, 40, 50, 60 and 70%.

Comparing the potency of the AHD ss-RNAi to the corresponding ss-RNAi compound without the AHD modifications or to a conventional ss-RNA results involves comparing the percent reduction in the target(s) falling within the ranges just defined for the AHD ss-RNAi of the present invention to the level of target suppression achieved by either the same ss-RNAi without the AHD modifications of to an ss-RNAi to the same target modified according to methods known in the art for ss-RNAi where the comparisons are based on using the same dose and conditions.

AHD ss-RNAi will produce a higher level of suppression of the intended target(s) compared to the other ss-RNAi compounds. Experimentally the effect on the target by the AHD ss-RNAi must be shown to be a statistically significantly higher percent reduction in the target than is the case for one or both of the comparator ss-RNAi compounds and the confidence intervals cannot overlap using common standards used in the field. This difference in suppressive activity on the target(s) also can be shown to be biologically significant by showing a statistically significant greater change is some other biologic parameter other than on the target itself on the part of the AHD ss-RNAi compared to the other ss-RNAi compounds under consideration. Such biologic parameters can include but are not limited to changes in the levels of 1, 2, 3, 4, 5, or >5 other molecules, to changes in cellular behavior such as proliferation, death, cellular morphology, cellular signaling, and secretion of modulatory substances. For general purposes the difference in reduction in target levels by the AHD ss-RNAi compared to one or both of the other ss-RNAi compounds can be described mathematically using the following formula: X %+Z(100%−X %)=Y % where X is the percent reduction in target suppression achieved by the AHD ss-RNAi of the present invention and where the percent reduction in the target falls within the ranges just described, Y is the percent reduction in target suppression achieved by either the same compound minus the AHD modifications or an ss-RNAi compound to the same target that is modified according to methods known in the art for ss-RNAi and Z is a negative reduction factor. The reduction factor being found within the ranges defined by a group of numbers equal to or numerically greater than 0.1, 0.3, 0.6, 2, 6, 20 and 40 but not greater than the next number in the series and not numerically higher than 50. So, for example, if X is 95% and Y is 23% then Z is −14.4 or if X is 40% and Y is 10% then Z is −0.5. The −14.4 is between 6 and 20 in the series while the −0.5 is between 0.3 and 0.6 considered as negative numbers. In the case of comparisons of ss-MiRs such differences in potency can be shown for 1, 2, 3, 4, 5 or >5 targets. In some instances the AHD -RNAi of the present invention will produce one of the levels of target suppression defined above at a particular dose and one or both of the comparator ss-RNAi compounds will not be able to produce that level of target suppression at any dose. This is clear evidence that the AHD ss-RNAi of the present invention is superior for targeting the cells for silencing.

In order to demonstrate that an ss-RNAi compound has an RNAi mechanism one or more of the assays found in the art for establishing RNAi activity can be shown to support this mechanism. These assays include but are not limited to the following: (1) Those that demonstrate that the ss-RNAi can be loaded into an argonaute protein involved in the RNAi mechanism and direct it to the intended target in a cell-free assay. Alternatively, the ss-RNAi-argonaute complex is isolated from cells and the isolate shown to bind the intended target. In addition, using an in vitro cell based assay the intended target of the ss-RNAi is shown to be suppressed following administration of the ss-RNAi to cells typically by means of a carrier or to cells in vivo with or without a carrier; (2) An equal to or greater than 30, 40, 50, 60, 70 80 or 90 percent reduction of an argonaute or argonaute-like protein known to be a component of the RNA mechanism in cells with at least one intended target of the ss-RNAi to be tested is shown to statistically significantly reduce the activity of the ss-RNAi against the target when administered to the cells compared to RNAi competent cells with that have taken up as much or more of the ss-RNAi. Cells can be treated with an inhibitor of a particular argonaute or argonaute-like protein to reduce its levels (such as an antisence oligo, siRNA or shRNA) or the cells may naturally not express the argonaute or argonaute-like protein or they may be genetically engineered to not express it; (3) The 5′-RACE assay showing that a ss-siRNA directs the cleavage of its RNA target at the expected site (opposite nucleosides 10 and 11 counting from the 5′-end) proves RNAi is the mechanism responsible for suppressing the expression of the target.

AHD modifications are most suitably applied in a sequence and/or position within the strand dependent manner. The most important location for accommodating modifications is starting at the third nucleoside position from the 5′-end through nucleoside positions 18-20. Accomodating modification can involve 2, 3, 4, 5, 6 or >6 of the nucleotides in an ss-RNAi. The overhang precursors added to strands of this length do not require accommodating helical design modifications. They simply have to be compatible with conventional siRNAi activity as established in the art. These modifications/substitutions (subsequently referred to as modifications or accommodating modifications) to the strand have two general properties. First, they increase the probability that a C3-endo pucker conformation will be assumed by 2, 3, 4, 5, 6, or >6 of the nucleosides in an ss-RNAi compared to an otherwise identical ss-RNAi where they are not used when measured in the unhybridized ss-RNAi using techniques established in the art. This increase in probability will include the the nucleoside containing the modification but often the modification will extend to the contiguous nucleosides containing a sugar or sugar substitute that is conformationally flexible. To a lesser extent this effect of an accommodating modification can also extend to varying degrees to other nearby nucleosides with respect to the two that are contiguous to the nucleoside with the modification when these nearby nucleosides have flexible sugars or sugar substitutes. Sugar and sugar analogs classified as “most flexible” include ribose, 2′-fluoro and 2′-0-methyl; those classified as “flexible” include HNA, FHNA, ANA, CeNA and F—CeNA; those classified as “semi-flexible” include CRN and CENA and the one classified as “rigid” is LNA. The most flexible are not accommodating modifications while the rest are.

Second, accommodating modifications preferably do not project any part of their structure into the major or minor grooves to such an extent that they would substantially sterically hinder the interaction of such a duplex with the RNAi machinery if the ss-RNAi with the modification were the guide strand present in a double stranded RNAi trigger. The size limits for any such projection can be determined from what is known about such projections inhibiting silencing by conventional RNAi triggers and the fact that the 2′-0-methyl modification is less well tolerated by ss-RNAi when compared to siRNA. More specifically, in the case of 2′ carbon accommodating modifications to ribose or to the corresponding carbon in a sugar substitute it is preferred that the structure be no larger than a 2′-0-methyl. Such alkyl groups project into the minor groove that is narrower than the major groove. The exceptions to this rule include the 5′-end terminal nucleoside and nucleoside(s), if any, in the overhang precursor. Here larger 2′-modifications such as those projecting from (2′-MOE or EA can be tolerated. Another exception is modifications to the nucleoside in the second position from the 5′-end of an ss-siRNA or ss-IMiR where a nucleoside modification of the type provided for herein and in the art that reduces off target effects due to the seed region acting as a targeting code can be used. Structures on bases, such as those conjugated to the C-5 position of some pyrimidine bases, can project in to the major groove in an A-type duplex. These are preferred to be smaller than a propynyl group. Propynl groups occupy approximately 53 cubic angstroms of space while the well-tolerated methyl group in this position occupies approximately 23 cubic angstroms of space.

The ribose sugar in a standard ribonucleoside (A, C, G and U) by itself or in a RNA strand made up of standard ribonucleotides has a greater probability of being in the C3′-endo pucker than in any other conformation, but this probability can be substantially changed due to other influences including the base in the ribonucleoside in question and the composition of nearby nucleosides when the ribonucleoside is in a strand. The ability of a ribose or other sugar or sugar substitute in a nucleoside to be conformationally influenced is a measure of its flexibility. A nucleoside in a RNA strand that has a standard purine base and a flexible sugar, for example, will have a sugar that has a significantly lower probability of being in the C3′-endo pucker conformation than is a comparable nucleoside with a standard pyrimidine base. This probability is even lower in a purine rich area of an RNA strand, for example, because of the influence of other near by nucleosides with purine bases.

Accommodating modification(s) in strands are required more frequently within and/or near to nucleosides with purines, and in particular within purine rich areas when compared to those having pyrimidine containing nucleosides or areas that are pyrimidine rich. The most flexible sugars of those provided herein include ribose, 2′-fluoro and 2′-0-methyl while at the other end of the spectrum is LNA. LNA is locked into a C3′-endo-like conformation by a short 2′-O, 4′C methylene bridge that makes it the most conformationally restricted sugar of those under consideration for AHD use. As a result LNA is essentially not very susceptible to the conformational influences of the contiguous nucleosides and conversely LNA can have the most conformational influence on contiguous nucleosides that have sugars other than LNA. LNA, however, does not exactly replicate the C3′-endo conformation of ribose and as a result must be used sparingly in accordance with the rules provided or the net effect can be a distortion of the desired ss-RNAi nucleoside sugar conformations rather than a promotion of it. The other nucleosides with conformationally restricted sugars such those with a longer 2′-O, 4′C ethylene bridge, for example CENA, have more flexibility than LNA and accordingly have less influence on contiguous nucleosides but also can have less of a distorting effect when used more frequently in ss-RNAi strands. The sugar substitutes which serve as accommodating modifications that are not conformationally constrained by a bridge-like structure, have a higher probability of having a C3′-endo conformation inducing effect on contiguous nucleosides than does ribose or 2′-fluoro, but less so than the sugar substitutes with a conformation constraining bridge.

Another type of accommodating sugar modification involves the use of nucleosides where the sugar has been replaced by one of certain six-membered ring structures where a C3′-endo-like conformation is the most probable conformation for the sugar. Further, nucleosides containing one of these sugars can increase the probability that contiguous nucleosides with equally or more flexible sugars will be in this conformation as well. These 6-membered rings include hexitol (HNA), 3′fluoro hexitol (FHNA), altritol (ANA) and cyclohexenyl rings including the fluoro cyclohexenyl variant (Seth et al., J Organic Chem 77: 5074, 2012). When the latter two sugar substitutes are found in nucleosides in an oligo strand they are referred to as cyclohexene nucleic acids (CeNA) and fluoro cyclohexene nucleic acids (F—CeNA) respectively. These modifications can be used to improve the accommodating characteristics of nucleosides with either pyrimidine or purine bases but particularly the latter for ss-RNAi use in accordance with the present invention. Nucleosides with one of these sugars can also have one of the accommodating base modifications provided for herein. Nucleosides with one of these 6-membered rings cannot exactly mimic the effects of ribose in the C3′-endo conformation so they have to be used sparingly in accordance with the rules provided. Other sugar substitutes meeting the criteria provided herein can also be used as accommodating modifications.

The presence of certain bases in a nucleoside will increase the probability that the C3′-endo conformation will be assumed by any ribose, 2′-0-methyl or 2′-fluoro sugar in the nucleoside and to a lesser extent other flexible accommodating sugar modifications in the nucleoside with the accommodating base modification as well as in contiguous nucleosides with flexible sugars. Such bases are suited for use in the present invention if they do not produce the type of steric hindrance problem just discussed where large projections into the major or minor grooves occur that can sterically hinder the RNAi mechanism. Examples of such bases suitable for use in the present invention include the U-like bases pseudouracil, 2-thiouracil and 5-methyluracil and the C-like bases 5-methylcytosine and 4-thiouracil. 4-thiouracil is an example of a C-like base that is complementary to G but unlike uracil does not form a wobble base with G. Pseudouracil, for example, has a more potent effect on the sugar conformation of upstream nucleosides than on the downstream ones such that it can affect the sugar conformation in 2 or 3 upstream purine nucleosides with flexible sugars while only affecting one down stream purine nucleoside with a flexible sugar.

A region in a strand with a concentration of purine nucleosides with A, for example -AAUA- or -AACA-, can promote more of a B-type or an unstacked local area in a helix rather than the A-type. This can be adjusted to favor the A-type helix or in the case of an ss-RNAi its ability to accommodate the RNAi mechanism, for example, by the replacement of the U with a U-like base or the C with a C-like base of the type just described. Thus, a purine rich region in an ss-RNAi sequence with one or more intervening and/or contiguous pyrimidine nucleosides with an accommodating base modification can be made more accommodating with respect to the RNAi mechanism. Single purine containing nucleosides can also benefit by being contiguous to or within one position from a nucleoside with an accommodating base. The strand can also be designed using these same accommodating bases to increase the ability of pyrimidine rich regions to promote the accommodation of the RNAi mechanism. In this case a lower frequency of nucleosides with such bases can be used because of the greater probability that of the nucleosides with pyrimidine will assume the C3′-endo conformation vs. comparable nucleosides with purine bases. Finally, accommodating bases can be used with the more flexible accommodating sugars to further increase the accommodating activity when a pyrimidine base is called for.

In addition to modifying endogenous miRNA guide strands according to AHD to generate ss-MiRs, ss-MiRs can be constructed by applying AHD to modular components that can be combined to form the active compound. This approach can increase the speed by which highly active ss-MiRs can be generated and can improve outcomes compared to ss-MiRs based on the entire sequence of endogenous miRNA guide strands. Moving in the 5′ to 3′ direction the modules are the following: (1) the phosphate and phosphate isosteres structures with a similar shape and size that can make up the molecular entity attached to the 5′ carbon of the 5′-end nucleoside; (2) the eight 5′end nucleosides that include the seed sequence; (3) the seed vehicle; and (4) the overhang precursor. The seed sequence used in an ss-MiR can be taken from an endogenous miRNA or be a novel sequence selected for its ability to direct the silencing a particular mRNA or set of mRNAs.

The sequence of the known miRNAs has been compiled in MiRBase (www.mirbase.org) that is incorporated herein by reference. MiRBase is the source for miRNA guide strand sequences for use in constructing the following: (1) ss-MIRs based on the entire endogenous sequence; (2) the 5′-end terminal 9 nucleosides for designing ss-MiRs based on the modular approach; and (3) determining the complimentary sequence for particular miRNA guides strands for use in designing ss-IMiRs.

### 2. Limiting Self-Interaction

Self-interaction in the form of self-dimer formation and hairpins on the part of a potential ss-RNAi can negatively affect the desired interactions between the strand and RNAi mechanism/RISC. Numerous oligo analysis software programs and online sites well known in the art provide the means to identify and calculate the strength of any self-dimers and hairpins for unmodified oligo sequences. The program used in the design of the ss-RNAi compounds of the present invention is OLIGO version 3.4 copyright by Wojciech Rychlk 1988-90. It has the advantage of allowing the salt concentration to be set to the physiologic level and the strand concentration set within the range of what is typically seen in cells for an effective guide strand. When this program indicates that at physiologicalic salt levels and appropriate ss-RNAi concentrationt a given strand does not exhibit self-interaction under physiologic conditions with either a negative or a positive kcal/mol value, then the strand sequence is assumed to have met an important but not sufficient criterion for being suitable for ss-RNAi use. If it shows a negative kcal/mol value, it is not preferred or rejected depending on the level of negativity with values lower than −2.0 resulting in rejection unless there is no other ss-RNAi sequence choice, for example, when the target is a miRNA guide strand. If a strand shows a positive value self-interaction value then any accommodating and/or nuclease resistance modifications that can produce a negative kcal/mol value should be avoided unless their effect can be offset, for example, with a mismatch or using on of the affinity reducing base modifications. Generally, an internal mismatch can be expected to result in a 5-10 degree centigrade drop in Tm. This should be sufficient to reverse most instances of unacceptable self-interaction. Nevertheless, in certain embodiments, ss-RNAi strands will be tested for self-interaction. Any mismatch(s) employed must not occur in the targeting code for a given type of ss-RNAi. The rules for mismatches in seq-siRNA and seq-IMiRs that apply to areas outside of the central region also apply to mismatches in ss-siRNA and ss-IMiRs. The limit is 1-3 mismatches restricted to certain positions and frequencies in the strand. In the case of ss-MiRs, the positioning of mismatches outside the seed sequence is not limited.

3. Providing Nuclease Resistance for ss-RNAi:

The rules for achieving nuclease resistance provided herein or in the art for seqRNAi strands must be further refined and modified for AHD ss-RNAi. This primarily involves limiting the modifications that promote nuclease resistance to those consistent with AHD. Fortunately a number of AHD modifications also promote nuclease resistance. An ss-RNAi compound can be divided into three general regions from the point of view of providing nuclease resistance. Once this basic requirement for nuclease resistance is established during the design process then other adjustments are made such as might be related to the nature of the targeting code and those required by Accommodating Helical Design. The three general regions are the central, the 3′-end and the 5′-end regions. They are generally protected from particular classes of enzymes as follows:
1) Protection of certain internal linkage sites from single strand endonuclease attack;
2) Protection of linkages between the terminal two or more nucleosides or nucleoside substitutes at the 3′-end of the strand from 3′-end exonuclease attack;
3) Protection of the linkage site at the 5′-end of the strand from 5′-end exonuclease attack.

The level of nuclease protection required is determined by how the ss-RNAi will be used. It has been shown in the art that ss-RNAi that has only 2′-fluoro nucleosides, exclusive of the overhang precursor, and with only phosphodiester linkages can function well both in vitro and in vivo when used with a protective carrier (Chorn et al., RNA 18: 1796-804, 2012; Haringsma et al., Nucleic Acids Res 40: 4125-36, 2012). When used with such protective carriers it is only necessary to use the accommodating modifications of the present invention in order to improve potency. Nevertheless additional nuclease resistance modifications can still be useful for extending the lifespan of the intact ss-RNAi in cells.

When protection from single strand endonucleases is being considered then each linkage is used to define a different linkage site. Thus, the 5′-end nucleoside of one linkage site can be the 3′ end nucleoside of another linkage site. In other words, the internal linkage sites are considered to be overlapping.

In the case of the 3′end and 5′-end protection rules for exonucleases, the linkage sites are defined by their position with respect to either the 3′ or 5′-end. Thus, there is no overlapping of linkage sites in these situations with respect to the application of the nuclease protection rules.

Linkage sites vary in their susceptability to nuclease attack. As mentioned previously, there are tissue, species and disease related differences in nuclease expression.

When a site is found to be more sensitive, the site can be further protected or another ss-RNAi directed to the same target but without the problem linkage can be used. Such sites can be identified by collecting treated cultured cells or removing the target tissue from an animal or patient, extracting the oligo and its fragments then determining which linkage site(s) were cleaved using liquid chromatography in combination with mass spectrometry using established methods.

The modifications used to generate a degree of nuclease resistance vary in both their effectiveness in resisting nuclease attack and in their impact on the desired suppressive activity on the intended target. 2′-fluoro generally has the least negative effect on activity particularly in the ss-siRNA and ss-IMiR targeting code but it is also the least nuclease protective.

The percentage of phosphorothioate linkages in a strand is important in situations where the oligo strand is to be distributed to tissues by way of the circulation. In general, having 70% or more of the linkages being phosphorothioate generally results in a minimal renal clearance and maximal distribution to tissues from the circulation. A greater number of phosphorothioate linkages, particularly when they fall within the ss-siRNA or ss-IMiR targeting code, however, can negatively affect the desired activity against the target. Hence, these two features need to be balanced or another ss-RNAi sequence to the target used that can achieve the required result.

Administration of an ss-RNAi oligo strand to cultured cells or to sites in the body that do not involve distribution to the target tissues through the circulation, such as the CNS and the fluid in the eye, generally can have only the phosphorothioate linkages needed for nuclease resistance.

There are 16 different possible linkage site combinations in RNA oligos made up of the standard 4 nucleosides. Of these, 8 are relatively resistant to endonuclease attack compared to the rest. These 8 are A-A, U-U, G-G, G-C, G-U, G-A, A-G and C-U. The other 8 that are relatively less resistant can be broken into three groups on this basis. All of 16 these linkage sites when unmodified are readily susceptible to 5′ and particularly 3′ exonuclease attack.

With respect to endonucleases, there are species and tissue type differences, in vivo in particular, where some of the 8 most resistant linkage sites can have a level of resistance that is similar to the commonly less resistant linkage sites. For example, mouse liver has nuclease activity that can cleave the C-U, G-G and G-U linkages sites in under an hour when they are only protected by a 2′-fluro in each of the two nucleosides. The time required for cleavage can be increased substantially by using a 2′-O-methyl for one of the two nucleosides in these linkage sites. When such unusually sensitive sites are shown to occur then modification(s) to the linkage site provided for by the rules that increase its resistence are applied or a different ss-RNAi to the same target that does not have this problem can be used.

Another situation where one or more of these commonly more resistant linkage sites can be relatively more susceptible to attack can occur when the subject being treated has a disorder where abzymes directed to nucleic acids are generated. These antibodies can have nucleic acid cleaving specificities that are different than normal nucleases. Again the solution in such a situation is to identify and protect the linkage site(s) being cleaved at an unusually higher rate or use an ss-RNAi to the same target but with a different sequence.

Ss-RNAi strands are preferably 16-22 nucleosides in length exclusive of the overhang precursor. It is preferred, particularly when it is desirable to reduce the number of phosphorothioate linkages in the central region of the strand, that greater than 50, 60, 70, 80 or 90% of the linkage sites exclusive of the overhang precursor be selected from the group C-G, A-C, A-U, A-A, U-U, G-G, G-C, G-U, G-A, A-G and C-U and most preferred that they be selected from the group A-A, U-U, G-G, G-C, G-U, G-A, A-G and C-U. The most preferred linkage sites are the latter 8 of 16 sites that are generally the most resistant to nuclease attack although there are species and tissue type differences where some of these linkage sites can have a level of resistance that is similar to the commonly less resistant linkage sites. When this is shown to occur then the only change to the rules for achieving nuclease resistance is that the linkage site(s) in the commonly more resistant group that is shown to be relatively less resistant in a particular species or tissue is that the linkage site(s) that proved to be less resistant is modified in accordance with the rules for less resistant linkage sites. This can involve the addition of a phosphorothioate linkage and/or adjusting one or both of the nucleosides that make up the unusually sensitive linkage site to have a more nuclease resistant modification such as changing a ribose to a 2′fluoro or a 2′-fluoro to a 2′-O-methyl unless otherwise provided for, for example mouse liver has nuclease activity that can cleave the C-U, G-G and G-U linkages sites in under an hour when they are only protected by a 2′-fluoro in each of the two nucleosides. The time required for cleavage can be increased substantially by using a 2′-O-methyl for one of the nucleosides in these linkage sites. Methods for determining which specific linkage site(s) are cleaved in particular tissues in particular species are well known in the art and typically involve liquid-chromatography-coupled with mass spectroscopy analysis. Depending on the type of ss-RNAi there are certain limitations to the use of these linkage site preferences. In the case of ss-MiRs the seed sequence is predetermined either by the endogenous miRNA to be mimicked or by the sequence found in the intended target(s) that will be the partner in the seed duplex in the case of a novel seed sequence. In the case of ss-siRNA the linkage site preference can be part of the selection process for an ss-RNAi against a particular target, but it may be difficult to both get the desired higher proportion of most resistant linkage sites and a low level of self-interaction. When there is such a conflict then having a strand with minimal self-interaction is more important. In the case of ss-IMiRs these linkage preference have to be largely discarded because there are minimal options with respect to sequence.

The following rules provide for the basic nuclease resistance for ss-RNAi strands. Other rules that are subsequently presented provide for modifications specific to a particular type of ss-RNAi, the inhibition of off-target effects and optimization of AHD. These rules supersede the basic nuclease rules when there is a different chemistry specified. As for seqRNAi, the linkages provided herein that could replace phosphorothioate outside of the linkages specific to overhang precursors can be used in ss-RNAi.

The internal linkage sites to be protected in order to establish nuclease resistance are defined by the ribonucleosides that bracket a given linkage. Thus, the frequency and positioning of the protective chemical modifications are affected by the underlying strand sequence.

For general use the linkage sites (reading 5′ to 3′) to be protected from single strand endoribonucleases from the generally most resistant group A-A, U-U, G-G, G-C, G-U, G-A, A-G and C-U are the following unless otherwise specified:
1) The 5′ nucleoside member of the linkage is ribose, 2′-fluoro or 2′-O-methyl. When it is 2′-O-methyl it is preferred that the nucleoside immediate upstream of it not be 2′-O-methyl. When the ss-RNAi is to be used in vivo without a protective carrier it is preferred that the 5′-end member of the linkage site be selected from 2′-fluoro and 2′-O-methyl.
2) The intervening linkage with the 3′ nucleoside in the linkage site is phosphodiester. The 3′ nucleoside member of the linkage site can have a sugar that is selected from the group consisting of ribose, 2′-fluoro and 2′-O-methyl. When it is 2′-O-methyl it is preferred that the nucleoside immediate downstream of it not be 2′-O-methyl. When the ss-RNAi is to be used in vivo without a protective carrier it is preferred that the 3′-end member of the linkage site be selected from 2′-fluoro and 2′-O-methyl where the selection is different than the one used for the 5′-end nucleoside in the linkage site. Further it is preferred that when the ss-RNAi is to be used in vivo without a protective carrier that either the linkage site under consideration have a phosphorothioate linkage or the contiguous linkage sites have phosphorothioate linkages.

For general use the generally less resistant linkage sites (reading 5′ to 3′) to be protected from single strand endoribonucleases ranked according to increasing resistance are those where:
1) A pyrimidine (U or C) containing ribonucleoside is followed by a purine (G or A) except C-G.
2) A linkage sites as defined by C-C and U-C.
3) A linkage sites as defined by C-G, A-C and A-U.

Unless otherwise specified the rules for protecting the indicated linkage sites (1-3 above) from single strand endoribonucleases are the following unless otherwise specified:
1) The 5′ nucleoside member of the linkage is 2′-fluoro or 2′-O-methyl. When it is 2′-O-methyl it is preferred that the nucleoside immediate upstream of it not be 2′-O-methyl.
2) The intervening linkage with the 3′ nucleoside in the linkage site is preferably phosphorothioate
3) The 3′ nucleoside member of the linkage site can have a sugar that is selected from the group consisting of 2′-fluoro and 2′-O-methyl. When it is 2′-O-methyl it is preferred that the nucleoside immediate downstream of it not be 2′-O-methyl.

The required 5′end exonuclease protection for an ss-siRNA or an ss-IMiR is provided by the following rules unless otherwise specified:
1) The 5′-end terminal nucleoside for the strand is selected from the group consisting of nucleosides with: 2′-fluoro, 2′-0-methyl, 2′-MOE, EA, LNA, CRN (R or Q monomer), UNA, HM, HNA, FHNA, CeNA, F—CeNA and CENA.
2) The 3′ member of the 5′-end terminal linkage site can have a sugar that is selected from the group consisting of ribose, 2′-fluoro, 2′-0-methyl, HM and CRN Q monomer where R₂ is selected from the group consisting of H, F, and OH).
3) The intervening linkages for the two 5′-end terminal linkage sites are preferred to be phosphorothioate if the ss-RNAi is to be used in vivo without a protective carrier.

The required 5′end exonuclease protection for an ss-MiR is provided by the following rules unless otherwise specified:
1) The 5′-end terminal nucleoside for the strand is selected from the group consisting of nucleosides with: 2′-fluoro, 2′-0-methyl, 2′-MOE, EA, LNA, CRN (R or Q monomer), UNA, HM, HNA, FHNA, CeNA, F—CeNA and CENA.
2) The 3′ member of the 5′-end terminal linkage site can have a sugar that is selected from the group consisting of ribose and 2′-fluoro.
3) The intervening linkages for the two 5′-end terminal linkage sites are preferred to be phosphorothioate.

The required 3′end exonuclease protection is provided by the following rules unless otherwise specified:
1) An overhang precursor comprising of at least two units is preferred for protection of the 3′-end. Three unit overhang precursor based on the sequences shown in Table 3 are most preferred and specific examples of modifications to such overhang precursors are provided in Table 4. The linkages used in overhang precursors can be any of those provided in the section by that name.
2) When there is a single unit overhang precursor the penultimate nucleoside at the 3′-end and the overhang precursor are individually selected from the group 2′-fluoro, 2′-O-methyl, 2′-MOE, LNA, EA, CRN, CENA, ANA, HNA, FHNA, CeNA and F—CeNA.
3) In the absence of a 3′-end overhang precursor the required 3′end protection can be provided by the use of two terminal nucleosides that are individually selected from the group 2′-fluoro, 2′-O-methyl, 2′-MOE, LNA, EA, CRN, CENA, ANA, HNA, FHNA, CeNA and F—CeNA.
4) When there is a single unit overhang precursor or in the absence of an overhang precursor the 5 consecutive 3′-end linkages are preferably phosphorothioate although when there is a single unit overhang precursor the terminal linkage can be phosphorothioate or selected from those provided for overhang precursors.
5) It is preferred that the 5 contiguous linkages from the 3′-end be phosphorothioate if the ss-RNAi is to be used in vivo without a protective carrier.

### 4. Modifications Underlying the Accommodating Helical Design (AHD):

Once the nuclease resistance rules have been applied to an ss-RNAi, the rules for AHD are applied and when there is a conflict the AHD rules take precedence. AHD rules, however, are optional for overhang precursors based on nucleosides and do not apply to non-nucleoside overhangs. AHD rules are optional for the two nucleosides in the most 5′-end position. Further, since the terminal 5′-end nucleoside of the strand is not involved in target recognition, it can have any base that does not produce steric hindrance problems with the RNAi mechanism.

Sugar and sugar analogs classified as most flexible include ribose, 2′-fluoro and 2′-0-methyl, flexible include HNA, FHNA, ANA, CeNA and F—CeNA, semi-flexible include CRN and CENA and rigid includes LNA. The most flexible are not accommodating modifications while the rest are.

The AHD rules for ss-siRNA and ss-IMiR are as follows unless otherwise specified:
1) Strands are preferred to not have more than 4 contiguous purines containing nucleosides outside of any overhang precursor. It is most preferred that they not have more than 2 contiguous purines.
2) The rules herein that provide for a 5′-end phosphate or phosphate mimics, isosteres or bioisosteres, the structure of the 5′-end nucleoside and the structure of the penultimate 5′-end nucleoside taken together can be used as a preferred option over not having a 5′-end phosphate or 5′-end phosphate mimic, isostere or bioisostere.
3) Any or all of the bases found in the ss-RNAi strand that are pyrimidine can be replaced, in a base by base manner, with another base with the same complementary base pairing preference (exclusive of wobble base pairing) where the base being substituted is an accommodating modification and where the sugar or sugar analog to which the base is conjugated preferably is among the most flexible (including ribose, 2′-fluoro, 2′-0-methyl) or flexible (including HNA, FHNA, ANA, CeNA and F—CeNA). Such accommodating bases include but are not limited to the group consisting of pseudouracil (U), 2-thiouracil (U), 4-thiouracil (C), 5-methyluracil (U) and 5-methylcytosine (C). The letter in parenthesis is the standard base that the accommodating base can replace.
4) It is preferred that strand areas that are comprised of five or more consecutive pyrimidines contain 1 or more accommodating modification(s).
5) LNA can be used as an accommodating inflexible sugar analog preferably outside of the targeting code but it is preferred that there be no more than 4 inflexible and semi-flexible sugar analogs and that there be at least one and most preferably at least two nucleosides between them. It is preferred that an LNA or a semi-flexible sugar analog be used in a series of at least three consecutive purine containing nucleosides.
6) CRN and CENA can be used as an accommodating semi-flexible sugar analog preferably outside of the targeting code but it is preferred that there be no more than 4 inflexible and semi-flexible sugar analogs and that there be at least one and most preferably at least two nucleosides between them. It is preferred that a CRN, CENA or an inflexible sugar analog be used in a series of at least three consecutive purine containing nucleosides.
7) Flexible accommodating sugars that are preferred to be used outside of the targeting code include but are not limited to HNA, FHNA, ANA, CeNA and F—CeNA but it is preferred that there be no more than a total of 6 inflexible, semi-flexible and flexible sugar analogs and that there be at least one and most preferably at least two nucleosides between them. It is preferred that a flexible sugar analog be used in a series of at least three consecutive purine containing nucleosides.
8) When a purine containing nucleoside with one of the most flexible sugars (ribose, 2′-fluoro and 2′-0-methyl) or a flexible sugar (HNA, FHNA, ANA, CeNA, and F—CeNA) is contiguous to a standard pyrimidine containing nucleoside with a flexible or most flexible sugar or sugar analogs then it is preferred that the standard pyrimidine be replaced with an accommodating base. Such bases include but are not limited to the group consisting of pseudouracil (U), 2-thiouracil (U), 4-thiouracil (C), 5-methyluracil (U) and 5-methylcytosine (C). The letter in parenthesis is the standard base that the replacement base can replace. Alternatively the contiguous nucleoside with a pyrimidine can have a rigid (such as LNA) or semi-flexible (such as CRN or sugar analog.
   No overhang precursor is required but overhang precursors with 1-4 units as provided for herein are preferred.

The AHD rules for ss-MiRs based on essentially complete endogenous miRNA guide strand sequences and are as follows unless otherwise specified:
1) The rules herein that provide for a 5′-end phosphate or phosphate mimics, isosteres or bioisosteres, the structure of the 5′-end nucleoside taken together can be used as a preferred option over not having a 5′-end phosphate or 5′-end phosphate mimic, isostere or bioisostere.
2) The sugar component of the nucleoside in the penultimate position from the 5′-end that is the first position in the targeting code is preferably 2′-fluoro or ribose.
3) Modifications can be made to the targeting code that increases the affinity between the ss-MiRs and its target(s) for the purpose of increasing activity. These modifications may or may not also be accommodating modifications but accommodating modifications are preferred. The modifications suitable for this purpose include those selected from the group consisting of 2′-fluoro, 2′-0-methyl, classic LNA, CRN, pseudouracil (replaces U), 2-thiouracil (replaces U), 4-thiouracil (replaces C), 5-methyluracil (replaces C) and 2,6-diaminopurine (replaces A). When CRN is used with the R monomer it is preferred that the X be oxygen and when the Q monomer is used it is preferred that X be oxygen, Y and Z be independently selected from the group O, S and CH₂ and R₂ is selected from hydrogen, hydroxyl or fluoro.
4) Any or all of the bases found in the ss-RNAi strand that are pyrimidine can be replaced, in a base by base manner, with another base with the same complementary base pairing preference (exclusive of wobble base pairing) where the base being substituted is an accommodating modification and where the sugar or sugar analog to which the base is conjugated preferably is among the most flexible (including ribose, 2′-fluoro, 2′-0-methyl) or flexible (including HNA, FHNA, ANA, CeNA and F—CeNA). Such accommodating bases include but are not limited to the group consisting of pseudouracil (U), 2-thiouracil (U), 4-thiouracil (C), 5-methyluracil (U) and 5-methylcytosine (C). The letter in parenthesis is the standard base that the accommodating base can replace.
5) It is preferred that strand areas that are comprised of five or more consecutive pyrimidines contain 1 or more accommodating modification(s).
6) LNA can be used as an accommodating inflexible sugar analog but it is preferred that there be no more than 3 inflexible and semi-flexible sugar analogs and that there be at least one and most preferably at least two nucleosides between them. It is preferred that an LNA or a semi-flexible sugar analog be used in a series of at least three consecutive purine containing nucleosides.
7) CRN and CENA can be used as an accommodating semi-flexible sugar analog but it is preferred that there be no more than 3 inflexible and semi-flexible sugar analogs and that there be at least one and most preferably at least two nucleosides between them. It is preferred that a CRN, CENA or an inflexible sugar analog be used in a series of at least three consecutive purine containing nucleosides. Further, CENA is not used in the targeting code.
8) Flexible accommodating sugars include but are not limited to HNA, FHNA, ANA, CeNA and F—CeNA but it is preferred that there be no more than a total of 6 inflexible, semi-flexible and flexible sugar analogs and that there be at least one and most preferably at least two nucleosides between them. It is preferred that a flexible sugar analog be used in a series of at least three consecutive purine containing nucleosides.
9) When a purine containing nucleoside with one of the most flexible sugars (ribose, 2′-fluoro and 2′-0-methyl) or a flexible sugar is contiguous to a standard pyrimidine containing nucleoside with a flexible or most flexible sugar or sugar analogs then it is preferred that the standard pyrimidine be replaced with an accommodating base. Such bases include but are not limited to the group consisting of pseudouracil (U), 2-thiouracil (U), 4-thiouracil (C), 5-methyluracil (U) and 5-methylcytosine (C). The letter in parenthesis is the standard base that the replacement base can replace. Alternatively the contiguous nucleoside with a pyrimidine can have a rigid (such as LNA) or semi-flexible (such as CRN or sugar analog.
10) No overhang precursor is required but overhang precursors with 1-4 units as provided for herein are preferred.
   5. Phosphate Mimics, Isosteres or Bioisosteres for Use in ss-RNAi:

Ss-RNAi of the present invention can be manufactured with a hydroxyl on the 5′ carbon of the 5′-end ribose or the 5′ carbon of certain modified ribose sugars or on the corresponding carbon in certain sugar substitutes. This 5′-carbon is illustrated in FIG. 13 with a ribose sugar. The ss-RNAi also can be manufactured phosphorylated or with a phosphate isostere or bioisostere (the terms phosphate mimic, phosphate isostere and phosphate bioisostere are used interchangeably) at these positions. Numerous phosphate bioisosteres are known in the literature. Depending on the cell/tissue type and species, the addition of a phosphate or phosphate bioisostere can improve the intended silencing activity but the degree to which this can happen, in part, is a function of how well the strand accommodates RISC and/or RISC loading components. AHD can have the effect, therefore, of reducing the importance of having a phosphate or phosphate isostere at the 5′-end of an ss-RNAi. The nature of the dephosphorylase activity in the target cells that are capable of removing phosphate or certain phosphate isoteres added to the ss-RNAi can also be of importance to achieving activity against the intended target.

Some of the chemical moieties suitable for conjugation to the 5′ carbon of the 5′-end ribose or modified ribose or the corresponding carbon in a sugar analog provided for herein can be selected from the group consisting of a hydroxyl group, a phosphate group, 5′-monophosphate [(HO)₂(O)P—O-5′], 5′-diphosphate[(HO)₂(O)P—O—P(HO)(O)—O-5′], 5′-triphosphate[(HO)₂(O)P—O—(HO)(O)P—O—P(HO)(O)—O-5′], 5′-guanosine cap (7-methylated or not methylated) [7m-G-O-5′-(HO)(O)P—O—(HO)(O)P—O—P(HO)(O)—O-5′], 5′-adenosine cap (Appp), any modified or unmodified nucleoside cap structure [N—O-5′-(HO)(O)P—O—(HO)(O)P—O—P(HO)(O)—O-5′], 5′-monothiophosphate (phosphorothioate) (HO)₂(S)P—O—5′, 5′-monodithiophosphate (phosphorodithioate) (HO)(HS)(S)P—O-5′, 5′-phosphorothiolate[(HO)₂(O)P—S-5′]; any additional combination of oxygen/sulfur replaced by monophosphate, diphosphate and triphosphates (e.g., 5′-α-thiotriphosphate, 5′-γ-thiotriphosphate, etc.), 5′-phosphoramidates[(HO)₂(O)P—NH-5′, (HO)(NH₂)(O)P—O-5′], 5′alkylphosphonates; R=alkyl=methyl, ethyl, isopropyl, propyl, etc., e.g., [RP(OH)(O)—O-5′-, (OH)₂(O)P-5′-CH₂—], 5′alkyletherphosphonates; (R=alkylether=methyoxymethyl (MeOCH₂—), ethoxymethyl, etc., e.g., RP(OH)(O)—O-5′, (WO 2004/007718) 5′methylenephosphonate (5′-MP) and 5′-(E)-vinylphosphonate (5′-VP). The 5′-end ribose also can be modified with a 5′,3′ diphosphate.

5′-VP is illustrated in FIG. 16. It can be used with a variety of nucleoside chemistries provided herein but in the prior art it is conjugated to a 2′-MOE thymine containing terminal 5′-end nucleoside (Lima et al., Cell 150: 883-894, 2012, WO 2011/139699 and WO 2011/139702).

The 5′ carbon of a ribose or ribose modified with certain 2′ modifications, such as 2′-fluoro, with a hydroxyl group in this position can also be phosphorylated in cells; for example, CIP1 has the ability to phosphorylate single strand RNA at this site. The ability of phosphorylases, such as CIP1, to recognize particular modified 5′-end nucleosides as targets for 5′-end phosphorylation at the 5′-carbon can be more restrictive in cells than the ability of phosphatases to recognize a variety of 5′-end nucleoside modifications for removal of any 5′-carbon phosphate or phosphate isosteres in this position. As a result some 5′-end nucleoside sugar modifications can inhibit phosphorylation of this site in cells while not inhibiting the action of phosphatases or similar enzymes in cells to remove any phosphate or phosphate isosteres in this position. This can occur when the bond between one of these moieties and the 5′ carbon is cleavable by the phosphatase or other enzyme capable of removing one of these moieties. Thus, the net effect in cells can be loss of a phosphate or phosphate isosteres added to an ss-RNAi during manufacturing due to the action of a dephosphorylating enzyme coupled with the relative inability of a cellular phosphorylase to add a phosphate to the site. The net result can be a reduction in the activity of the ss-RNAi

Not to be bound by theory, one solution to this dilemma is to use a phosphate isostere that is conjugated to the 5′ carbon by a bond that cannot be readily cleaved. Two examples of phosphate isosteres with resistant linkages are 5′-MP and 5′-VP. 5′-MP is less similar to natural phosphate than 5′-VP and is less active in supporting ss-RNAi activity. Depending on the ss-RNAi composition of matter a 5′-VP containing ss-RNAi at best can have comparable activity compared to the same ss-RNAi but with natural phosphate but it can also be less active than an ss-RNAi that retains its natural phosphate in cells. 2′-MOE is an example of a modification to the 5′-end nucleoside of a ss-RNAi that is poorly recognized by enzymes such as CIP1 that can add phosphate to a 5′carbon of a 5′-end nucleoside that more closely resembles native ribose such as 2′-fluoro. 2′-MOE, however, is an example of a 5′-end ss-RNAi modification that can be recongnized by dephosphorylating enzymes capable of removing phosphate isostere recognizable by the phosphatase and with a cleavable bond from a 5′ carbon of an oligoribonucleotide. 2′-MOE is, therefore, well suited for use with the 5′-VP phosphate mimic.

Another solution is to manufacture an ss-RNA with a 5′-end phosphate or phosphate isosteres conjugated to a 5′end nucleoside with cleavable bonds where the 5′end terminal nucleoside(s) are poorly recognized by the dephosphorylating enzymes present in the target cells. The net result being that the manufactured 5′-end phosphate or phosphate isostere is retained long enough for the ss-RNAi to be associated with a component of the RNAi mechanism, such as a argonaute protein, that can protect it from enzymatic attack.

Ss-RNAi of the present invention optionally can be manufactured with the 5′ carbon position conjugated to a phosphate or a phosphate isostere where the 5′-end and/or penultimate 5′-end nucleoside inhibits any dephosphorylation activity in cells otherwise capable of removing the phosphate or phosphate isostere. Nucleoside modifications(s) poorly recognized by enzymes that can cause this type of dephosphorylation can be selected from the group consisting of CeNA, F—CeNA, CENA, AENA, LNA or the CRN modification with either the R or Q monomer shown in FIG. 9. UNA can also be used in the terminal but not the penultimate 5′-end position. CENA, AENA, CeNA and 2′-O-methyl are not used in the penultimate 5′ nucleoside position in ss-MiRs. In the case of the CeNA and F—CeNA six membered rings the carbon corresponding to the 5′ carbon in ribose is position 5 of the 6 membered ring where the base “B” shown in FIG. 12 projects from carbon in position 1 with the count occurring clockwise for both CeNA and F—CeNA. When the CRN Q monomer is selected R₂ is selected from the group consisting of H, F and OH.

Unless otherwise specified the rules for generating an ss-siRNA or ss-IMiR when a phosphate or phosphate isostere is to be used at the 5′-end are as follows:
1) A phosphate or phosphate isostere selected from those provided herein is conjugated to the 5′ carbon of the sugar or sugar substitute of the 5′-terminal nucleoside.
2) The sugar or sugar substitute for the 5′ end terminal nucleoside is selected from the group consisting of 2′-fluoro, 2′-O-methyl, UNA, HM, EA, CeNA, F—CeNA, CENA, AENA, LNA and CRN with either the R or Q monomer. When the CRN Q monomer is selected R₁ is the phosphate or phosphate isostere and R₂ is selected from the group consisting of H, F and OH. When the CRN R monomer is selected R₃ is the phosphate or phosphate isostere. When LNA is used it can be any of the variants shown in FIG. 10 (classic, thio, alpha or amino) and the base can be any of those provided for herein (standard or accommodating) including T. 2′-MOE or EA can also be used as the5′-end terminal sugar. In these cases a phosphate isostere with a linkage to the 5′ carbon is resistant to attack by cellular dephosphoryating enzymes when the ss-RNAi is to be used in vivo is preferably used with EA and more preferably used with 2′-MOE. These isosteres with dephosphorylase resistant linkages include 5′-MP and 5′-VP.
3) The sugar or sugar substitute for the penultimate 5′ end terminal nucleoside is selected from the group consisting of 2′-fluoro, 2′-O-methyl, HM, CeNA, F—CeNA, CENA, AENA, LNA and CRN with either the R or Q monomer. When the CRN Q monomer is selected R₂ is selected from the group consisting of H, F and OH.
4) When the ss-RNAi compound is to be used in vivo without at protective carrier the linages between the terminal three nucleosides are preferably phosphorothioate.

Unless otherwise specified the rules for generating an ss-MiR when a phosphate or phosphate isostere is to be used at the 5′-end are as follows:
1) A phosphate or phosphate isostere selected from those provided herein is conjugated to the 5′ carbon of the sugar or sugar substitute of the 5′-terminal nucleoside.
2) The sugar or sugar substitute for the 5′ end terminal nucleoside is selected from the group consisting of 2′-fluoro, 2′-O-methyl, HM, UNA, CeNA, F—CeNA, CENA, AENA, LNA and CRN with either the R or Q monomer. When the CRN Q monomer is selected R₁ is the phosphate or phosphate isostere and R₂ is selected from the group consisting of H, F and OH. When the CRN R monomer is selected R₃ is the phosphate or phosphate isostere. When LNA is used it can be any of the variants shown in FIG. 10 (classic, thio, alpha or amino).). 2′-MOE or EA can also be used as the 5′-end terminal sugar. In these cases a phosphate isostere with a linkage to the 5′ carbon is resistant to attack by cellular dephosphoryating enzymes when the ss-RNAi is to be used in vivo is preferably used with EA and more preferably used with 2′-MOE. These isosteres with dephosphorylase resistant linkages include 5′-MP and 5′-VP.
3) The sugar or sugar substitute for the penultimate 5′ end terminal nucleoside is selected from the group consisting of 2′-fluoro, HM, F—CeNA, and CRN with either the R or Q monomer. When the CRN Q monomer is selected R₂ is selected from the group consisting of H, F and OH while Y and Z are preferably CH.
4) When the ss-RNAi compound is to be used in vivo without at protective carrier the linages between the terminal three nucleosides are preferably phosphorothioate.
   6. Modular Design Approach for ss-MiRs—Overview:

Ss-MiRs can be constructed using the entire guide strand sequence for the miRNA to be mimicked or a modular design approach can be used. When the modular design is used there are 2 or 3 modules consisting of the 5′-end terminal 9 nucleosides as found in the guide strand of the miRNA to be mimicked, the seed vehicle and the optional overhang precursor. Depending on the miRNA nucleosides 2-7 or 2-8 make up the seed sequences that directly engages the target sequence. The 9^{th} nucleoside from the 5′-end, while not formerly part of the seed sequence, can sometimes influence target binding by the seed sequence. For this reason it is included in the modular design. The terminal 5′-end nucleoside in a miRNA guide strand similarly is not part of the seed sequence and it is not thought to effect the binding of the seed sequence to its target because when the guide strand is bound to an argonaute protein this nucleoside is not available for binding to a complimentary target. Thus, any of the standard bases provided herein can be used in this nucleoside, but in general the base appearing in the miRNA guide strand is used in this position.

7. Modular Design Approach for ss-MiRs—9 Nucleoside 5′-End Module:

MiRbase provides the sequences for the 5′-end 9 nucleosides for endogenous human miRNAs that will be used to generate ss-MiR mimics of a particular naturally occurring miRNA. These sequences can be modified in accordance with the following: (1) nuclease resistance rules; (2) AHD rules; (3) rules for increasing binding of the seed sequence to its targets to boost activity; and (4) the rules for adding phosphate or a phosphate isostere to the 5′ carbon of the 5′-terminal nucleoside sugar or sugar substitute.

8. Modular Design Approach for ss-MiRs—Seed Vehicle Module:

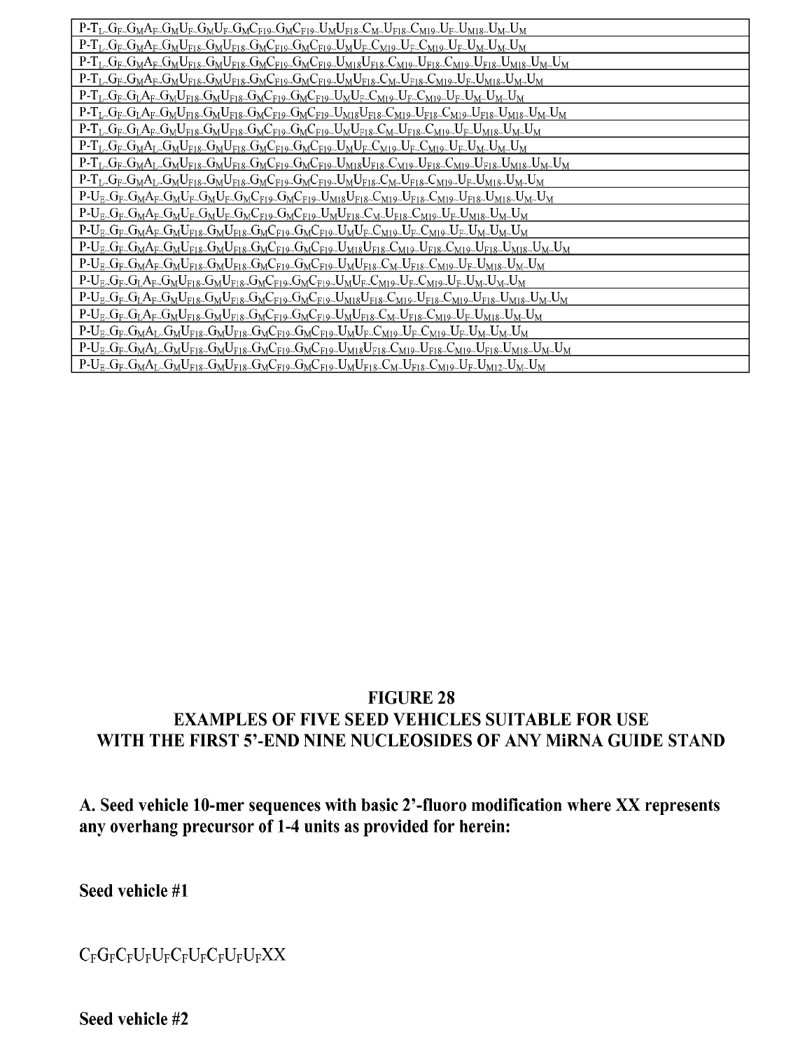

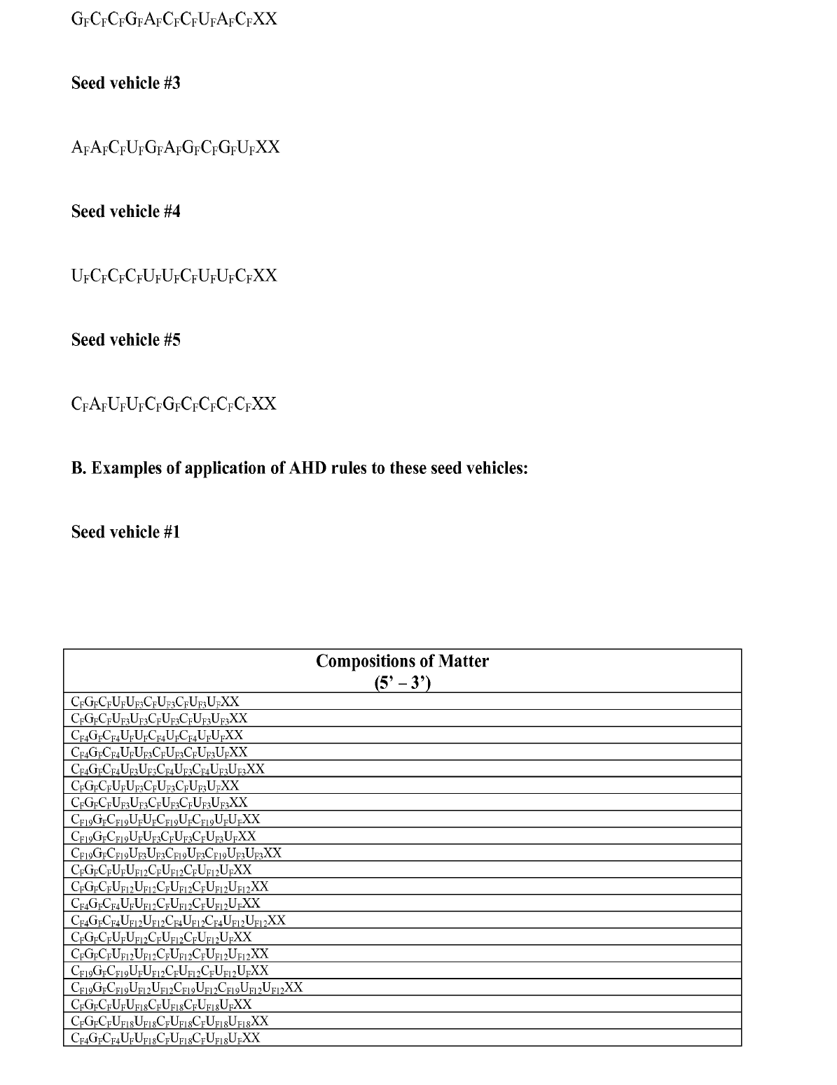

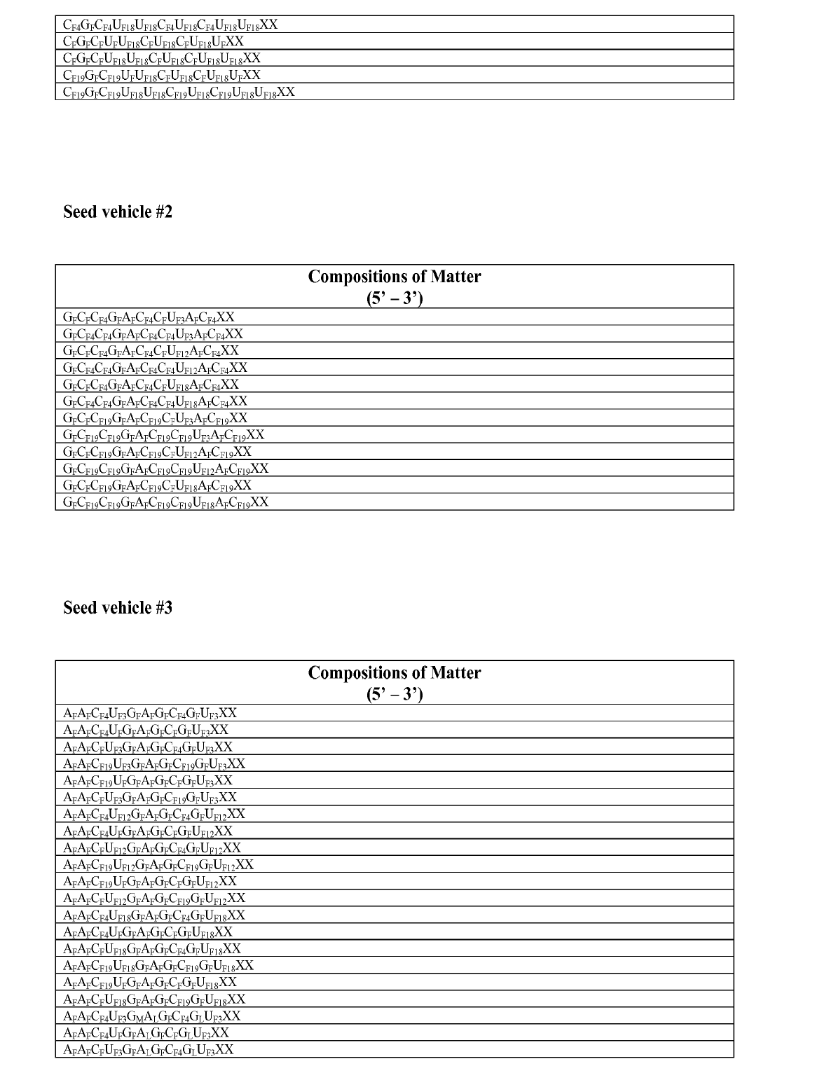

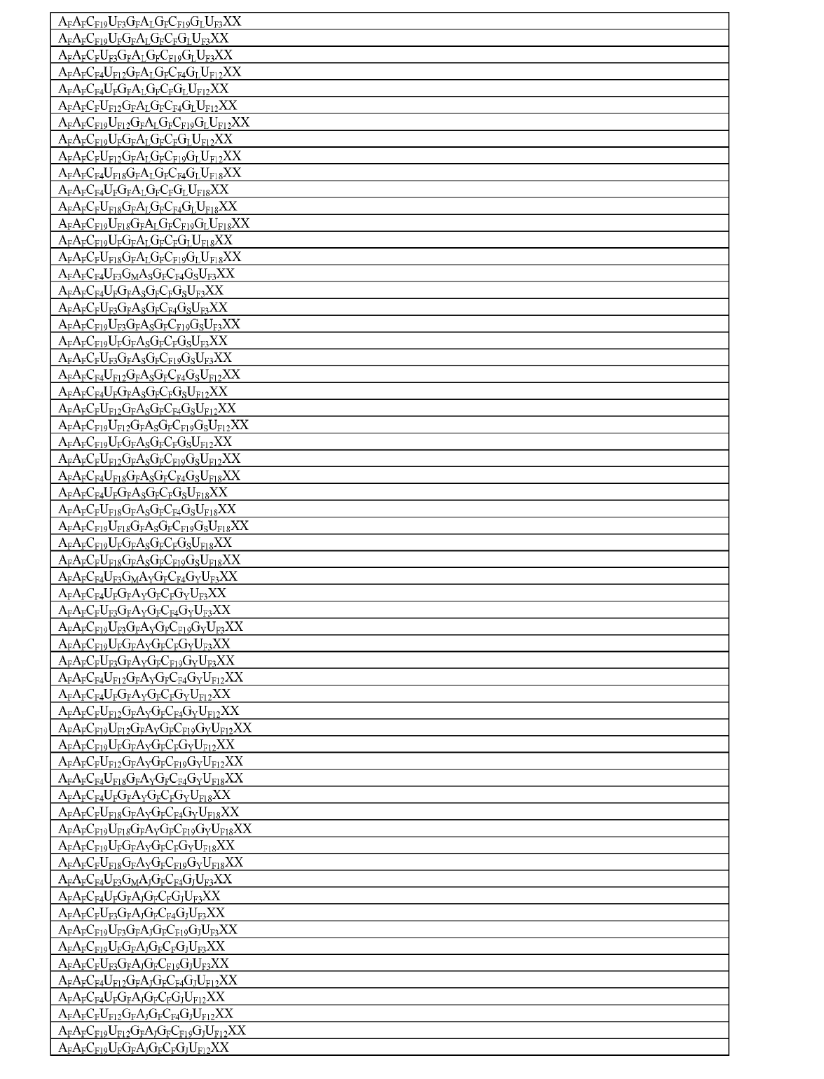

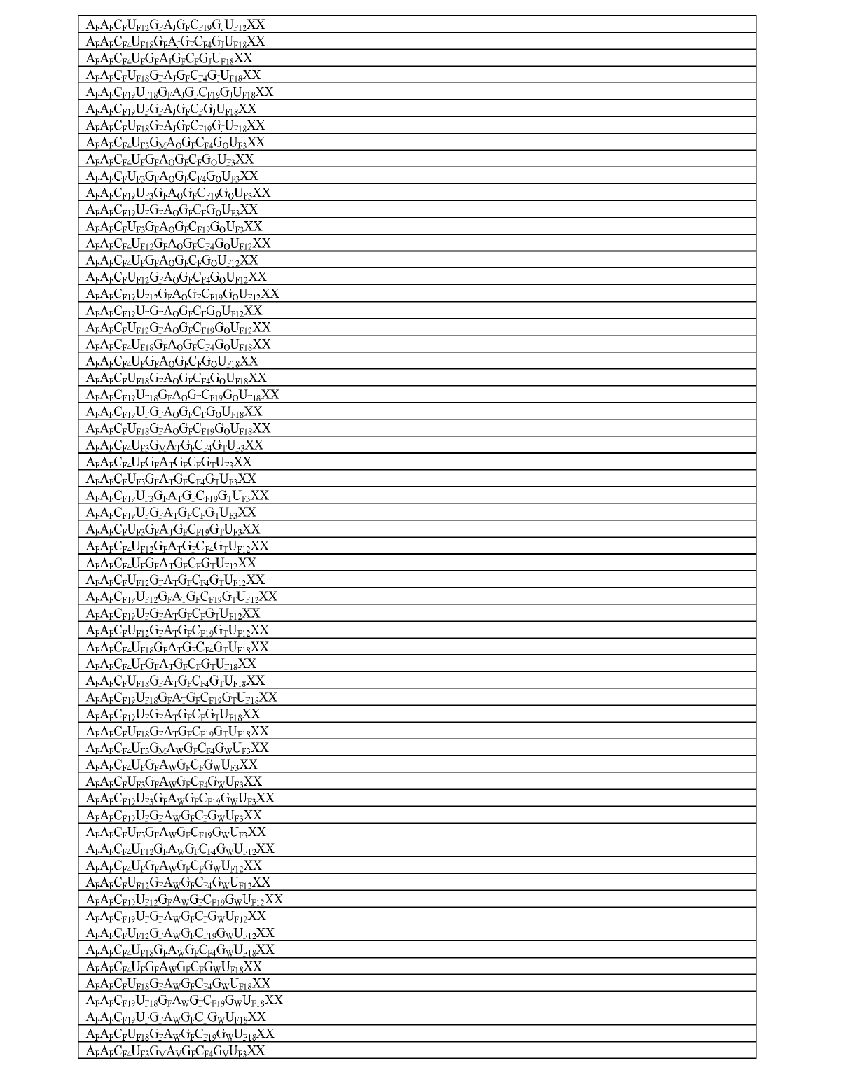

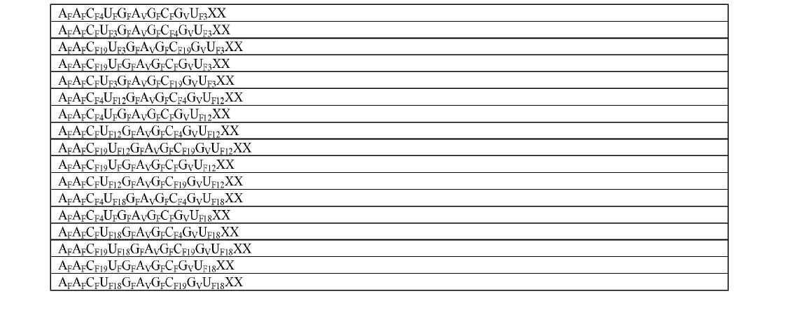

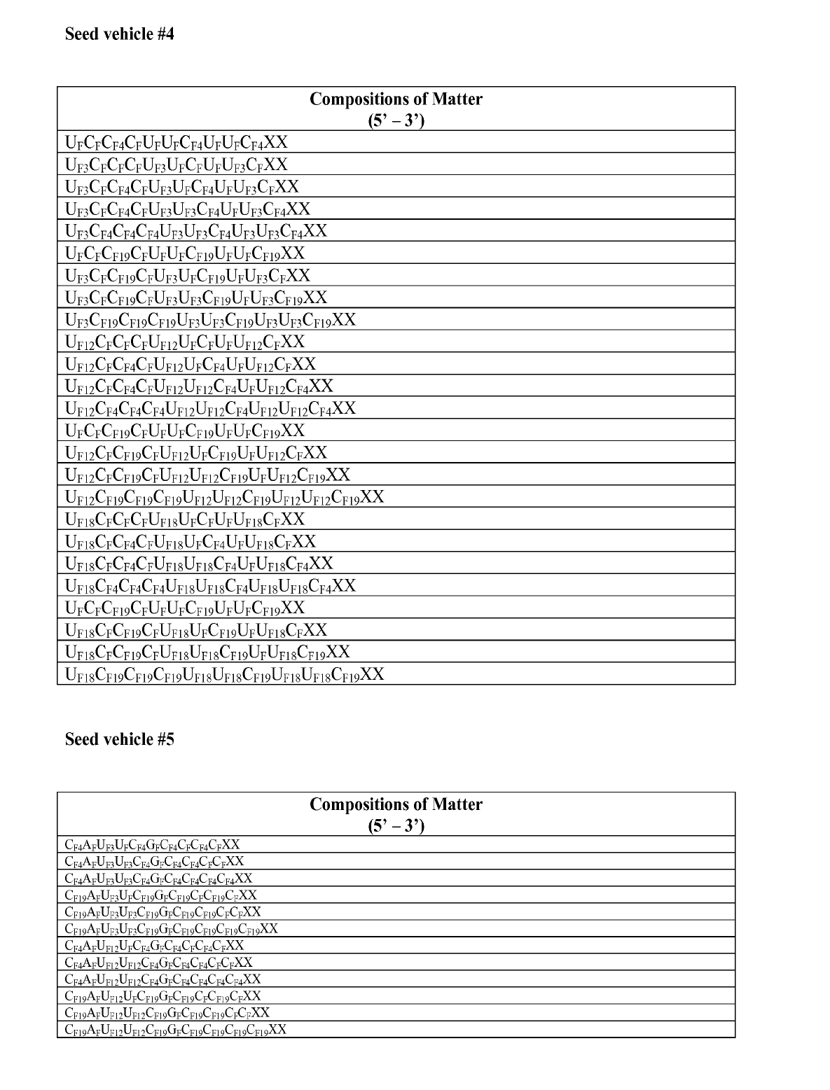

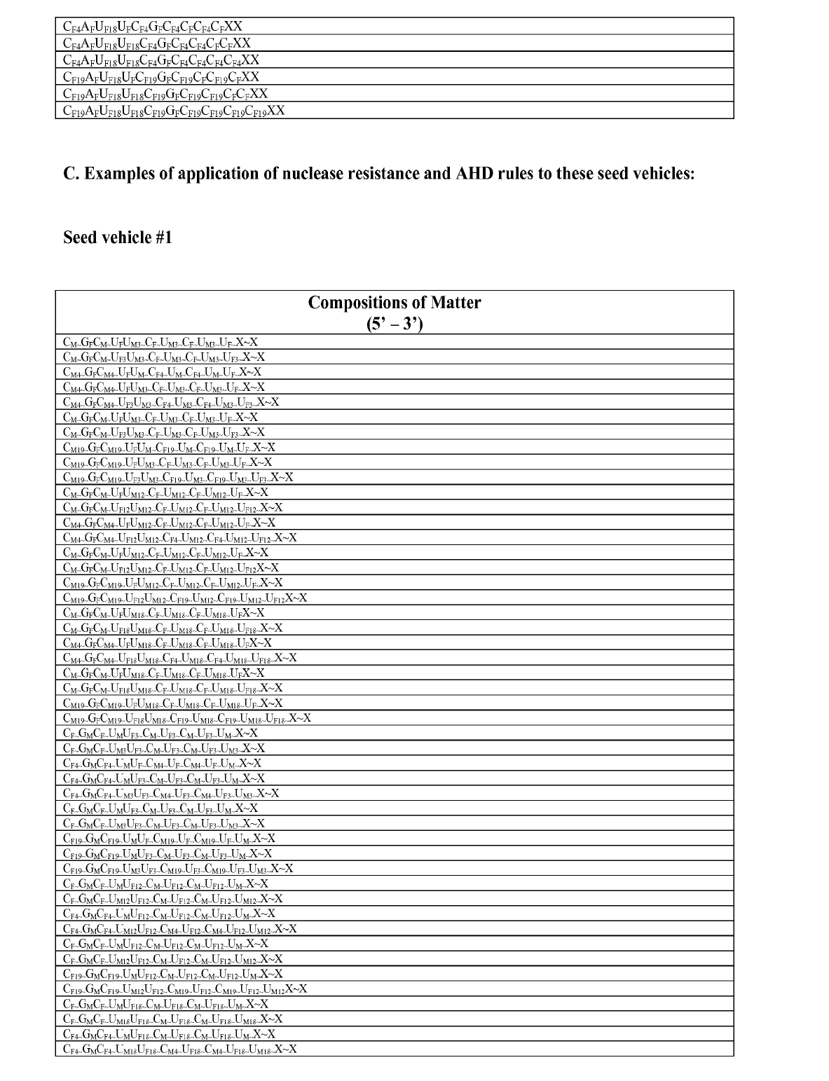

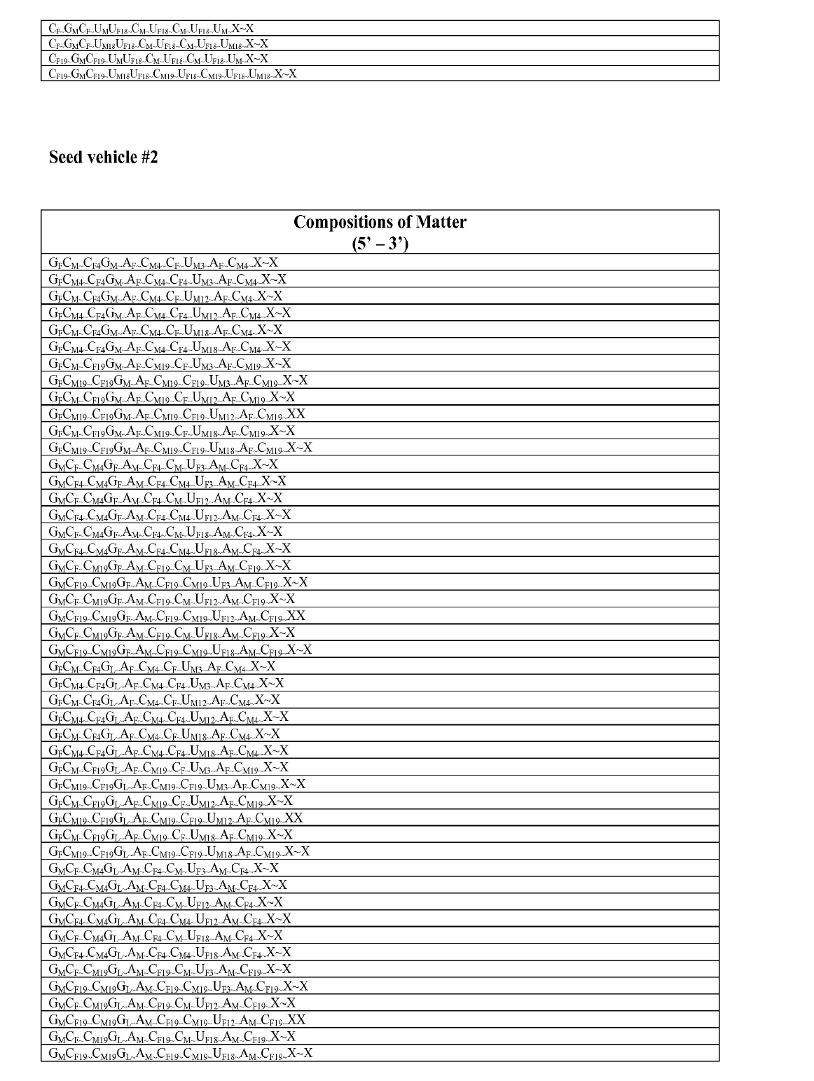

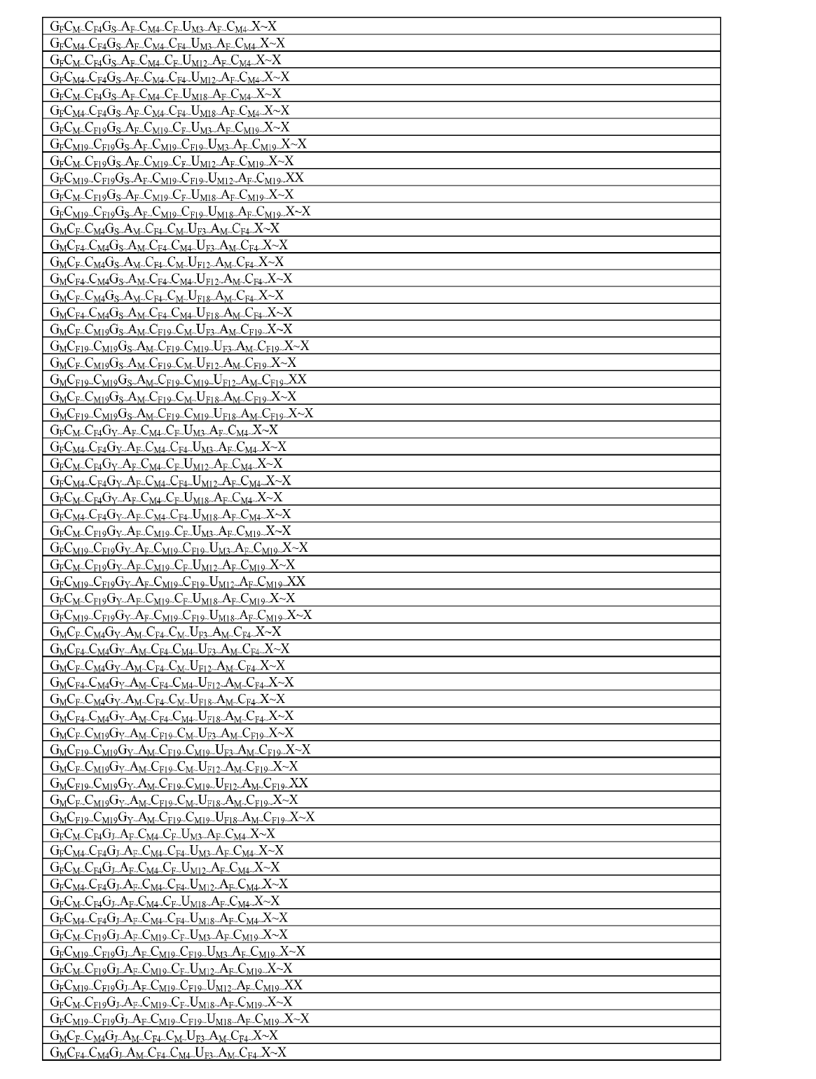

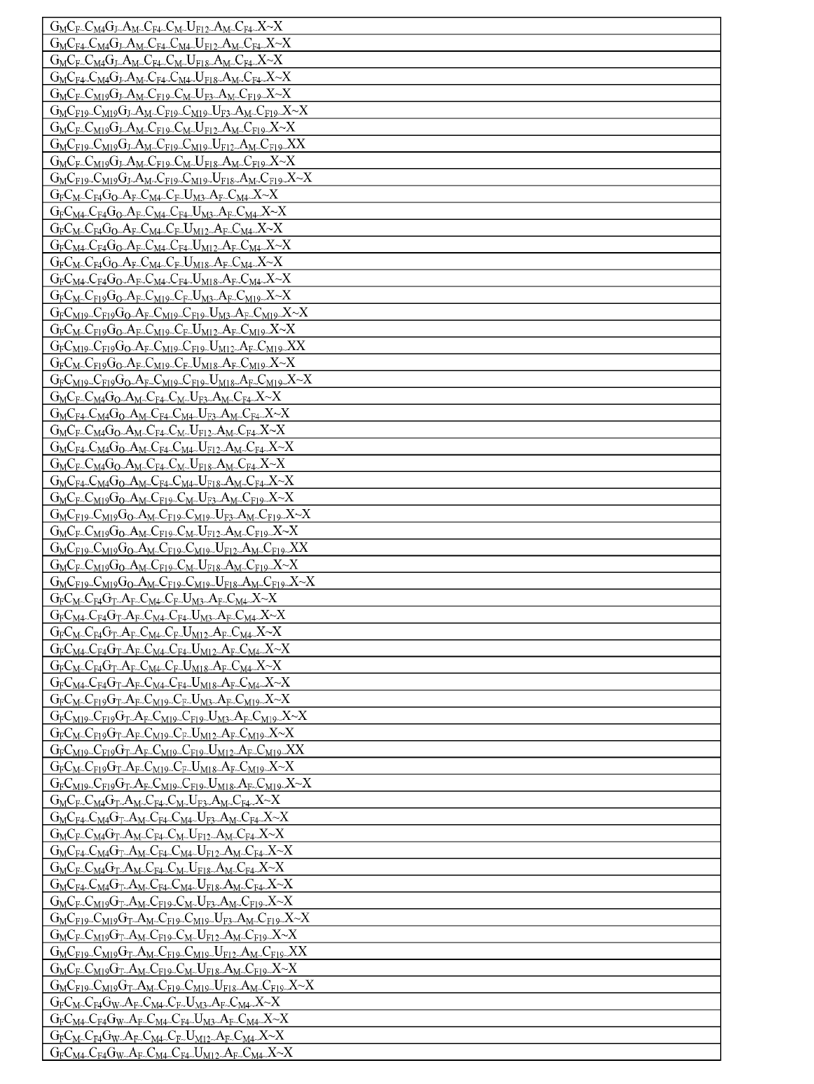

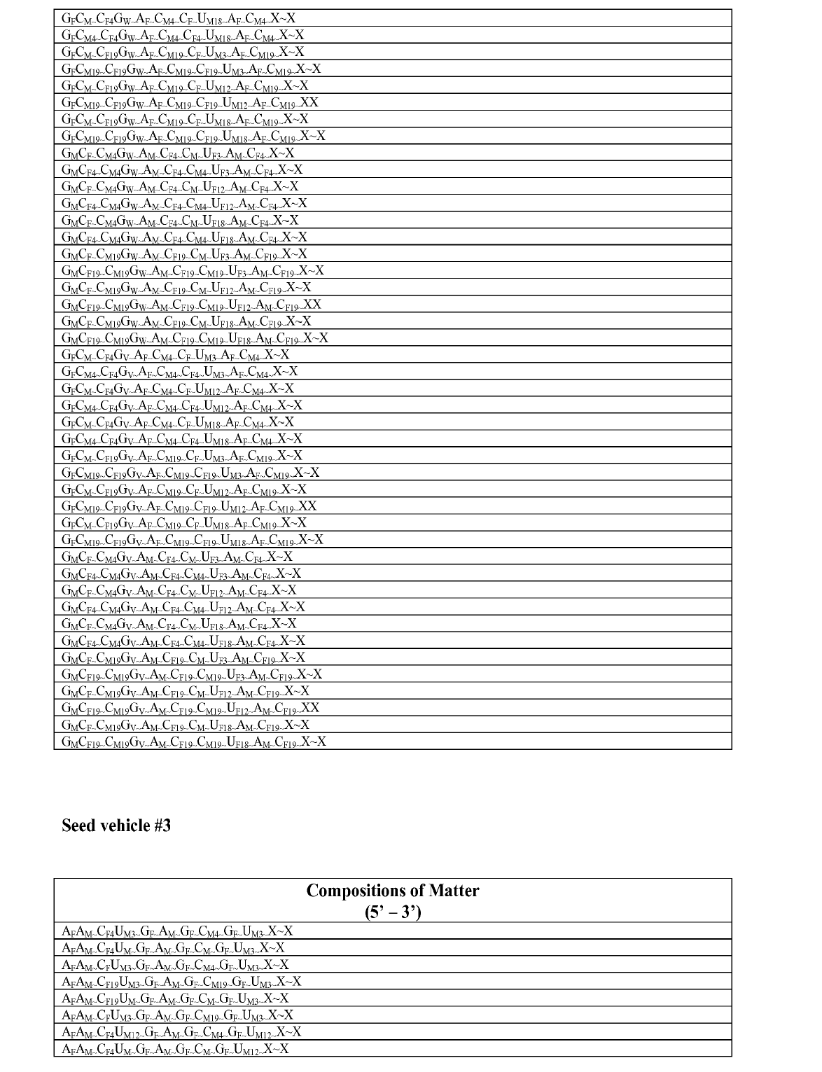

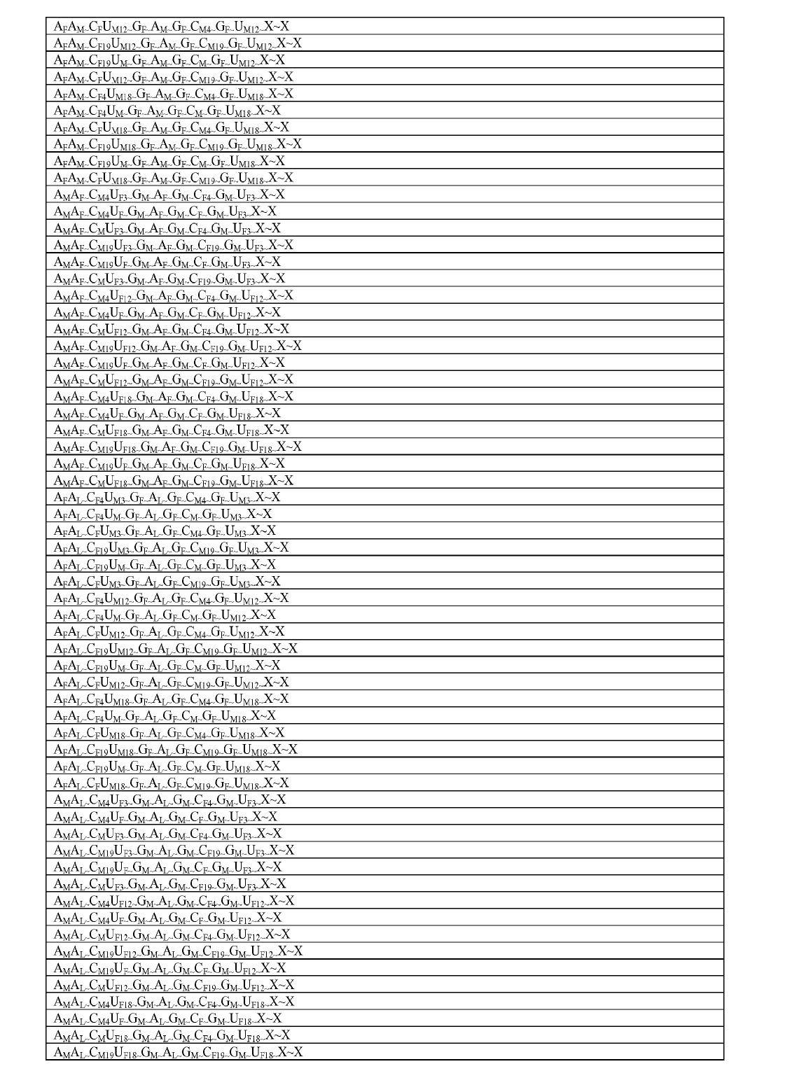

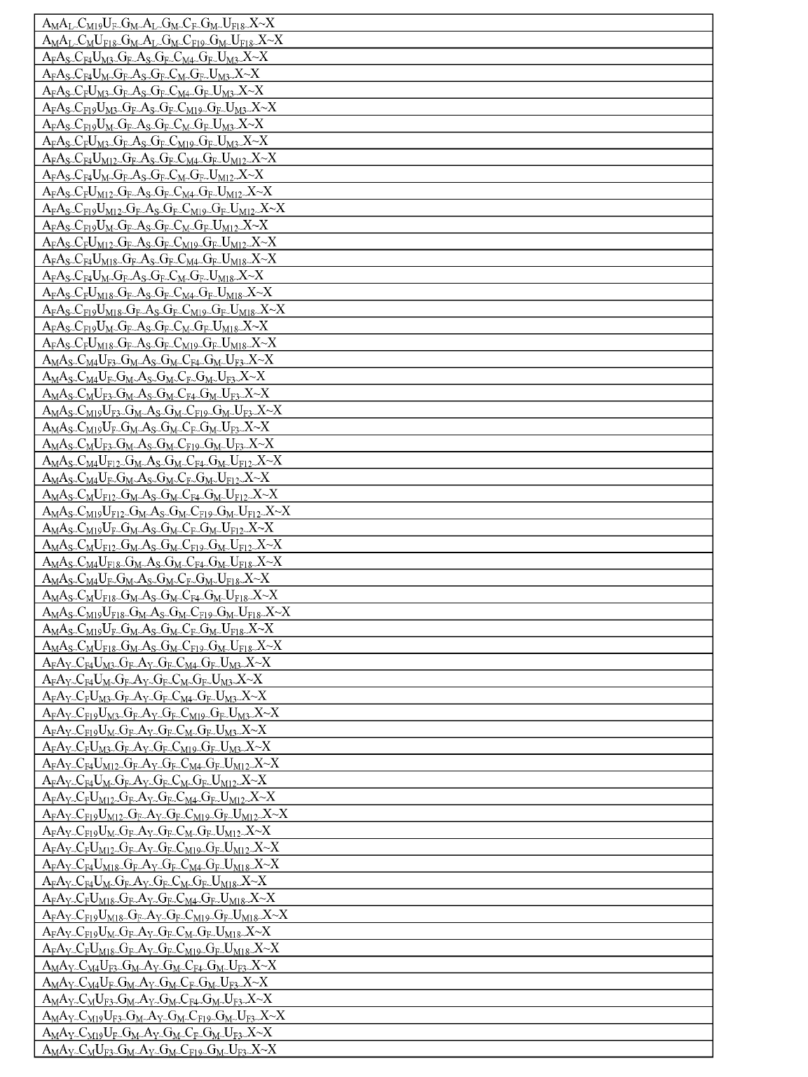

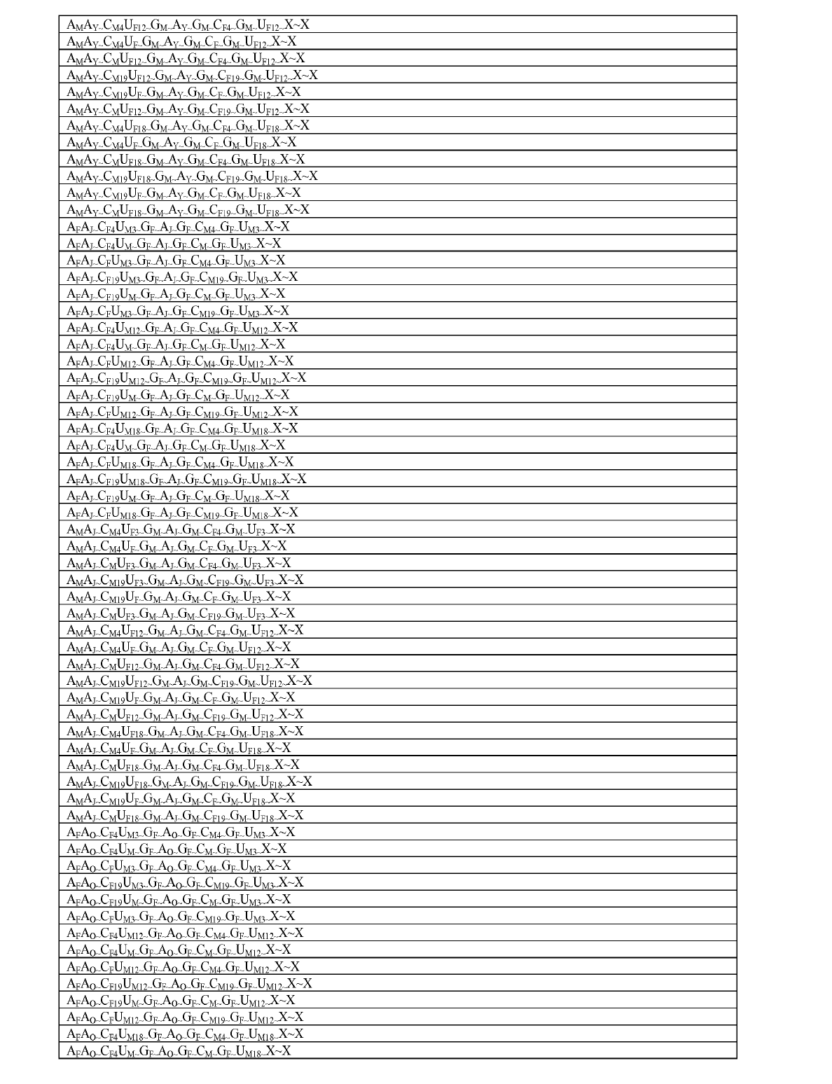

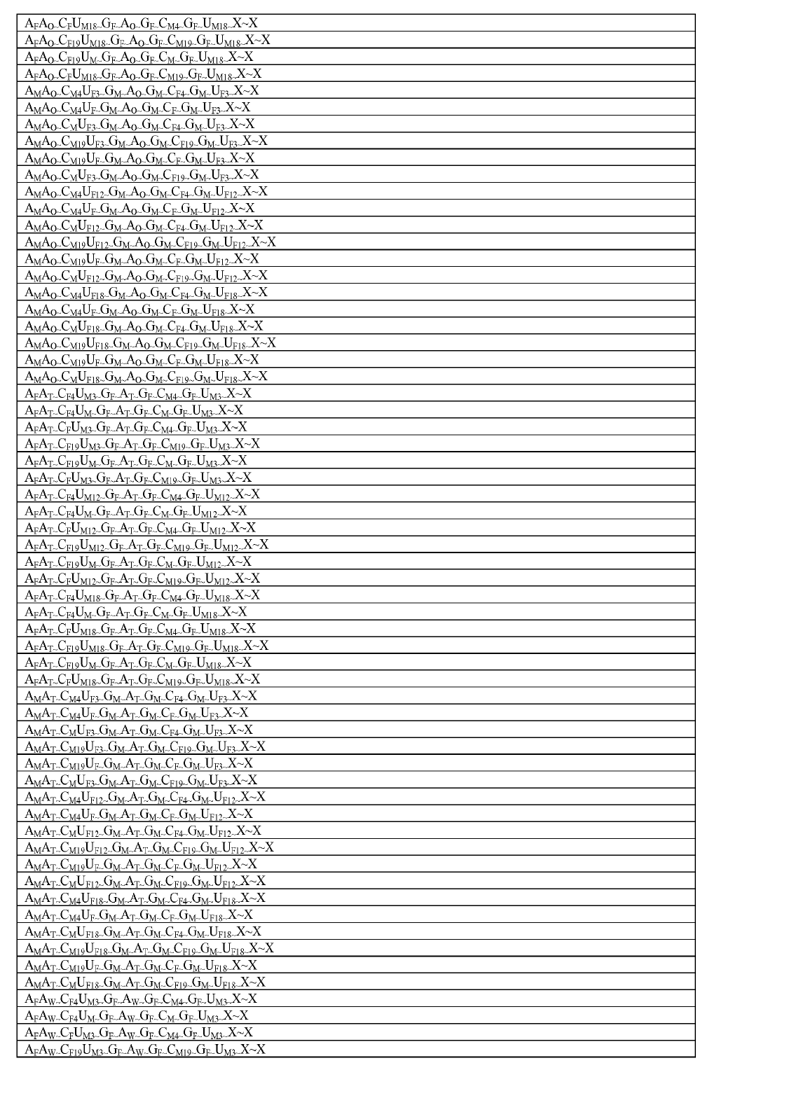

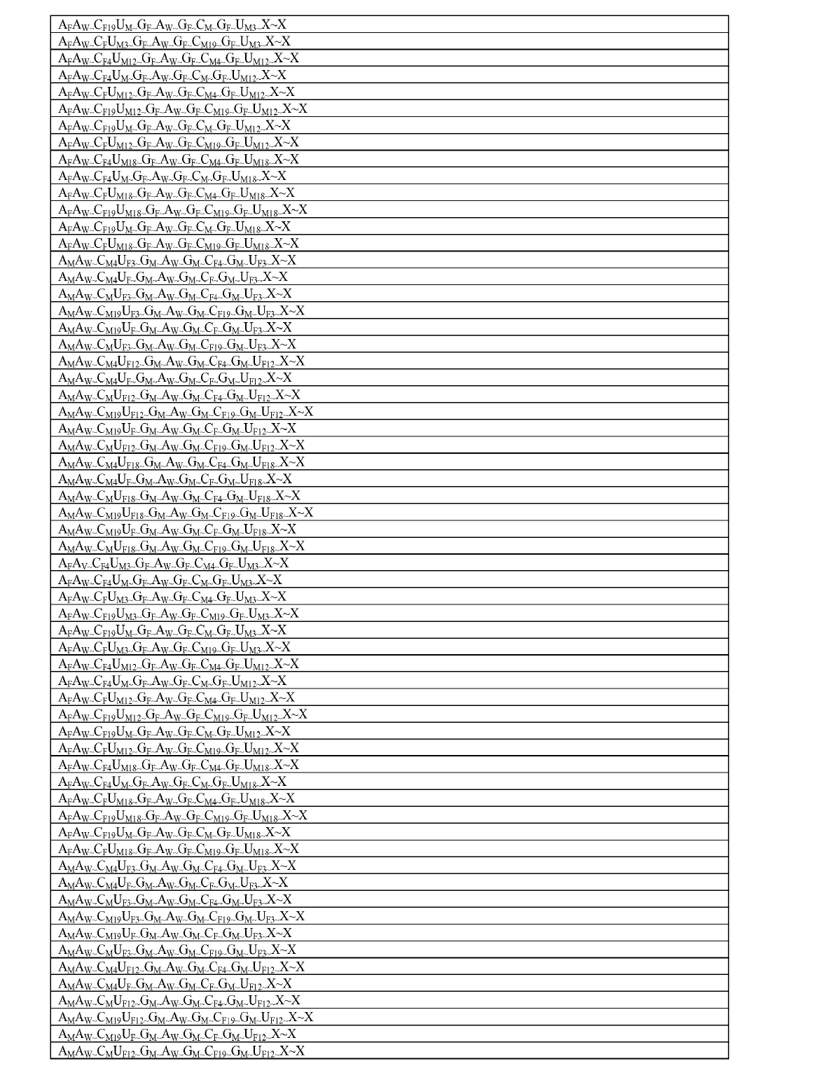

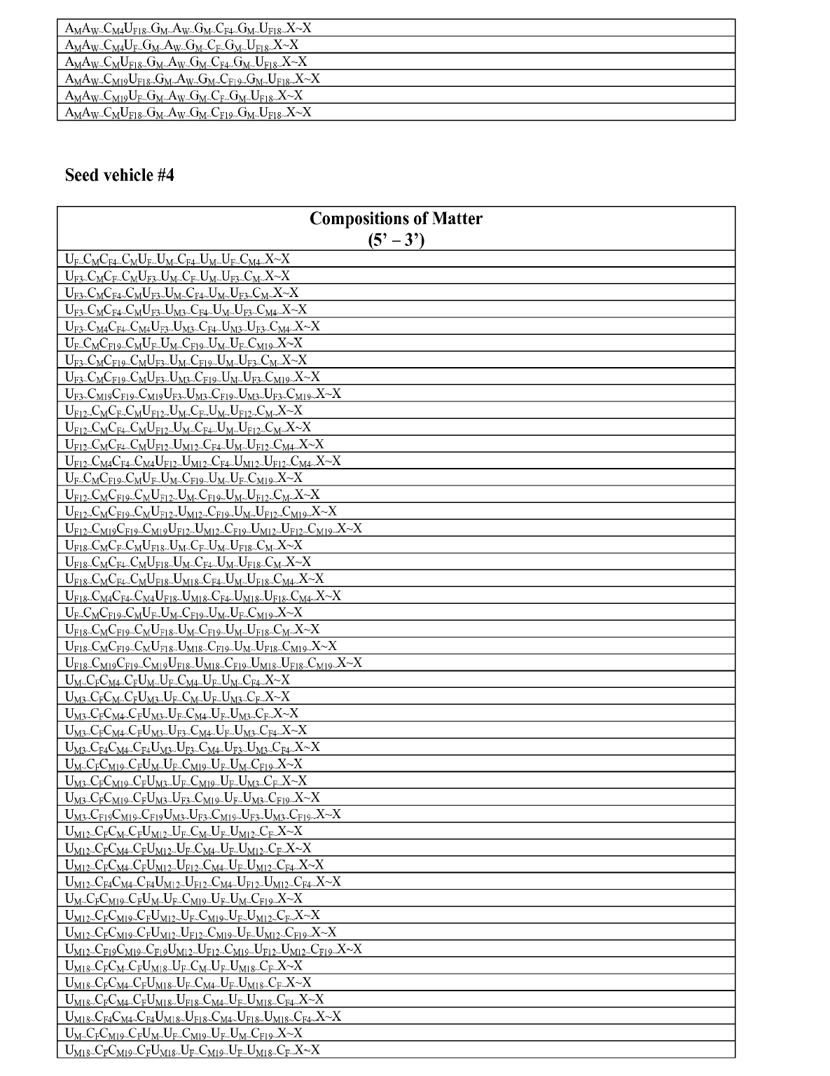

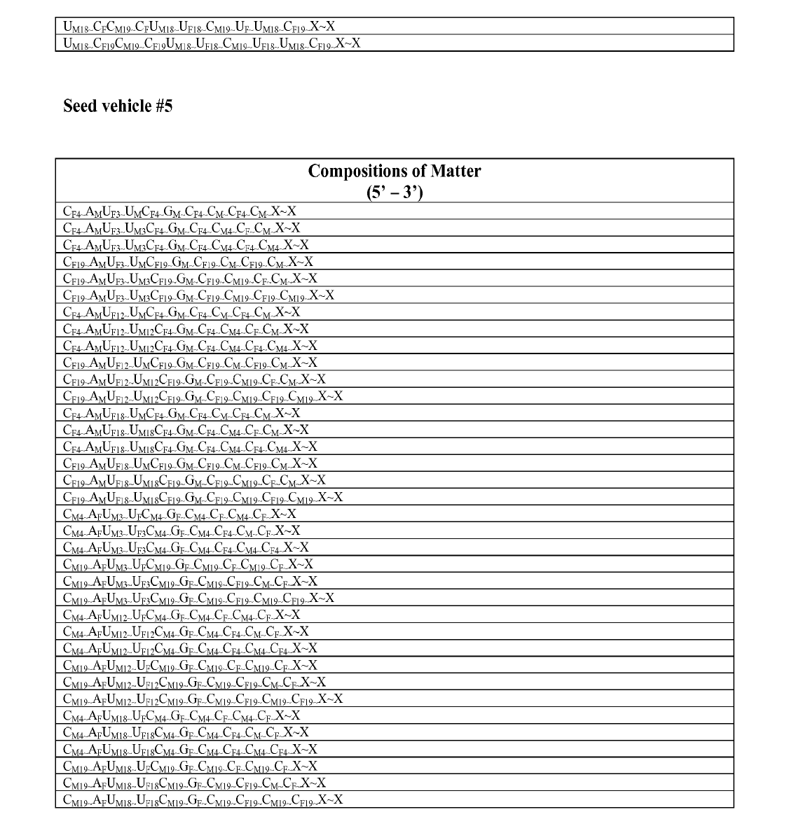

Ss-RNAi strands are preferably 16-22 nucleosides in length exclusive of any overhang precursor; therefore, the seed vehicle portion of an ss-MiR will be 8-14 nucleosides in length. Table 2 provides examples of 11-mer root (unmodified) seed vehicle sequences while FIG. 28 provides examples of unmodified and fully modified 10-mer seed vehicles. Any given seed vehicle can be paired with any 9 nucleoside 5′end module.

Basic nuclease resistance is provided to seed vehicles by the same rules that apply to endonuclease protection for AHD ss-RNAi strands generally. In addition the rules found in the section “Modifications underlying the accommodating helical design (AHD)” are applied to seed vehicles. Table 2 provides examples where root seed vehicle sequences taken from Table 1 are modified in accordance with these rules. The base associated with the 3′-end nucleoside of a specific 9-nucleoside long 5′-end sequence and the 5′-end nucleoside found in a specific seed vehicle will from a new linkage site that will be modified according to the applicable rules.

Table 2 provides a series of 11-mer root seed vehicle sequences where the 5′-end nucleoside in a commonly less nuclease resistant linkage site is indicated by a subscript X or Y to the right of the letter indicating the base found in the nucleoside. Two contiguous less resistant linkage sites can both be protected by one LNA or CRN and thus count as one less resistant linkage site. A “Y” rather than an “X” indicates such sites. The sequences shown in Table 1 are samples and are not an exhaustive list of those that meet the stipulated criteria. These sample sequences can be modified according to the rules provided. Examples, of modified versions of these sequences are provided in Table 2. The exemplary sequences have been modified on the basis of the nuclease resistance and the accommodating helical design rules. The examples provided in Table 2 are not exhaustive. The seed vehicle selected for use in a particular ss-MiR should be one that does not result in self-interaction by the strand in the form of self-dimer or hairpin formation under physiological conditions.

TABLE 1
SAMPLE 11-mer ROOT SEED VEHICLE SEQUENCES

| Root | | |
|---|---|---|
| Sequence | Sequence* | Seq ID |
| No. | 5′-3′ | No. |
| 1 | GUUₓCUUUₓCUUU | |
| 2 | AGCUUₓCUUₓCUU | |
| 3 | GGUₓCUU_{Y}A_{Y}UUUₓG | |
| 4 | GGUₓGUU_{Y}C_{Y}A_{Y}UUU | |
| 5 | UUₓCUUUₓCUUUU | |
| 6 | GCₓGCUUₓCUₓCUU | |
| 7 | UUUUUUₓCUₓCUU | |
| 8 | GUUUUₓCUUUₓCU | |
| 9 | GUUₓCUₓCUUₓCUₓC | |
| 10 | CUUUUₓGUUₓCUₓC | |
| 11 | CUUₓCUUₓCUUUₓC | |
| 12 | GUUₓCUUₓGUUUₓC | |
| 13 | CₓGGUUUₓCₓGUUU | |
| 14 | GCₓGCₓCUUUₓCUU | |
| 15 | GU_{Y}C_{Y}CUUₓGCUUₓC | |
| 16 | CUU_{Y}C_{Y}CUUₓCUUₓC | |
| 17 | GCₓCUₓCUUUU_{Y}C_{Y}C | |
| 18 | GCₓGUU_{Y}C_{Y}CUUₓCU | |
| 19 | GGGU_{Y}C_{Y}GUUUUU | |
| 20 | GCₓGC_{Y}C_{Y}GA_{Y}UUUU | |
| 21 | GCU_{Y}C_{Y}C_{Y}CUUUₓCU | |
| 22 | AGUₓCUUUUₓCUU | |
| 23 | AGGCUUUₓCUUU | |
| 24 | GCₓGCUₓCUₓCUUₓC | |
| 25 | GUUU_{Y}C_{Y}GUU_{Y}C_{Y}GA | |
| 26 | GUUₓGCₓGUUₓCUₓA | |
| 27 | GUUₓCU_{Y}C_{Y}GUₓCUₓC | |
| 28 | GGC_{Y}C_{Y}GA_{Y}C_{Y}CU_{Y}A_{Y}C | |
| 29 | GU_{Y}C_{Y}C_{Y}CUUₓCUUₓC | |
| 30 | GC_{Y}C_{Y}CU_{Y}C_{Y}C_{Y}CUₓGA | |

* A subscript “X” follows the 5′ nucleo-
side in one of the 8 commonly less resistant
linkage site unless two or three such linkage
sites are contiguous. The 5′ nucleosides in
such contiguous linkage sites are followed by a
subscript “Y”. Further, contiguous linkage
sites having three “Y” designations will
count as one less resistant linkage site when
the nucleoside in the center position is LNA or
CRN. In either of these two cases the nucleo-
sides with a Y designation that are contiguous
to the one with the LNA or CRN modification will
be treated according to the rules provided for
the more stable linkage sites (i.e. those
selected from the group A-A, U-U, G-G, G-C,
G-U, G-A, A-G and C-U.

TABLE 2
SAMPLE MODIFIED 11-merROOT SEED VEHICLES

| (Root Sequence No.) | (Root Sequence |
|---|---|
| Sequence | No.) Sequence |
| 5′-3′ | 5′-3′ |
| (1) GUU_{F}~C_{F}UUU_{F}~C_{F}UUU | (2) AGCUU_{F}~ |
| | C_{F}UU_{F}~C_{F}UU |
| (1) GU~U_{F}~C_{F}U~UU_{F}~C_{F}U~U~U | |
| (1) G_{F}U_{F}~U_{F}~C_{F}U_{F}~U_{F}U_{F}~C_{F}U_{F}~U_{F}~U_{F} | |
| (1) GUU_{L}C_{F}UUU_{L}C_{F}UUU | |
| (1) G_{F}U_{F}~U_{L}~C_{F}U_{F}~U_{F}U_{L}~C_{F}U_{F}~U_{F}~U_{F} | |
| (1) GU₁₂U_{F}~C_{F}UU₁₂U_{F}~C_{F}UU₁₂U | |
| (1) GU₁₂~U_{F}~C_{F}U~U₁₂U_{F}~C_{F}U~U₁₂~U | |
| (1) G_{F}U_{F12}~U_{F}~C_{F}U_{F}~U_{F12}U_{F}~C_{F}U_{F}~U_{F12}~U_{F} | |
| (1) G_{F}U₁₂~U_{F}~C_{F}U_{F}~U₁₂U_{F}~C_{F}U_{F}~U₁₂~U_{F} | |
| (1) GU₁₈U_{F}~C_{F}UU₁₈U_{F}~C_{F}UU₁₈U | |
| (1) GU₁₈~U_{F}~C_{F}U~U₁₈U_{F}~C_{F}U~U₁₈~U | |
| (1) G_{F}U_{F18}~U_{F}~C_{F}U_{F}~U_{F18}U_{F}~C_{F}U_{F}~U_{F18}~U_{F} | |
| (1) G_{F}U₁₈~U_{F}~C_{F}U_{F}~U₁₈U_{F}~C_{F}U_{F}~U₁₈~U_{F} | |
| (1) GU₁₂U_{L}C_{F}UU₁₂U_{L}C_{F}UU₁₂U | |
| (1) GU₁₂~U_{L}~C_{F}U~U₁₂U_{L}~C_{F}U~U₁₂~U | |
| (1) G_{F}U_{F12}~U_{L}~C_{F}U_{F}~U_{F12}U_{L}~C_{F}U_{F}~U_{F12}~U_{F} | |
| (1) G_{F}U₁₂~U_{L}~C_{F}U_{F}~U₁₂U_{L}~C_{F}U_{F}~U₁₂~U_{F} | |
| (1) GU₁₈U_{L}C_{F}UU₁₈U_{L}C_{F}UU₁₈U | |
| (1) GU₁₈~U_{L}~C_{F}U~U₁₈U_{L}~C_{F}U~U₁₈~U | |
| (1) G_{F}U_{F18}~U_{L}~C_{F}U_{F}~U_{F18}U_{L}~C_{F}U_{F}~U_{F18}~U_{F} | |
| (1) G_{F}Uₗ₈~U_{L}~C_{F}U_{F},~U₁₈U_{L}~C_{F}U_{F}Uₗ₈~U_{F} | |
| (1) GU₃U_{F}~C_{F}UU₃U_{F}~C_{F}UU₃U | |
| (1) GU₃~U_{F}~C_{F}U~U₃U_{F}~C_{F}U~U₃~U | |
| (1) G_{F}U₃~U_{F}~C_{F}U_{F}~U₃U_{F}~C_{F}U_{F}~U₃~U_{F} | |
| (1) G_{F}U_{F3}~U_{F}~C_{F}U~U_{F3}U_{F}~C_{F}U_{F}~U_{F3}~U_{F} | |
| (1) GU₃U_{L}C_{F}UU₃U_{L}C_{F}UU₃U | |
| (1) GU₃~U_{L}~C_{F}U~U₃U_{L}~C_{F}U~U₃~U | |
| (1) G_{F}U_{F3}~U_{L}~C_{F}U_{F}~U_{F3}U_{L}~C_{F}U_{F}~U_{F3}~U_{F} | |
| (1) G_{F}U₃~U_{L}~C_{F}U_{F}~U₃U_{L}~C_{F}U~U₃~U_{F} | |
| (1) GU₃U_{F}~C_{F4}UU₃U_{F}~C_{F4}UU₃U | |
| (1) GU₃~U_{F}~C_{F4}U~U₃U_{F}~C_{F4}U~U₃~U | |
| (1) G_{F}U_{F3}~U_{F}~C_{F4}U_{F}~U_{F3}U_{F}~C_{F4}U_{F}~U_{F3}~U_{F} | |
| (1) G_{F}U₃~U_{F}~C₄U_{F}~U₃U_{F}~C₄U_{F}~U₃~U_{F} | |
| (1) GU₃U_{L}C₄UU₃U_{L}C₄UU₃U | |
| (1) GU₃~U_{L}~C₄U~U₃U_{L}~C₄U~U₃~U | |
| (1) G_{F}U_{F3}~U_{L}~C_{F4}U_{F}~U_{F3}U_{L}~C_{F4}U_{F}~U_{F3}~U_{F} | |
| (1) G_{F}U₃~U_{L}~C₄U_{F}~U₃U_{L}~C₄U_{F}~U₃~U_{F} | |
| (1) GU₃U_{F}~C_{F19}UU₃U_{F}~C_{F19}UU₃U | |
| (1) GU₃~U_{F}~C_{F19}U~U₃U_{F}~C_{F19}U~U₃~U | |
| (1) G_{F}U_{F3}~U_{F}~C_{F19}U_{F}~U_{F3}U_{F}~C_{F19}U_{F}~U_{F3}~U_{F} | |
| (1) G_{F}U_{F3}~U_{F}~C₁₉U_{F}~U₃U_{F}~C₁₉U_{F}~U₃~U_{F} | |
| (1) GU₃U_{L}C₁₉UU₃U_{L}C₁₉UU₃U | |
| (1) GU₃~U_{L}~C₁₉U~U₃U_{L}~C₁₉U~U₃~U | |
| (1) G_{F}U_{F3}~U_{L}~C_{F19}U_{F}~U_{F3}U_{L}~C_{F19}U_{F}~U_{F3}~U_{F} | |
| (1) G_{F}U₃~U_{L}~C₁₉U_{F}~U₃U_{L}~C₁₉U_{F}~U₃~U_{F} | |
| (3) GGU_{X}CUU_{Y}A_{Y}UUU_{X}G | (4) GGU_{X}GUU_{Y} |
| | C_{Y}A_{Y}UUU |

9. Overhang Precursors Suitable for Use as the Overhang Module for ss-MiRs Generated Using the Modular Approach as Well as Being Suitable for Use in ss-RNAi More Generally.

The overhang precursors provided in the section by that name can be used with ss-RNAi. The overhang precursors shown in Table 4 are based on the root sequences shown in Table 3, however, are among those that are preferred when the goal is to maximize the IC₅₀ and/or duration of the intended silencing activity. The root sequences shown in Table 3 are 3 nucleoside sequences that have two linkage sites both of which are found in the member generally most nuclease resistant group. These are preferred where possible but in instances where all of the preferred overhang precursors result in ss-RNAi strands with unacceptable self-interaction then other overhang precursor sequences can be used when modified in accordance with the rules. When possible it is preferred that these substitute overhang precursors have an—N×N×N design where x is an accommodating modification and N is a nucleoside. The linkage between the nucleoside members of one of the less resistant linkage sites is required to be one that is nuclease resistant. In the case of linkage sites found in the more resistant group the intervening linkage can be nuclease resistant if desired. The nuclease resistant linkages available for use in overhang precursors are provided in the section by that name

In Table 2 LNA refers to the classic LNA form shown in the upper left figure in FIG. 10. The 3′ end nucleoside in the overhang precursor that is illustrated as a ribose can also be a 2′-fluoro or 2′-O-methyl. The letters in parenthesis indicate which contiguous nucleoside bases will form a relatively more stable linkage site with the most 5′ nucleoside of the overhang precursor and these more stable linkage sites are preferred if they do not promote unacceptable self-interaction by the strand as described herein. If there is no subscript after the parenthesis the indicated nucleoside can have any of the chemistries provided by the ss-RNAi rules for achieving nuclease resistance. If it has a K subscript then the indicated nucleoside will have the CENA modification. The use of CENA in this position allows any nucleoside base to be contiguous to the 3′end of the strand to which that overhang precursor is being added. The dash indicates that when one of these nucleoside bases is contiguous to the overhang precursor then the linkage can be phosphodiester, but it is not required to be phosphodiester. When a contiguous nucleoside with a different base is present, it is preferred that this linkage be nuclease resistant. The overhang precursor selected for use in a particular ss-RNAi is required to be one that does not result in unacceptable self-interaction by the strand in the form of self-dimer or hairpin formation under physiological conditions. The criteria for self-interaction acceptability or rejection are presented in the section providing the rules for AHD.

The linkages between many of the overhang precursor nucleosides are illustrated in the table as phosphodiester or phosphorothioate but this is not required and another linkage provided for overhang precursors can be used in one or more linkage sites. When the linkage is methylborane phosphine, phosphonoacetate or thiophosphonoacetate the contiguous nucleosides modifications in the overhang precursor can include 2′-deoxyribose.

TABLE 3
ROOT OVERHANG PRECURSOR SEQUENCES
FOR USE WITH ss-RNAi

| Root | |
|---|---|
| Sequence | Sequence* |
| No. | 5′-3′ |
| 1 | GUU |
| 2 | UUU |
| 3 | CUU |
| 4 | GCU |
| 5 | AGU |
| 6 | AGC |
| 7 | AAA |
| 8 | AGA |
| 9 | GAA |

TABLE 4
PREFERRED OVERHANG PRECURSORS FOR USE WITH ss-RNAi

| (Root Sequence No.) | (Root Sequence No.) | (Root Sequence No.) |
|---|---|---|
| (contiguous | (contiguous | (contiguous |
| nucleoside base) | nucleoside base) | nucleoside base) |
| Sequence 5′~3′ | Sequence 5′~3′ | Sequence 5′~3′ |
| (1) (G or A)-G_{E}U_{E}U_{R} | (2) (G, U or C)-U_{E}U_{E}U_{R} | (3) (G)-C_{E}U_{E}U_{R} |
| (1) (G or A)-G_{E}~U_{E}~U_{R} | (2) (G, U or C)-U_{E}~U_{E}~U_{R} | (3) (G)-C_{E}~U_{E}~U_{R} |
| (1) (G or A)-G_{R}U_{E}U_{R} | (2) (G, U or C)-U_{R}U_{E}U_{R} | (3) (G)-C_{R}U_{E}U_{R} |
| (1) (G or A)-G_{R}~U_{E}~U_{R} | (2) (G, U or C)-U_{R}~U_{E}~U_{R} | (3) (G)-C_{R}~U_{E}~U_{R} |
| (1) (A,G,C or U)_{K}-G_{R}U_{E}U_{R} | (2) (A,G,C or U)_{K}-U_{R}U_{E}U_{R} | (3) (A,G,C or U)_{K}-C_{R}U_{E}U_{R} |
| (1) (A,G,C or U)_{K}-G_{R}~U_{E}~U_{R} | (2) (A,G,C or U)_{K}-U_{R}~U_{E}~U_{R} | (3) (A,G,C or U)_{K}-C_{R}~U_{E}~U_{R} |
| (1) (G or A)-G_{O}U_{O}U_{R} | (2) (G, U or C)-U_{O}U_{O}U_{R} | (3) (G)-C_{O}U_{O}U_{R} |
| (1) (G or A)-G_{O}~U_{O}~U_{R} | (2) (G, U or C)-U_{O}~U_{O}~U_{R} | (3) (G)-C_{O}~U_{O}~U_{R} |
| (1) (G or A)-G_{O}U_{O}U_{R} | (2) (G, U or C)-U_{R}U_{O}U_{R} | (3) (G)-C_{R}U_{O}U_{R} |
| (1) (G or A)-G_{R}~U_{O}~U_{R} | (2) (G, U or C)-U_{R}~U_{O}~U_{R} | (3) (G)-C_{R}~U_{O}~U_{R} |
| (1) (A,G,C or U)_{K}-G_{R}U_{O}U_{R} | (2) (A,G,C or U)_{K}-U_{R}U_{O}U_{R} | (3) (A,G,C or U)_{K}-C_{R}U_{O}U_{R} |
| (1) (A,G,C or U)_{K}-G_{R}~U_{O}~U_{R} | (2) (A,G,C or U)_{K}-U_{R}~U_{O}~U_{R} | (3) (A,G,C or U)_{K}-C_{R}~U_{O}~U_{R} |
| (1) (G or A)-G_{T}U_{T}U_{R} | (2) (G, U or C)-U_{T}U_{T}U_{R} | (3) (G)-C_{T}U_{T}U_{R} |
| (1) (G or A)-G_{T}~U_{T}~U_{R} | (2) (G, U or C)-U_{T}~U_{T}~U_{R} | (3) (G)-C_{T}~U_{T}~U_{R} |
| (1) (G or A)-G_{R}U_{T}U_{R} | (2) (G, U or C)-U_{R}U_{T}U_{R} | (3) (G)-C_{R}U_{T}U_{R} |
| (1) (G or A)-G_{R}~U_{T}~U_{R} | (2) (G, U or C)-U_{R}~U_{T}~U_{R} | (3) (G)-C_{R}~U_{T}~U_{R} |
| (1) (A,G,C or U)_{K}-G_{R}U_{T}U_{R} | (2) (A,G,C or U)_{K}-U_{R}U_{T}U_{R} | (3) (A,G,C or U)_{K}-C_{R}U_{T}U_{R} |
| (1) (A,G,C or U)_{K}-G_{R}~U_{T}~U_{R} | (2) (A,G,C or U)_{K}-U_{R}~U_{T}~U_{R} | (3) (A,G,C or U)_{K}-C_{R}~U_{T}~U_{R} |
| (1) (G or A)-G_{Y}U_{Y}U_{R} | (2) (G, U or C)-U_{Y}U_{Y}U_{R} | (3) (G)-C_{Y}U_{Y}U_{R} |
| (1) (G or A)-G_{Y}~U_{Y}~U_{R} | (2) (G, U or C)-U_{Y}~U_{Y}~U_{R} | (3) (G)-C_{Y}~U_{Y}~U_{R} |
| (1) (G or A)-G_{R}U_{Y}U_{R} | (2) (G, U or C)-U_{R}U_{Y}U_{R} | (3) (G)-C_{R}U_{Y}U_{R} |
| (1) (G or A)-G_{R}~U_{Y}~U_{R} | (2) (G, U or C)-U_{R}~U_{Y}~U_{R} | (3) (G)-C_{R}~U_{Y}~U_{R} |
| (1) (A,G,C or U)_{Y}-G_{R}U_{Y}U_{R} | (2) (A,G,C or U)_{Y}-U_{R}U_{Y}U_{R} | (3) (A,G,C or U)_{Y}-C_{R}U_{Y}U_{R} |
| (1) (A,G,C or U)_{Y}-G_{R}~U_{Y}~U_{R} | (2) (A,G,C or U)_{Y}-U_{R}~U_{Y}~U_{R} | (3) (A,G,C or U)_{Y}-C_{R}~U_{Y}~U_{R} |
| (1) (G or A)-G_{S}U_{S}U_{R} | (2) (G, U or C)-U_{S}U_{S}U_{R} | (3) (G)-C_{S}U_{S}U_{R} |
| (1) (G or A)-G_{S}~U_{S}~U_{R} | (2) (G, U or C)-U_{S}~U_{S}~U_{R} | (3) (G)-C_{S}~U_{S}~U_{R} |
| (1) (G or A)-G_{R}U_{S}U_{R} | (2) (G, U or C)-U_{R}U_{S}U_{R} | (3) (G)-C_{R}U_{S}U_{R} |
| (1) (G or A)-G_{R}~U_{S}~U_{R} | (2) (G, U or C)-U_{R}~U_{S}~U_{R} | (3) (G)-C_{R}~U_{S}~U_{R} |
| (1) (A,G,C or U)_{K}-G_{R}U_{S}U_{R} | (2) (A,G,C or U)_{K}-U_{R}U_{S}U_{R} | (3) (A,G,C or U)_{K}-C_{R}U_{S}U_{R} |
| (1) (A,G,C or U)_{K}-G_{R}~U_{S}~U_{R} | (2) (A,G,C or U)_{K}-U_{R}~U_{S}~U_{R} | (3) (A,G,C or U)_{K}-C_{R}~U_{S}~U_{R} |
| (1) (G or A)-G_{J}U_{J}U_{R} | (2) (G, U or C)-U_{J}U_{J}U_{R} | (3) (G)-C_{J}U_{J}U_{R} |
| (1) (G or A)-G_{J}~U_{J}~U_{R} | (2) (G, U or C)-U_{J}~U_{J}~U_{R} | (3) (G)-C_{J}~U_{J}~U_{R} |
| (1) (G or A)-G_{R}U_{J}U_{R} | (2) (G, U or C)-U_{R}U_{J}U_{R} | (3) (G)-C_{R}U_{J}U_{R} |
| (1) (G or A)-G_{R}~U_{J}~U_{R} | (2) (G, U or C)-U_{R}~U_{J}~U_{R} | (3) (G)-C_{R}~U_{J}~U_{R} |
| (1) (A,G,C or U)_{K}-G_{R}U_{J}U_{R} | (2) (A,G,C or U)_{K}-U_{R}U_{J}U_{R} | (3) (A,G,C or U)_{K}-C_{R}U_{J}U_{R} |
| (1) (A,G,C or U)_{K}-G_{R}~U_{J}~U_{R} | (2) (A,G,C or U)_{K}-U_{R}~U_{J}~U_{R} | (3) (A,G,C or U)_{K}-C_{R}~U_{J}~U_{R} |
| (1) (G or A)-G_{H}U_{H}U_{R} | (2) (G, U or C)-U_{H}U_{H}U_{R} | (3) (G)-C_{H}U_{H}U_{R} |
| (1) (G or A)-G_{H}~U_{H}~U_{R} | (2) (G, U or C)-U_{H}~U_{H}~U_{R} | (3) (G)-C_{H}~U_{H}~U_{R} |
| (1) (G or A)-G_{R}U_{H}U_{R} | (2) (G, U or C)-U_{R}U_{H}U_{R} | (3) (G)-C_{R}U_{H}U_{R} |
| (1) (G or A)-G_{R}~U_{H}~U_{R} | (2) (G, U or C)-U_{R}~U_{H}~U_{R} | (3) (G)-C_{R}~U_{H}~U_{R} |
| (1) (A,G,C or U)_{K}-G_{R}U_{H}U_{R} | (2) (A,G,C or U)_{K}-U_{R}U_{H}U_{R} | (3) (A,G,C or U)_{K}-C_{R}U_{H}U_{R} |
| (1) (A,G,C or U)_{K}-G_{R}~U_{H}~U_{R} | (2) (A,G,C or U)_{K}-U_{R}~U_{H}~U_{R} | (3) (A,G,C or U)_{K}-C_{R}~U_{H}~U_{R} |
| (1) (G or A)-G_{L}U_{L}U_{R} | (2) (G, U or C)-U_{L}U_{L}U_{R} | (3) (G)-C_{L}U_{L}U_{R} |
| (1) (G or A)-G_{L}~U_{L}~U_{R} | (2) (G, U or C)-U_{L}~U_{L}~U_{R} | (3) (G)-C_{L}~U_{L}~U_{R} |
| (1) (G or A)-G_{R}U_{L}U_{R} | (2) (G, U or C)-U_{R}U_{L}U_{R} | (3) (G)-C_{R}U_{L}U_{R} |
| (1) (G or A)-G_{R}~U_{L}~U_{R} | (2) (G, U or C)-U_{R}~U_{L}~U_{R} | (3) (G)-C_{R}~U_{L}~U_{R} |
| (1) (A,G,C or U)_{K}-G_{R}U_{L}U_{R} | (2) (A,G,C or U)_{K}-U_{R}U_{L}U_{R} | (3) (A,G,C or U)_{K}-C_{R}U_{L}U_{R} |
| (1) (A,G,C or U)_{K}-G_{R}~U_{L}~U_{R} | (2) (A,G,C or U)_{K}-U_{R}~U_{L}~U_{R} | (3) (A,G,C or U)_{K}-C_{R}~U_{L}~U_{R} |
| (1) (A,G,C or U)_{K}-G_{R}U_{F}U_{R} | (2) (A,G,C Or U)_{K}-U_{R}U_{F}U_{R} | (3) (A,G,C Or U)_{K}-C_{R}U_{F}U_{R} |
| (1) (A,G,C or U)_{K}-G_{R}~U_{F}~U_{R} | (2) (A,G,C or U)_{K}-U_{R}~U_{F}~U_{R} | (3) (A,G,C Or U)_{K}-C_{R}~U_{F}~U_{R} |
| (1) (G or A)-G_{R}U_{R}U_{R} | (2) (G or U)-U_{R}U_{R}U_{R} | (3) (G)-C_{R}U_{R}U_{R} |
| (1) (G or A)-G_{R}~U_{R}~U_{R} | (2) (G or U)-U_{R}~U_{R}~U_{R} | (3) (G)-C_{R}~U_{R}~U_{R} |
| (1) (G or A)_{F}-G_{R}U_{F}U_{R} | (2) (G or U)_{F}-U_{R}U_{F}U_{R} | (3) (G)_{F}-C_{R}U_{F}U_{R} |
| (1) (G or A)_{F}-G_{R}~U_{F}~U_{R} | (2)(G or U)_{F}-U_{R}~U_{F}~U_{R} | (3) (G)_{F}-C_{R}~U_{F}~U_{R} |
| (1) (G or A)-G_{R}U_{R3}U_{R} | (2)(G or U)-U_{R}U_{R3}U_{R} | (3) (G)-C_{R}U_{R3}U_{R} |
| (1) (G or A)-G_{R}~U_{R3}~U_{R} | (2) (G or U)-U_{R}~U_{R3}~U_{R} | (3) (G)-C_{R}~U_{R3}~U_{R} |
| (1) (G or A)-G_{R}U_{F3}U_{R} | (2) (G or U)-U_{R}U_{F3}U_{R} | (3) (G)-C_{R}U_{F3}U_{R} |
| (1) (G or A)-G_{R}~U_{F3}~U_{R} | (2)(G or U)-U_{R}~U_{F3}~U_{R} | (3) (G)-C_{R}~U_{F3}~U_{R} |
| (1) (G or A)_{F}-G_{R}U_{F3}U_{R} | (2)(G or U)_{F}-U_{R}U_{F3}U_{R} | (3) (G or U)_{F}-C_{R}U_{F3}U_{R} |
| (1) (G or A)_{F}-G_{R}~U_{F3}~U_{R} | (2)(G or U)_{F}-U_{R}~U_{F3}~U_{R} | (3) (G or U)_{F}-C_{R}~U_{F3}~U_{R} |
| (1) (G or A)-G_{R}U_{R12}U_{R} | (2) (G or U)-U_{R}U_{R12}U_{R} | (3) (G)-C_{R}U_{R12}U_{R} |
| (1) (G or A)-G_{R}~U_{R12}~U_{R} | (2)(G or U)-U_{R}~U_{R12}~U_{R} | (3) (G)-C_{R}~U_{R12}~U_{R} |
| (1) (G or A)_{F}-G_{R}U_{F12}U_{R} | (2) (G or U)_{F}-U_{R}U_{F12}U_{R} | (3) (G or U)_{F}-C_{R}U_{F12}U_{R} |
| (1) (G or A)_{F}-G_{R}~U_{F12}~U_{R} | (2) (G or U)_{F}-U_{R}~U_{F12}~U_{R} | (3) (G or U)_{F}-C_{R}~U_{F12}~U_{R} |
| (1) (A,G,C or U)_{K}-G_{R}U_{F3}U_{R} | (2) (A,G,C or U)_{K}-U_{R}U_{F3}U_{R} | (3) (A,G,C or U)_{K}-C_{R}U_{F3}U_{R} |
| (1) (A,G,C or U)_{K}-G_{R}~U_{F3}~U_{R} | (2) (A,G,C or U)_{K}-U_{R}~U_{F3}~U_{R} | (3) (A,G,C or U)_{K}-C_{R}~U_{F3}~U_{R} |
| (1) (A,G,C or U)_{K}-G_{R}U_{F12}U_{R} | (2) (A,G,C or U)_{K}-U_{R}U_{F12}U_{R} | (3) (A,G,C or U)_{K}-C_{R}U_{F12}U_{R} |
| (1) (A,G,C or U)_{K}-G_{R}~U_{F12}~U_{R} | (2) (A,G,C or U)_{K}-U_{R}~U_{F12}~U_{R} | (3) (A,G,C or U)_{K}-C_{R}~U_{F12}~U_{R} |
| (1) (U)_{R3}~G_{R}U_{R3}U_{R} | (2) (U)_{R3}-U_{R}U_{R3}U_{R} | (3) (U)_{R3}~C_{R}U_{R3}U_{R} |
| (1) (U)_{R3}~G_{R}~U_{R3}~U_{R} | (2) (U)_{R3}-U_{R}~U_{R3}U_{R} | (3) (U)_{R3}~C_{R}~U_{R3}~U_{R} |
| (1) (U)_{F3}~G_{R}U_{F3}U_{R} | (2) (U)_{F3}-U_{R}U_{F3}U_{R} | (3) (U)_{F3}~C_{R}U_{F3}U_{R} |
| (1) (U)_{F3}~G_{R}~U_{F3}~U_{R} | (2) (U)_{F3}-U_{R}~U_{F3}~U_{R} | (3) (U)_{F3}~C_{R}~U_{F3}~U_{R} |
| (1) (U)_{R12}~G_{R}U_{R12}U_{R} | (2) (U)_{R12}-U_{R}U_{R12}U_{R} | (3) (U)_{R12}~C_{R}U_{R12}U_{R} |
| (1) (U)_{R12}~G_{R}~U_{R12}~UR | (2) (U)_{R12}-U_{R}~U_{R12}~U_{R} | (3) (U)_{R12}~C_{R}~U_{R12}~U_{R} |
| (1) (U)_{F12}~G_{R}U_{F12}U_{R} | (2) (U)_{F12}-U_{R}U_{F12}U_{R} | (3) (U)_{F12}~C_{R}U_{F12}U_{R} |
| (1) (U)_{F12}~G_{R}~U_{F12}~U_{R} | (2) (U)_{F12}-U_{R}~U_{F12}~U_{R} | (3) (U)_{F12}~C_{R}~U_{F12}~U_{R} |
| (4) (G or A)-G_{E}C_{E}U_{R} | (5) (G or A)-A_{E}G_{E}U_{R} | (6) (G or A)-A_{E}G_{E}C_{R} |
| (4) (G or A)-G_{E}~C_{E}~U_{R} | (5) (G or A)-A_{E}~G_{E}~U_{R} | (6) (G or A)-A_{E}~G_{E}~C_{R} |
| (4) (G or A)-G_{R}C_{E}U_{R} | (5) (G or A)-A_{R}G_{E}U_{R} | (6) (G or A)-A_{R}G_{E}C_{R} |
| (4) (G or A)-G_{R}~C_{E}~U_{R} | (5) (G or A)-A_{R}~G_{E}~U_{R} | (6) (G or A)-A_{R}~G_{E}~C_{R} |
| (4) (A,G,C or U)_{K}-G_{R}C_{E}U_{R} | (5) (A,G,C or U)_{K}-A_{R}G_{E}U_{R} | (6) (A,G,C or U)_{K}-A_{R}G_{E}C_{R} |
| (4) (A,G,C or U)_{K}-G_{R}~C_{E}~U_{R} | (5) (A,G,C or U)_{K}-A_{R}~G_{E}~U_{R} | (6) (A,G,C or U)_{K}~A_{R}~G_{E}~C_{R} |
| (4) (G or A)-G_{O}C_{O}U_{R} | (5) (G or A)-A_{O}G_{O}U_{R} | (6) (G or A)-A_{O}G_{O}C_{R} |
| (4) (G or A)-G_{O}~C_{O}~U_{R} | (5) (G or A)-A_{O}~G_{O}~U_{R} | (6) (G or A)-A_{O}~G_{O}~C_{R} |
| (4) (G or A)-G_{R}C_{O}U_{R} | (5) (G or A)-A_{R}G_{S}U_{R} | (6) (G or A)-A_{R}G_{O}C_{R} |
| (4) (G or A)-G_{R}~C_{O}~U_{R} | (5) (G or A)-A_{R}~G_{O}~U_{R} | (6) (G or A)-A_{R}~G_{O}~C_{R} |
| (4) (A,G,C or U)_{K}-G_{R}C_{O}U_{R} | (5) (A,G,C or U)_{K}-A_{R}G_{O}U_{R} | (6) (A,G,C or U)_{K}-A_{R}G_{O}C_{R} |
| (4) (A,G,C or U)_{K}-G_{R}~C_{O}~U_{R} | (5) (A,G,C or U)_{K}-A_{R}~G_{O}~U_{R} | (6) (A,G,C or U)_{K}-A_{R}~G_{O}~C_{R} |
| (4) (G or A)-G_{T}C_{T}U_{R} | (5) (G or A)-A_{T}G_{T}U_{R} | (6) (G or A)-A_{T}G_{T}C_{R} |
| (4) (G or A)-G_{T}~C_{T}~U_{R} | (5) (G or A)-A_{T}~G_{T}~U_{R} | (6) (G or A)-A_{T}~G_{T}~C_{R} |
| (4) (G or A)-G_{R}C_{T}U_{R} | (5) (G or A)-A_{R}G_{T}U_{R} | (6) (G or A)-A_{R}G_{T}C_{R} |
| (4) (G or A)-G_{R}~C_{T}~U_{R} | (5) (G or A)-A_{R}~G_{T}~U_{R} | (6) (G or A)-A_{R}~G_{T}~C_{R} |
| (4) (A,G,C or U)_{K}-G_{R}C_{T}U_{R} | (5) (A,G,C or U)_{K}-A_{R}G_{T}U_{R} | (6) (A,G,C or U)_{K}-A_{R}G_{T}C_{R} |
| (4) (A,G,C or U)_{K}-G_{R}~C_{T}~U_{R} | (5) (A,G,C or U)_{K}-A_{R}~G_{T}~U_{R} | (6) (A,G,C or U)_{K}-A_{R}~G_{T}~C_{R} |
| (4) (G or A)-G_{Y}C_{Y}U_{R} | (5) (G or A)-A_{Y}G_{Y}U_{R} | (6) (G or A)-A_{Y}G_{Y}C_{R} |
| (4) (G or A)-G_{Y}~C_{Y}~U_{R} | (5) (G or A)-A_{Y}~G_{Y}~U_{R} | (6) (G or A)-A_{Y}~G_{Y}~C_{R} |
| (4) (G or A)-G_{R}U_{R}U_{R} | (5) (G or A)-A_{R}G_{Y}U_{R} | (6) (G or A)-A_{R}G_{Y}C_{R} |
| (4) (G or A)-G_{R}~C_{Y}~U_{R} | (5) (G or A)-A_{R}~G_{Y}~U_{R} | (6) (G or A)-A_{R}~G_{Y}~C_{R} |
| (4) (A,G,C or U)_{Y}-G_{R}C_{Y}U_{R} | (5) (A,G,C or U)_{Y}-A_{R}G_{Y}U_{R} | (6) (A,G,C or U)_{Y}-A_{R}G_{Y}C_{R} |
| (4) (A,G,C or U)_{Y}-G_{R}~C_{Y}~U_{R} | (5) (A,G,C or U)_{Y}-A_{R}~G_{Y}~U_{R} | (6) (A,G,C or U)_{Y}-A_{R}~G_{Y}~C_{R} |
| (4) (G or A)-G_{S}C_{S}U_{R} | (5) (G or A)-A_{S}G_{S}U_{R} | (6) (G or A)-A_{S}G_{S}C_{R} |
| (4) (G or A)-G_{S}~C_{S}~U_{R} | (5) (G or A)-A_{S}~G_{S}~U_{R} | (6) (G or A)-A_{S}~G_{S}~C_{R} |
| (4) (G or A)-G_{R}C_{S}U_{R} | (5) (G or A)-A_{R}G_{S}U_{R} | (6) (G or A)-A_{R}G_{S}C_{R} |
| (4) (G or A)-G_{R}~C_{S}~U_{R} | (5) (G or A)-A_{R}~G_{S}~U_{R} | (6) (G or A)-A_{R}~G_{S}~C_{R} |
| (4) (A,G,C or U)_{K}-G_{R}C_{S}U_{R} | (5) (A,G,C or U)_{K}-A_{R}G_{S}U_{R} | (6) (A,G,C or U)_{K}-A_{R}G_{S}C_{R} |
| (4) (A,G,C or U)_{K}-G_{R}~C_{S}~U_{R} | (5) (A,G,C or U)_{K}-A_{R}~G_{S}~U_{R} | (6) (A,G,C or U)_{K}-A_{R}~G_{S}~C_{R} |
| (4) (G or A)-G_{J}C_{J}U_{R} | (5) (G or A)-A_{J}G_{J}U_{R} | (6) (G or A)-A_{J}G_{J}C_{R} |
| (4) (G or A)-G_{J}~C_{J}~U_{R} | (5) (G or A)-A_{J}~G_{J}~U_{R} | (6) (G or A)-A_{J}~G_{J}~C_{R} |
| (4) (G or A)-G_{R}C_{J}U_{R} | (5) (G or A)-A_{R}G_{J}U_{R} | (6) (G or A)-A_{R}G_{J}C_{R} |
| (4) (G or A)-G_{R}~C_{J}~U_{R} | (5) (G or A)-A_{R}~G_{J}~U_{R} | (6) (G or A)-A_{R}~G_{J}~C_{R} |
| (4) (A,G,C or U)_{K}-G_{R}C_{J}U_{R} | (5) (A,G,C or U)_{K}-A_{R}G_{J}U_{R} | (6) (A,G,C or U)_{K}-A_{R}G_{J}C_{R} |
| (4) (A,G,C or U)_{K}-G_{R}~C_{J}~U_{R} | (5) (A,G,C or U)_{K}-A_{R}~G_{J}~U_{R} | (6) (A,G,C or U)_{K}-A_{R}~G_{J}~C_{R} |
| (4) (G or A)-G_{H}C_{H}U_{R} | (5) (G or A)-A_{H}G_{H}U_{R} | (6) (G or A)-A_{H}G_{H}C_{R} |
| (4) (G or A)-G_{H}~C_{H}~U_{R} | (5) (G or A)-A_{H}~G_{H}~U_{R} | (6) (G or A)-A_{H}~G_{H}~C_{R} |
| (4) (G or A)-G_{R}C_{H}U_{R} | (5) (G or A)-A_{R}G_{H}U_{R} | (6) (G or A)-A_{R}G_{H}C_{R} |
| (4) (G or A)-G_{R}~C_{H}~U_{R} | (5) (G or A)-A_{R}~G_{H}~U_{R} | (6) (G or A)-A_{R}~G_{H}~C_{R} |
| (4) (A,G,C or U)_{K}-G_{R}C_{H}U_{R} | (5) (A,G,C or U)_{K}-A_{R}G_{H}U_{R} | (6) (A,G,C or U)_{K}-A_{R}G_{H}C_{R} |
| (4) (A,G,C or U)_{K}-G_{R}~C_{H}~U_{R} | (5) (A,G,C or U)_{K}-A_{R}~G_{H}~U_{R} | (6) (A,G,C or U)_{K}-A_{R}~G_{H}~C_{R} |
| (4) (G or A)-G_{L}C_{L}U_{R} | (5) (G or A)-A_{L}G_{L}U_{R} | (6) (G or A)-A_{L}G_{L}C_{R} |
| (4) (G or A)-G_{L}~C_{L}~U_{R} | (5) (G or A)-A_{L}~G_{L}~U_{R} | (6) (G or A)-A_{L}~G_{L}~C_{R} |
| (4) (G or A)-G_{R}C_{L}U_{R} | (5) (G or A)-A_{R}G_{L}U_{R} | (6) (G or A)-A_{R}G_{L}C_{R} |
| (4) (G or A)-G_{R}~C_{L}~U_{R} | (5) (G or A)-A_{R}~G_{L}~U_{R} | (6) (G or A)-A_{R}~G_{L}~C_{R} |
| (4) (A,G,C or U)_{K}-G_{R}C_{L}U_{R} | (5) (A,G,C or U)_{K}-A_{R}G_{L}U_{R} | (6) (A,G,C or U)_{K}-A_{R}G_{L}C_{R} |
| (4) (A,G,C or U)_{K}-G_{R}~C_{L}~U_{R} | (5) (A,G,C or U)_{K}-A_{R}~G_{L}~U_{R} | (6) (A,G,C or U)_{K}-A_{R}~G_{L}~C_{R} |
| (4) (A,G,C or U)_{K}-G_{R}C_{F}U_{R} | (5) (A,G,C or U)_{K}-A_{R}G_{F}U_{R} | (6) (A,G,C or U)_{K}-A_{R}G_{F}C_{R} |
| (4) (A,G,C or U)_{K}-G_{R}~C_{F}~U_{R} | (5) (A,G,C or U)_{K}-A_{R}~G_{F}~U_{R} | (6) (A,G,C or U)_{K}-A_{R}~G_{F}~C_{R} |
| (4) (G or A)-G_{R}C_{R}U_{R} | (5) (G or A)-A_{R}G_{R}U_{R} | (6) (G or A)-A_{R}G_{R}C_{R} |
| (4) (G or A)-G_{R}~C_{R}~U_{R} | (5) (G or A)-A_{R}~G_{R}~U_{R} | (6) (G or A)-A_{R}~G_{R}~C_{R} |
| (4) (G or A)_{F}-G_{R}C_{F}U_{R} | (5) (G or A)_{F}-A_{R}G_{F}U_{R} | (6) (G or A)_{F}-A_{R}G_{F}C_{R} |
| (4) (G or A)_{F}-G_{R}~C_{F}~U_{R} | (5) (G or A)_{F}-A_{R}~G_{F}~U_{R} | (6) (G or A)_{F}-A_{R}~G_{F}~C_{R} |
| (4) (G or A)-G_{R}C_{R4}U_{R} | (5) (U)_{R3}~A_{R}GU_{R} | (6) (U)_{R3}~A_{R}GC_{R} |
| (4) (G or A)-G_{R}~C_{R4}~U_{R} | (5) (U)_{R3}~A_{R}~G~U_{R} | (6) (U)_{R3}~A_{R}~G~C_{R} |
| (4) (G or A)-G_{R}C_{F4}U_{R} | (5) (U)_{F3}~A_{R}G_{F}U_{R} | (6) (U)_{F3}~A_{R}G_{F}C_{R} |
| (4) (G or A)-G_{R}~C_{F4}U_{R} | (5) (U)_{F3}~A_{R}~G_{F}~U_{R} | (6) (U)_{F3}~A_{R}~G_{F}~C_{R} |
| (4) (G or A)_{F}~G_{R}C_{F4}U_{R} | | |
| (4) (G or A)_{F}~G_{R}~C_{F4}~U_{R} | | |
| (4) (A,G,C or U)_{K}-C_{R}U_{F4}U_{R} | | |
| (4) (A,G,C or U)_{K}-G_{R}~C_{F4}~U_{R} | | |
| (4) (U)_{R3}~G_{R}C_{R4}U_{R} | | |
| (4) (U)_{R3}~G_{R}~C_{R4}~U_{R} | | |
| (4) (U)_{F3}~G_{R}C_{F4}U_{R} | | |
| (4) (U)_{F3}~G_{R}~C_{F4}~U_{R} | | |
| (7) (G or A)-A_{E}A_{E}A_{R} | (8) (G or A)-A_{E}G_{E}A_{R} | (9) (G or A)-G_{E}A_{E}A_{R} |
| (7) (G or A)-A_{E}~A_{E}~A_{R} | (8) (G or A)-A_{E}~G_{E}~A_{R} | (9) (G or A)-G_{E}~A_{E}~A_{R} |
| (7) (G or A)-A_{R}A_{E}A_{R} | (8) (G or A)-A_{R}G_{E}A_{R} | (9) (G or A)-G_{R}A_{E}A_{R} |
| (7) (G or A)-A_{R}~A_{E}~A_{R} | (8) (G or A)-A_{R}~G_{E}~A_{R} | (9) (G or A)-G_{R}~A_{E}~A_{R} |
| (7) (A,G,C or U)_{K}-A_{R}A_{E}A_{R} | (8) (A,G,C or U)_{K}-A_{R}G_{E}A_{R} | (9) (A,G,C or U)_{K}-G_{R}A_{E}A_{R} |
| (7) (A,G,C or U)_{K}-A_{R}~A_{E}~A_{R} | (8) (A,G,C or U)_{K}-A_{R}~G_{E}~A_{R} | (9) (A,G,C or U)_{K}-G_{R}~A_{E}~A_{R} |
| (7) (G or A)-A_{O}A_{O}A_{R} | (8) (G or A)-A_{O}G_{O}A_{R} | (9) (G or A)-G_{O}A_{O}A_{R} |
| (7) (G or A)-A_{O}~A_{O}~A_{R} | (8) (G or A)-A_{O}~G_{O}~A_{R} | (9) (G or A)-G_{O}~A_{O}~A_{R} |
| (7) (G or A)-A_{R}A_{O}A_{R} | (8) (G or A)-A_{R}G_{O}A_{R} | (9) (G or A)-G_{R}A_{O}A_{R} |
| (7) (G or A)-A_{R}~A_{O}~A_{R} | (8) (G or A)-A_{R}~G_{O}~A_{R} | (9) (G or A)-G_{R}~A_{O}~A_{R} |
| (7) (A,G,C or U)_{K}-A_{R}A_{O}A_{R} | (8) (A,G,C or U)_{K}-A_{R}G_{O}A_{R} | (9) (A,G,C or U)_{K}-G_{R}AoA_{R} |
| (7) (A,G,C or U)_{K}-A_{R}~A_{O}~A_{R} | (8) (A,G,C or U)_{K}-A_{R}~G_{O}~A_{R} | (9) (A,G,C or U)_{K}-G_{R}~A_{O}~A_{R} |
| (7) (G or A)-A_{T}A_{T}A_{R} | (8) (G or A)-A_{T}G_{T}A_{R} | (9) (G or A)-G_{T}A_{T}A_{R} |
| (7) (G or A)-A_{T}~A_{T}~A_{R} | (8) (G or A)-A_{T}~G_{T}~A_{R} | (9) (G or A)-G_{T}~A_{T}~A_{R} |
| (7) (G or A)-A_{R}A_{T}A_{R} | (8) (G or A)-A_{R}G_{T}A_{R} | (9) (G or A)-G_{R}A_{T}A_{R} |
| (7) (G or A)-A_{R}~A_{T}~A_{R} | (8) (G or A)-A_{R}~G_{T}~A_{R} | (9) (G or A)-G_{R}~A_{T}~A_{R} |
| (7) (A,G,C or U)_{K}-A_{R}A_{T}A_{R} | (8) (A,G,C or U)_{K}-A_{R}G_{T}A_{R} | (9) (A,G,C or U)_{K}-G_{R}A_{T}A_{R} |
| (7) (A,G,C or U)_{K}-A_{R}~A_{T}~A_{R} | (8) (A,G,C or U)_{K}-A_{R}~G_{T}~A_{R} | (9) (A,G,C or U)_{K}-G_{R}~A_{T}~A_{R} |
| (7) (G or A)-A_{Y}A_{Y}A_{R} | (8) (G or A)-A_{Y}G_{Y}A_{R} | (9) (G or A)-G_{Y}A_{Y}A_{R} |
| (7) (G or A)-A_{Y}~A_{Y}~A_{R} | (8) (G or A)-A_{Y}~G_{Y}~A_{R} | (9) (G or A)-G_{Y}~A_{Y}~A_{R} |
| (7) (G or A)-A_{R}A_{Y}A_{R} | (8) (G or A)-A_{R}G_{Y}A_{R} | (9) (G or A)-G_{R}A_{Y}A_{R} |
| (7) (G or A)-A_{R}~A_{Y}~A_{R} | (8) (G or A)-A_{R}~G_{Y}~A_{R} | (9) (G or A)-G_{R}~A_{Y}~A_{R} |
| (7) (A,G,C or U)_{Y}-A_{R}A_{Y}A_{R} | (8) (A,G,C or U)_{Y}-A_{R}G_{Y}A_{R} | (9) (A,G,C or U)_{Y}-G_{R}A_{Y}A_{R} |
| (7) (A,G,C or U)_{Y}-A_{R}~A_{Y}~A_{R} | (8) (A,G,C or U)_{Y}-A_{R}~G_{Y}~A_{R} | (9) (A,G,C or U)_{Y}-G_{R}~A_{Y}~A_{R} |
| (7) (G or A)-A_{S}A_{S}A_{R} | (8) (G or A)-A_{S}G_{S}A_{R} | (9) (G or A)-G_{S}A_{S}A_{R} |
| (7) (G or A)-A_{S}~A_{S}~A_{R} | (8) (G or A)-A_{S}~G_{S}~A_{R} | (9) (G or A)-G_{S}~A_{S}~A_{R} |
| (7) (G or A)-A_{R}A_{S}A_{R} | (8) (G or A)-A_{R}G_{S}A_{R} | (9) (G or A)-G_{R}A_{S}A_{R} |
| (7) (G or A)-A_{R}~A_{S}~A_{R} | (8) (G or A)-A_{R}~G_{S}~A_{R} | (9) (G or A)-G_{R}~A_{S}~A_{R} |
| (7) (A,G,C or U)_{K}-A_{R}A_{S}A_{R} | (8) (A,G,C or U)_{K}-A_{R}G_{S}A_{R} | (9) (A,G,C or U)_{K}-G_{R}A_{S}A_{R} |
| (7) (A,G,C or U)_{K}-A_{R}~A_{S}~A_{R} | (8) (A,G,C or U)_{K}-A_{R}~G_{S}~A_{R} | (9) (A,G,C or U)_{K}-G_{R}~A_{S}~A_{R} |
| (7) (G or A)-A_{J}A_{J}A_{R} | (8) (G or A)-A_{J}G_{J}A_{R} | (9) (G or A)-G_{J}A_{J}A_{R} |
| (7) (G or A)-A_{J}~A_{J}~A_{R} | (8) (G or A)-A_{J}~G_{J}~A_{R} | (9) (G or A)-G_{J}~A_{J}~A_{R} |
| (7) (G or A)-A_{R}A_{J}A_{R} | (8) (G or A)-A_{R}G_{J}A_{R} | (9) (G or A)-G_{R}A_{J}A_{R} |
| (7) (G or A)-A_{R}~A_{S}~A_{R} | (8) (G or A)-A_{R}~G_{S}~A_{R} | (9) (G or A)-G_{R}~A_{S}~A_{R} |
| (7) (A,G,C or U)_{K}-A_{R}A_{J}A_{R} | (8) (A,G,C or U)_{K}-A_{R}G_{J}A_{R} | (9) (A,G,C or U)_{K}-G_{R}A_{J}A_{R} |
| (7) (A,G,C or U)_{K}-A_{R}~A_{S}~A_{R} | (8) (A,G,C or U)_{K}-A_{R}~G_{S}~A_{R} | (9) (A,G,C or U)_{K}-G_{R}~A_{S}~A_{R} |
| (7) (G or A)-A_{H}A_{H}A_{R} | (8) (G or A)-A_{H}G_{H}A_{R} | (9) (G or A)-G_{H}A_{H}A_{R} |
| (7) (G or A)-A_{H}~A_{H}~A_{R} | (8) (G or A)-A_{H}~G_{H}~A_{R} | (9) (G or A)-G_{H}~A_{H}~A_{R} |
| (7) (G or A)-A_{R}A_{H}A_{R} | (8) (G or A)-A_{R}G_{H}A_{R} | (9) (G or A)-G_{R}A_{H}A_{R} |
| (7) (G or A)-A_{R}~A_{H}~A_{R} | (8) (G or A)-A_{R}~G_{H}~A_{R} | (9) (G or A)-G_{R}~A_{H}~A_{R} |
| (7) (A,G,C or U)_{K}-A_{R}A_{H}A_{R} | (8) (A,G,C or U)_{K}-A_{R}G_{H}A_{R} | (9) (A,G,C or U)_{K}-G_{R}A_{H}A_{R} |
| (7) (A,G,C or U)_{K}-A_{R}~A_{H}~A_{R} | (8) (A,G,C or U)_{K}-A_{R}~G_{H}~A_{R} | (9) (A,G,C or U)_{K}-G_{R}~A_{H}~A_{R} |
| (7) (G or A)-A_{L}A_{L}A_{R} | (8) (G or A)-A_{L}G_{L}A_{R} | (9) (G or A)-G_{L}A_{L}A_{R} |
| (7) (G or A)-A_{L}~A_{L}~A_{R} | (8) (G or A)-A_{L}~G_{L}~A_{R} | (9) (G or A)-G_{L}~A_{L}~A_{R} |
| (7) (G or A)-A_{R}A_{L}A_{R} | (8) (G or A)-A_{R}G_{L}A_{R} | (9) (G or A)-G_{R}A_{L}A_{R} |
| (7) (G or A)-A_{R}~A_{L}~A_{R} | (8) (G or A)-A_{R}~G_{L}~A_{R} | (9) (G or A)-G_{R}~A_{L}~A_{R} |
| (7) (A,G,C or U)_{K}-A_{R}A_{L}A_{R} | (8) (A,G,C or U)_{K}-A_{R}G_{L}A_{R} | (9) (A,G,C or U)_{K}-G_{R}A_{L}A_{R} |
| (7) (A,G,C or U)_{K}-A_{R}~A_{L}~A_{R} | (8) (A,G,C or U)_{K}-A~G_{L}~A_{R} | (9) (A,G,C or U)_{K}-G_{R}~A_{L}~A_{R} |
| (7) (A,G,C or U)_{K}-A_{R}A_{F}A_{R} | (8) (A,G,C or U)_{K}-A_{R}G_{F}A_{R} | (9) (A,G,C or U)_{K}-G_{R}A_{F}A_{R} |
| (7) (A,G,C or U)_{K}-A_{R}~A_{F}~A_{R} | (8) (A,G,C or U)_{K}-A_{R}~G_{F}~A_{R} | (9) (A,G,C or U)_{K}-G_{R}~A_{F}~A_{R} |
| (7) (G or A)-A_{R}A_{R}A_{R} | (8) (G or A)-A_{R}G_{R}A_{R} | (9) (G or A)-G_{R}A_{R}A_{R} |
| (7) (G or A)-A_{R}~A_{R}~A_{R} | (8) (G or A)-A_{R}~G_{R}~A_{R} | (9) (G or A)-G_{R}~A_{R}~A_{R} |
| (7) (G or A)-A_{R}A_{F}A_{R} | (8) (G or A)-A_{R}G_{F}A_{R} | (9) (G or A)-G_{R}A_{F}A_{R} |
| (7) (G or A)-A_{R}~A_{F}~A_{R} | (8) (G or A)-A_{R}~G_{R}~A_{R} | (9) (G or A)-G_{R}~A_{F}~A_{R} |
| (7) (G or A)_{F}-A_{R}A_{F}A_{R} | (8) (G or A)_{F}-A_{R}G_{F}A_{R} | (9) (G or A)_{F}-G_{R}A_{F}A_{R} |
| (7) (G or A)_{F}-A_{R}~A_{F}~A_{R} | (8) (G or A)_{F}-A_{R}~G_{F}~A_{R} | (9) (G or A)_{F}-G_{R}~A_{F}~A_{R} |
| (7) (U)_{R3}~A_{R}A_{R}A_{R} | (8) (U)_{R3}~A_{R}G_{R}A_{R} | (9) (U)_{R3}~G_{R}A_{R}A_{R} |
| (7) (U)_{R3}~A_{R}~A_{R}~A_{R} | (8) (U)_{R3}~A_{R}~G_{R}~A_{R} | (9) (U)_{R3}~G_{R}~A_{R}~A_{R} |
| (7) (U)_{F3}~A_{R}A_{F}A_{R} | (8) (U)_{F3}~A_{R}G_{F}A_{R} | (9) (U)_{F3}~G_{R}A_{F}A_{R} |
| (7) (U)_{F3}~A_{R}~A_{R}~A_{R} | (8) (U)_{F3}~A_{R}~G_{R}~A_{R} | (9) (U)_{F3}~G_{R}~A_{R}~A_{R} |
| (7) (U)_{R12}~A_{R}A_{R}A_{R} | (8) (U)_{R12}~A_{R}G_{R}A_{R} | (9) (U)_{R12}~G_{R}A_{R}A_{R} |
| (7) (U)_{R12}~A_{R}~A_{R}~A_{R} | (8) (U)_{R12}~A_{R}~G_{R}~A_{R} | (9) (U)_{R12}~G_{R}~A_{R}~A_{R} |
| (7) (U)_{F12}~A_{R}A_{F}A_{R} | (8) (U)_{F12}~A_{R}G_{F}A_{R} | (9) (U)_{F12}~G_{R}A_{F}A_{R} |
| (7) (U)_{F12}~A_{R}~A_{R}~A_{R} | (8) (U)_{F12}~A_{R}~G_{F}~A_{R} | (9) (U)_{F12}~G_{R}~A_{F}~A_{R} |

10. Means to Increase the Binding Affinity of the Seed Sequence of an ss-MiR with its Targets

The ability of an ss-MiR to effect the expression of its targets is, in part, a function of the level of the binding affinity between the seed sequence and its target sequence. Hence, it is possible to increase the activity of an ss-MiR by altering the nucleosides in the seed region using modifications that promote binding affinity. A number of examples are provided in Table 5.

TABLE 5
NUCLEOSIDE MODIFICATIONS SUITABLE FOR USE IN THE SEED SEQUENCES
OF SS-MIRs TO INCREASE THEIR BINDING AFFINITY WITH THEIR TARGETS

| | Degrees Change in | |
|---|---|---|
| Modification | Tm per Modification | Comments |
| 2′-fluoro | plus 1.0-plus 2.0 | |
| 2′-0-methyl | plus 0.5-plus 1.0 | |
| LNA | plus 2.0-plus 9.0 | LNA with adenine has about one-half of the |
| | | stabilizing effect of LNAs with other standard |
| | | bases. Using 2,6-diaminopurine or replacing a |
| | | complementary uracil containing nucleoside with |
| | | an LNA with a thymine base can reverse this. |
| | | Using a 2-thiothymine replacement for a thymine |
| | | can increase the affinity of a LNA brining it to the |
| | | upper end of the indicated Tm range. |
| 2,6-diaminopurine | plus 1.0-plus 3.5 | Replacement of adenine with 2,6-diaminopurine |
| | | increases the Tm. It can be paired with any of the |
| | | sugar modifications or substitutes provided for |
| | | herein to form a nucleoside. The complementary |
| | | partner nucleoside can have uracil or thymine. |
| 2-thiouracil | plus 1.0-plus 3.0 | The complimentary nucleoside in the target strand |
| | | must contain adenine rather than guanine when the |
| | | goal is to optimize stability. The most stabilizing |
| | | nucleosides have 2-thiouridine paired with LNA |
| | | where the use of this base further increases the |
| | | stabilizing effect of LNA. |
| 4-thiouracil | plus 3.0-plus 6.0 | 4-thiouracil can be paired with any of the sugar |
| | | modifications or sugar substitutes provided for |
| | | herein to form a nucleoside. The complimentary |
| | | nucleoside in the partner strand must contain |
| | | guanine rather than adenine when the goal is to |
| | | increase stability particularly relative to a U:G |
| | | wobble base. The most stabilizing nucleosides |
| | | have 4-thiouracil paired with LNA where the use |
| | | of this base further increases the stabilizing effect |
| | | of the LNA modification. |
| 5-methylcytosine | plus 1.0 | |
| Pseudouracil | plus 1.0-plus 2.0 | |
| 2-thiothymine | plus 2.0-plus 6.0 | 2-thiothymine can be paired with any of the sugar |
| | | modifications or sugar substitures provided for |
| | | herein to form a nucleoside. The most stabilizing |
| | | nucleosides have 2-thiothymine paired with LNA |
| | | where the use of this base further increases the |
| | | stabilizing effect of LNA. |

* Tm is measured in degrees centigrade under physiologic conditions. The numbers provided are approximations and the actual effects on Tm are influenced by a number of parameters including but not limited to the length of the strand, the position of the modification in the duplex and the presences of other modifications in the strand. The effects on Tm with a complementary strand are more pronounced when the modification is used in an internal position in the strand vs. at the end. Thus, the lower end of the range is applicable to the terminal nucleosides unless otherwise specified.

11. Means to Limit Off Target Effects Applicable to ss-siRNA and ss-IMiRs:
a) Any stimulation of the innate immune response can be inhibited either by the preferred method of changing the ribose of one or more uracil, adenosine or guanine containing nucleoside to 2′-fluoro as needed or the less preferred method of using the 2′-0-methyl modification in 1-3 nucleosides in the strand irrespective of their base if the preferred method does not provide a satisfactory result. 2′-0-methyl can be used in the overhang precursor for this purpose. It is also preferred that a 2′-0-methyl not be used in position 14 counting from the 5′-end.
b) Off-target effects due to the seed region directing the strand to act in an ss-MiR fashion can be inhibited by using one or two of the following methods as needed: (1) a 2′-0-methyl in position 2 from the 5′-end; (2) 1-3 CENA modified nucleosides inserted in the seed sequence with inclusion of position 3 from the 5′-end of the strand being preferred; (3) UNA or abasic modification used preferably in position 7 from the 5′-end of the strand; and (4) replacing an adenine containing nucleoside with a modification selected from the group N2-propyl-2-aminopurine or N2-cyclopentyl-2-aminopurine and/or a replacing a guanine containing nucleoside with a N2-cyclopentylguanine modification where one or more of these modifications can be used in position(s) 2 and/or 7 from the 5′-end of the strand. These modified bases can be used with any of the sugars or sugar substitutes provided herein consistent with the rules.
   12. Means to Limit Off-Target Effects by ss-MiRs:
a) Any stimulation of the innate immune response can be inhibited either by the preferred method of changing the ribose of one or more uracil, adenosine or guanine containing nucleoside to 2′-fluoro as needed or the less preferred method of using the 2′-0-methyl modification in 1-3 nucleosides in the strand irrespective of their base if the preferred method does not provide a satisfactory result. 2′-0-methyl can be used in the overhang precursor for this purpose. It is also preferred that a 2′-0-methyl not be used in position 14 counting from the 5′-end.
b) Off-target effects due to AGO-2 based catalytic silencing activity producing an off-target effect can be inhibited by replacing the nucleosides in positions 10 and/or 11 with one or two mismatches with the unintended target and/or replacing then with modified nucleosides that are selected from the group 2,4-dichlorobenzene, 3-methyluracil, abasic, UNA, 5,6-dihydrouracil and N⁴,N⁴-dimethylcytosine. These modified bases can be used with any of the sugars or sugar substitutes provided herein consistent with the rules.

### N. Overhang Precursors

Several groups have described variations in overhang design/chemistry that can affect the duration of the silencing effect of conventional siRNA. These same structures can be used as overhang precursors in ssRNAi strands. Zhang et al., (Bioorganic & Medicinal Chemistry 17: 2441, 2009), for example, showed that two nucleoside 3′-end overhangs with morpholine rings replacing the ribose in both the sense and guide strands or just the guide strands of conventional siRNA can result in a longer lasting silencing effect than the same siRNA with the standard dTdT overhangs. Strapps et al., (Nucl Acids Res 38: 4788, 2010), in another example, found that the dTdT overhangs were associated with a significantly reduced silencing period both in vitro and in vivo compared to the other overhang types tested. The latter consisted of the following: two 2′-0-methyl uridines; two 2′-0-methyl modified nucleosides complementary to the RNA target; or unmodified ribonucleosides complementary to the RNA target. Differences in duration of effect were found to not be due to either a difference in IC₅₀ values or to variable degrees of maximal target silencing. These data suggest that ribonucleosides may have a stronger binding to the PAZ domain than deoxyribonucleosides.

Numerous other 3′-end overhang precursor chemistries can promote seqRNAi activity and nuclease resistance. These include but are not limited to the following where the indicated nucleoside analog chemistries can be used with any of the normal bases: (1) 2′-0-Methyl; (2) 2′-fluoro; (3) FANA; (4) 2′-0-methyoxyethyl (5) any of the four types of LNA shown in FIG. 10; (6) morpholino; (7) tricyclo-DNA (Ittig et al., Artif DNA, PNA & XNA 1: 9, 2010); (8) ribo-difluorotoluyl (Xia et al., ACS Chem Biol 1: 176, 2006); (9) 4′-thioribonucleotides (Hoshika et al., Chem Bio Chem 8: 2133, 2007); (10) 2′-0-methyl-4′-thioribonucleotide (Takahashi et al., Nucleic Acids Res 37: 1353, 2009; Matsuda, Yakugaku Zasshi 131: 285, 2011); (11) altritol-nucleoside (ANA) shown in FIG. 5 (Fisher et al., Nucleic Acids Res 35: 1064, 2007); (12) cyclohexenyl nucleoside (CeNA) (Nauwelaerts et al., J Am Chem Soc 129; 9340, 2007; (13) piperazine (U.S. Pat. No. 6,841,675); (14) 5-bis(aminoethyl)aminoethylcarbamoylmethyl-2′-deoxyuridine or 5-bis(aminoethyl)aminoethylcarbamoylmethyl-thymidine (Masud et al., Bioorg Med Chem Lett 21: 715, 2010); (15) either of the two types of CRN shown in FIG. 9; (16) UNA shown in FIG. 4; (17) HM shown in FIG. 3; (18) EA shown in FIG. 6; (19) ALN shown in FIG. 10; or (20) arabinonucleoside.

The nucleosides used in overhang precursors in ssRNAi strands can be used in various combinations in 3′-end overhangs and are preferably joined together and to the adjacent non-overhang nucleoside by a nuclease resistant linkage such as phosphorothioate, phosphonoacetate, thiophosphonoacetate, methylborane phosphine, amide, carbamate or urea (Sheehan et al., Nucleic Acids Res 31: 4109, 2003; Krishna & Caruthers, J Amer Chem Soc 133: 9844, 2011; Iwase et al., Nucleic Acids Symposium Series 50: 175, 2006; Iwase et al., Nucleosides Nucleotides Nucleic Acids 26: 1451, 2007; Iwase et al., Nucleic Acids Symposium Series 53: 119, 2009; Ueno et al. Biochem Biophys Res Comm 330: 1168, 2005). In addition unmodified nucleosides can be used in overhangs when they are joined together using these linkages but preferably not phosphorothioate with ribonucleosides. These linkages can also be used in 5′-end overhangs but preferably the nucleosides are limited to the following: (1) 2′-0-Methyl; (2) 2′-fluoro; (3) FANA; and (4) RNA (native ribose). In the case of seqMiRs, however, such 5′-end modifications have to be evaluated for their effects on what mRNAs will be targeted for silencing.

Further, 3′-end overhang precursors can be comprised of certain hydrophobic aromatic moieties. For example, those that are comprised of one to three units containing two six membered rings joined by phosphodiester or one of the other linkages just listed where the unit(s) are attached to the oligonucleotide by the same linkage and when multiple units are used they are also joined by the same linkage. Two unit structures are preferred. Suitable ring structures include benzene, pyridine, morpholine and piperazine (U.S. Pat. No. 6,841,675). Structures based on the benzene and pyridine rings have been previously described for 3′-end overhang use in conventional siRNA by Ueno et al., (Bioorg Med Chem Lett 18:194, 2008; Bioorganic & Medicinal Chemistry 17: 1974, 2009). Specifically, these units are 1,3-bis(hydroxymethyl)benzene, 1,3-bis(hydroxymethyl)pyridine and 1,2-bis(hydroxymethyl)benzene. These are also suitable for seqRNAi use as overhang precursors.

O. Figures Exemplifying ss-RNAi and seqRNAi Compounds of the Present Invention

The application of the ss-RNAi architecture dependent algorithms, including those engendering AHD, to illustrative ss-MiR, ss-IMiR and ss-siRNA examples are provided in the figures. The code for various modifications shown in the figures with ss-RNA compositions of matter are shown in FIG. 1. When a CRN R monomer is designated in an ss-RNAi composition of matter (W) then unless otherwise stated it is to be assumed that the component labeled X in FIG. 9 is an oxygen. When a CRN Q monomer is designated in an ss-RNAi composition of matter (V) then unless otherwise stated it is to be assumed that the component labeled X in FIG. 9 is oxygen, Y and Z are CH and R₂ is a hydroxyl. This is to provide specificity to these compounds and is not meant to imply these are the preferred modifications to the CRN R or Q sugar substitutes. When LNA (L) is specified in the figures with ss-RNAi composition of matter it is classic LNA shown in FIG. 10 A at the top (β-D-ribo configuration).

Examples of ss-MiR compositions of matter based on the use of the modular design are provided in FIG. 17B that involve the use of the terminal 8 or 8 nucleosides from the 5′-end miR-34a-5p modified as provided by the rules herein. The miR-34a sequence is the same for both humans and for mouse. Ss-IMiR compositions of matter directed against human/murine miR-34a are provided in FIG. 18. Ss-siRNA compositions of matter directed to human/murine PTEN are shown in FIG. 19 and human/non-human primate/murine p53 ss-siRNA compositions of matter are provided in FIG. 20.

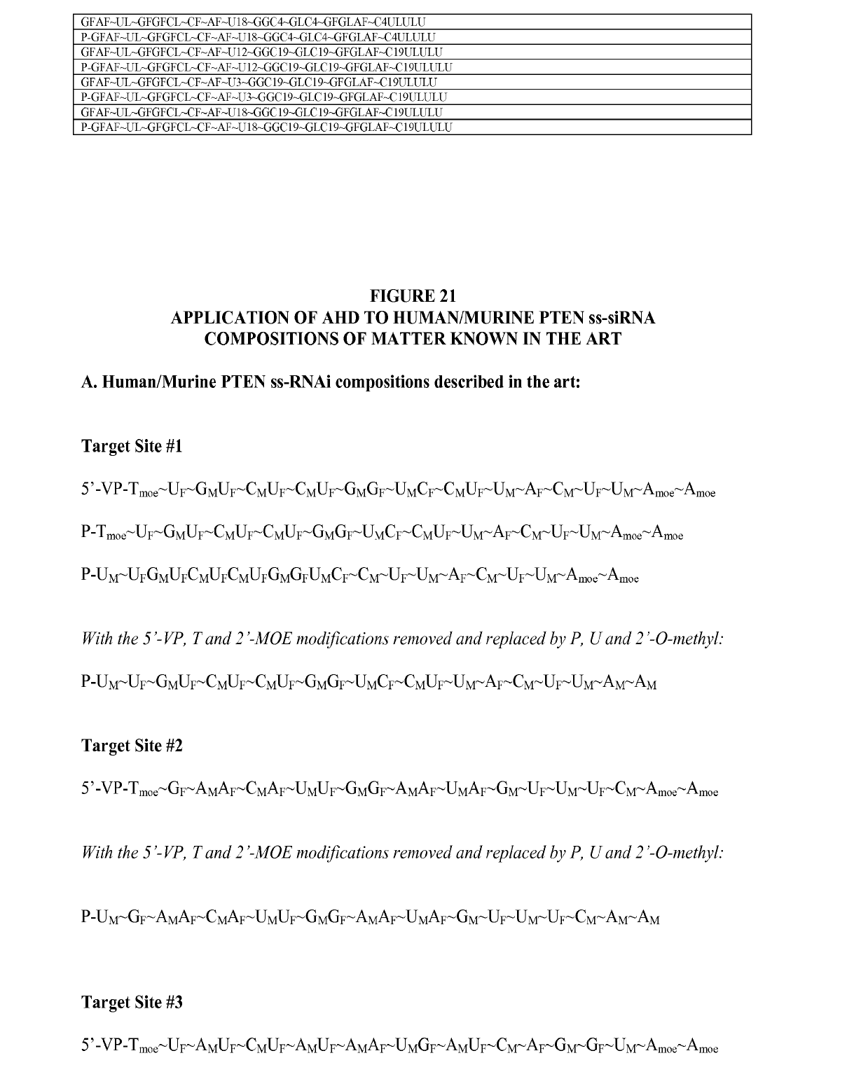

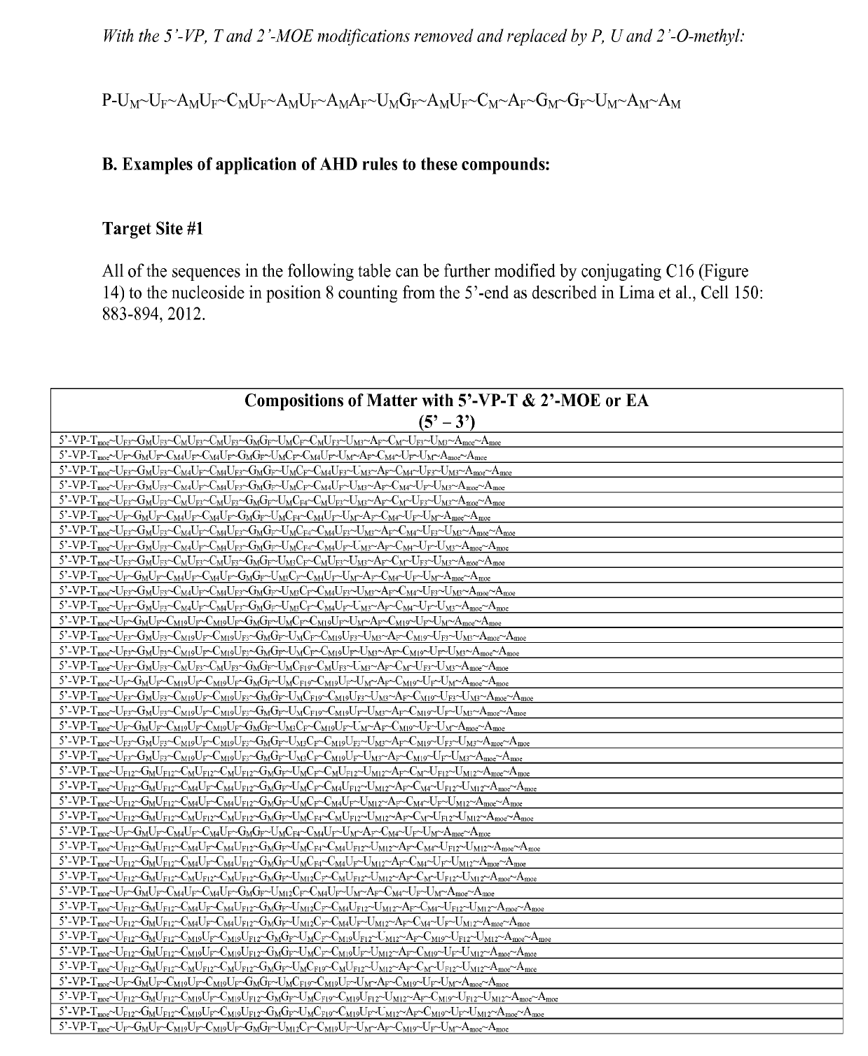

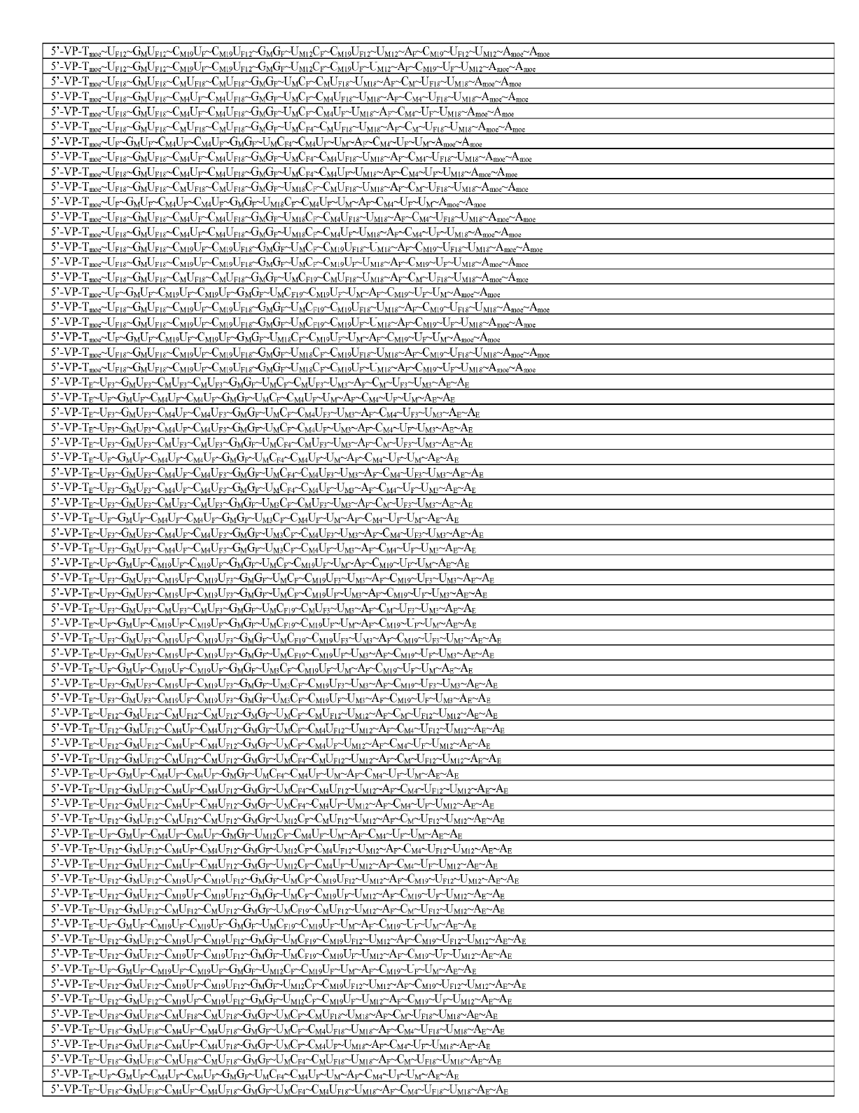

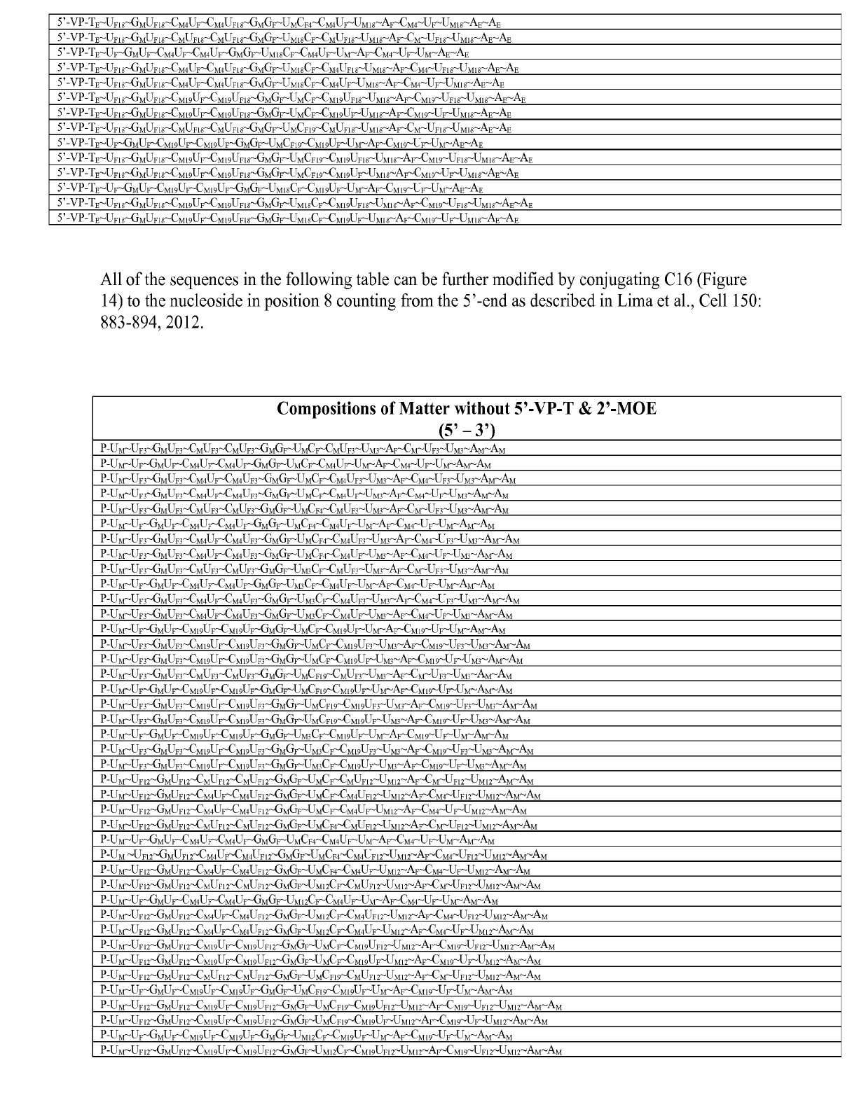

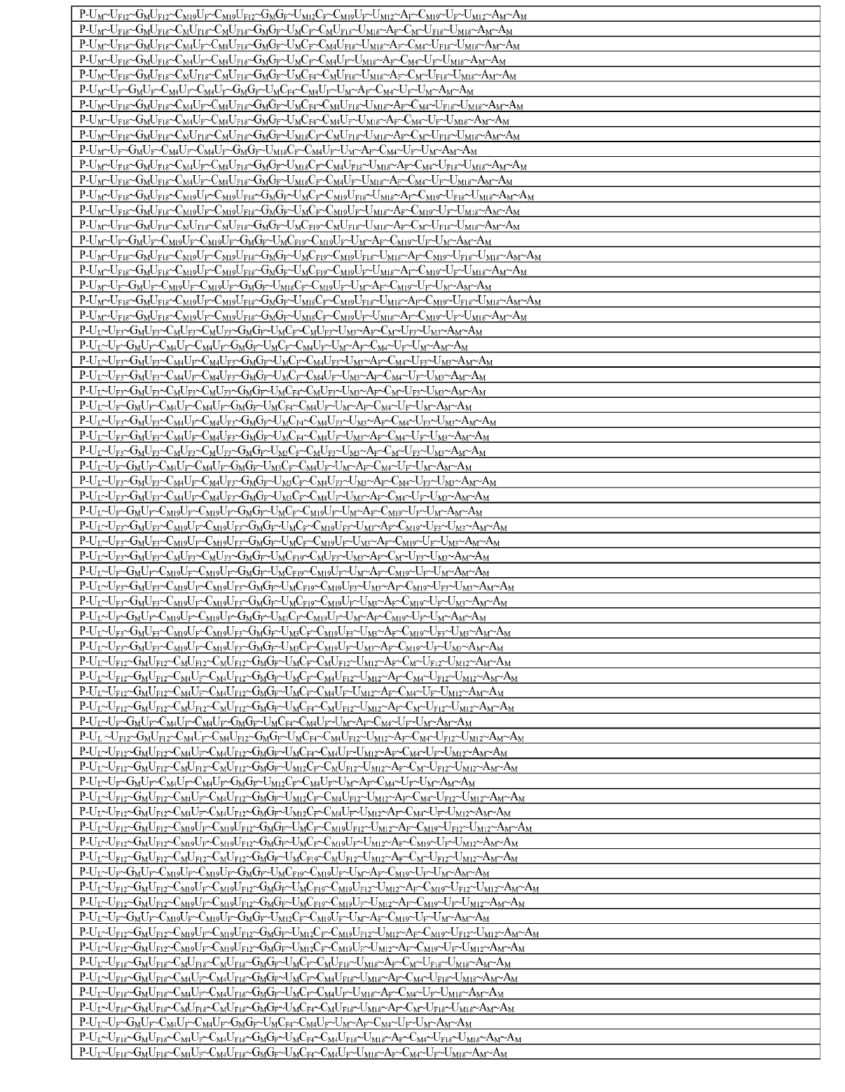

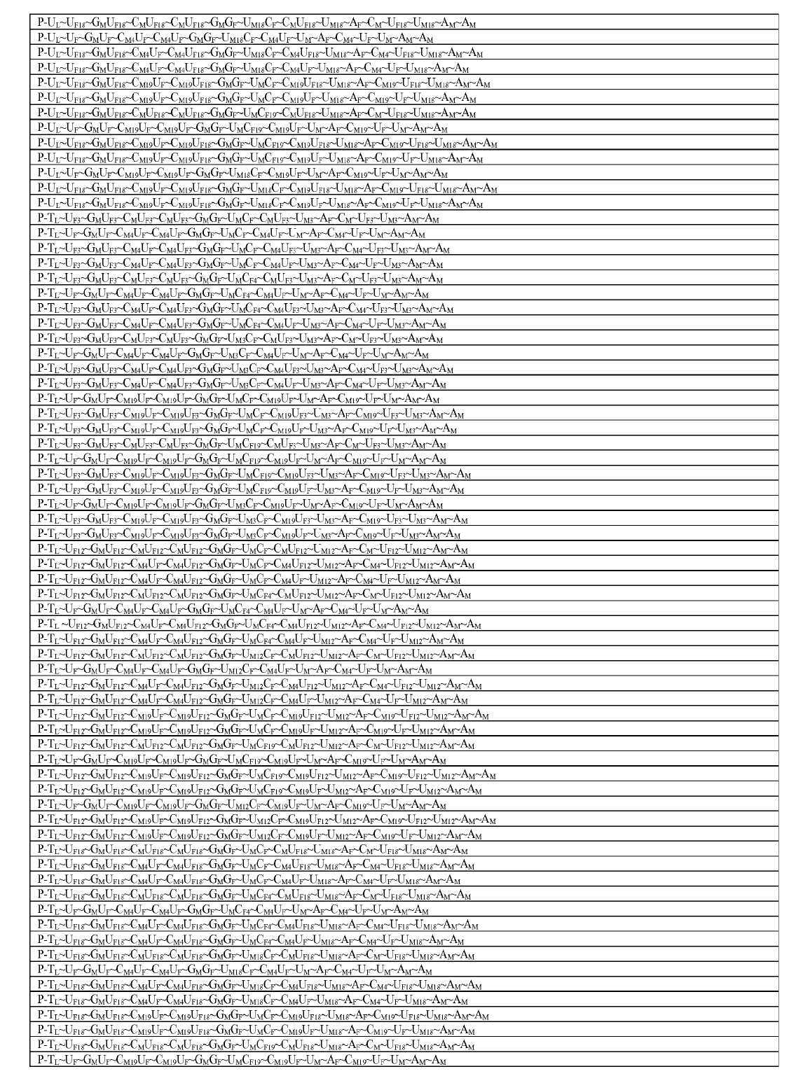

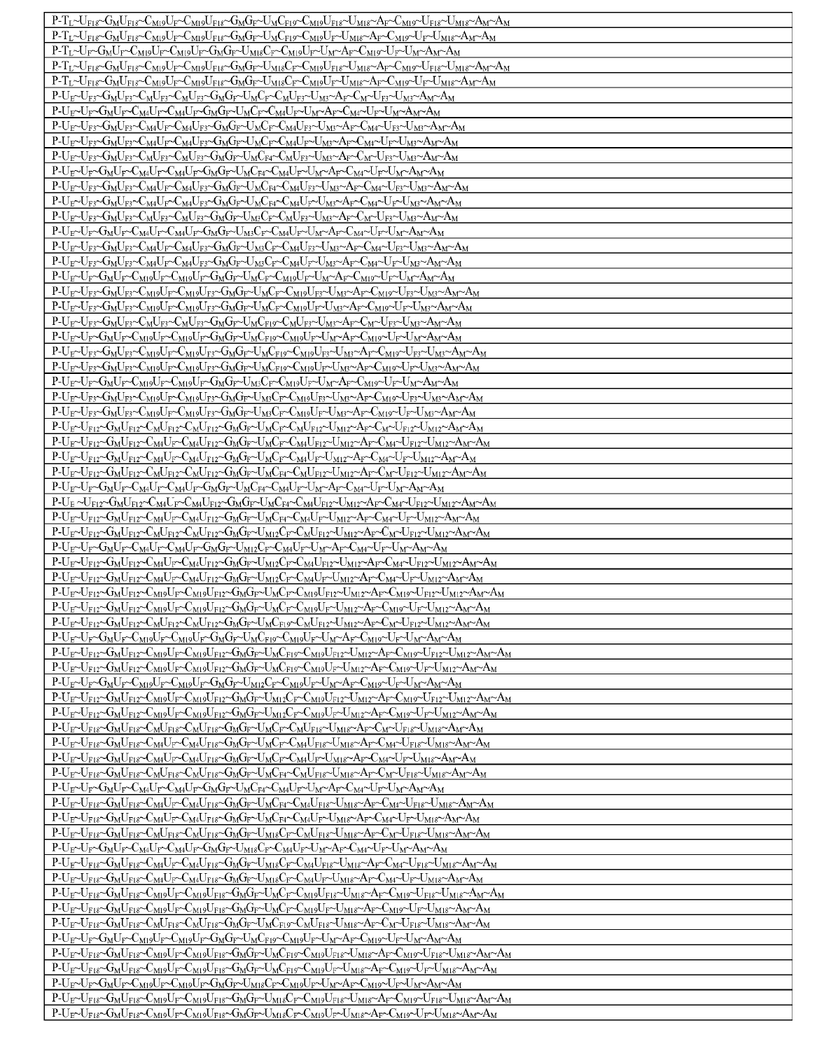

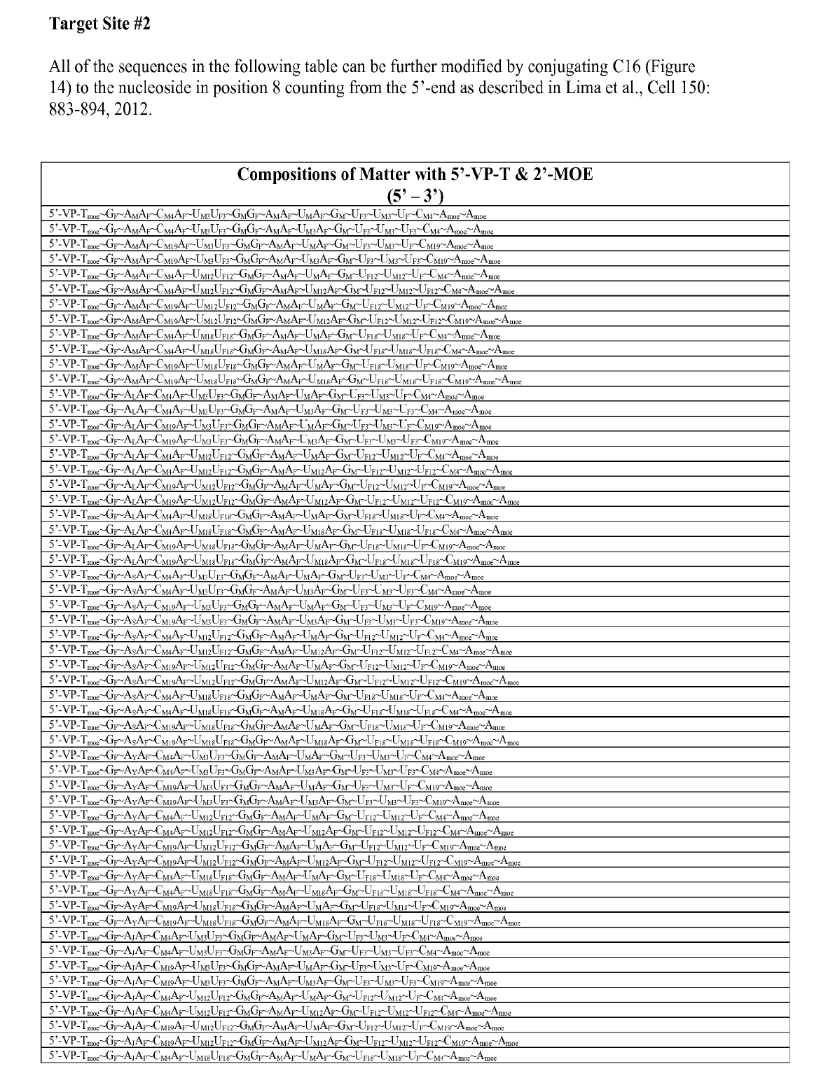

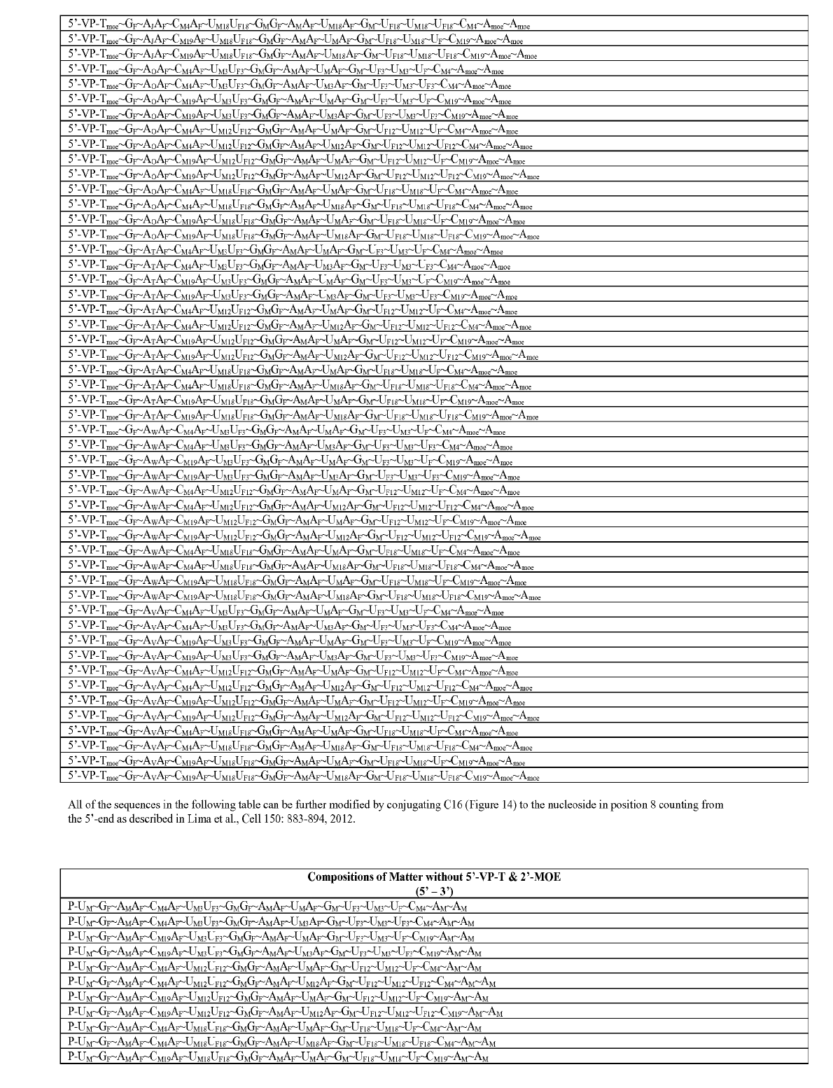

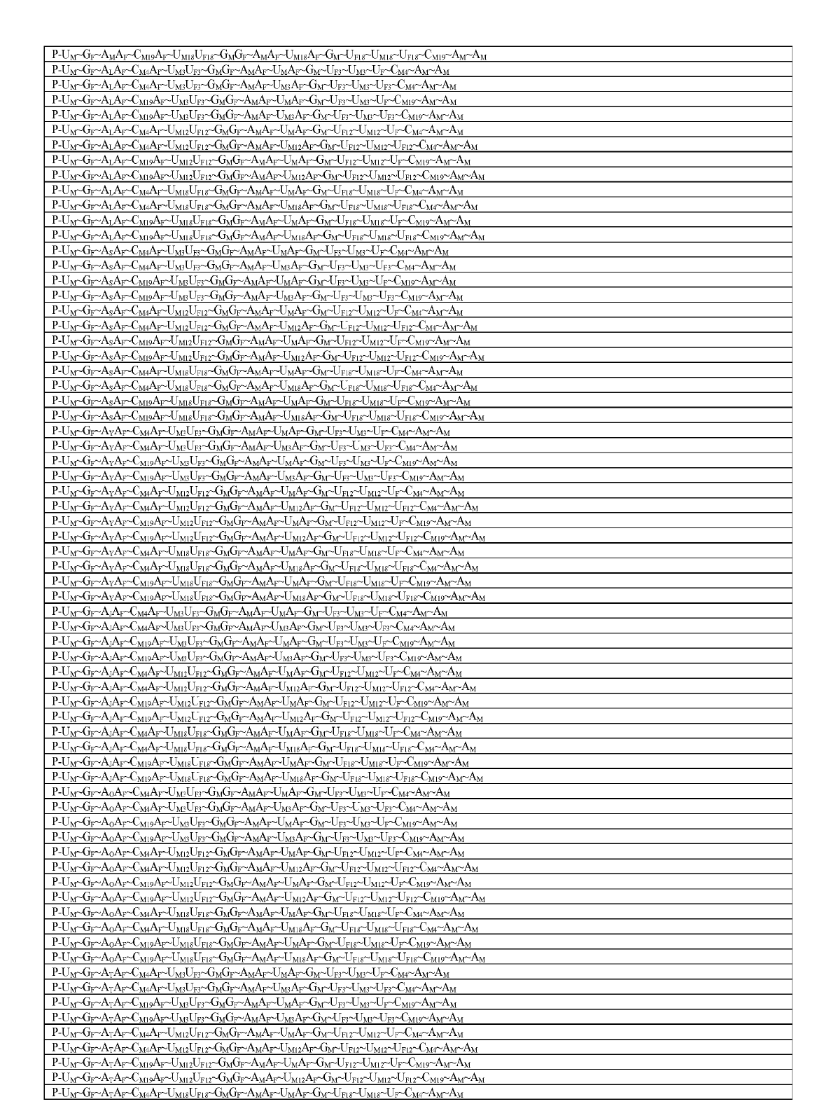

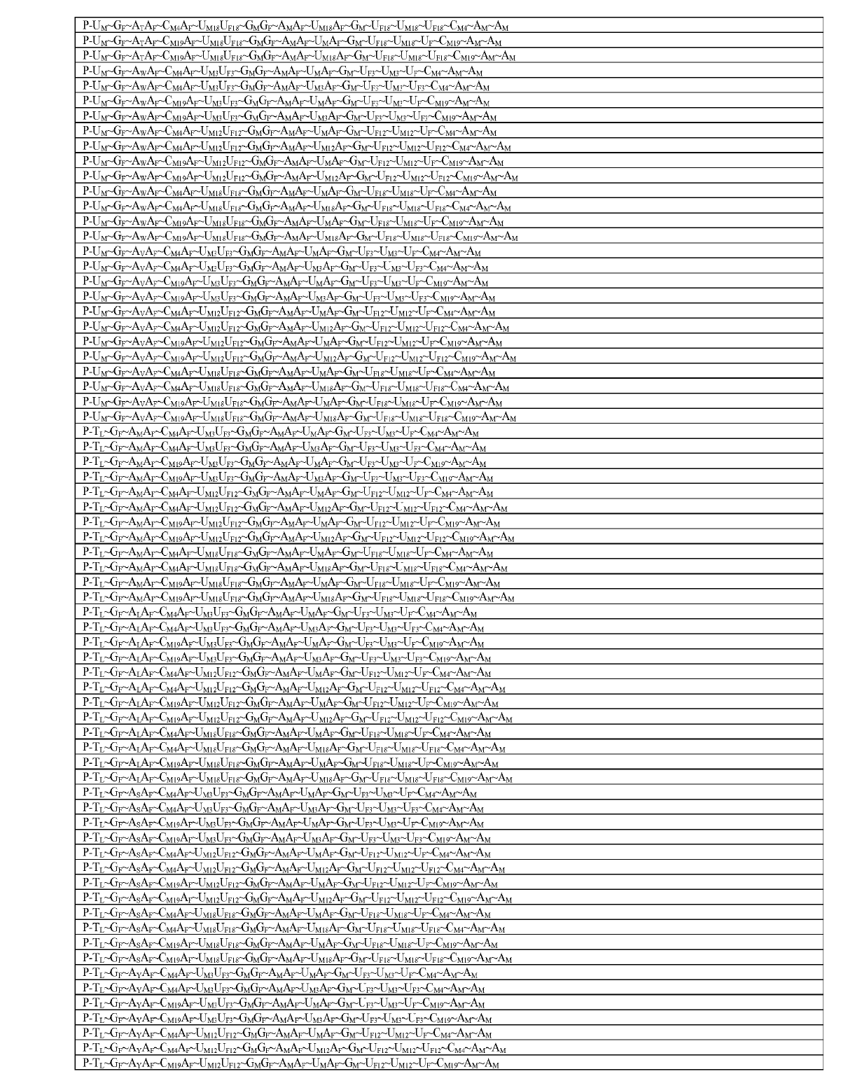

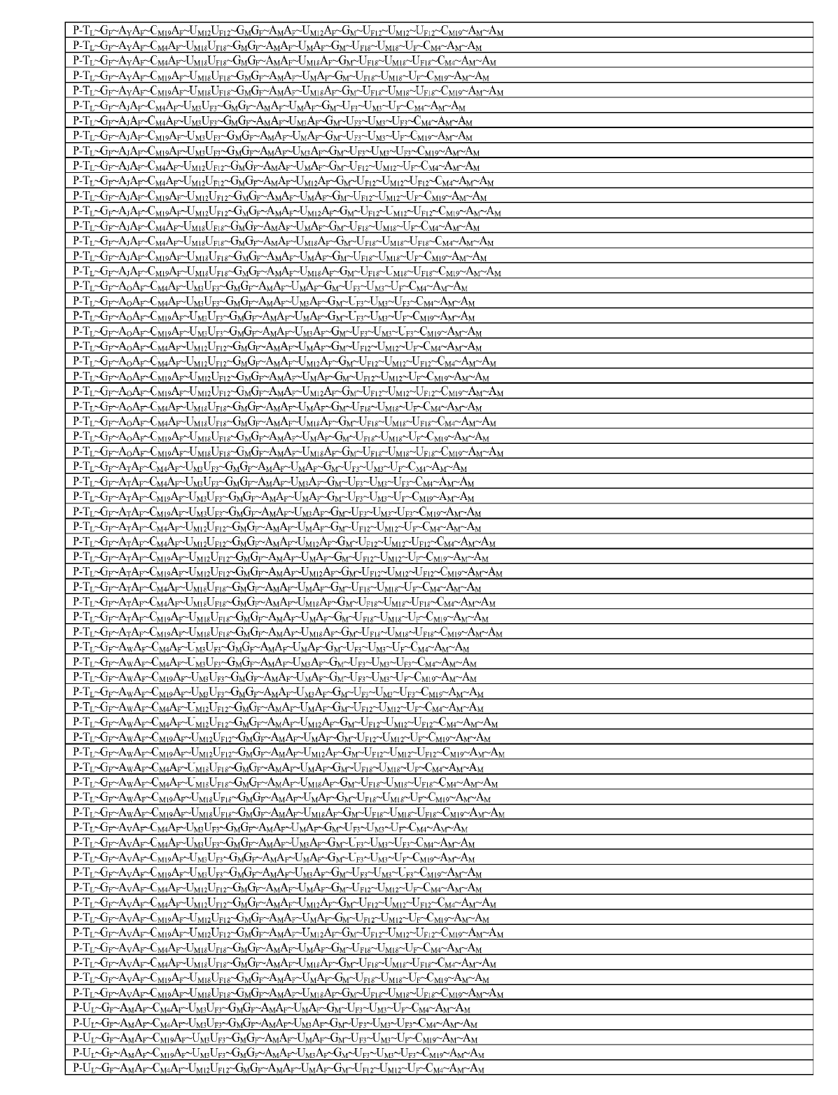

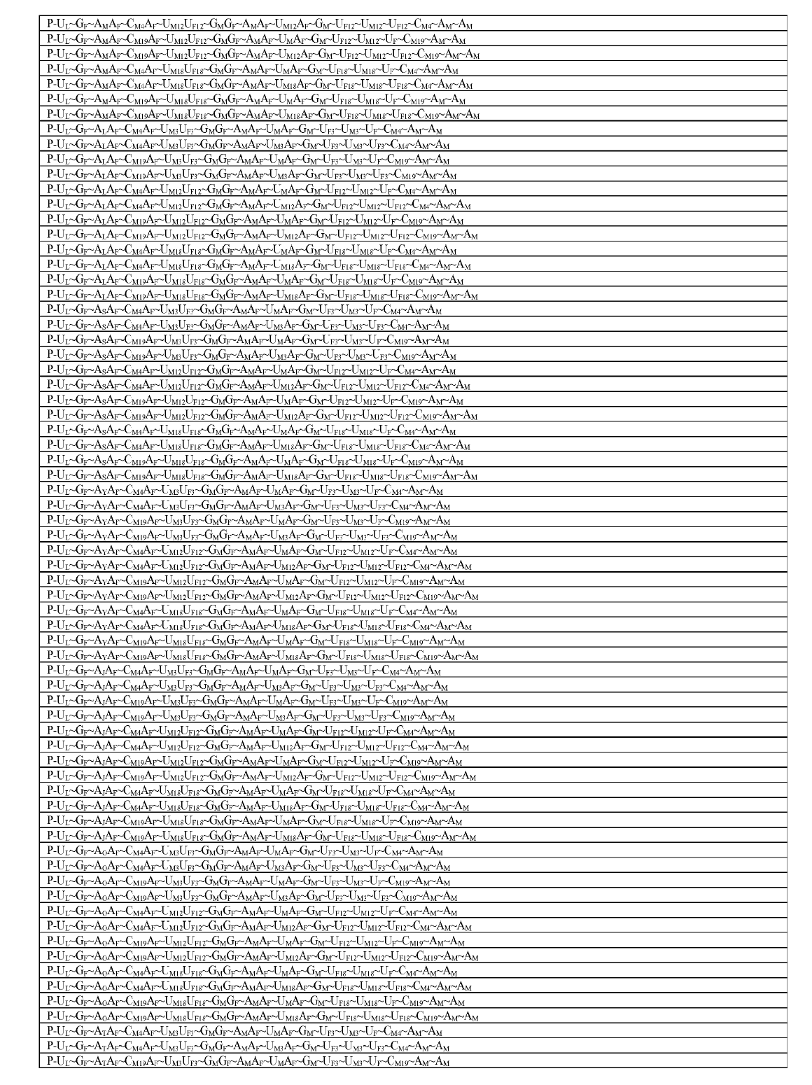

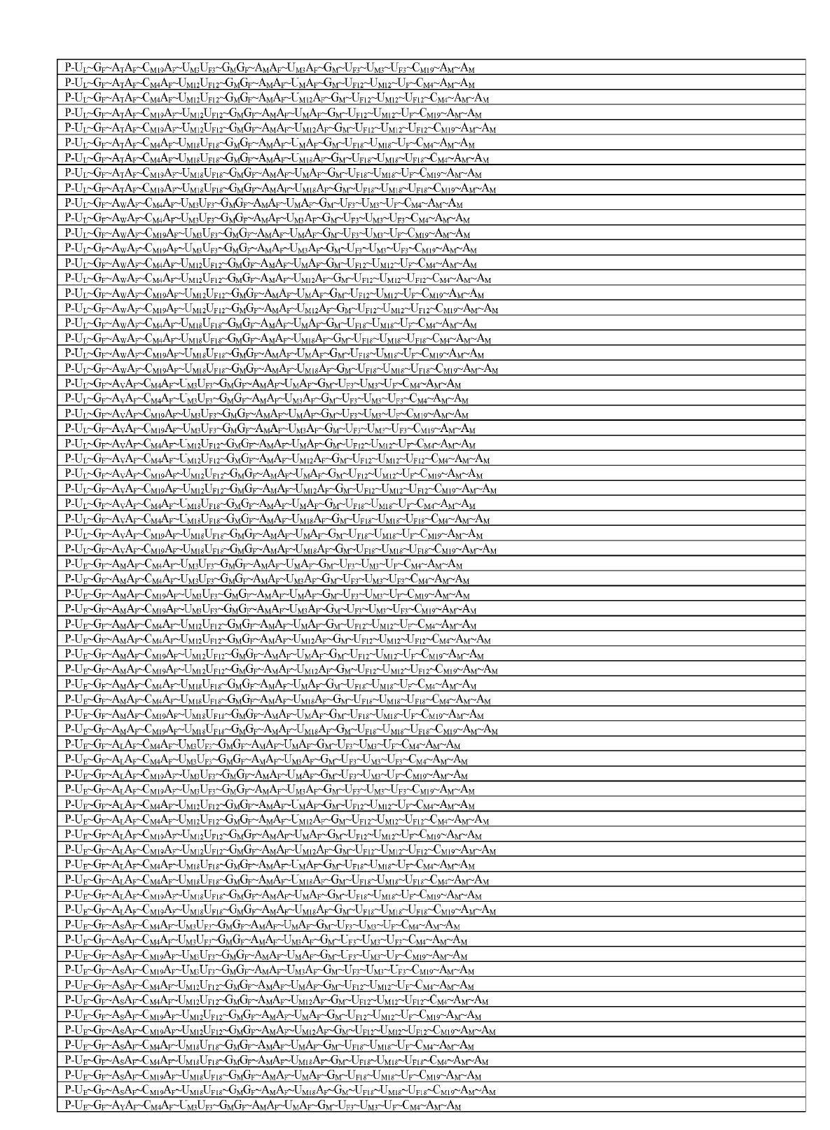

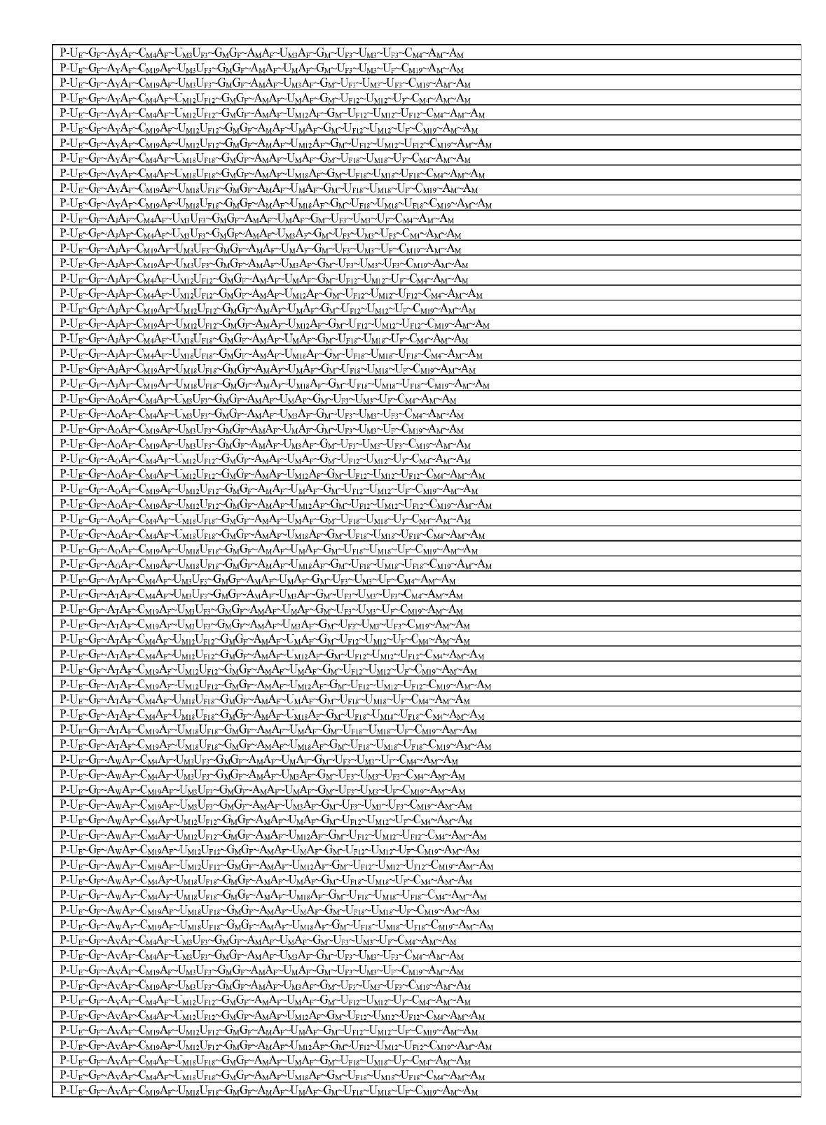

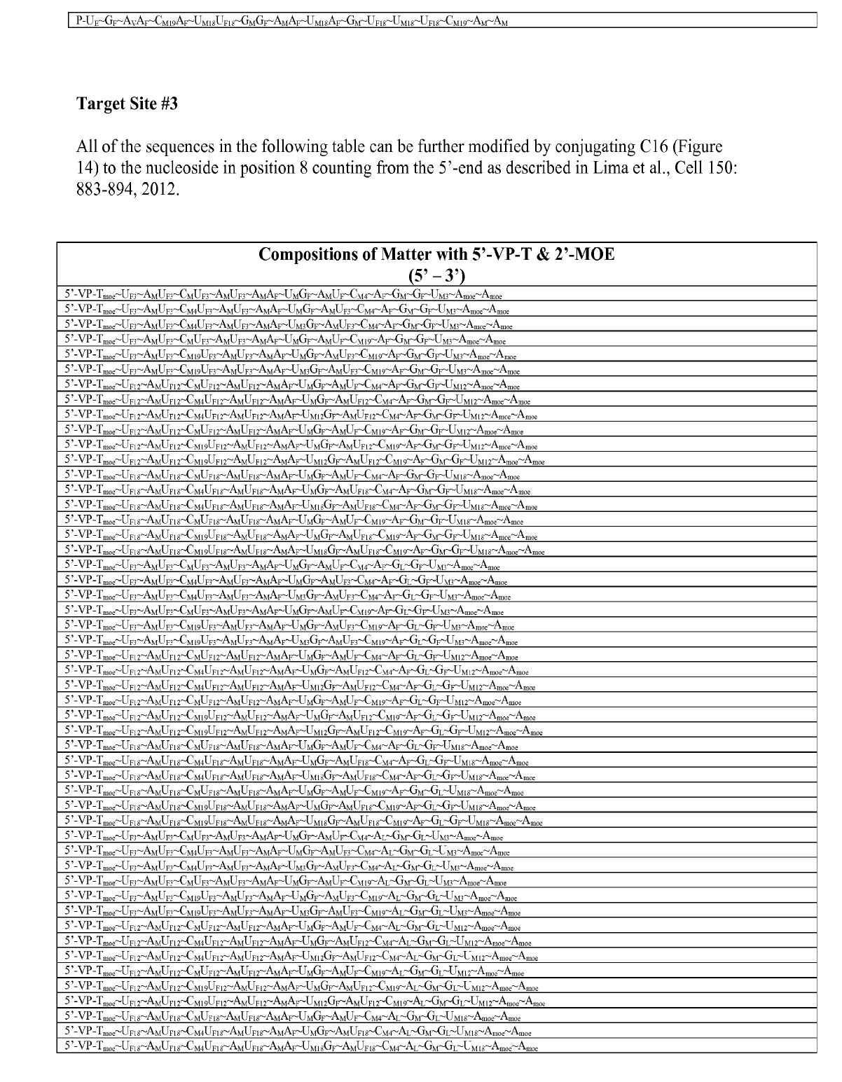

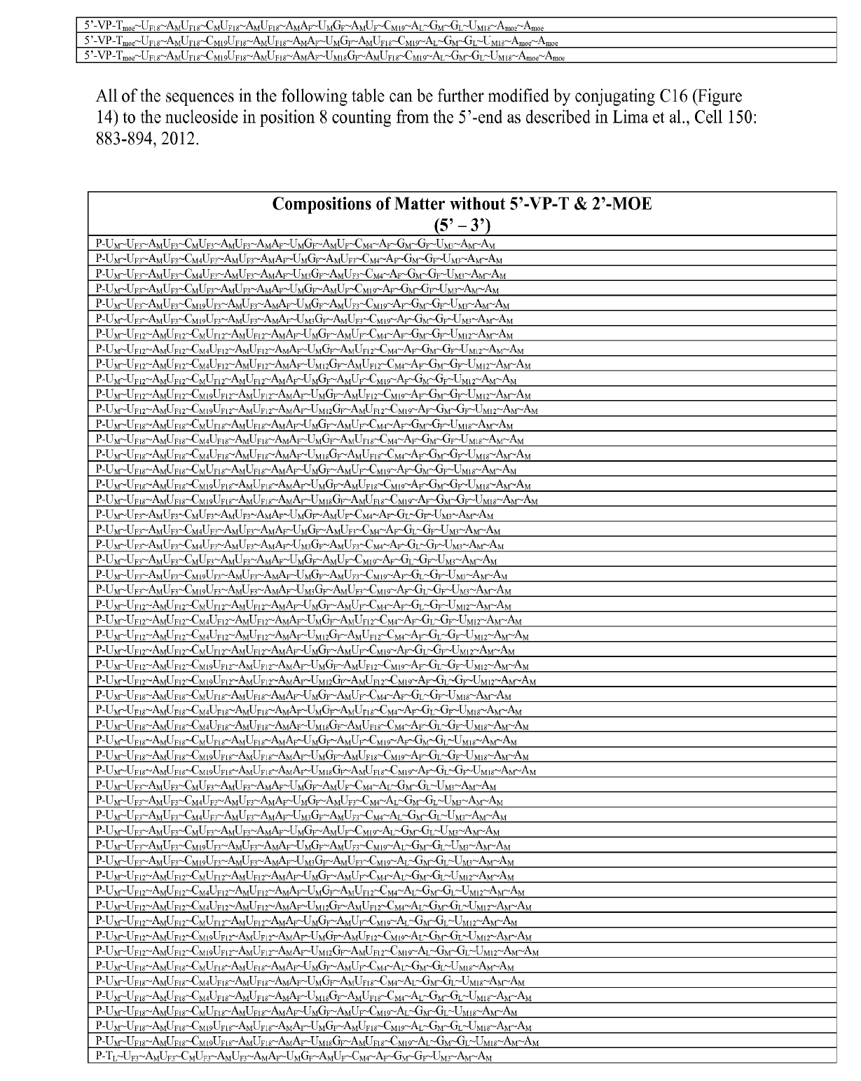

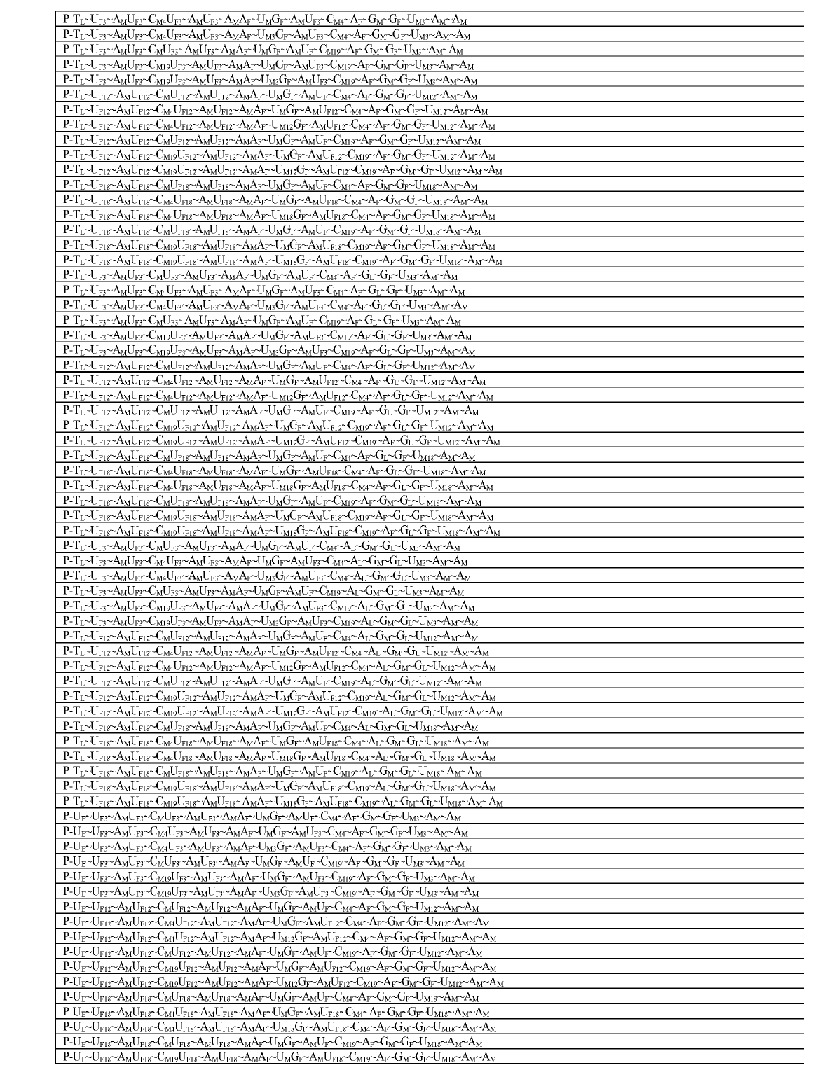

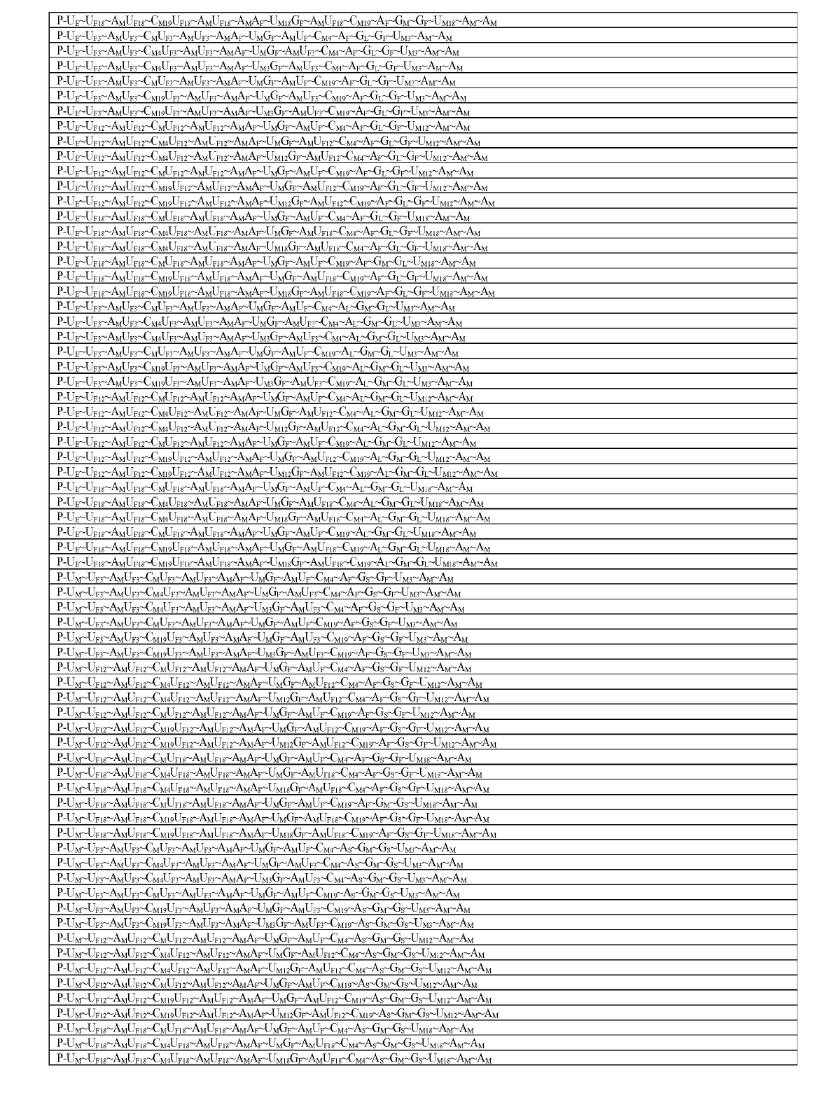

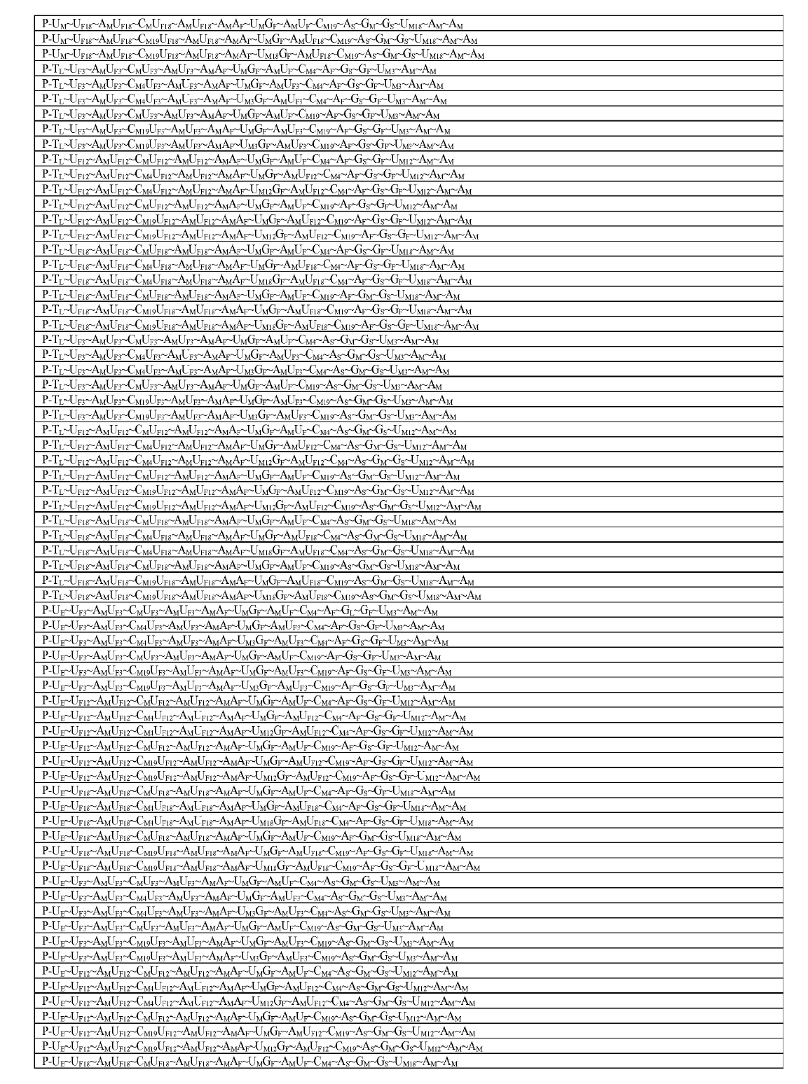

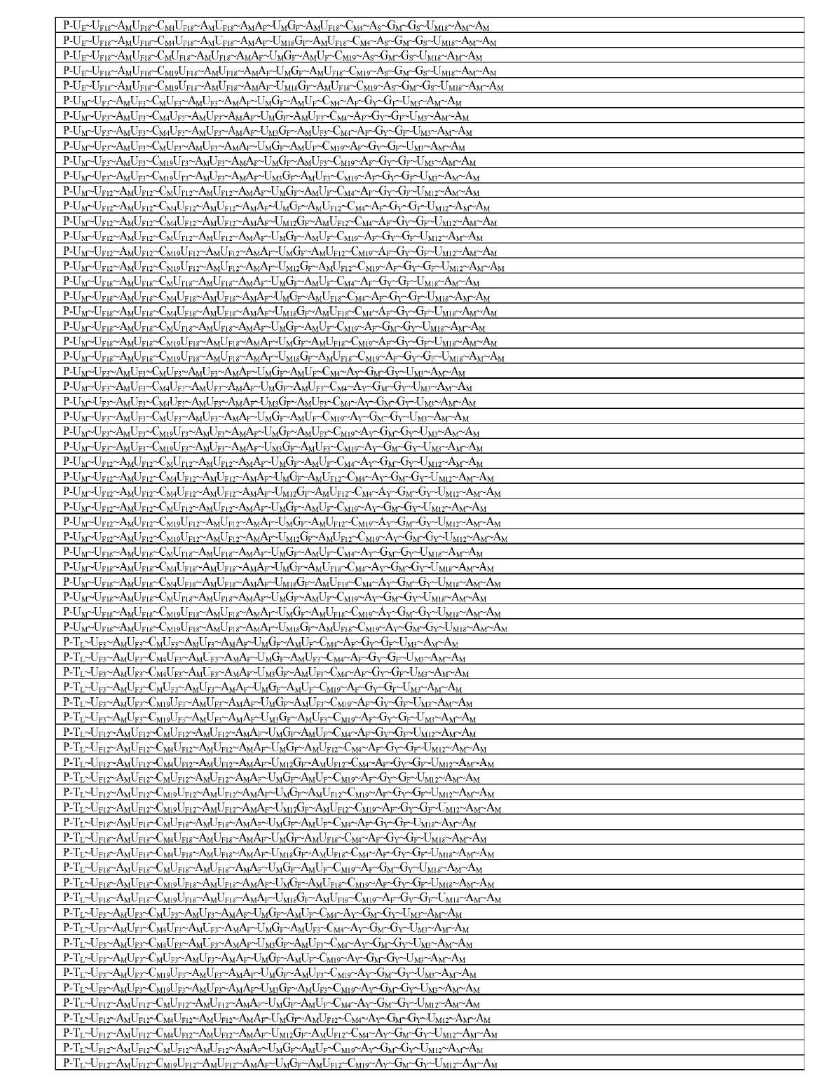

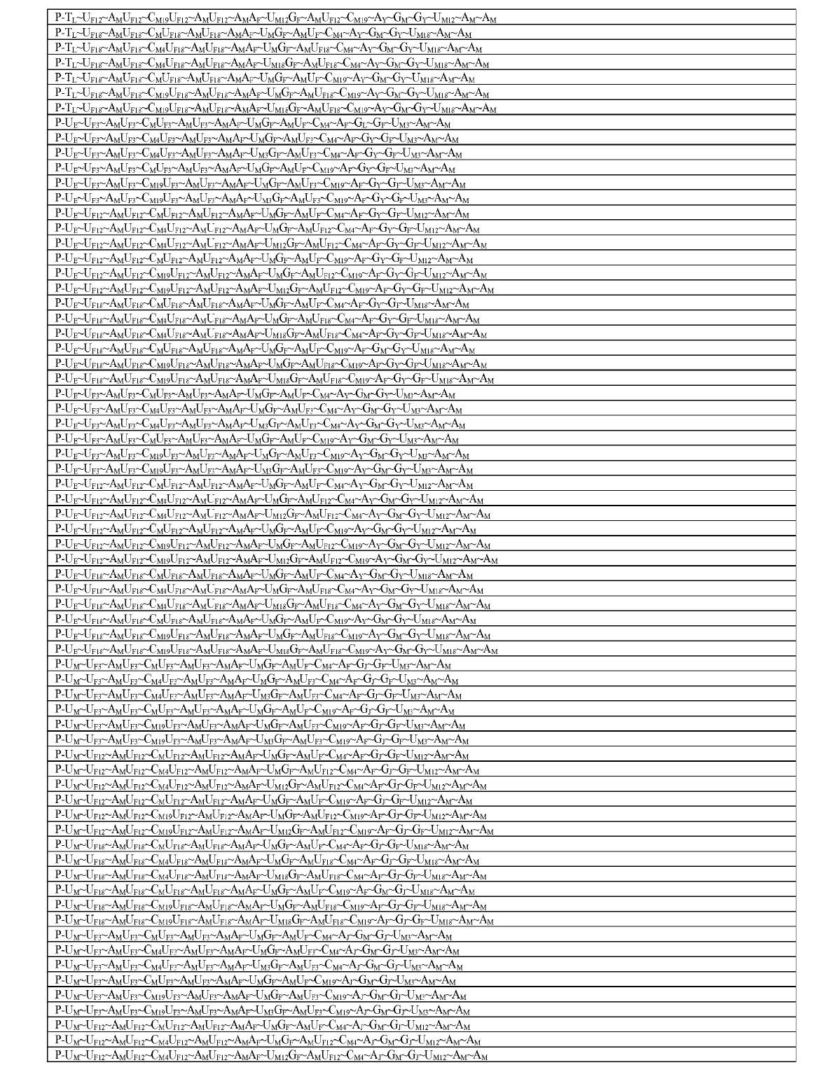

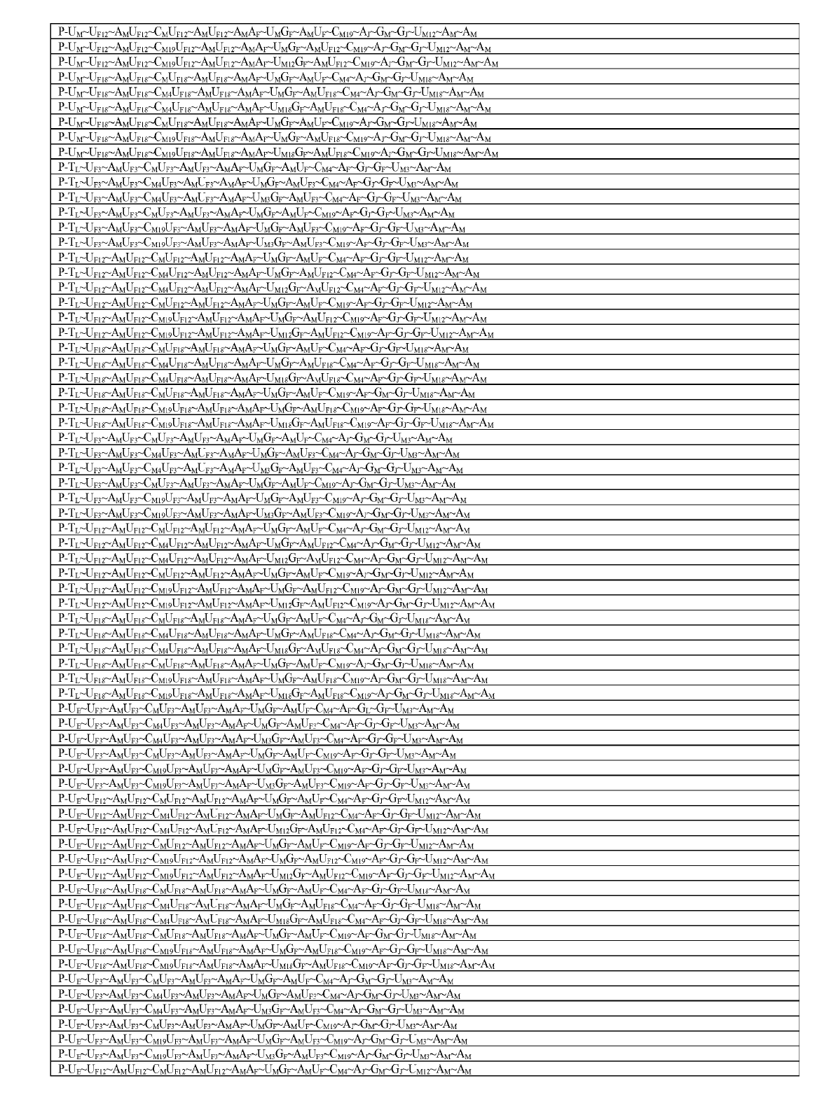

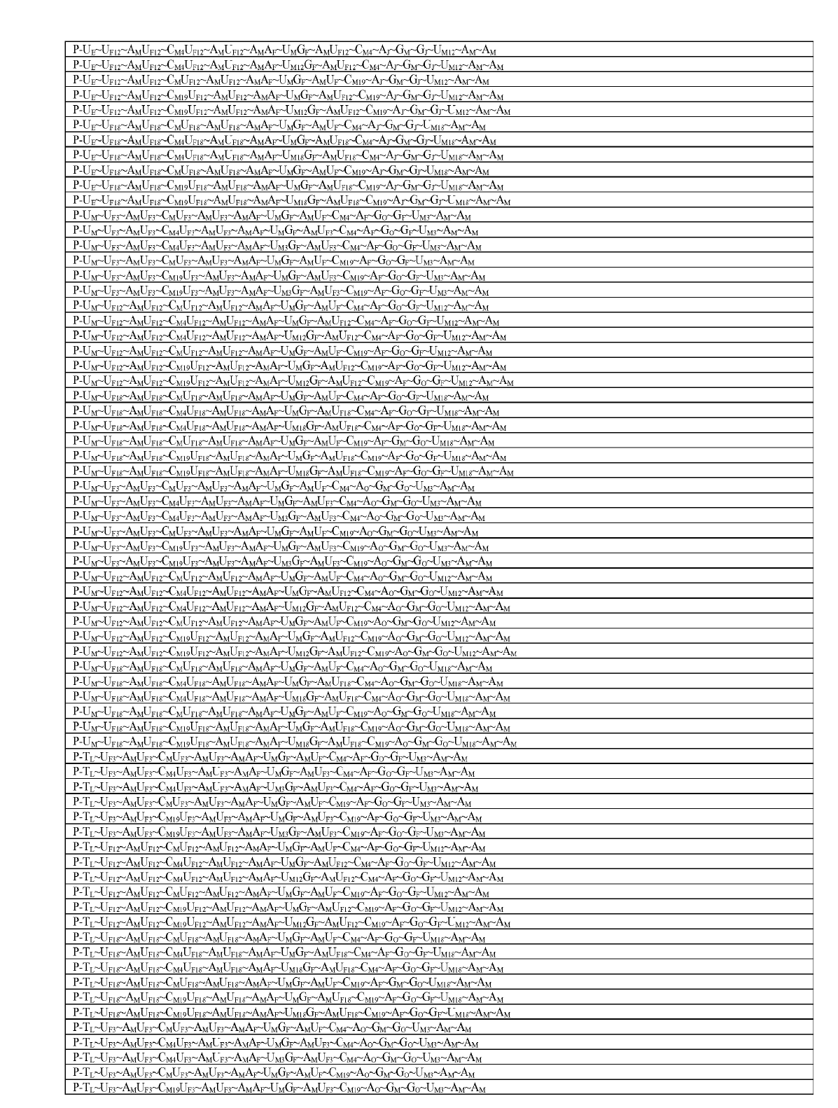

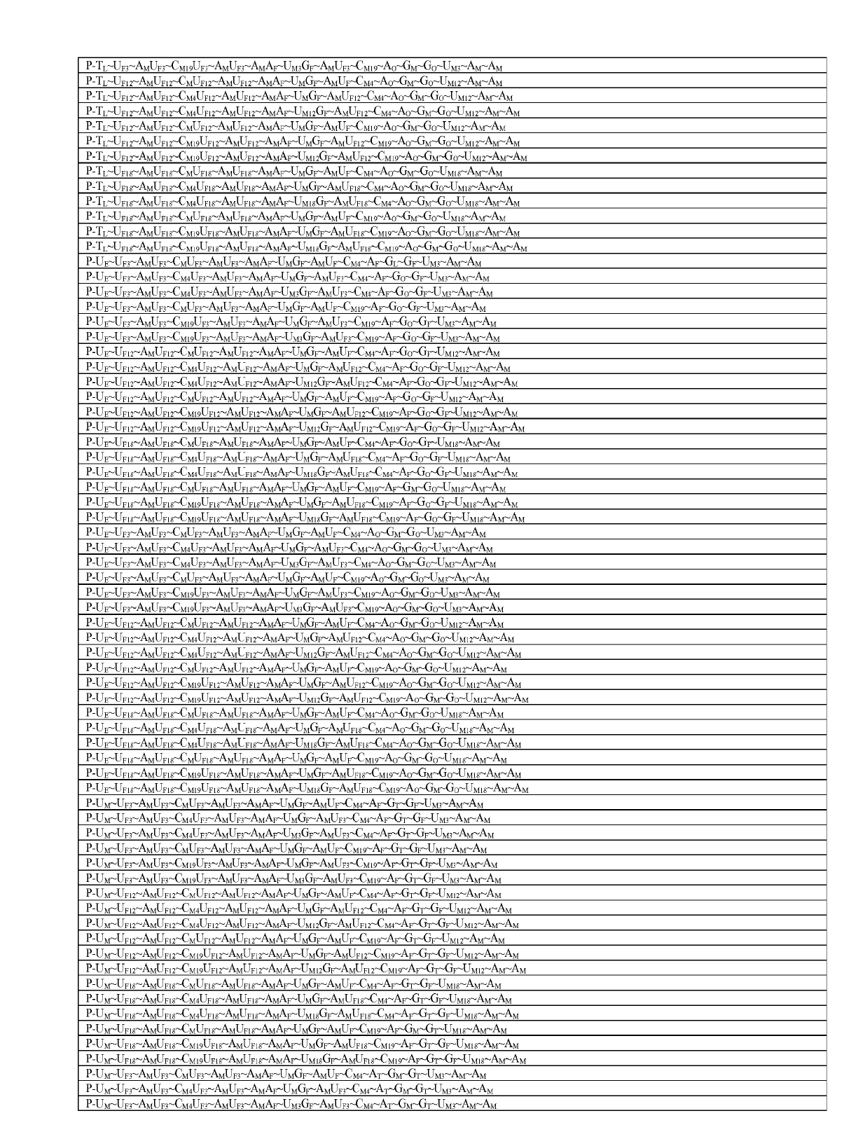

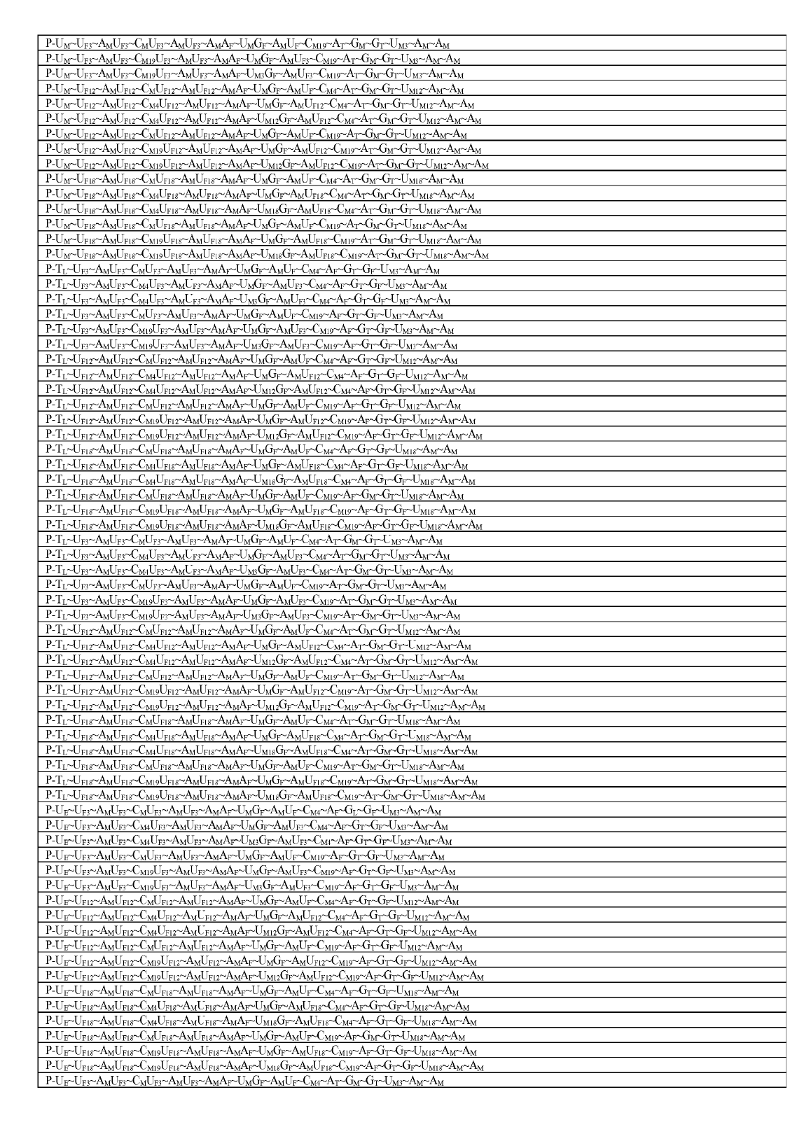

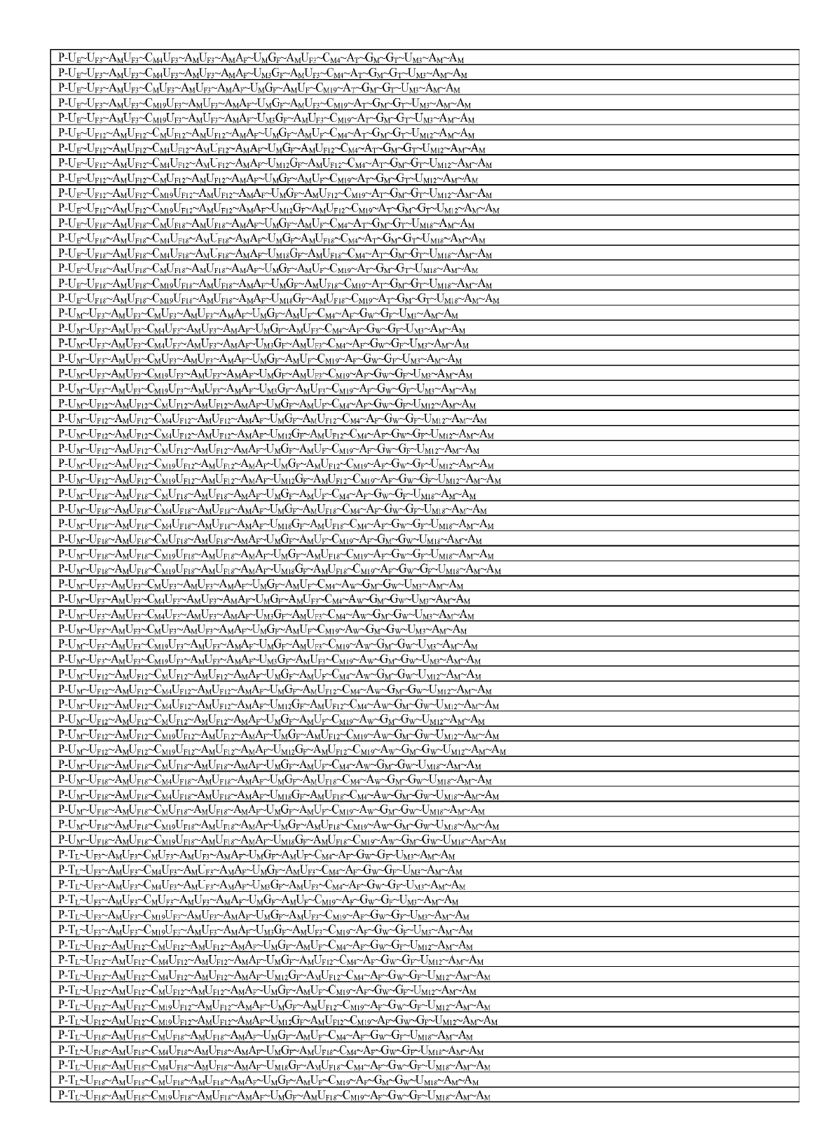

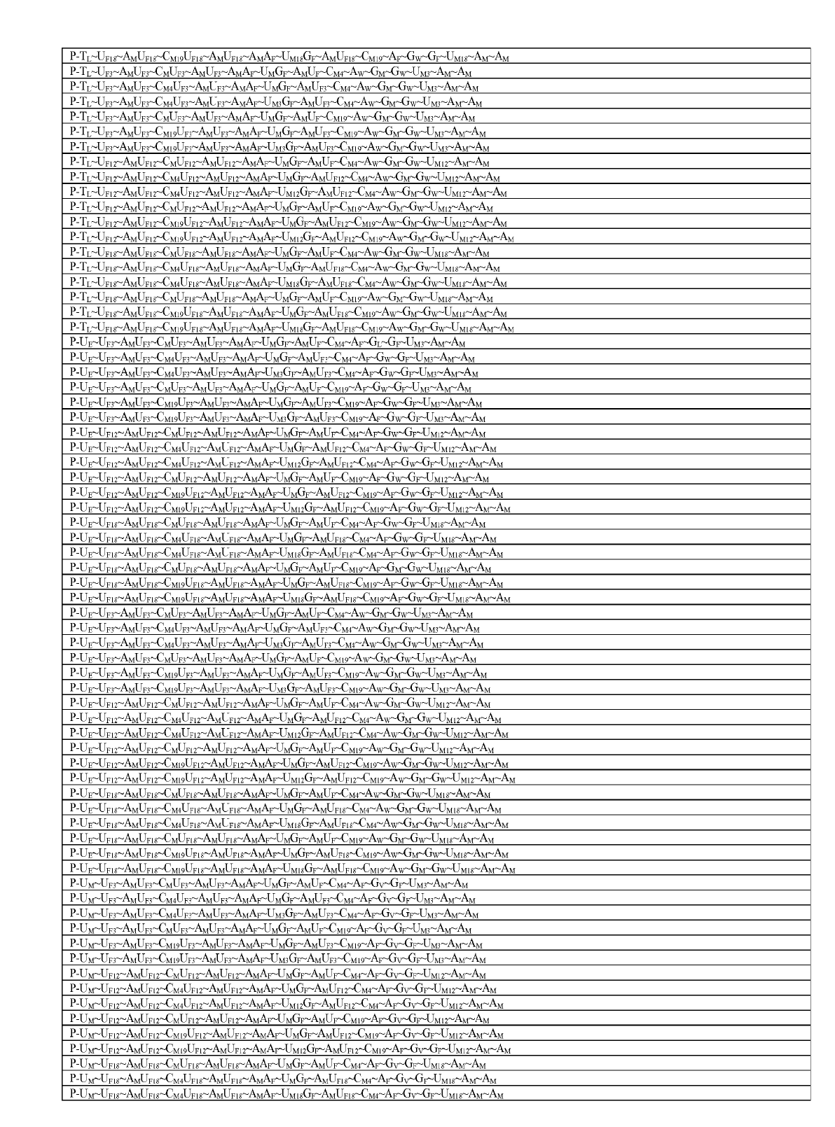

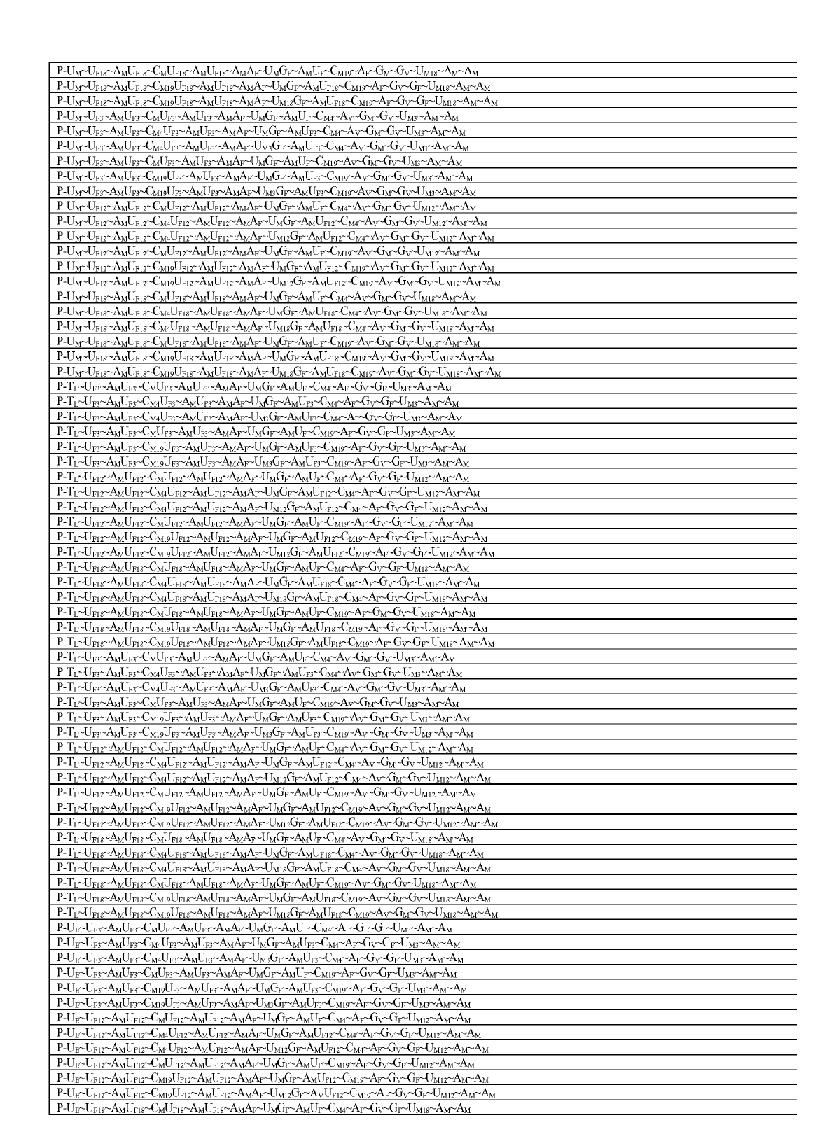

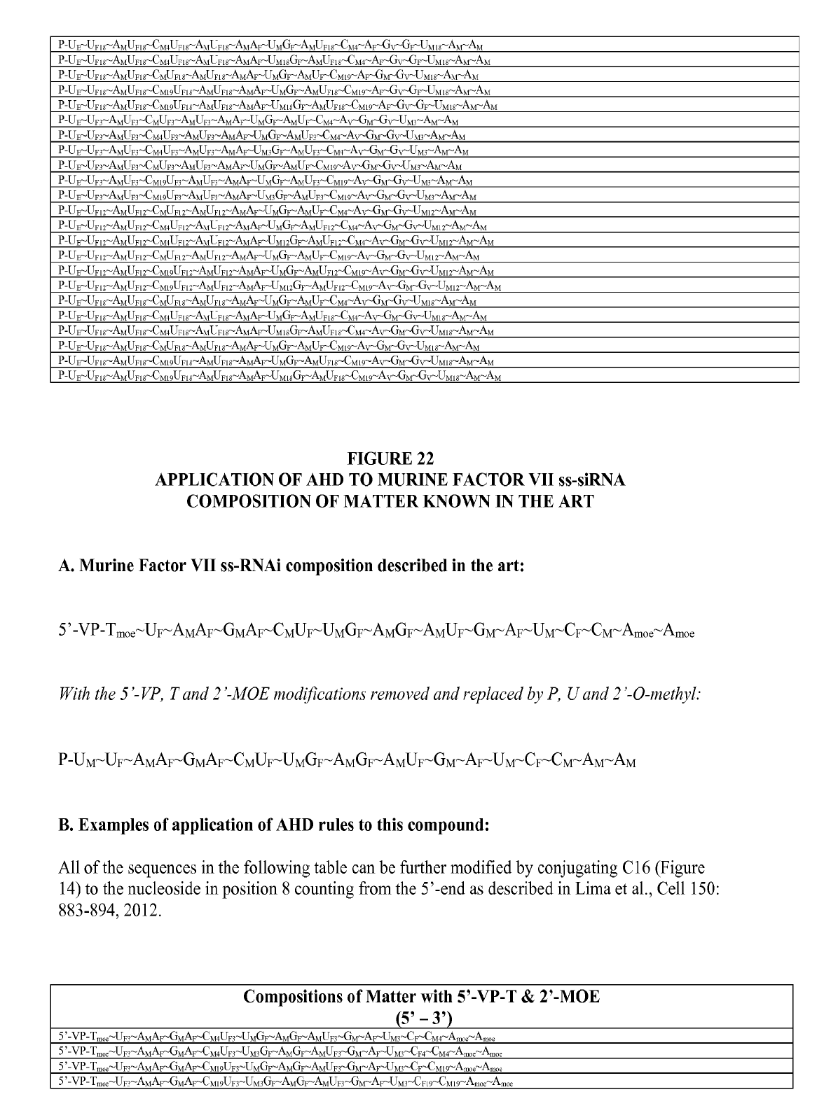

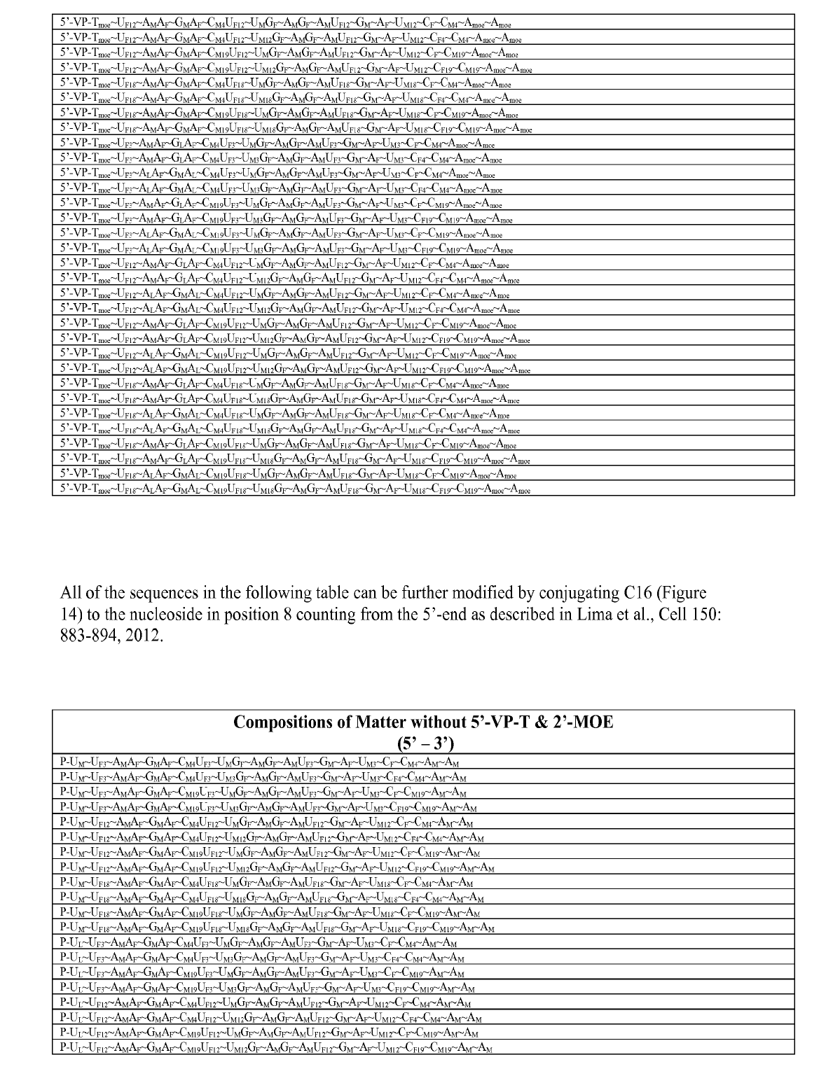

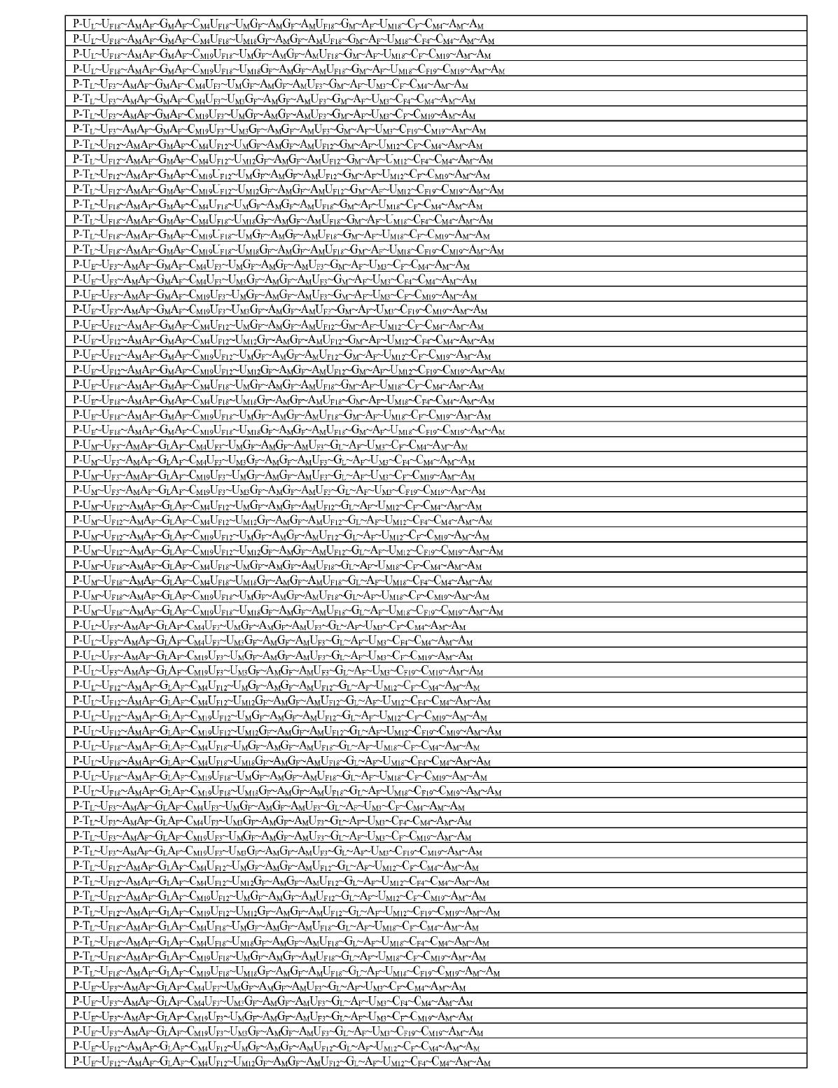

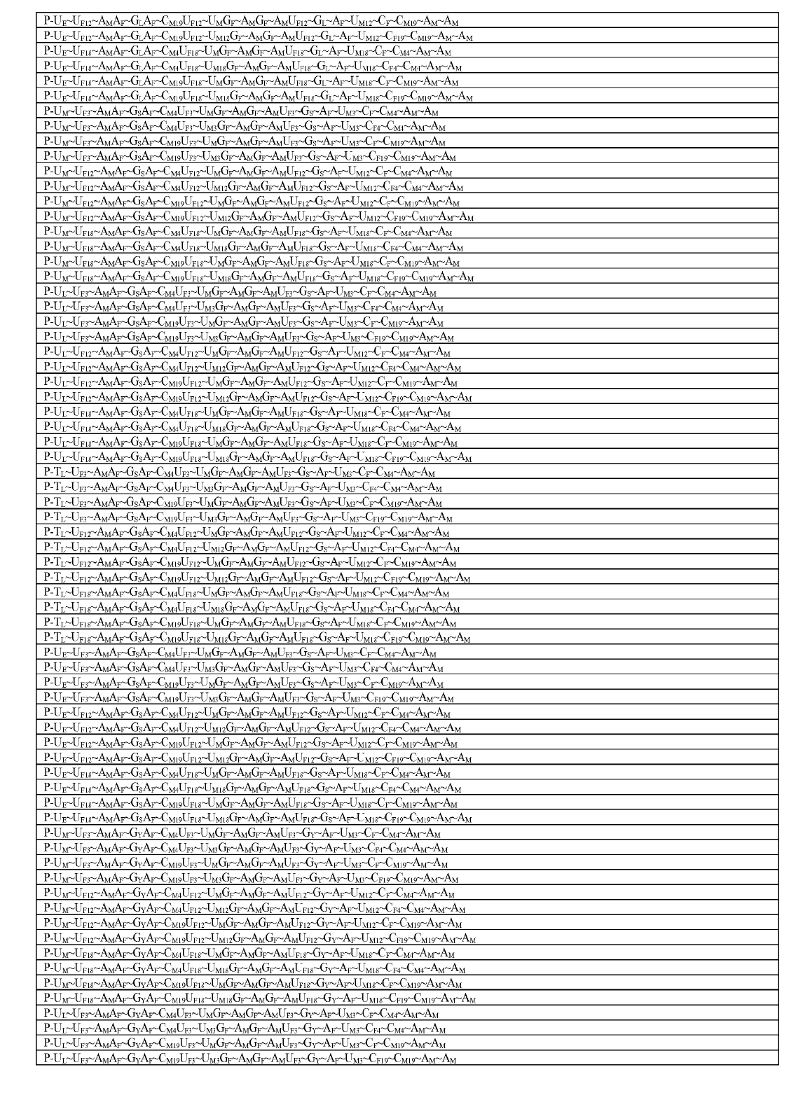

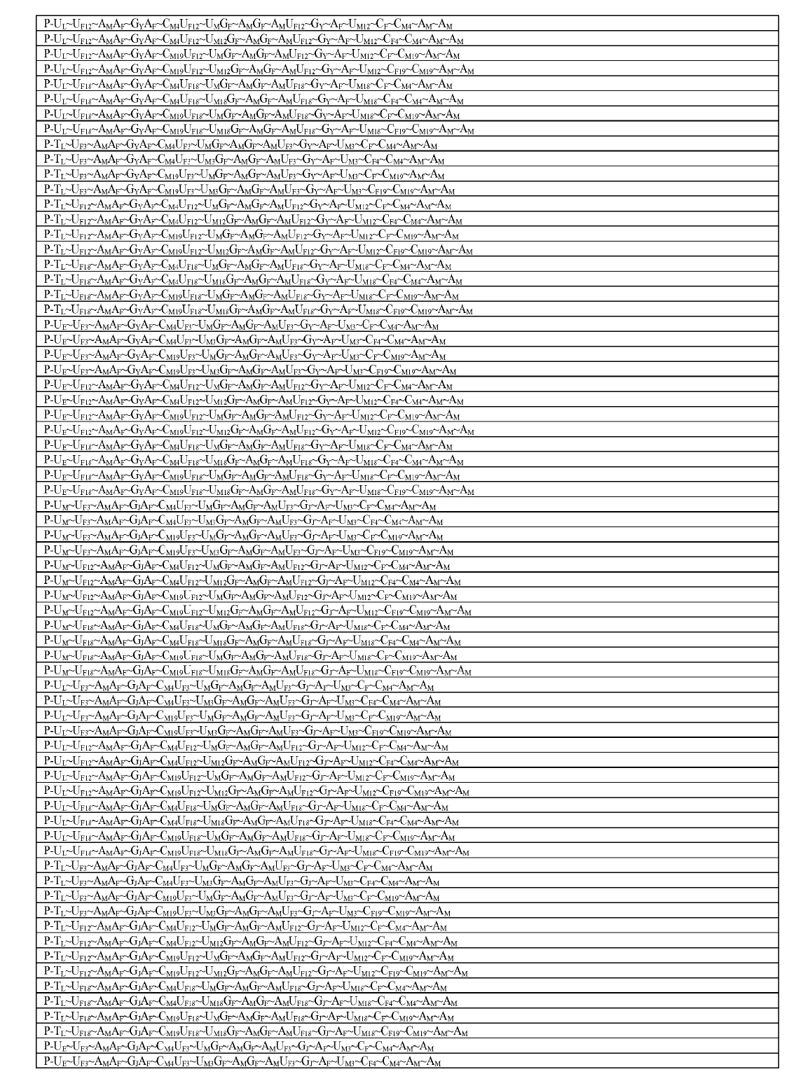

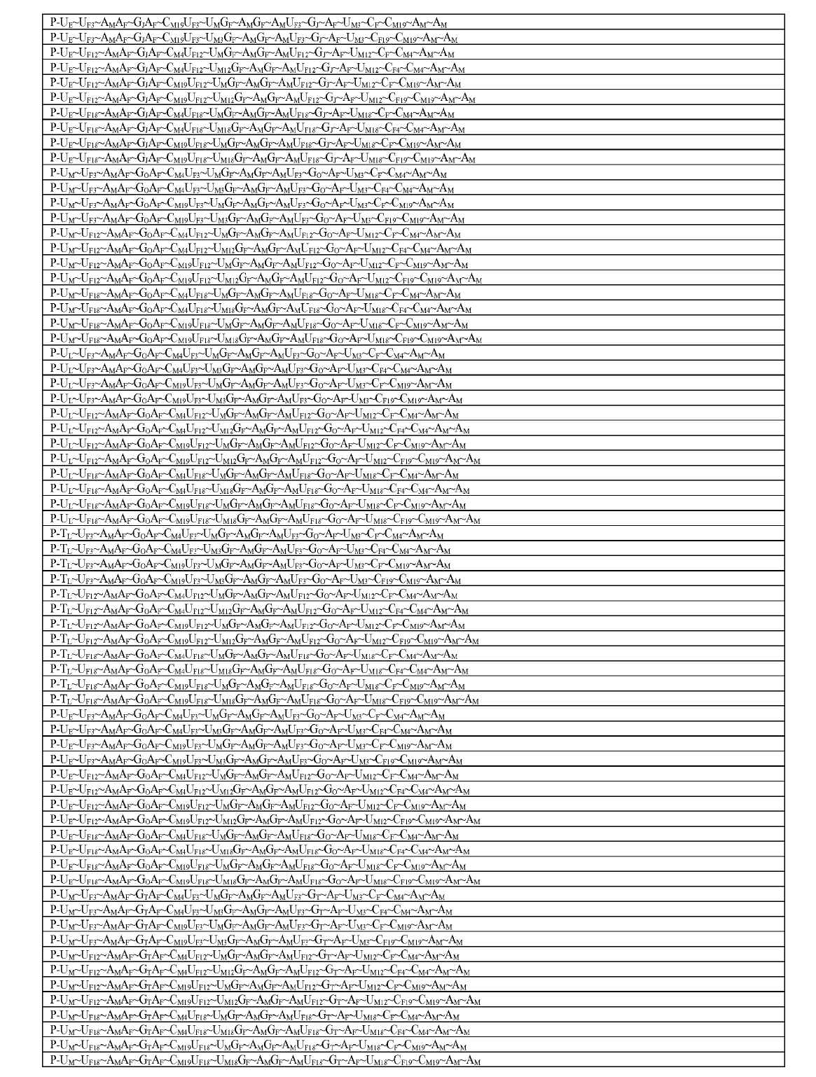

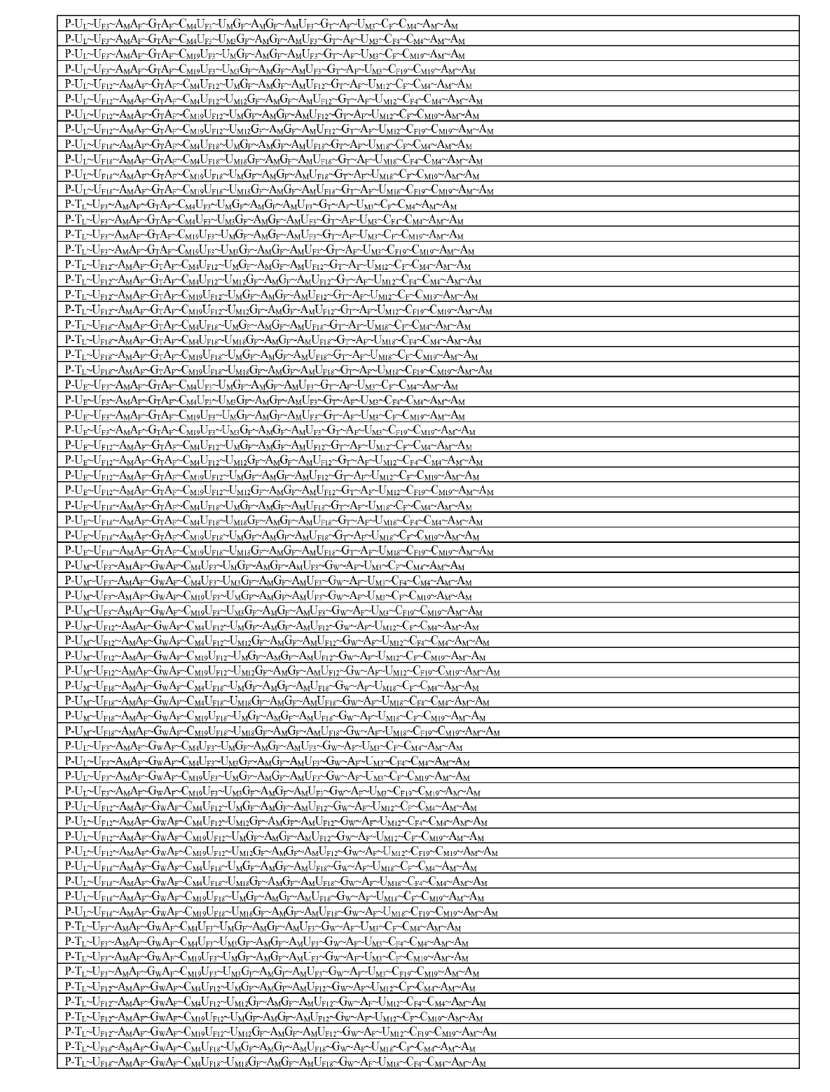

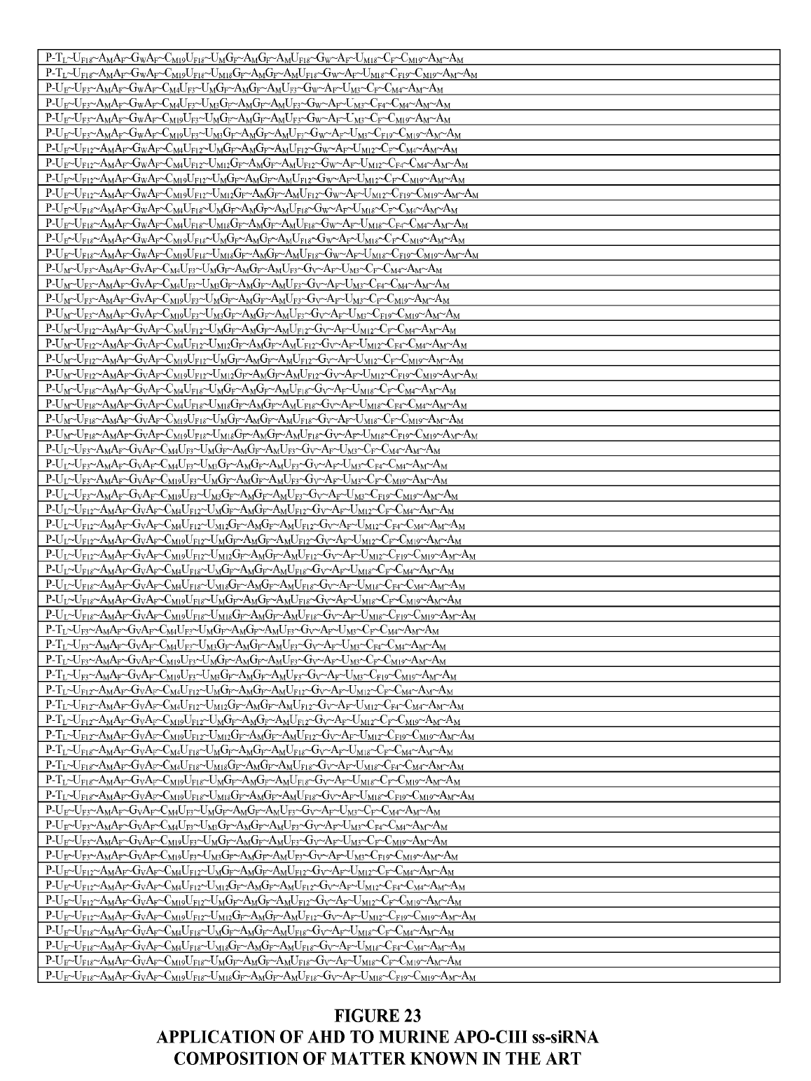

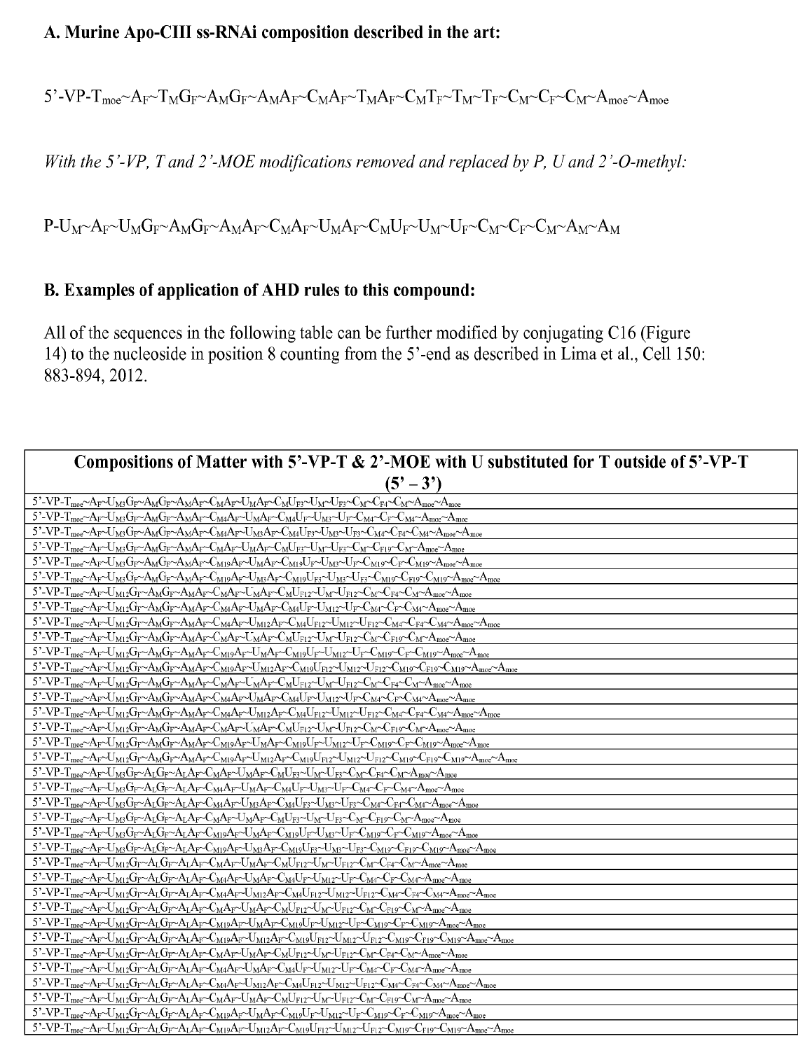

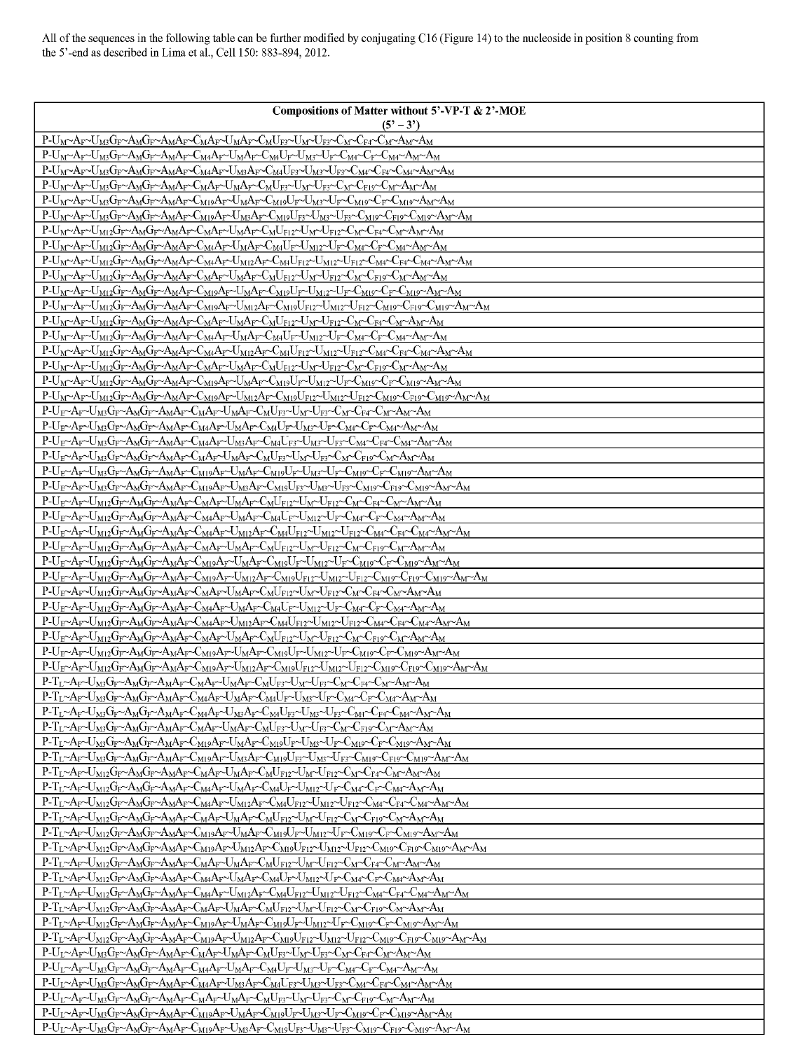

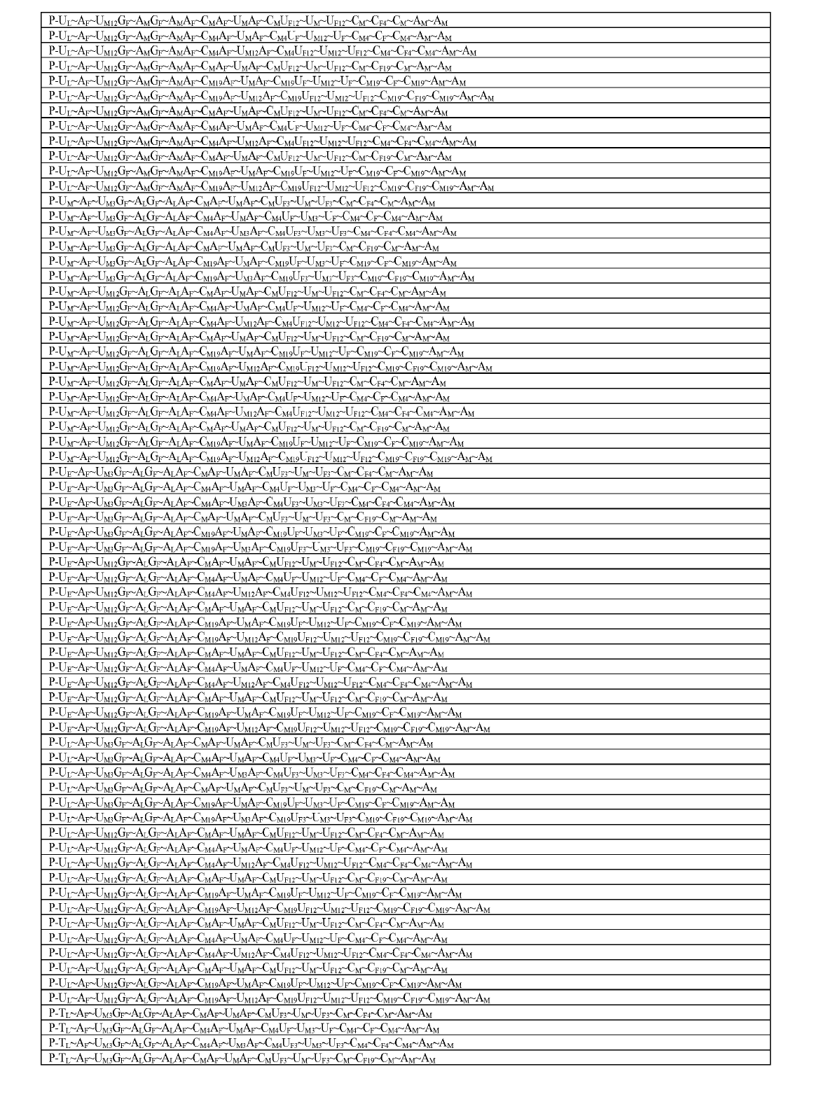

FIG. 21B applies the AHD rules of the present invention to ss-RNAi compounds directed to human and murine PTEN that are known in the art while FIG. 21C applies both the nuclease resistance and AHD rules to these compounds. FIG. 22B applies the AHD rules of the present invention to an ss-RNAi compound directed to murine Factor VII that is known in the art while FIG. 21C applies both the nuclease resistance and AHD rules to this compound. FIG. 23B applies the AHD rules of the present invention to an ss-RNAi compound directed to murine Apo-CIII that is known in the art while FIG. 23C applies both the nuclease resistance and AHD rules to this compound. Any of these compounds can also have C16, illustrated in FIG. 15, conjugated to the nucleoside in position 8 from the 5′-end where the U is replaced with a T.

The compounds in the art shown in FIGS. 21, 22 and 23 are all from the same reference (Lima et al., Cell 150: 883-894, 2012). The first of the three PTEN compounds listed under Target Site 1# in FIG. 21 was tested in mice and shown to have modest activity when used with the 5′-MP 5′-end. The presumably superior 5′-VP version shown in the figure was not tested. The second compound listed was stable in mouse liver except for the 5′-end phosphate that was removed in under 6 hours in this organ. It was not active in mice and the authors assumed the phosphate loss was responsible for the inactivity. It was active in tissue culture. The third compound in this group worked well in tissue culture but was rapidly fragmented in liver cells in animals. The compounds listed under Target Sites #2 and #3 were shown to be very effective at reducing PTEN in the liver.

As pointed out previously herein 2′-MOE used at the 5′-end of an ss-RNAi can have a negative effect on the ability of cellular phosphorylases that can add phosphate to the 5′ carbon of a ribonucleotide but not have a negative effect on dephosphorylating enzymes that can remove phosphate from this position. Thus, a phosphate isostere such as 5′-MP or 5′-VP that has a resistant bond that cannot be cleaved by known dephosphorylating enzymes is an essential feature. In addition, depending on the ss-RNAi sequence 5′-VP, which is more active at promoting ss-RNAi activity than 5′-MP is at best equivalent to natural phosphate in this position and can be inferior to it. As a result 5′-VP-Tₘₒₑ and 3′-end terminal nucleosides with 2′-MOE are not used in the comparator parent ss-RNAi for comparison to ss-RNAi of the same sequence modified using AHD. Further, although 2′-MOE has the C3′-endo conformation when present in an oligoribonucleotide it cannot function as an AHD modification because of the large size of the 2′-methyoxyethyl group and because it has no special ability to promote the C3′-endo conformation in contiguous nucleosides with more flexible or flexible sugars or sugar analogs. It is acceptable for use, however, at the 5′-end with a dephosphoryating enzyme resistant phosphate isostere and in the overhang precursors.

The compound shown in FIG. 22 targets Factor VII. It and the compound shown in FIG. 23 were used to confirm the usefulness of the 5′-VP modification when applied to a 5′-end terminal 2′-MOE nucleoside. The ss-RNAi compound in FIG. 23 that is directed to Apo-CIII is shown in the Lima et al. (2012) publication as having T rather than U in the sequence generally. This is assumed to be an error and the sequence for comparative purposes is adjusted to have U instead of T. Thymidine is not well tolerated in ss-RNAi and thymine-containing ribonucleosides are particularly subject to enzymatic attack. Although the ss-RNAi 5-VP containing compounds used by Lima et al. were capable of a high level of activity in terms of suppressing their targets in animal livers they had very poor potency as evidence by the very high doses needed to achieve this effect (range 100-300 mg/kg). They also appeared to be even less active in other organ types tested. All of these compounds were administered to animals without a carrier.

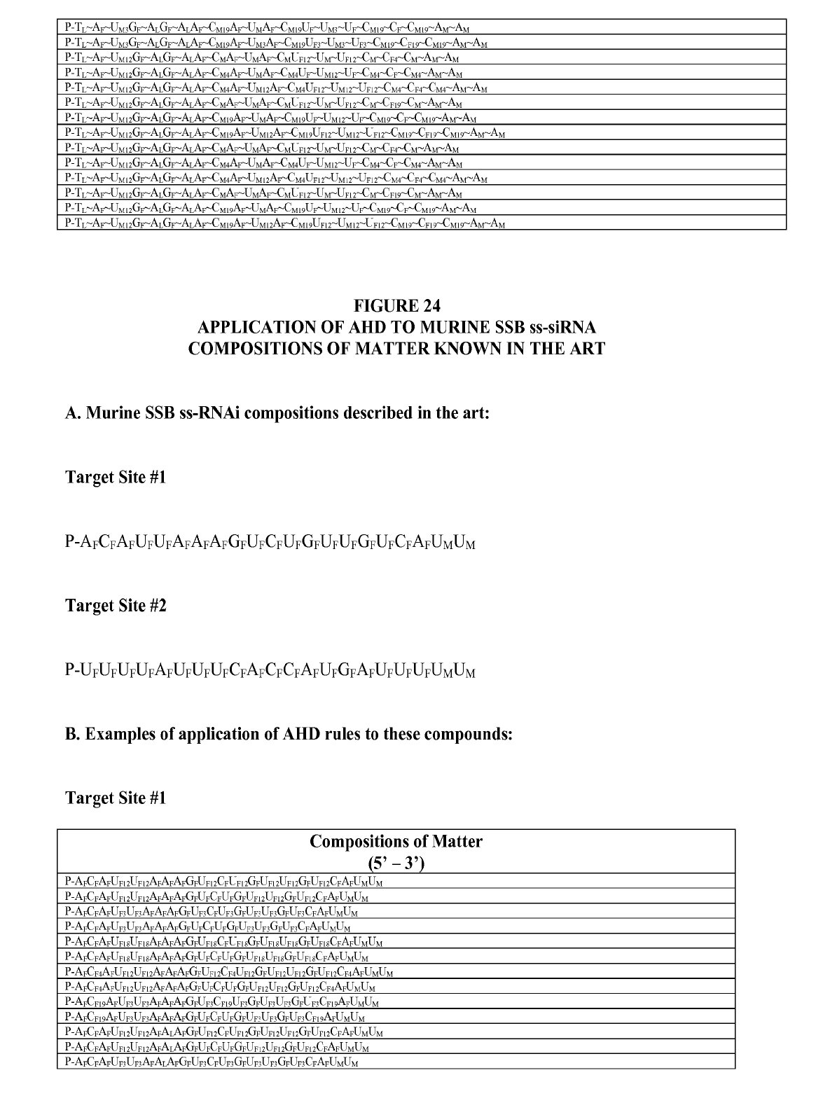

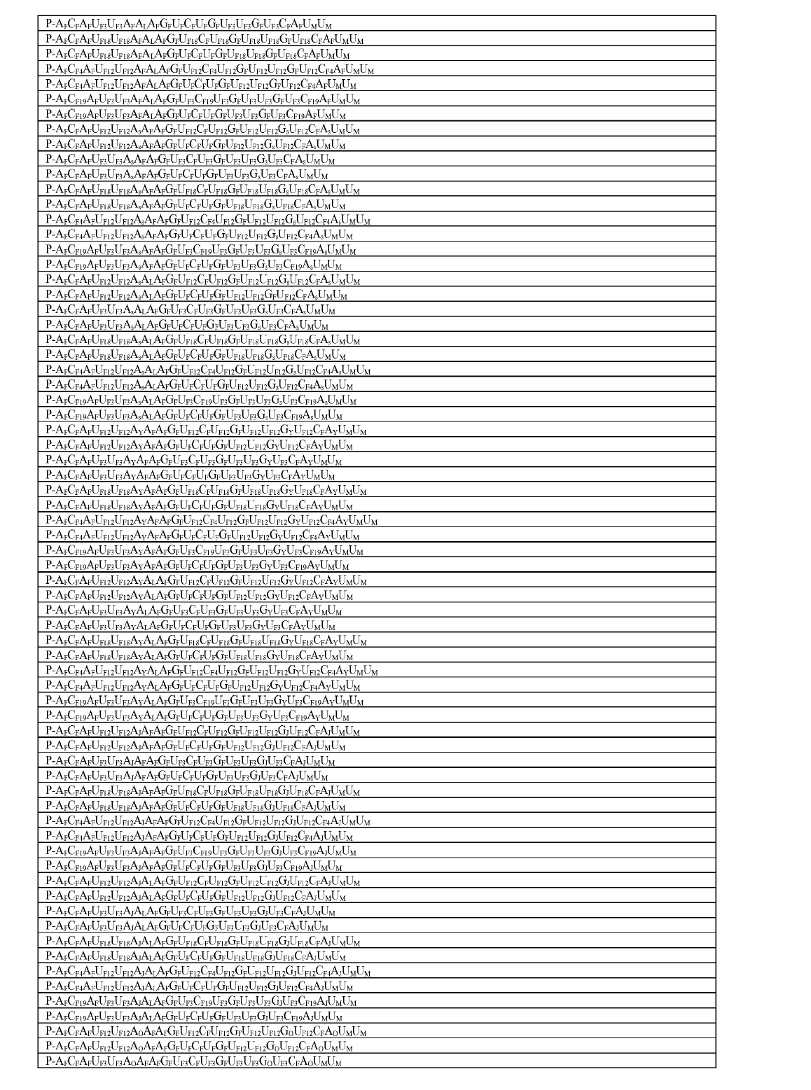

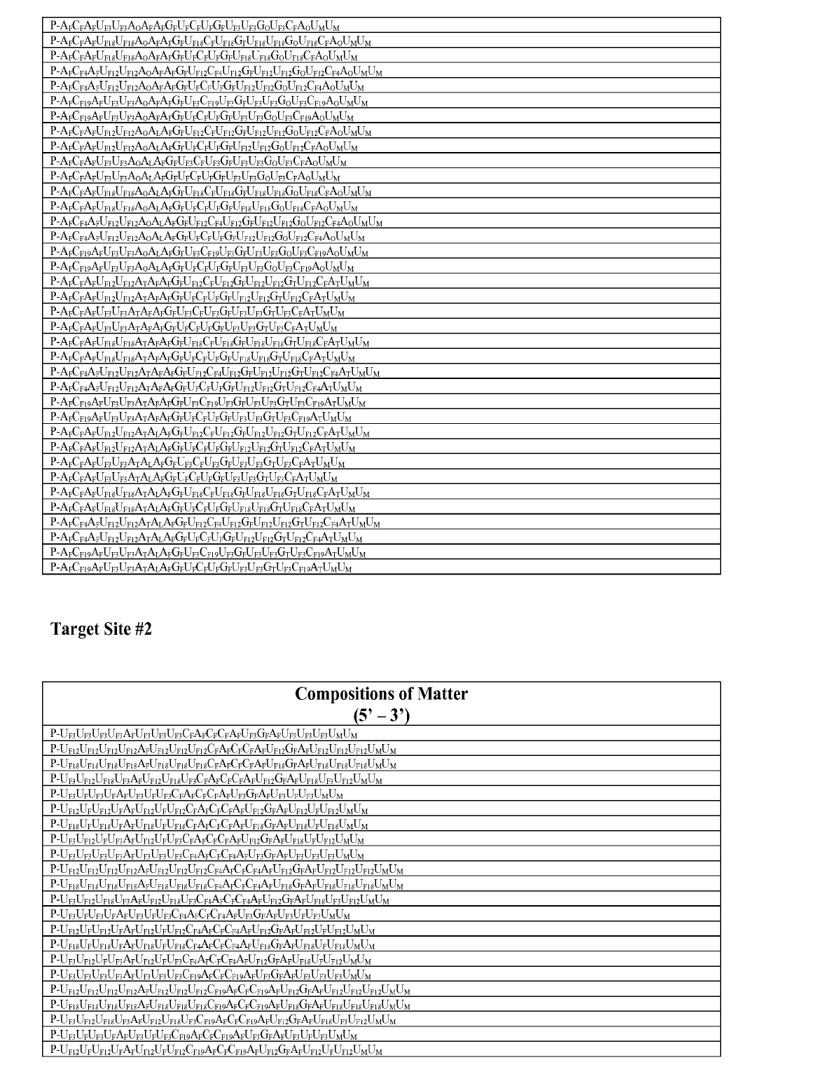

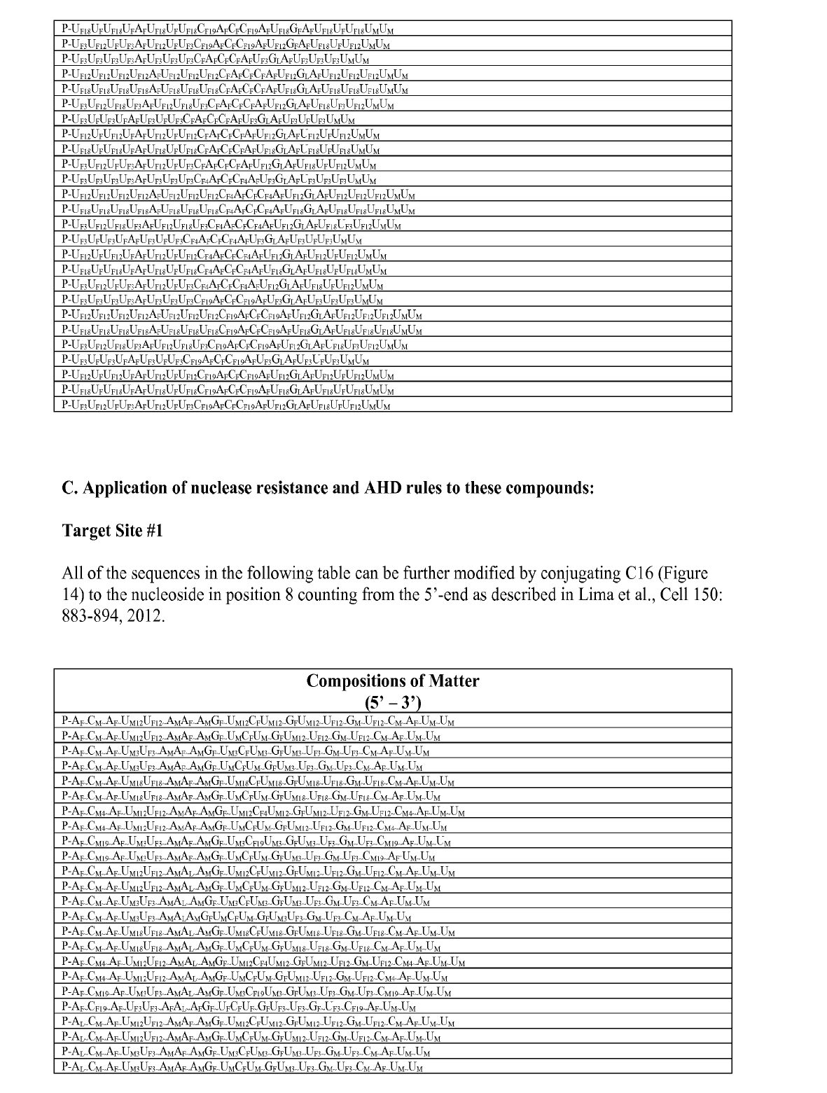

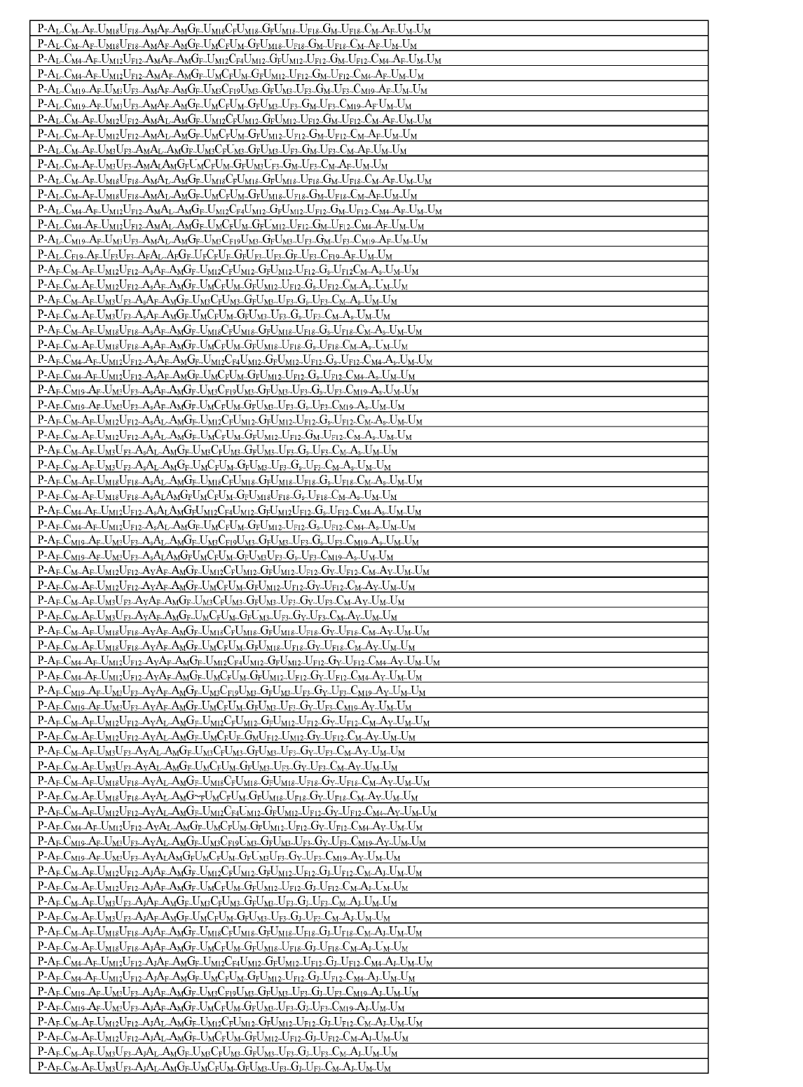

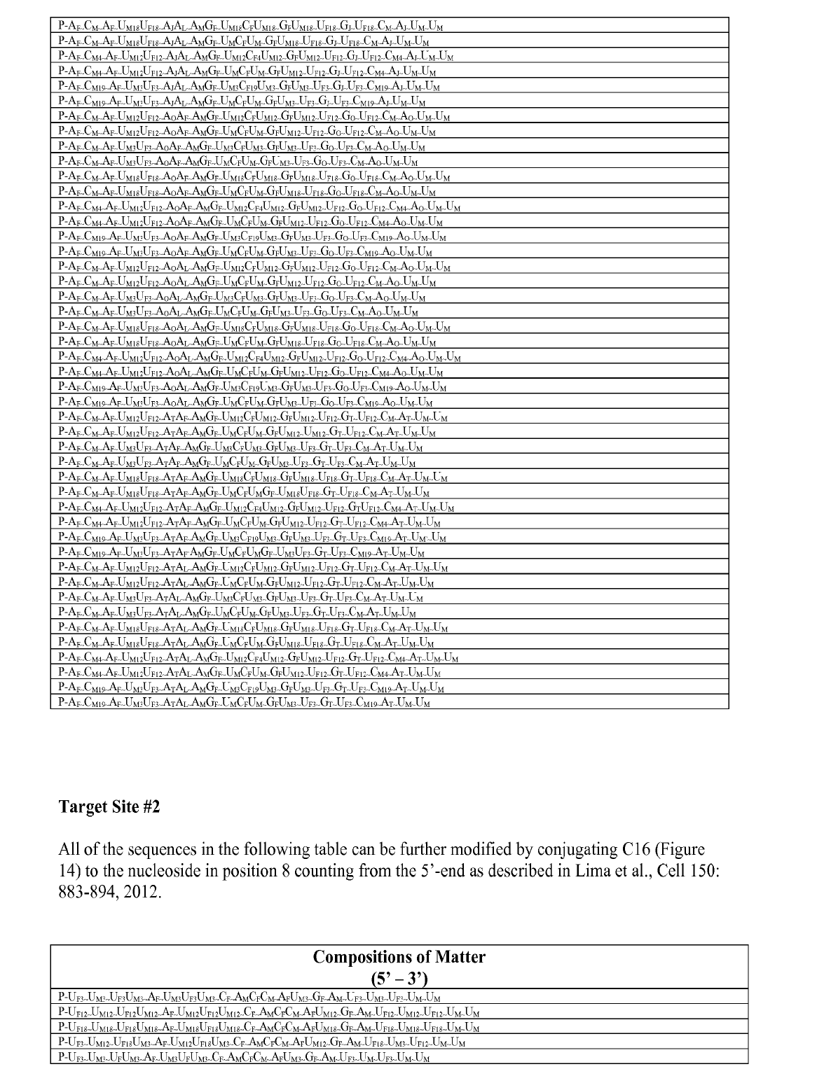

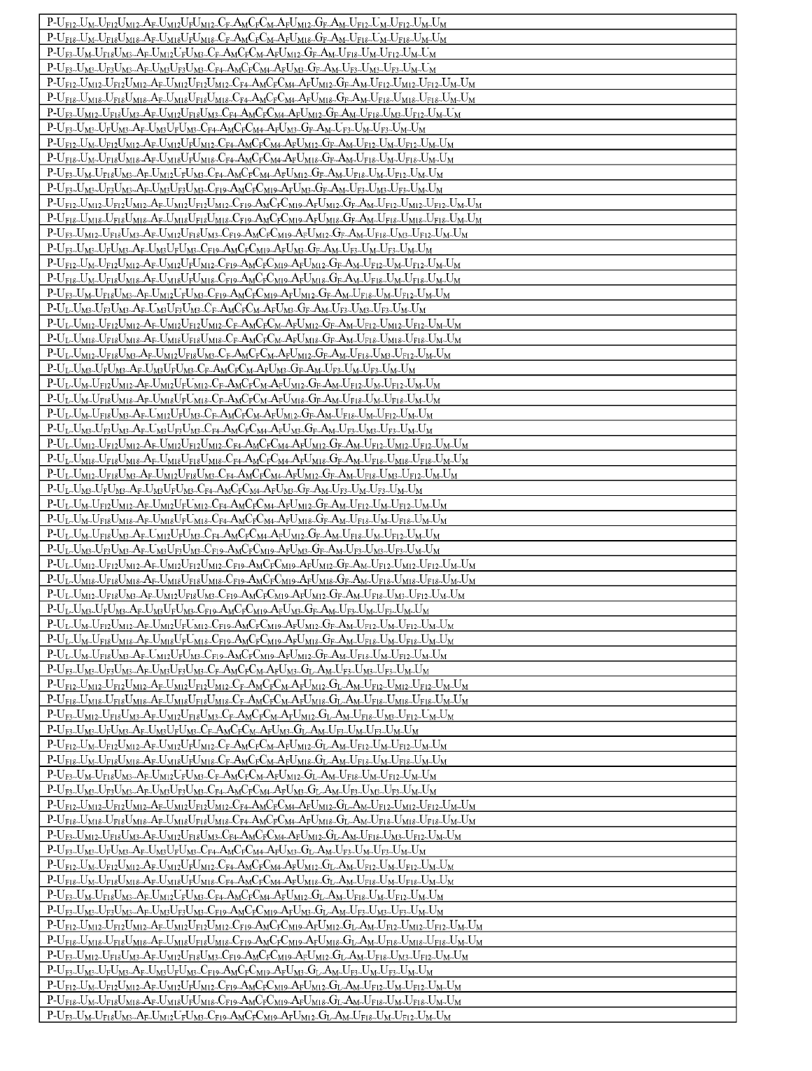

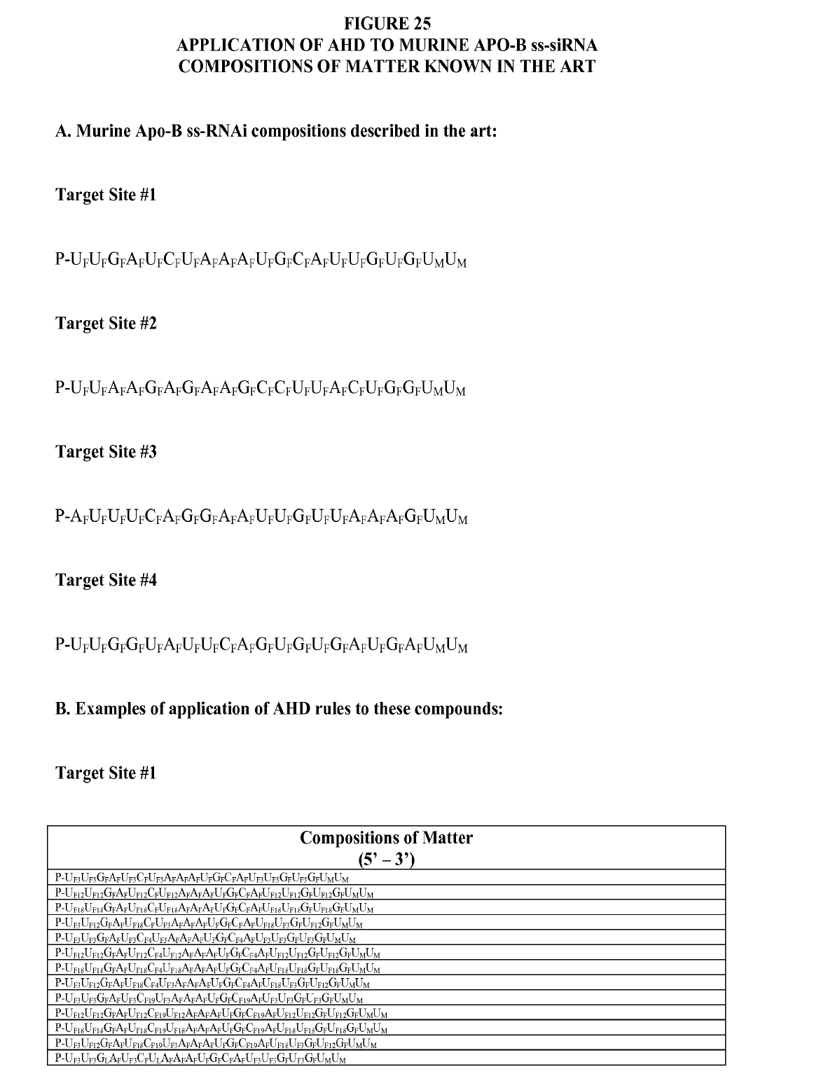

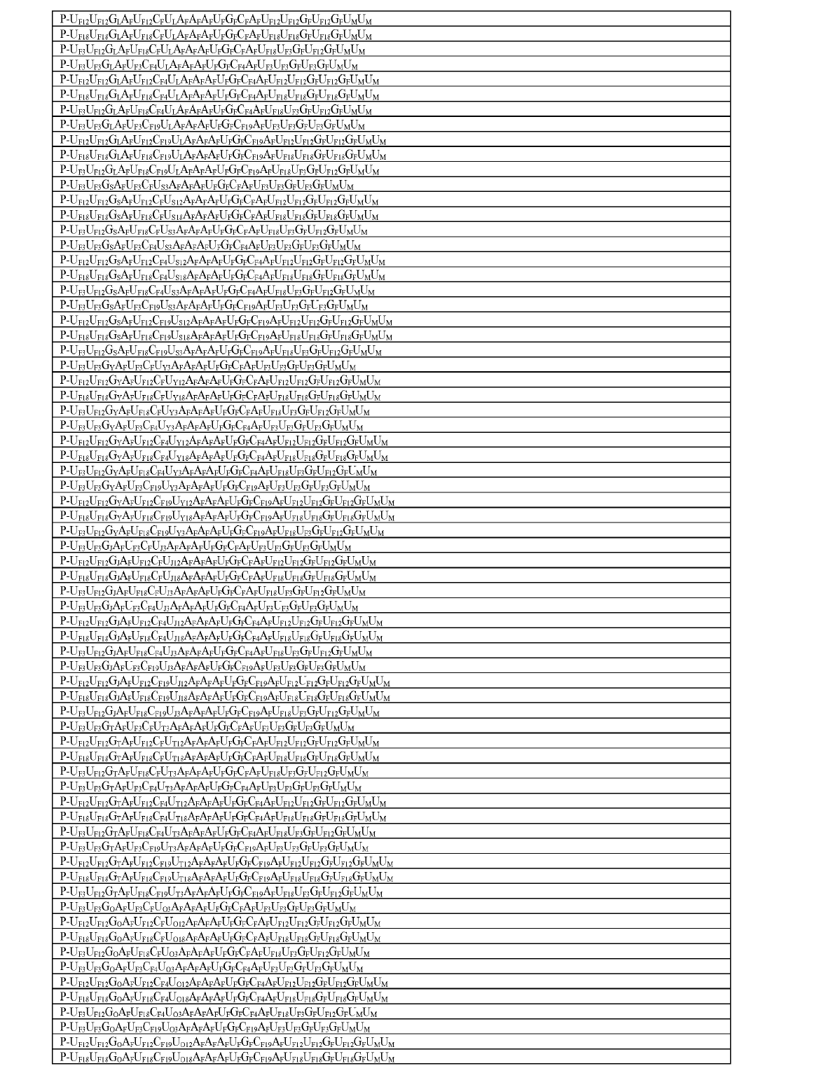

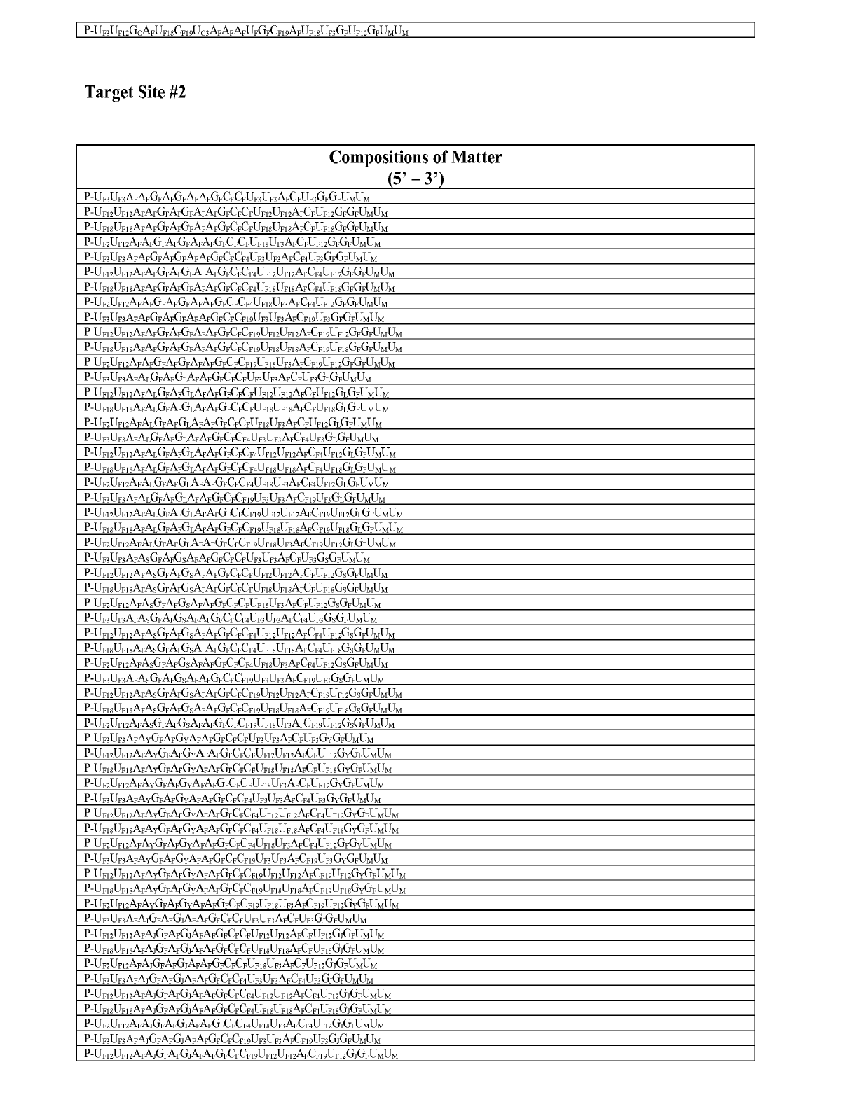

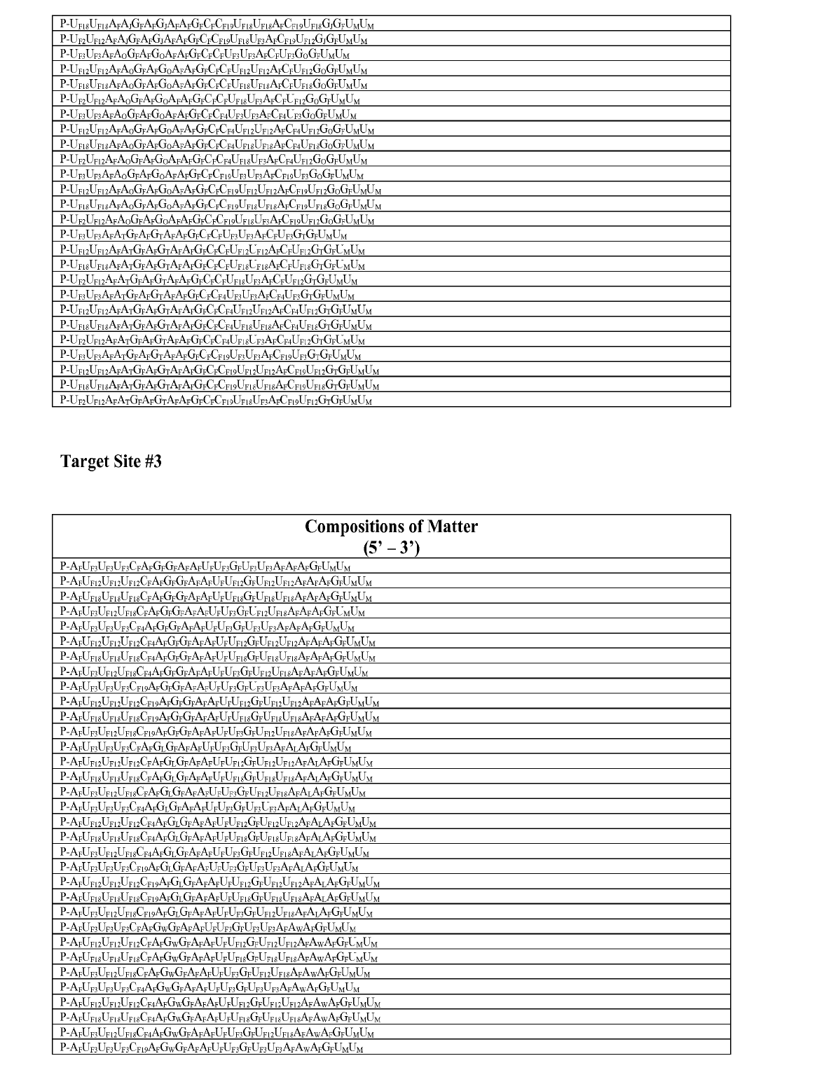

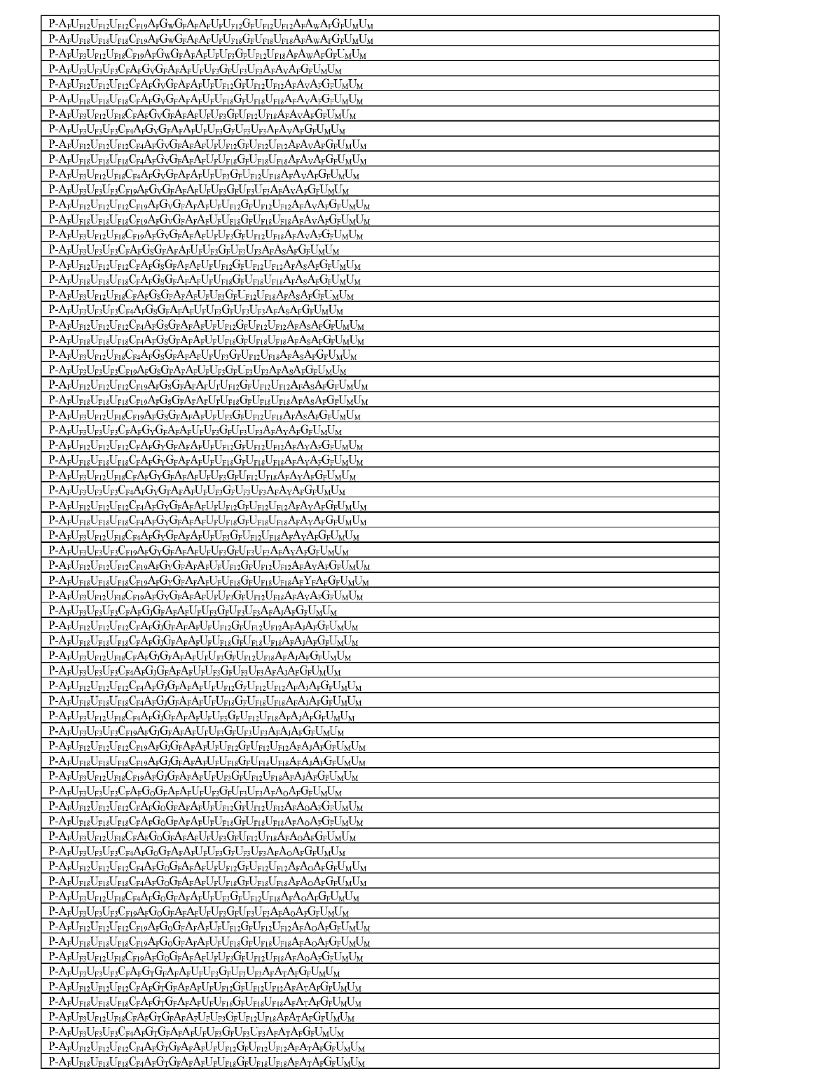

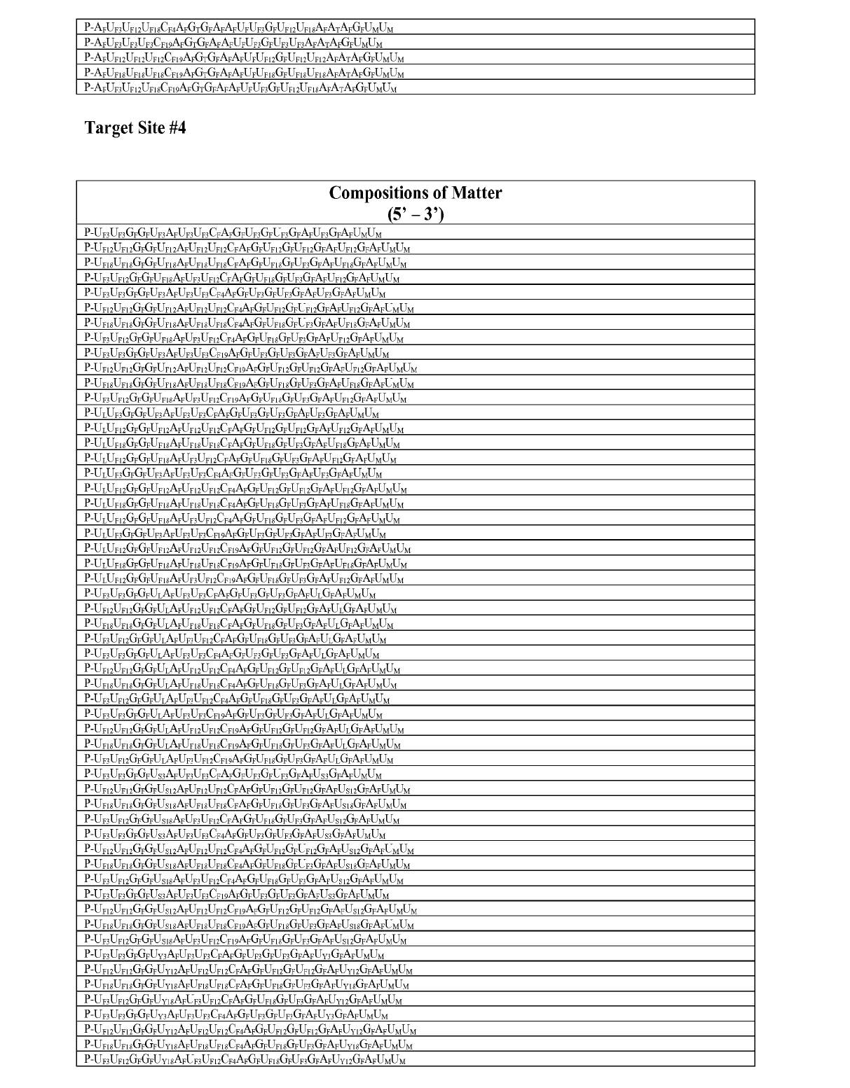

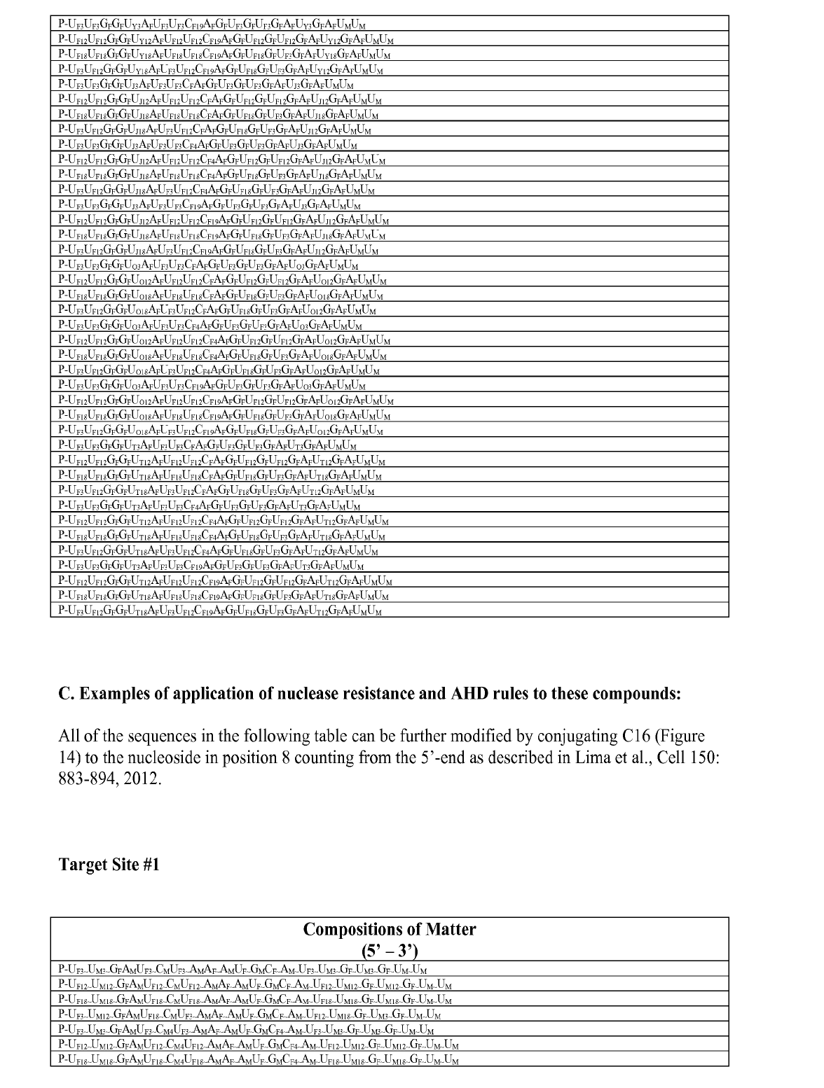

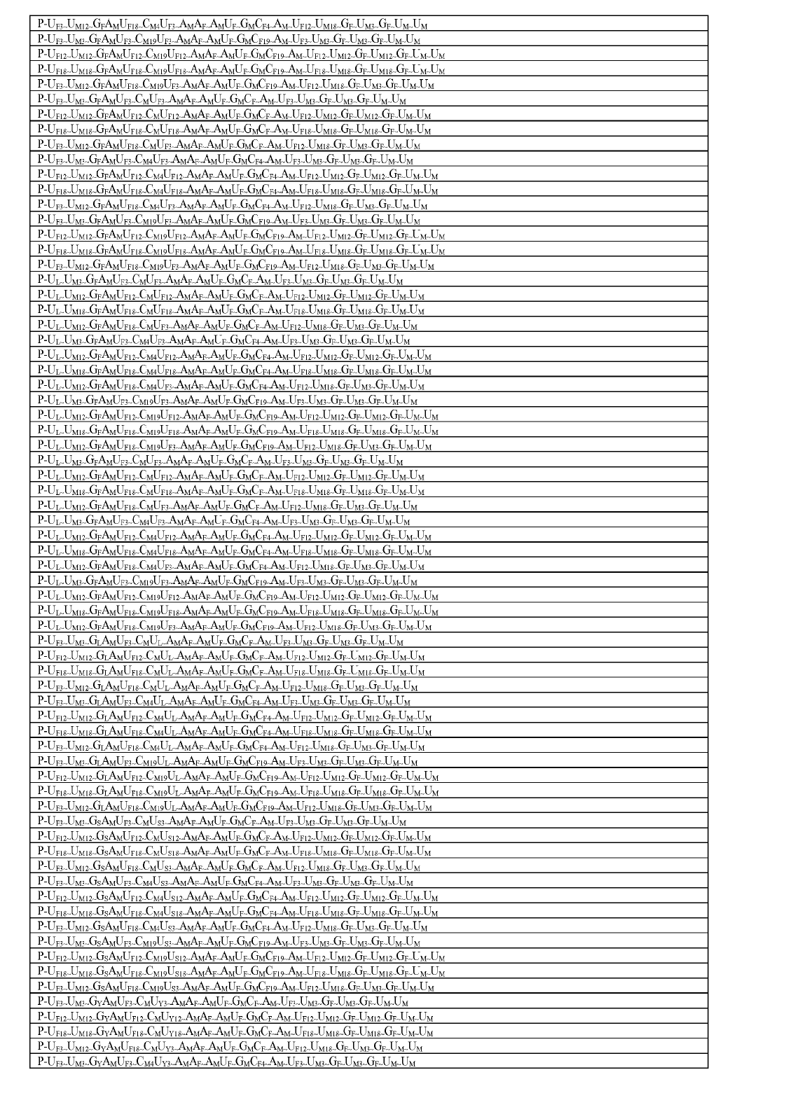

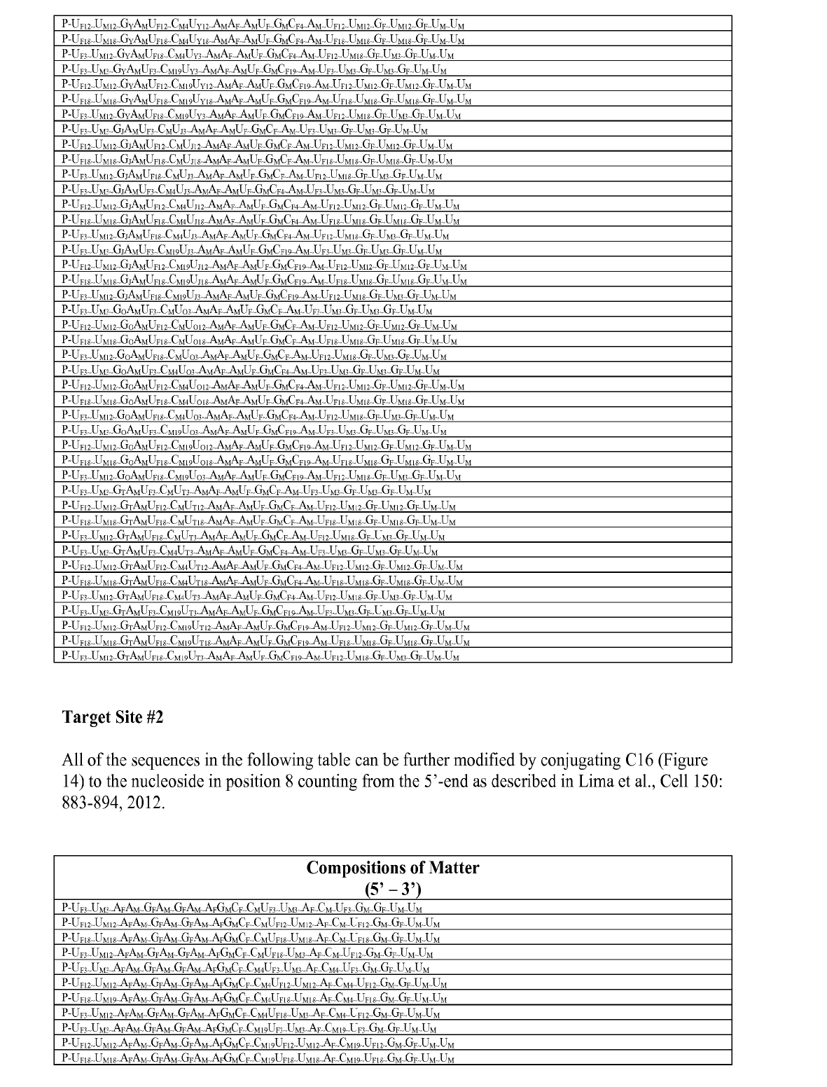

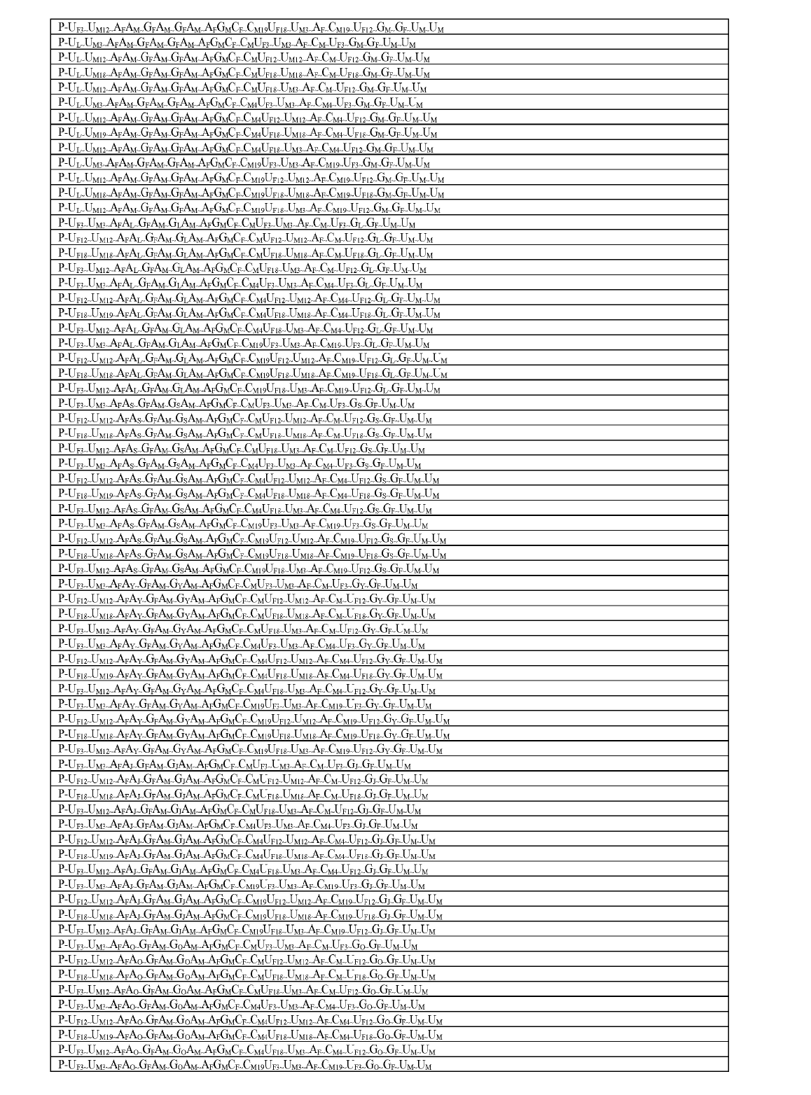

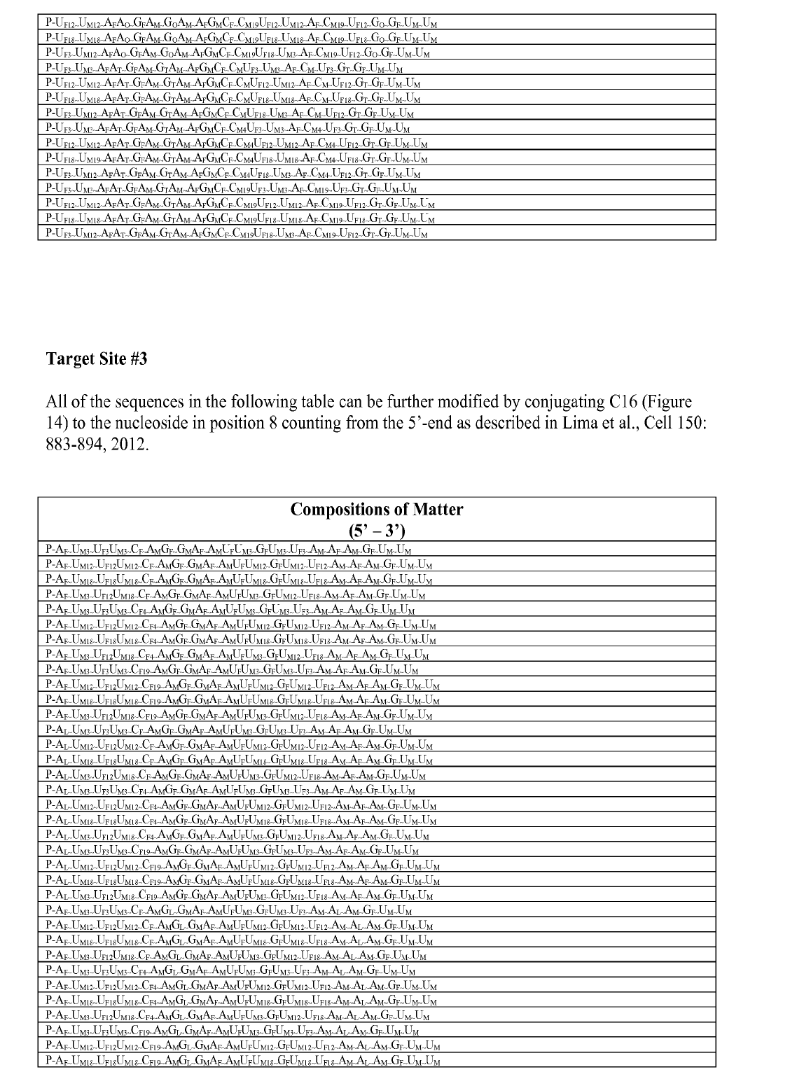

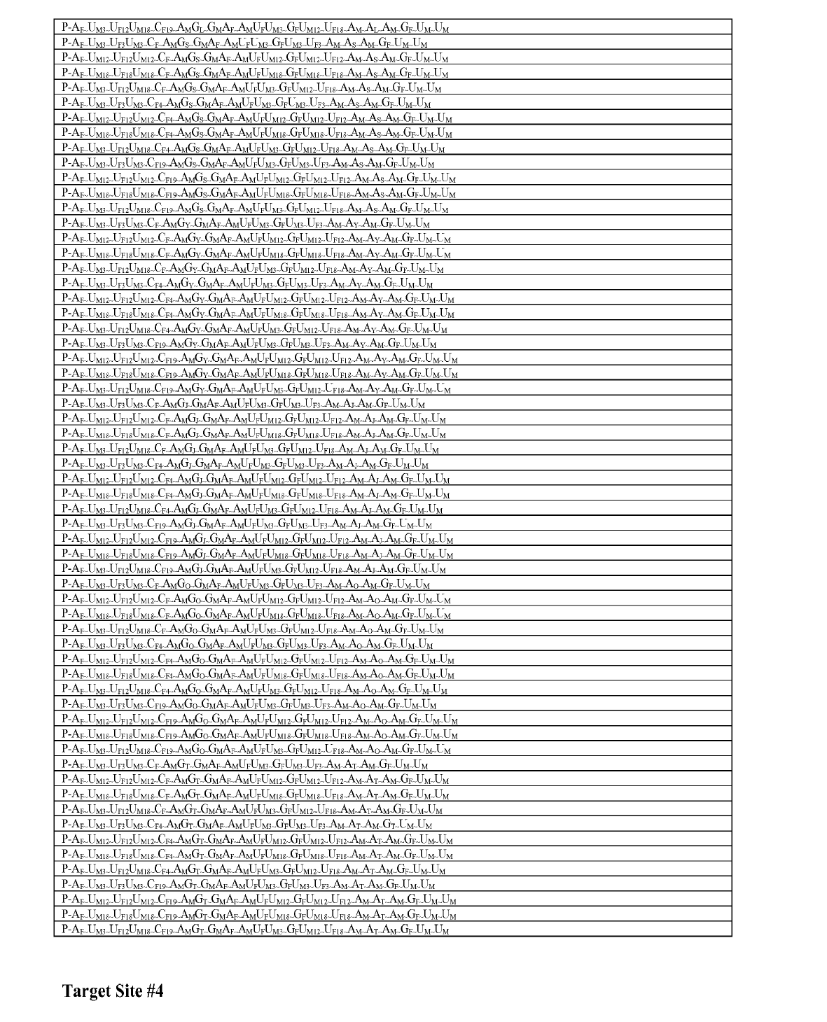

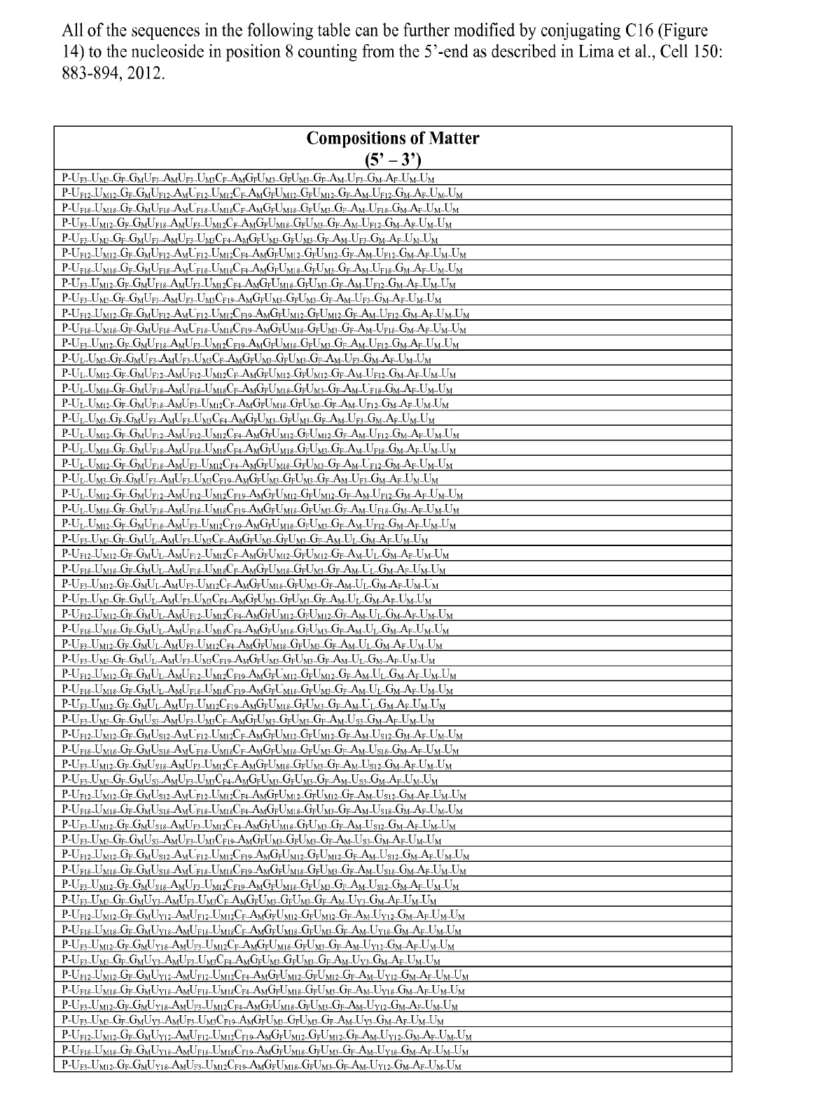

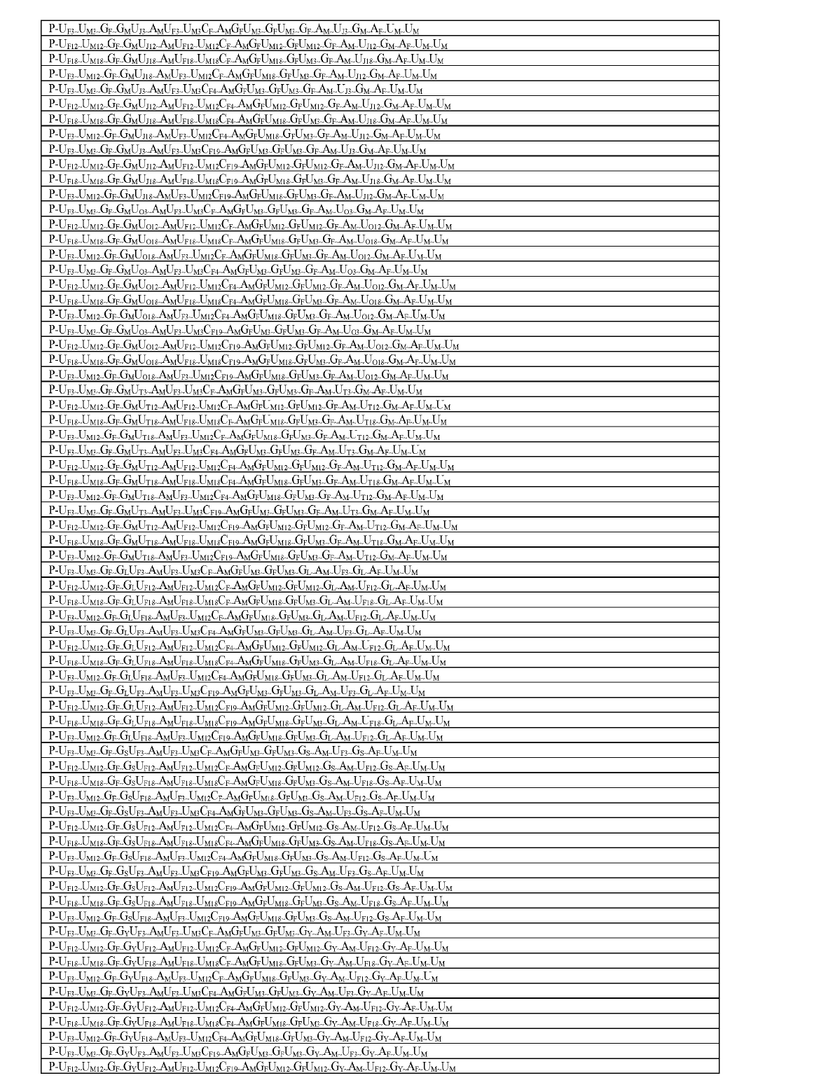

Ss-RNAi compounds known in the art and shown in FIGS. 24A and 25A are directed to murine SsB and Apo-B respectively (Haringsma et al., Nucleic Acids Res 40: 4125-36, 2012). The SsB Target Site #1 listed in FIG. 24A has been changed at the penultimate nucleoside from the 3′-end not counting the UU overhang precursor from a U as required by Table 1 in the paper to a C to match the quoted sequence with accession number NM_{—}009278 referenced by the table. The individual strands in this publication were not sufficiently stabilized against nuclease attack to be administered to animals without a carrier. Hence, when activity in mice was tested lipid nanoparticles were used. Consequently, simple addition of AHD modifications (FIG. 24B and FIG. 25B) to these compounds will not make them suitable for use in animals without a protective carrier as they will not be rendered sufficiently nuclease resistant. Thus, comparisons between these compounds and those with only AHD modifications can be compared in vitro using the standard transfection reagents needed to get oligos into cells in culture or carriers suitable for use in animals must be used for in vivo comparisons. Examples are provided where compounds with the same sequences are both stabilized for in vivo use without a carrier using the methods provided herein and have AHD modifications. These appear in FIG. 24C and FIG. 25C.

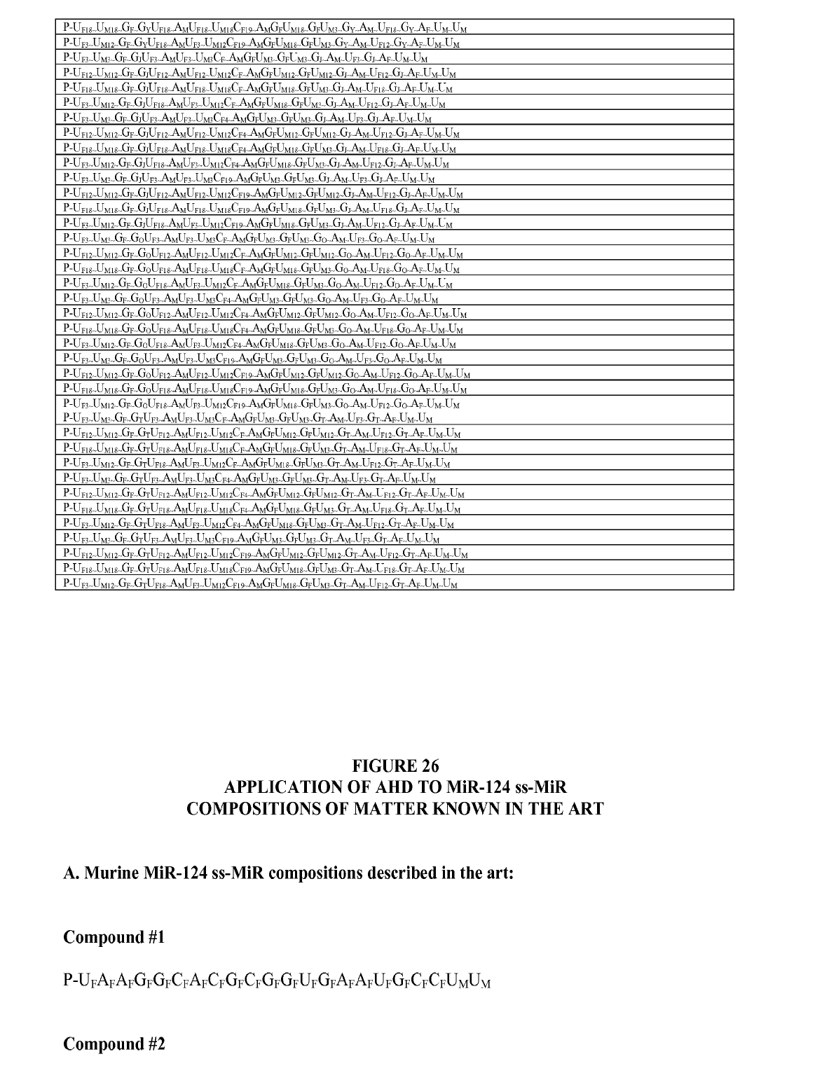

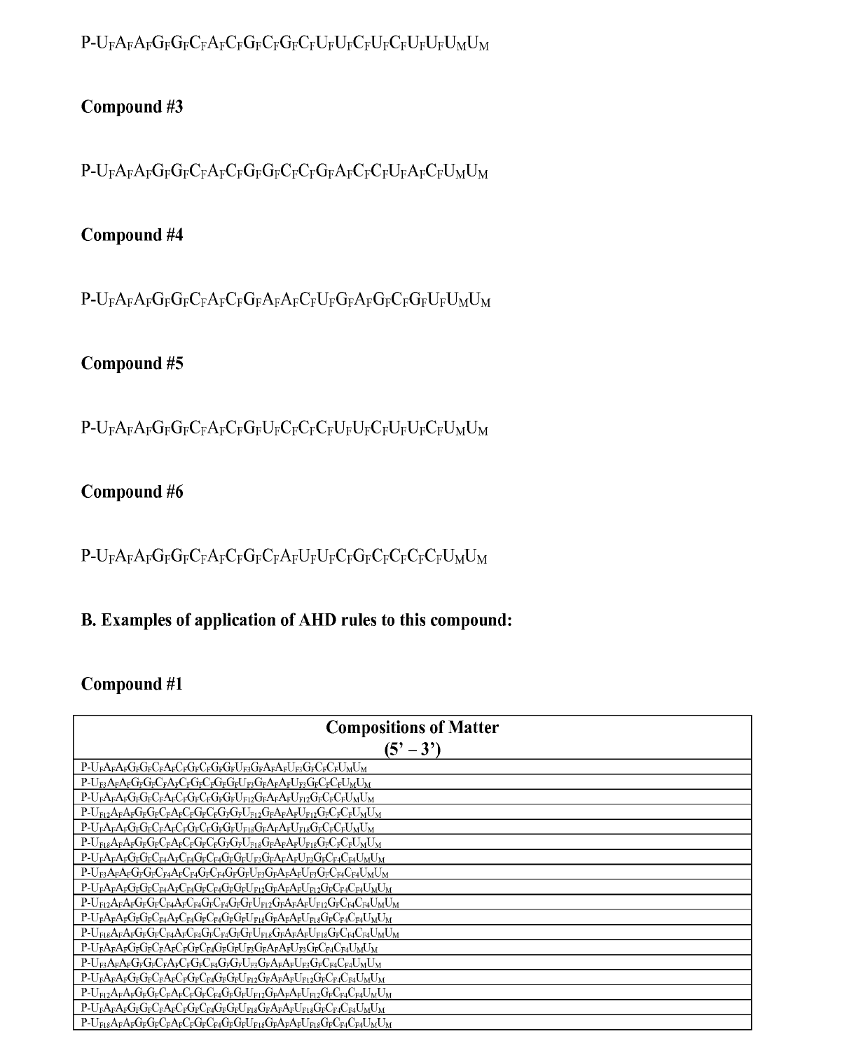

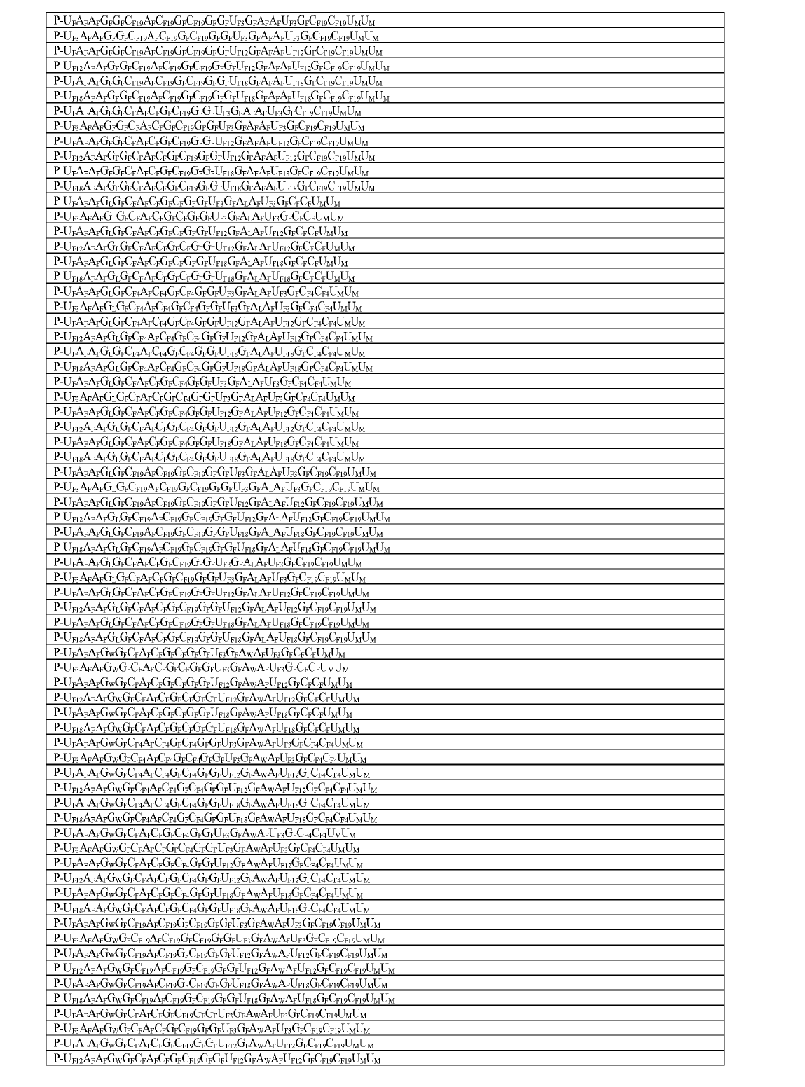

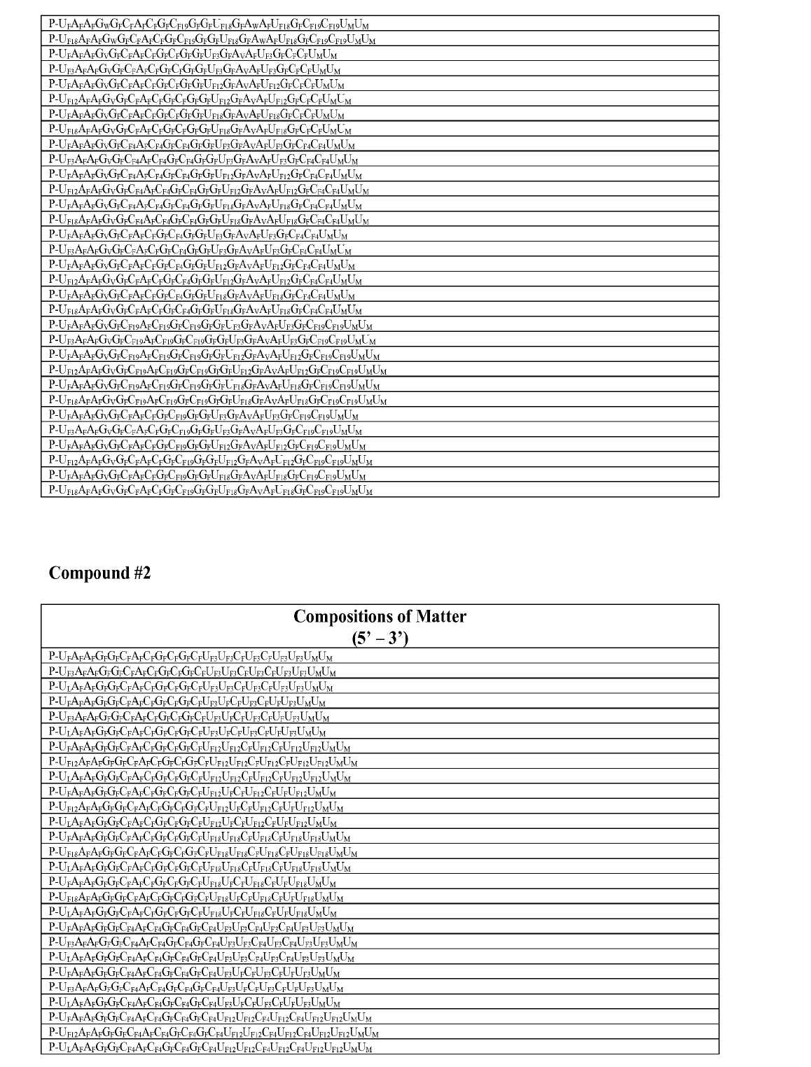

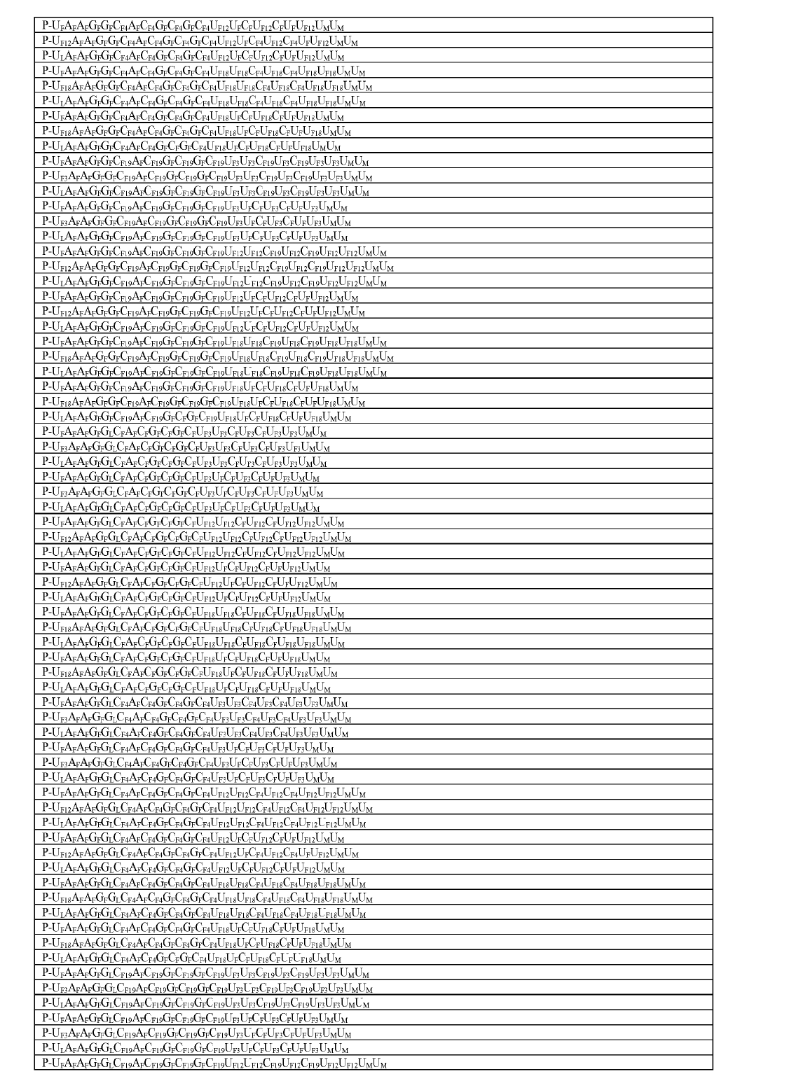

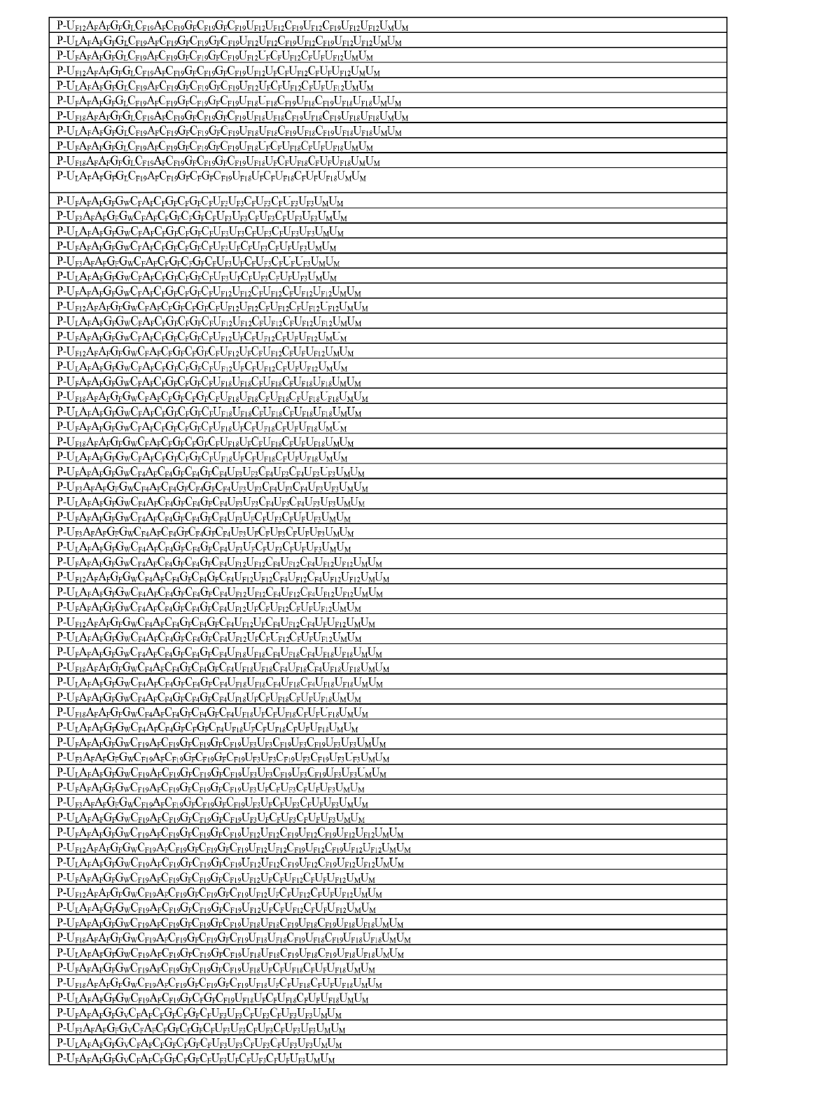

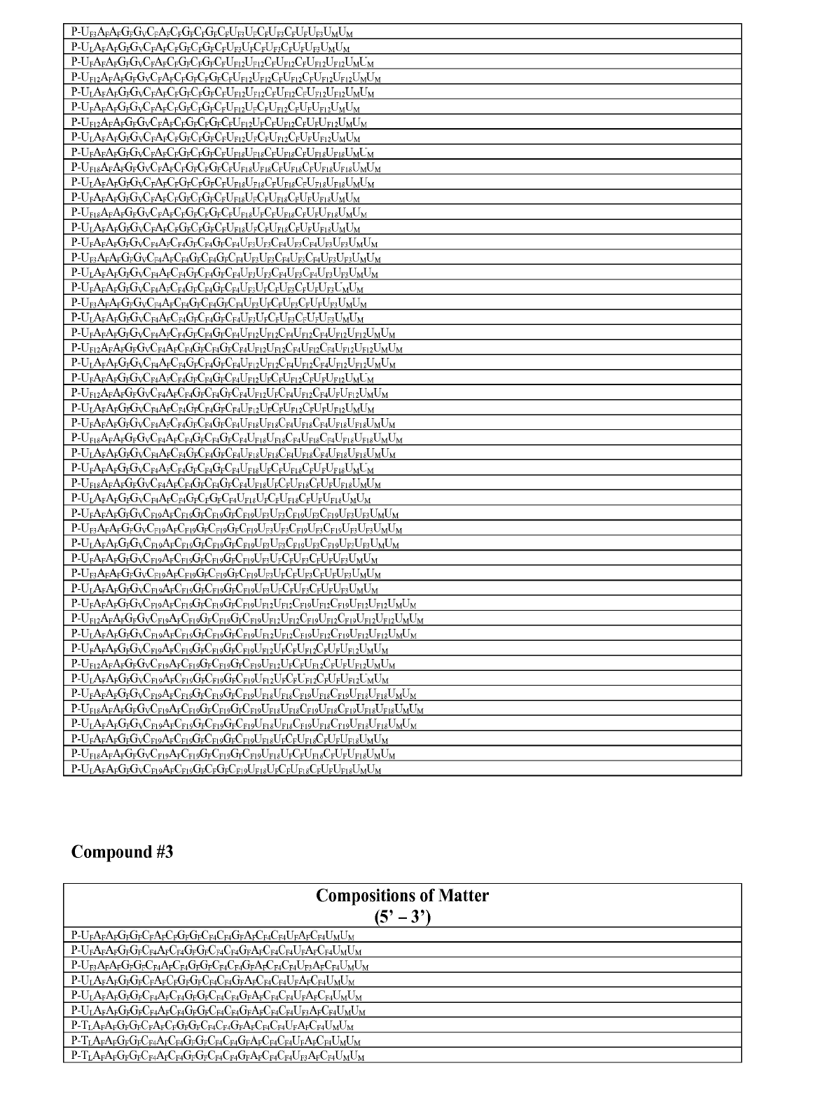

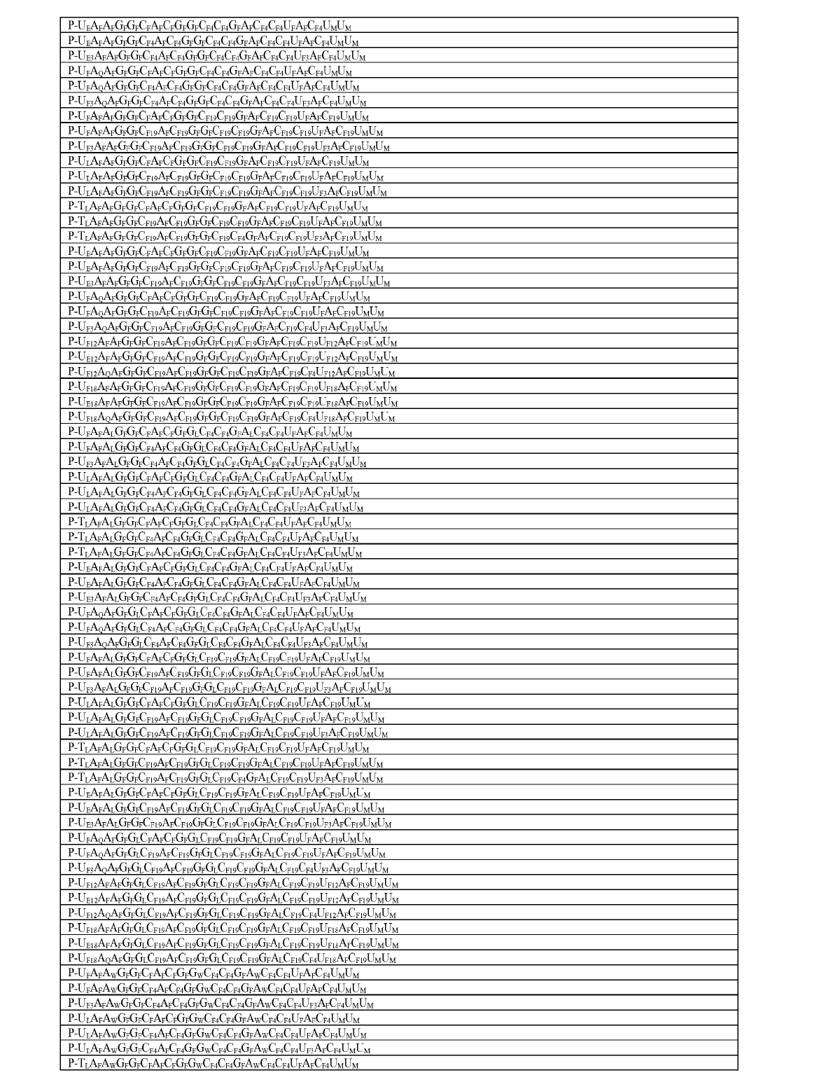

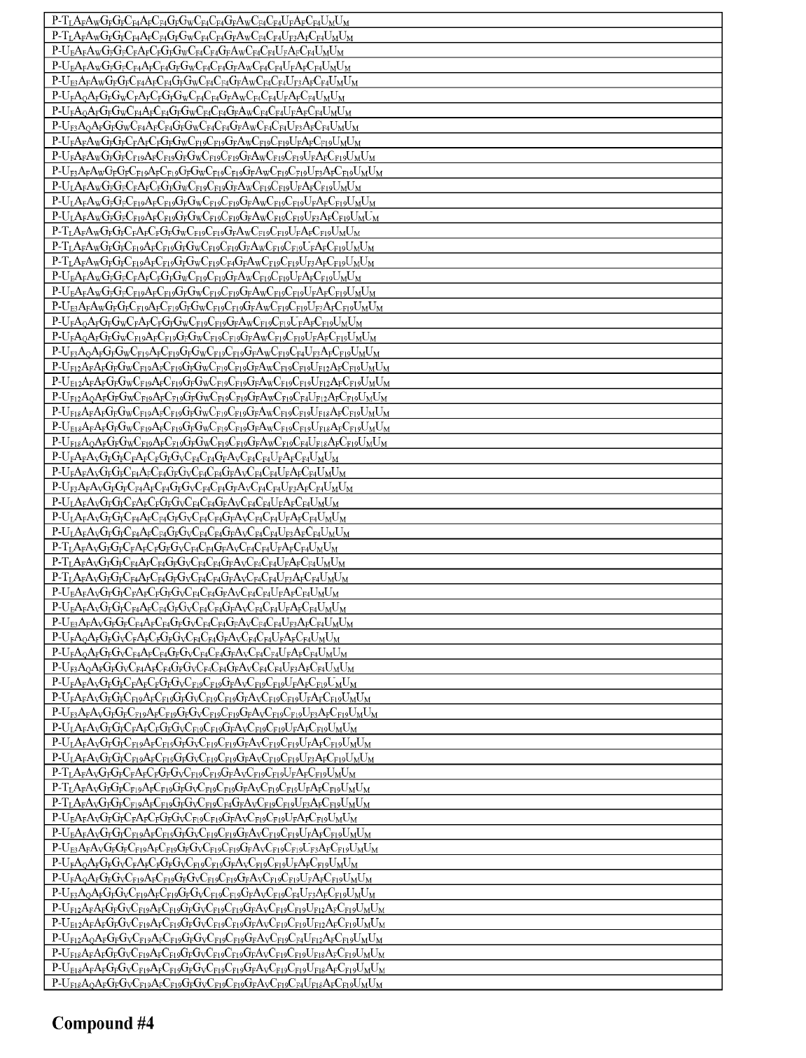

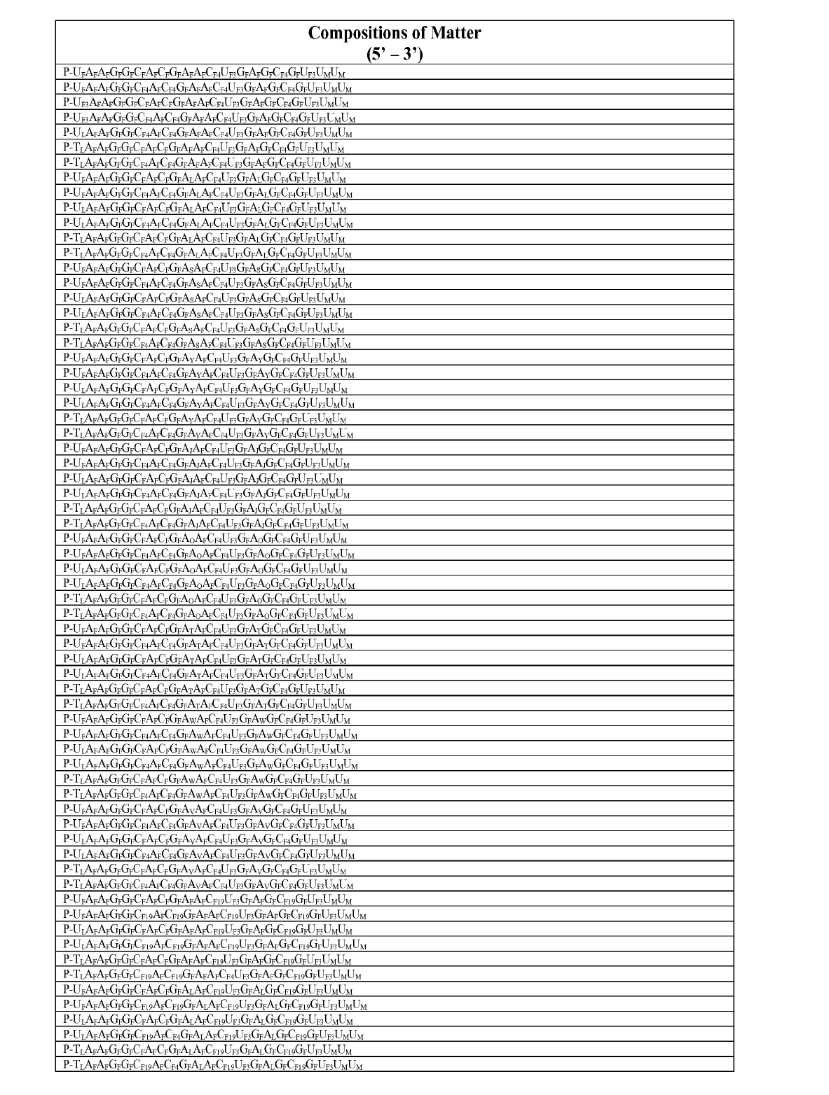

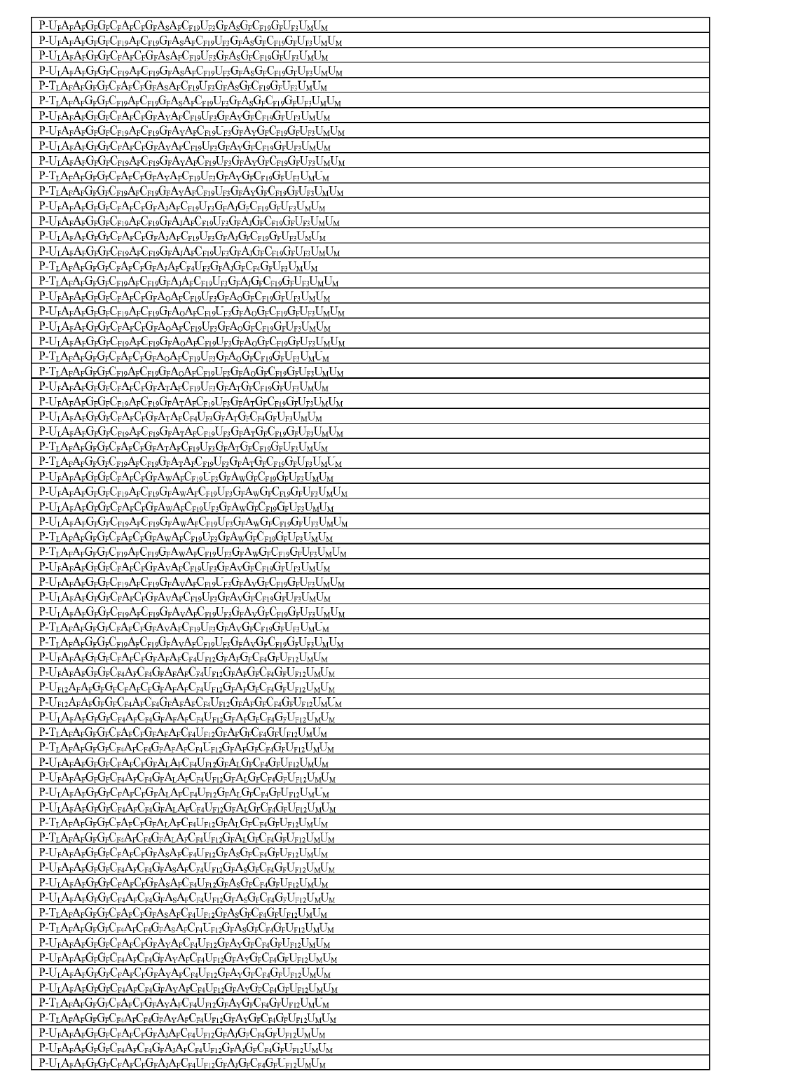

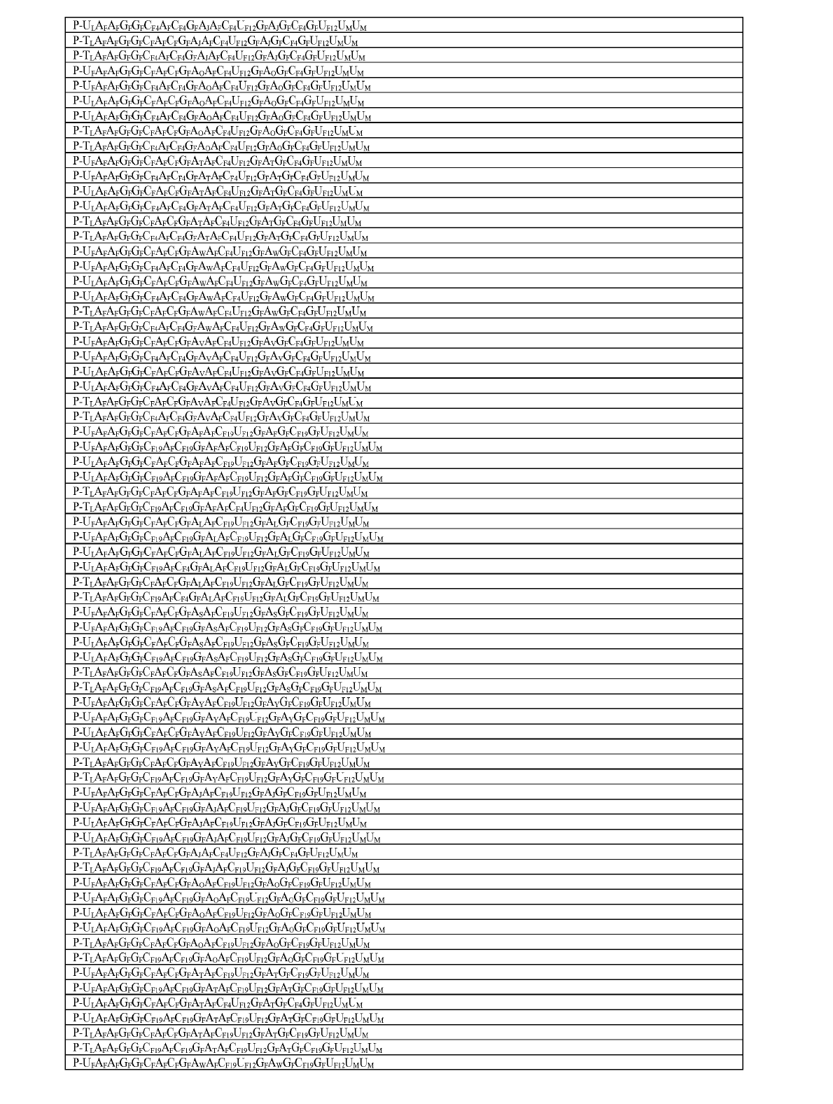

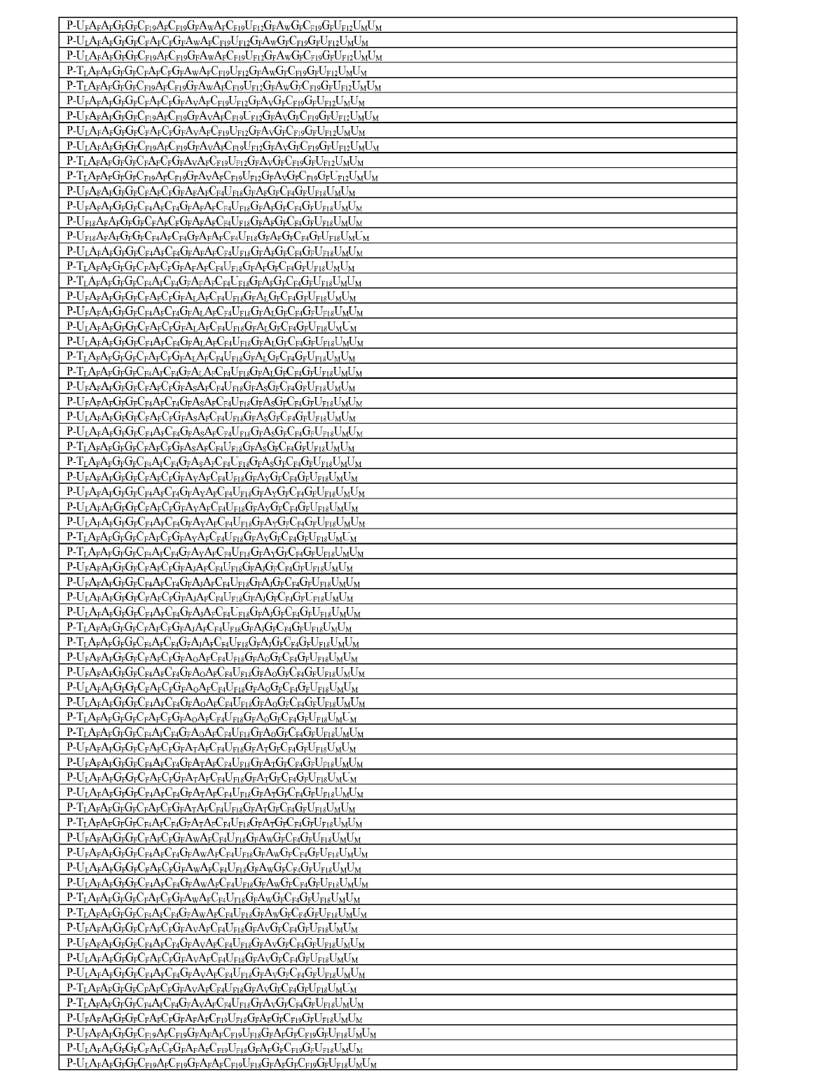

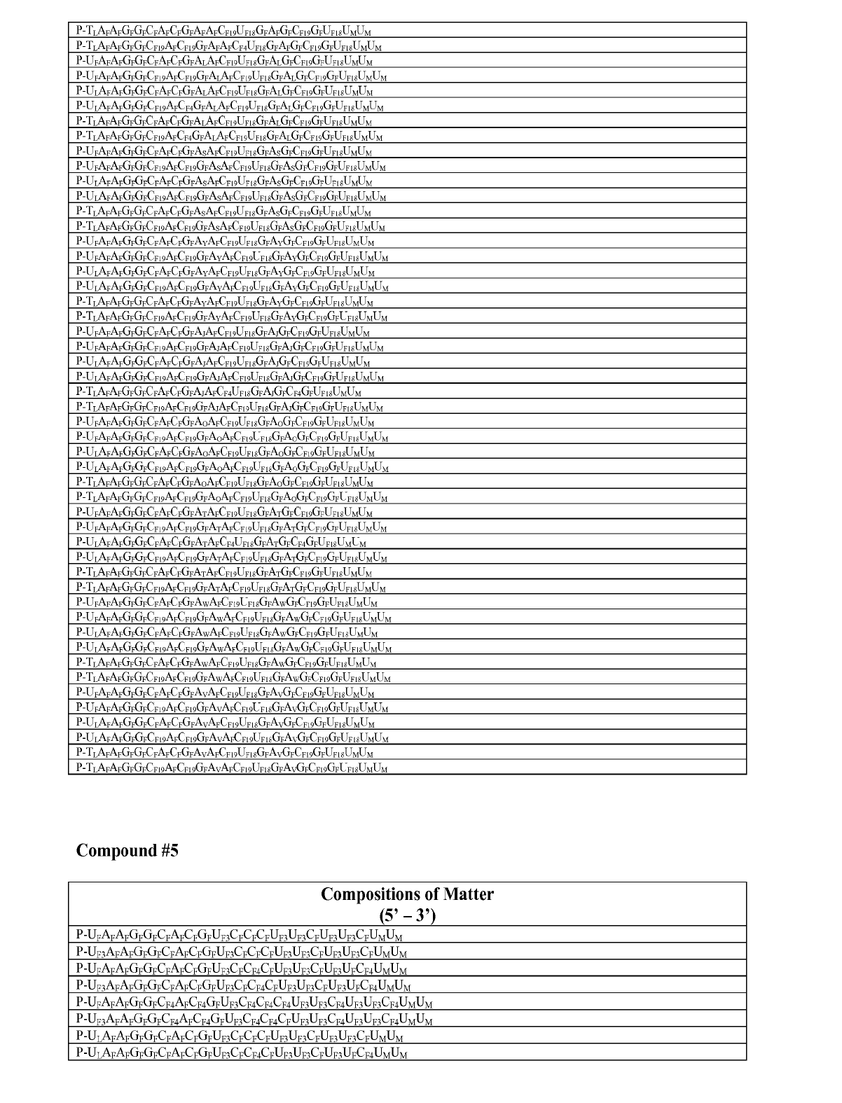

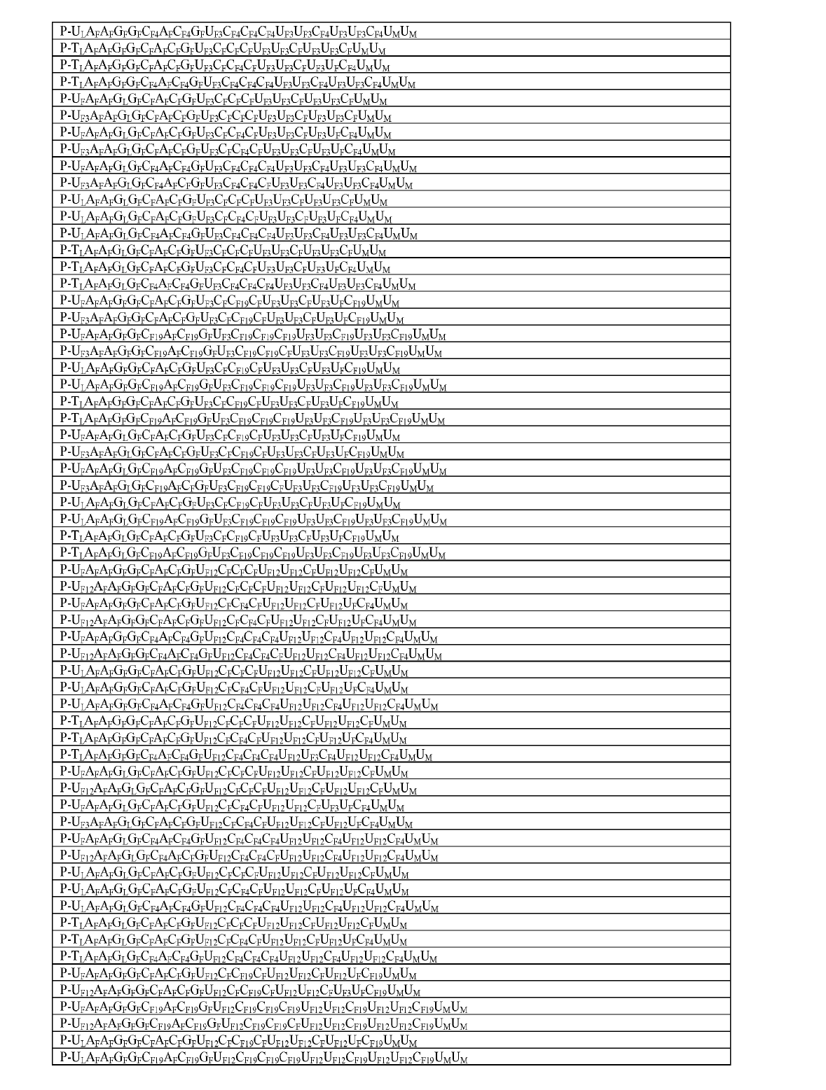

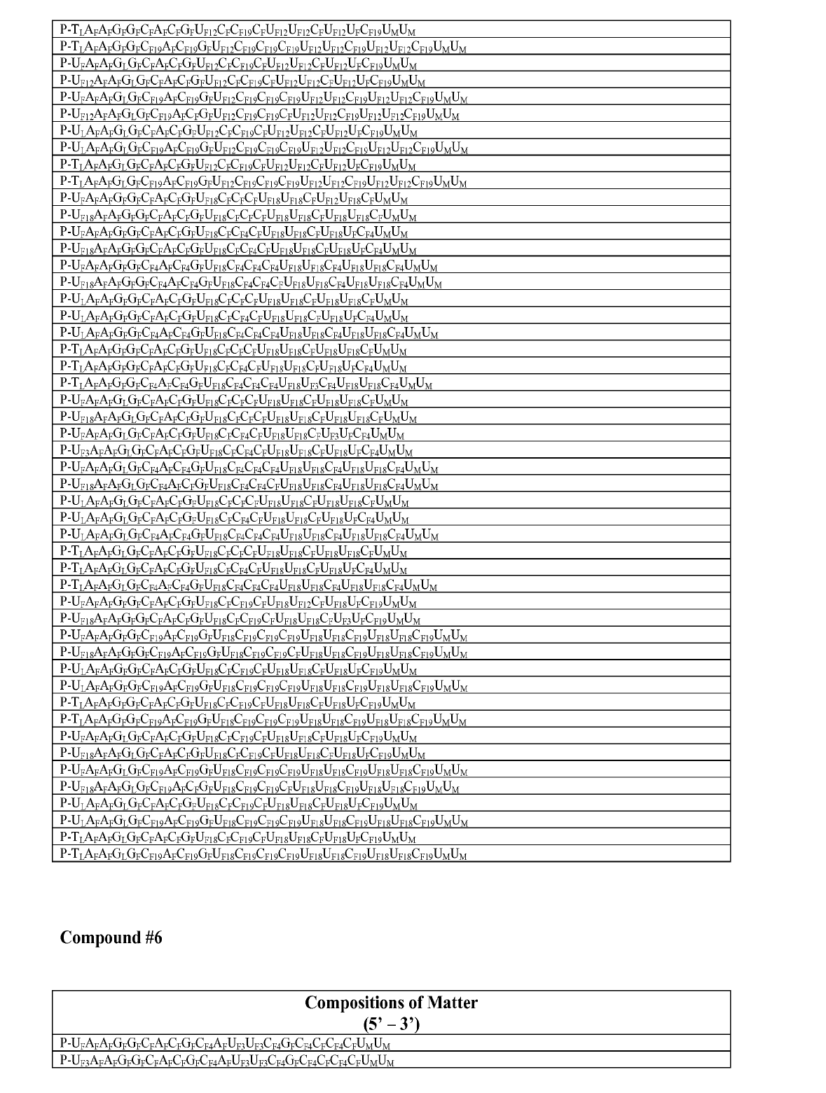

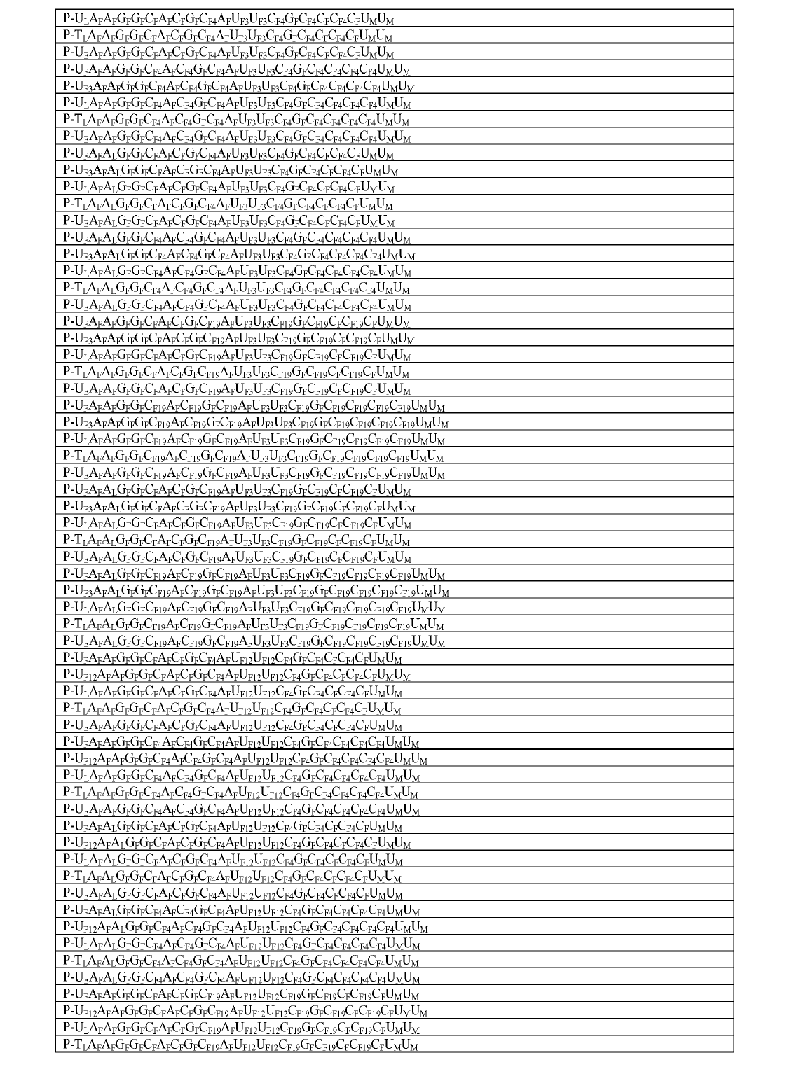

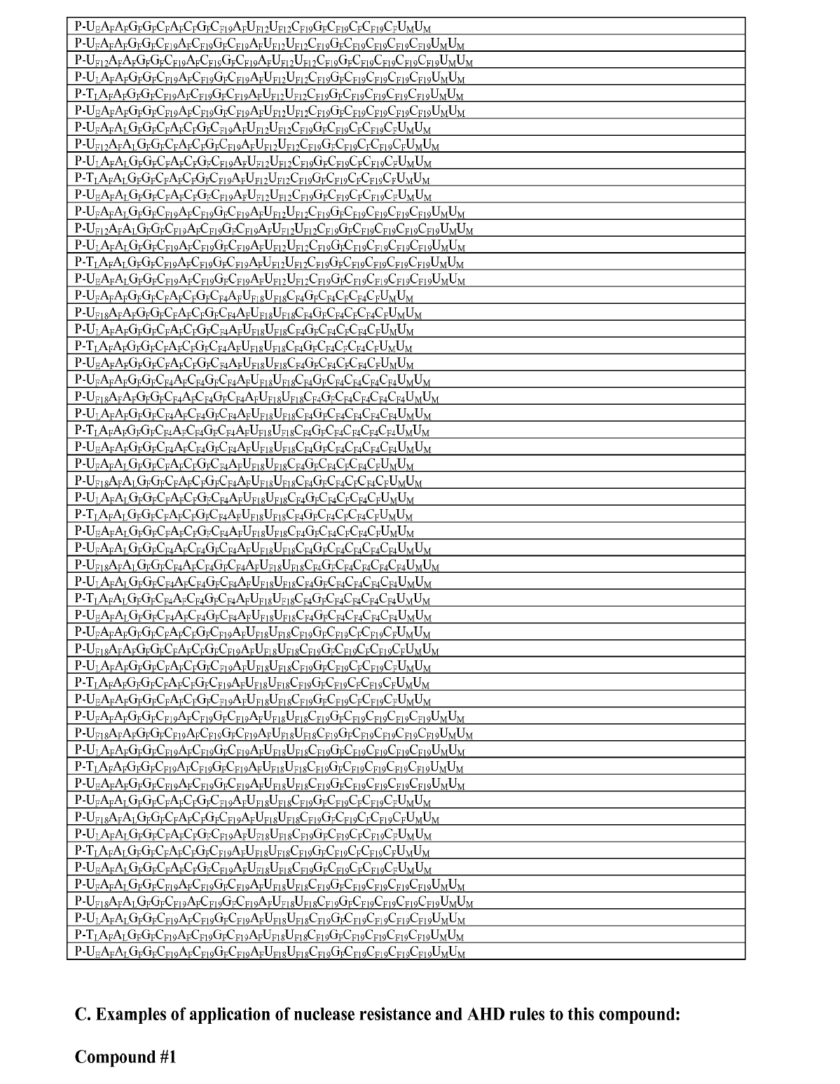

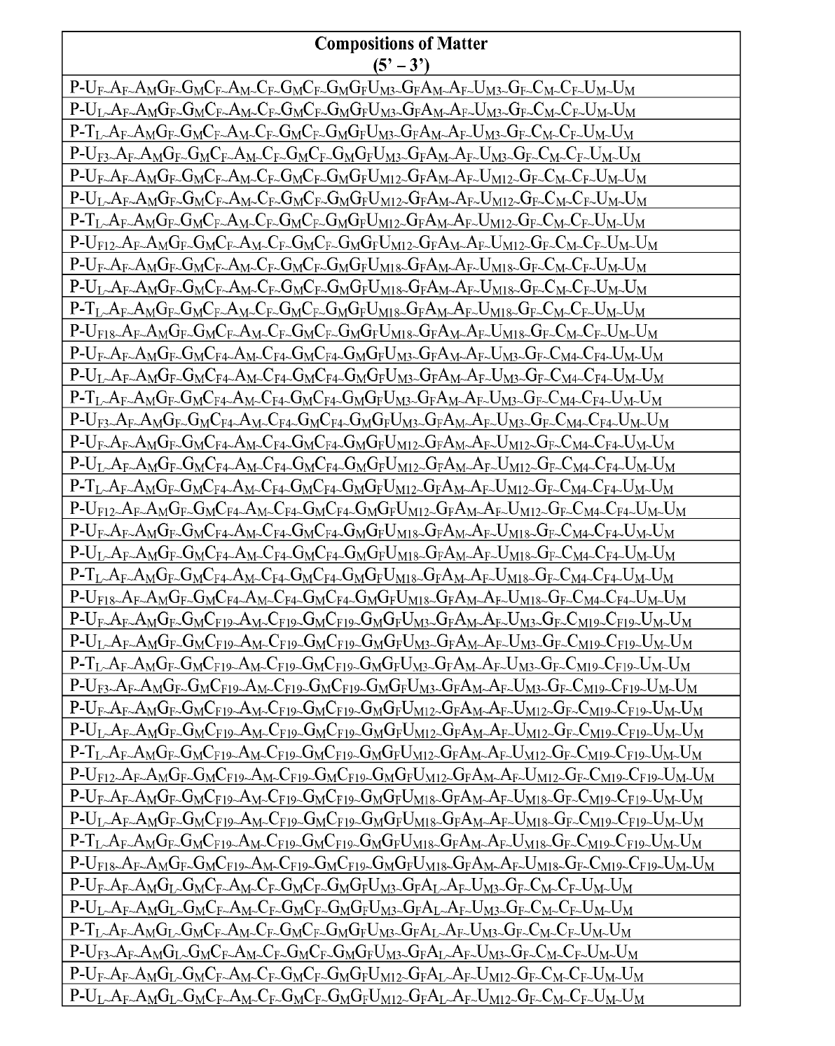

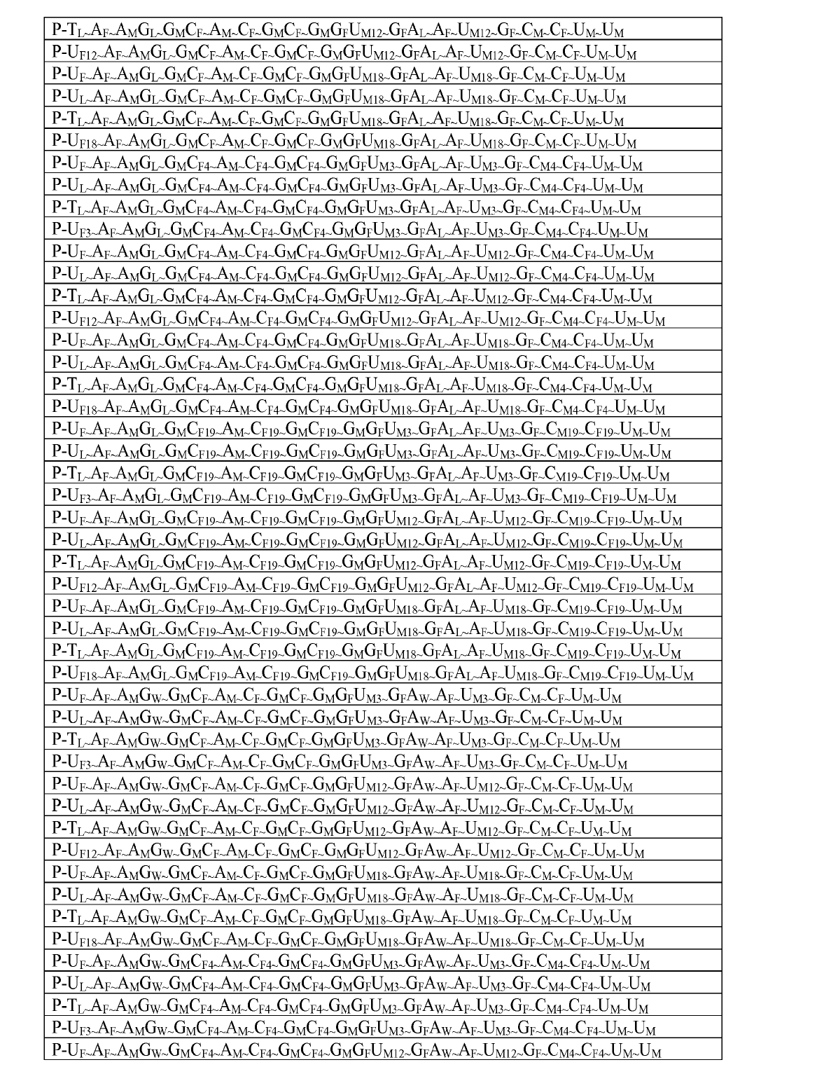

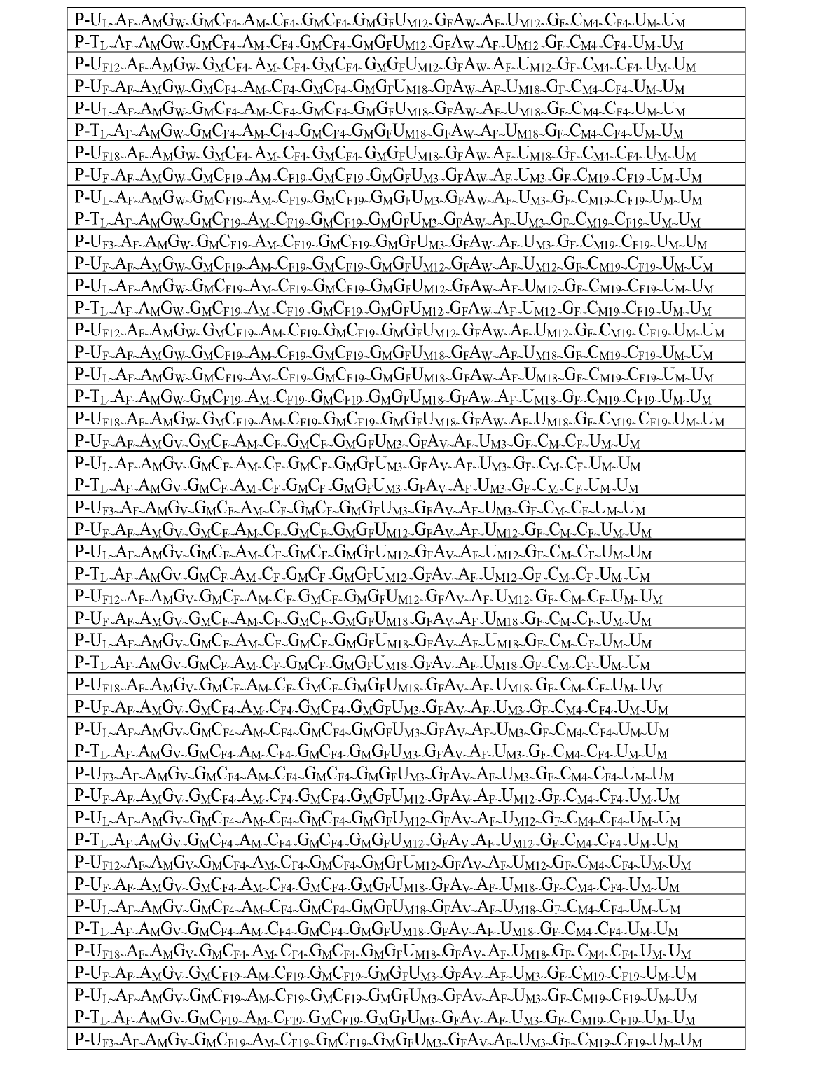

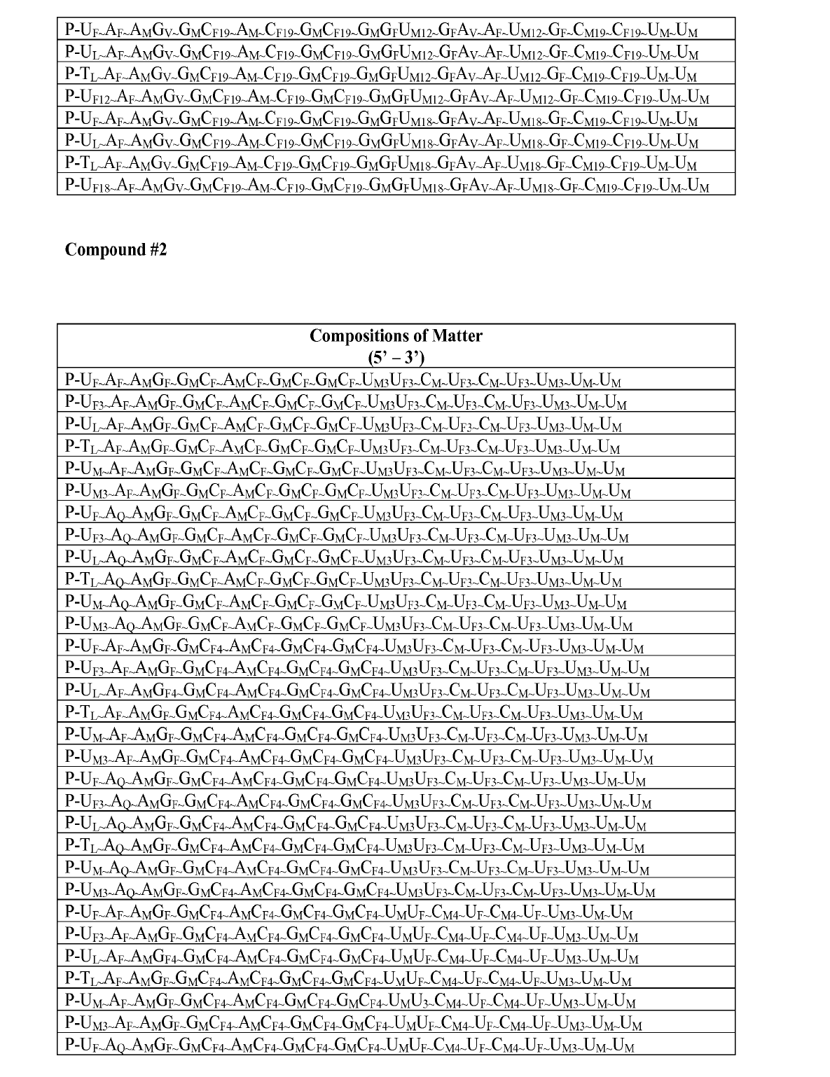

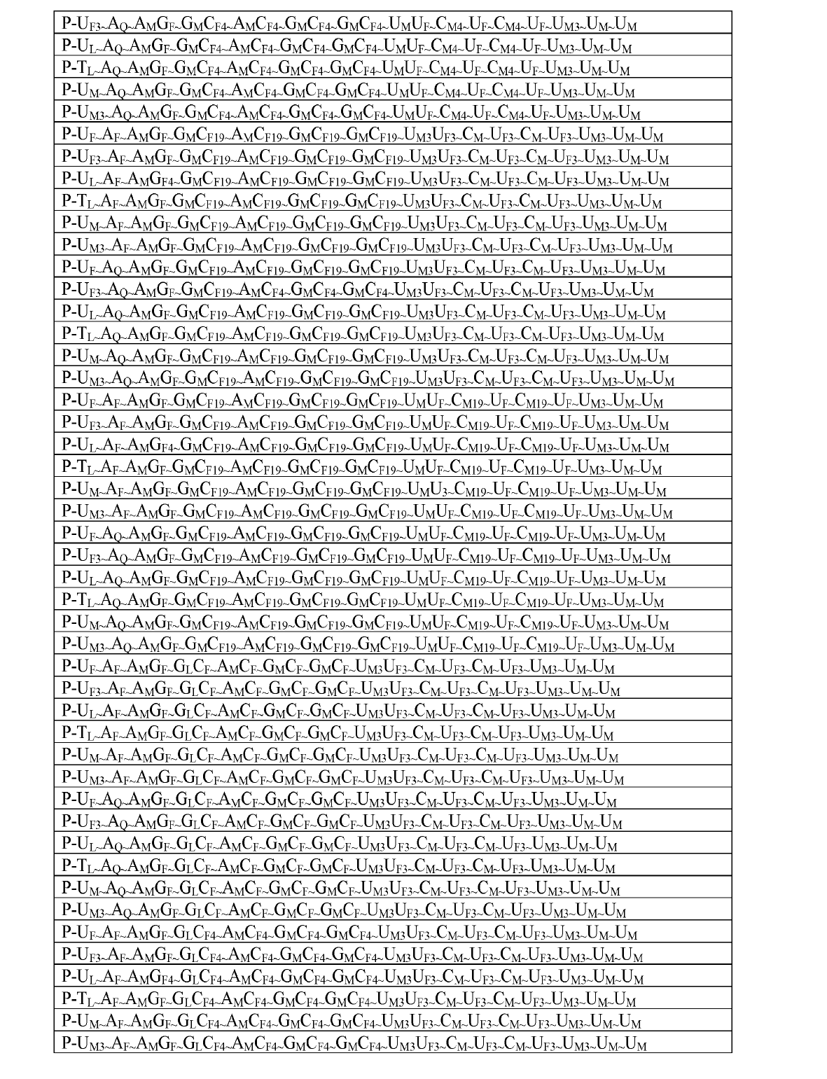

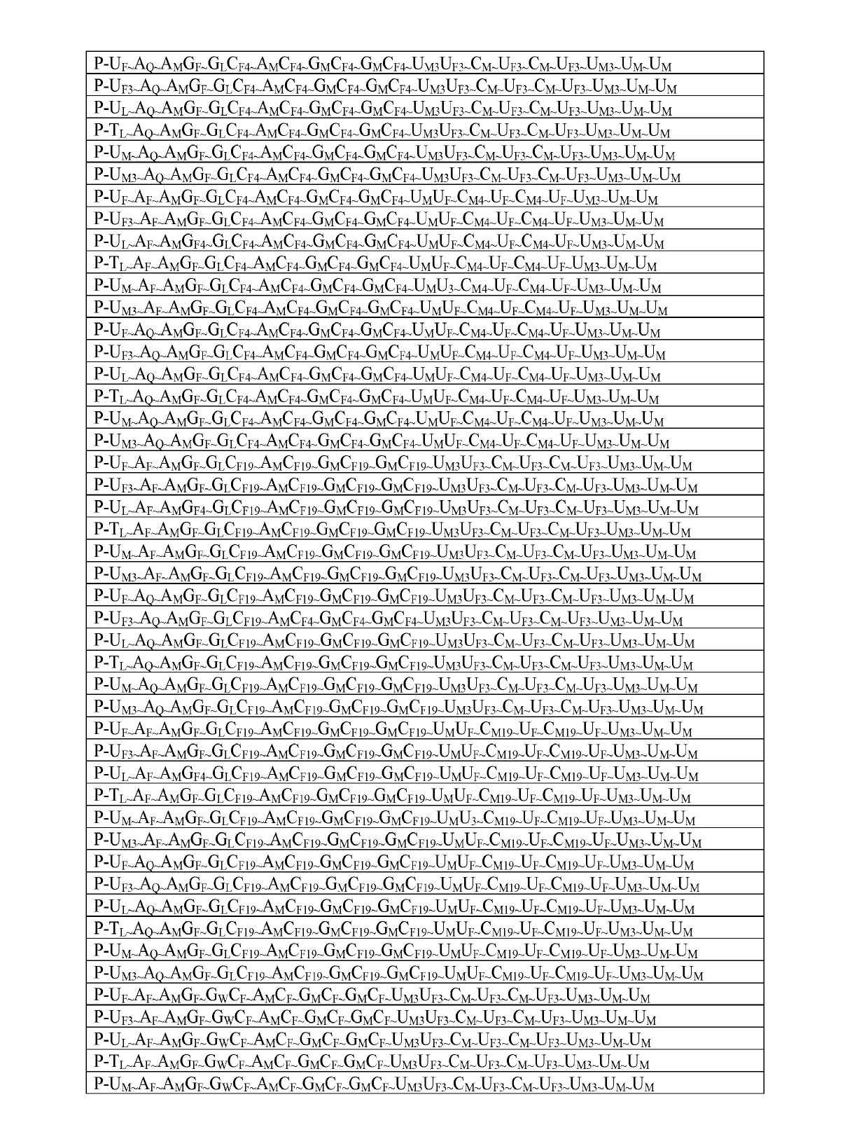

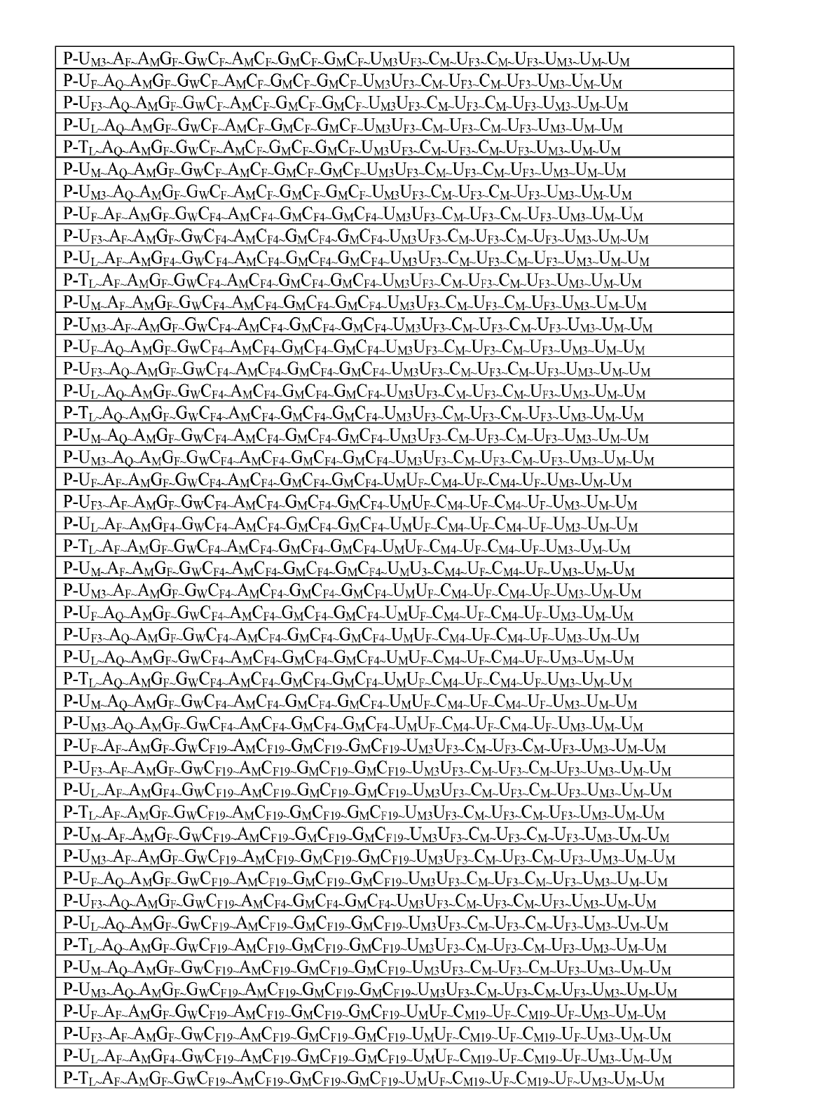

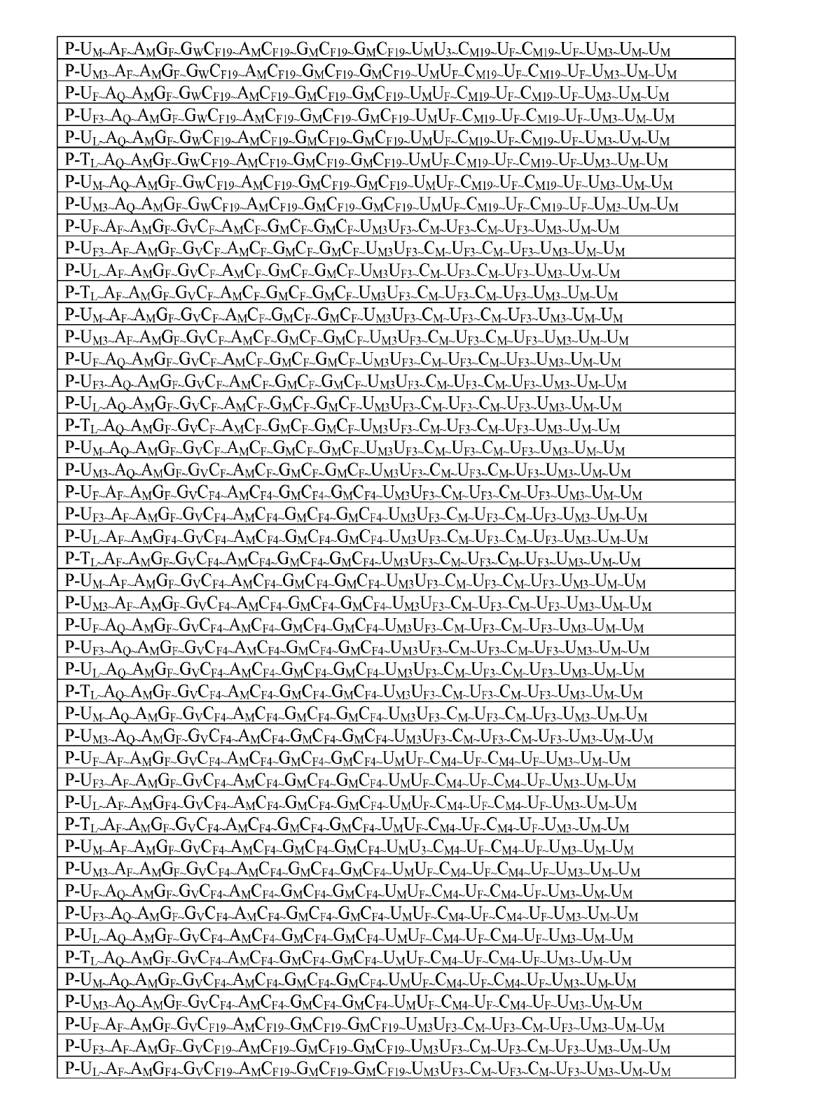

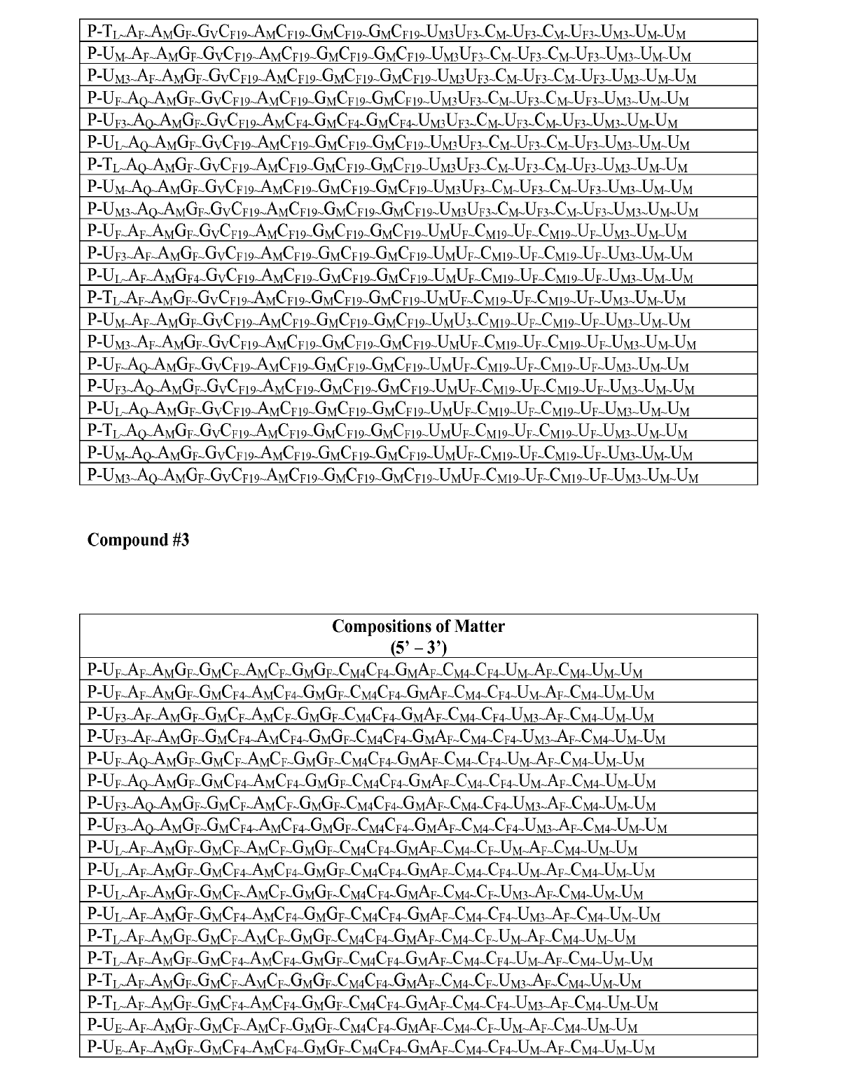

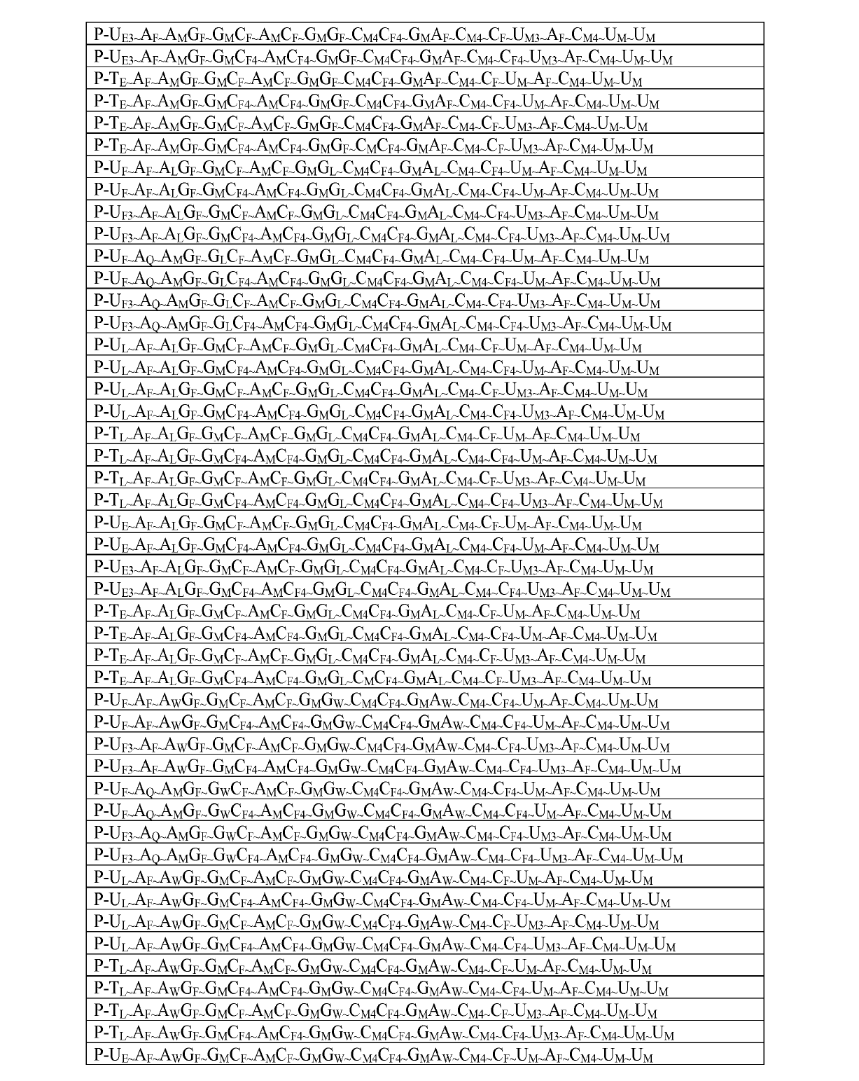

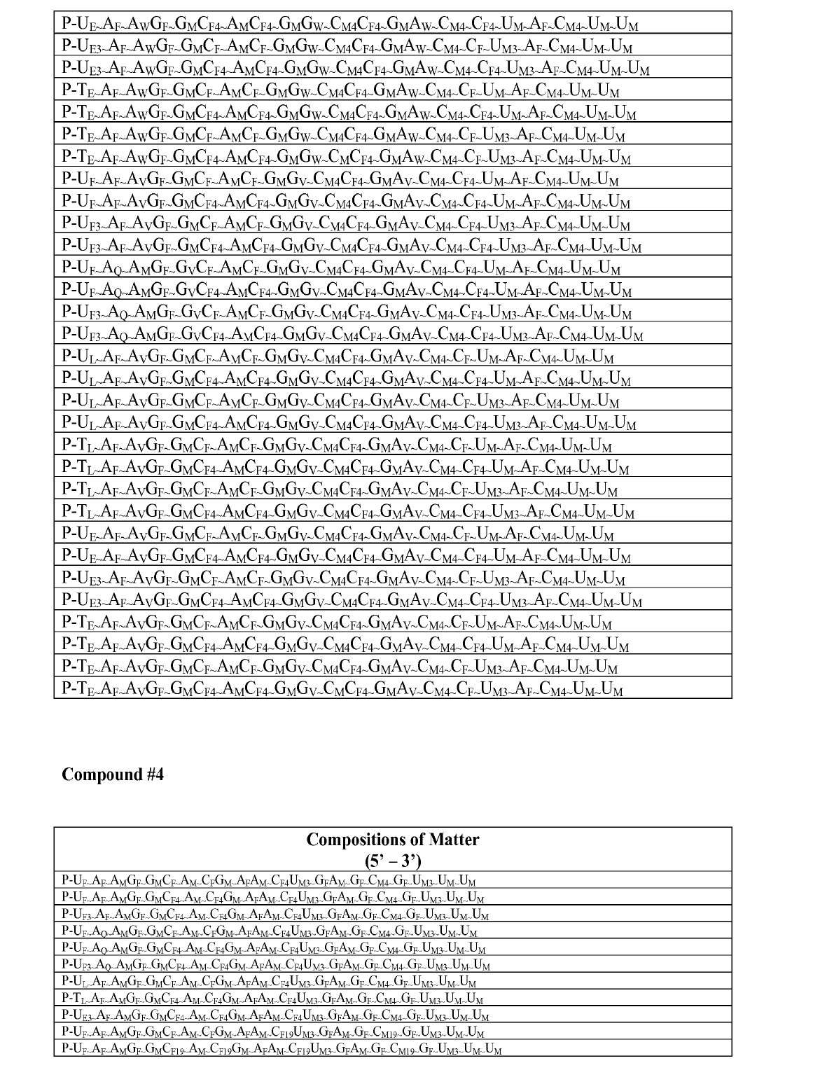

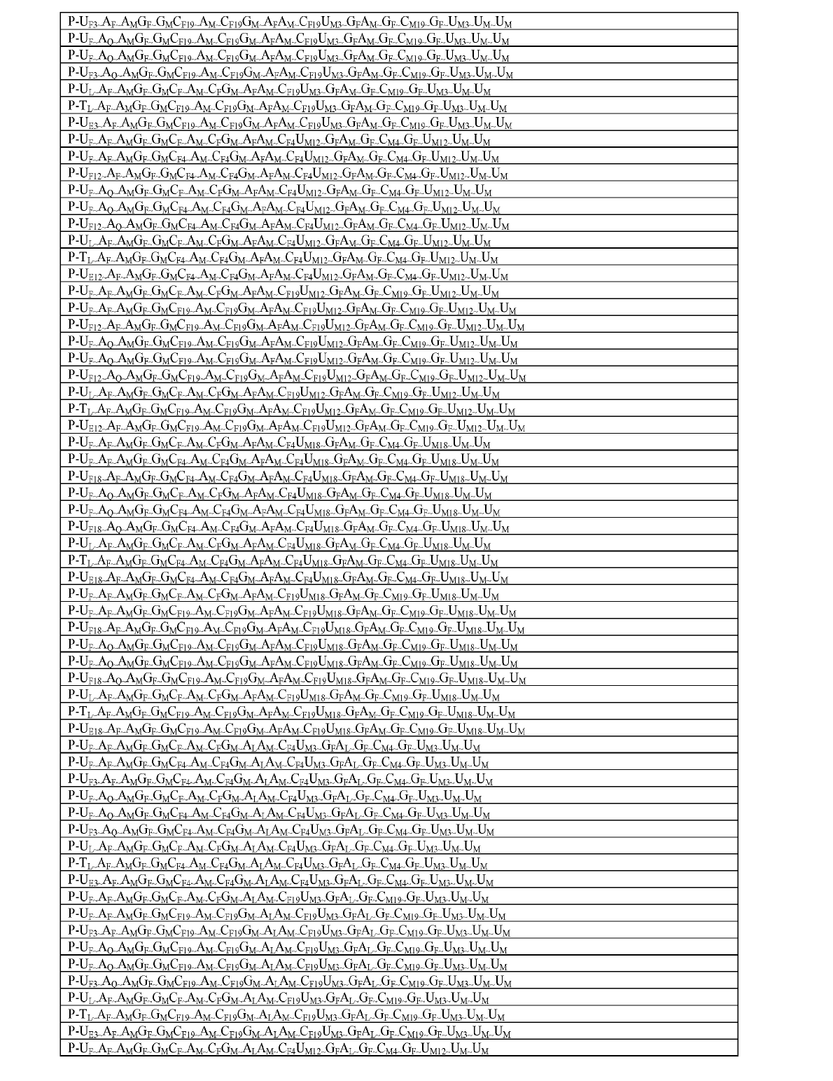

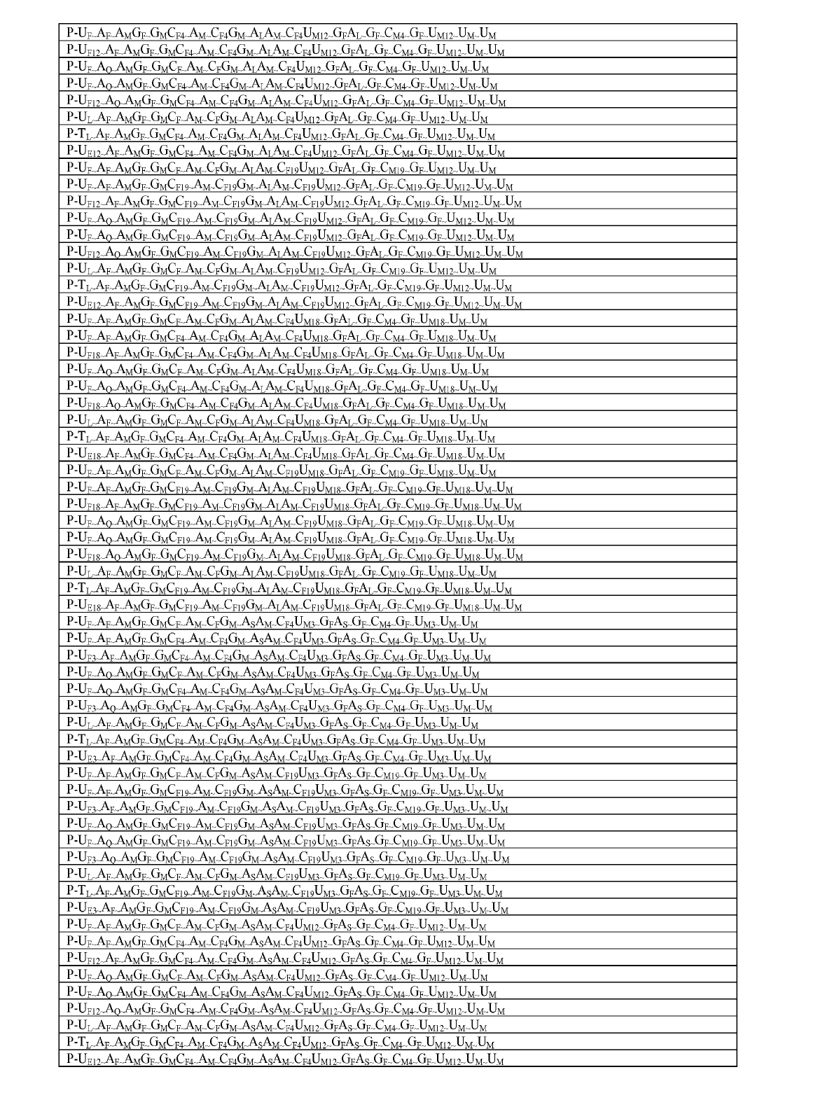

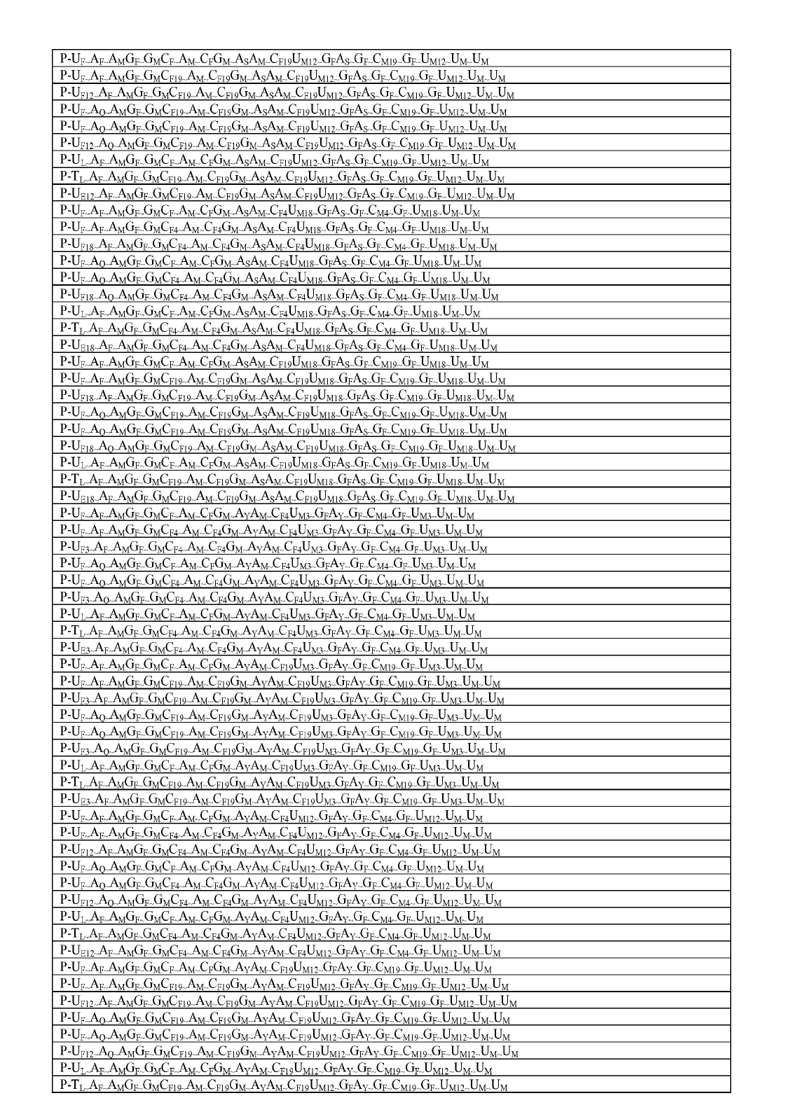

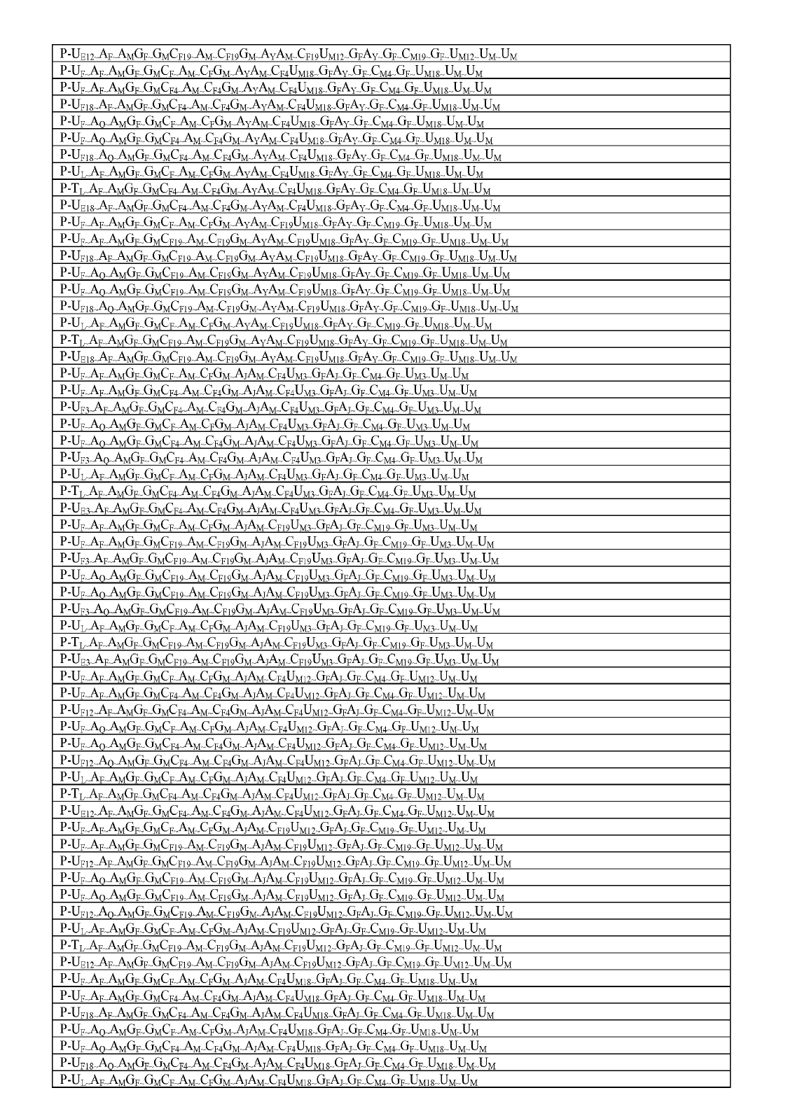

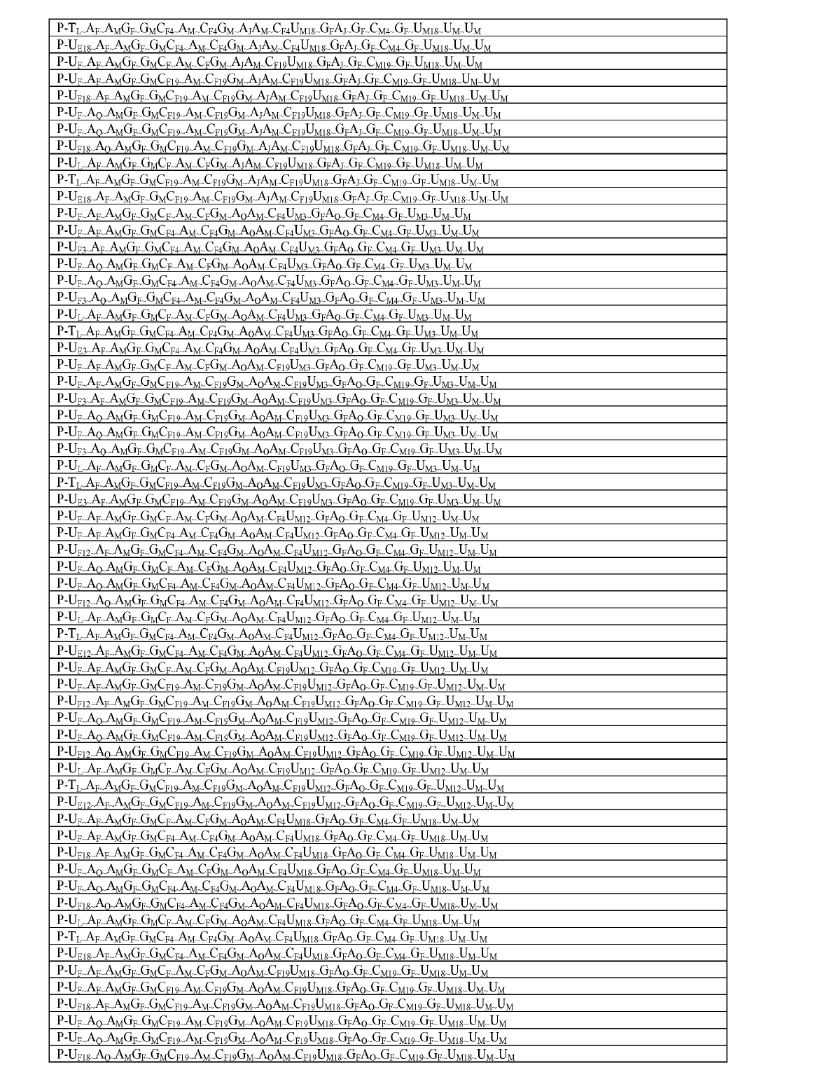

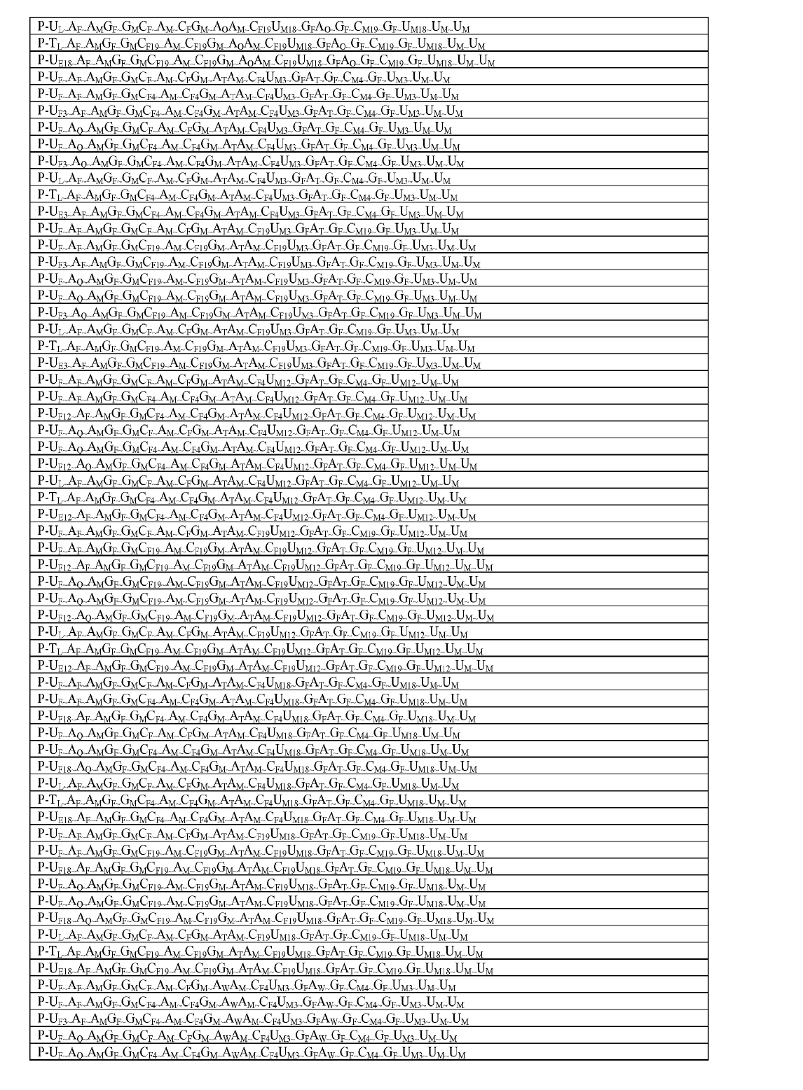

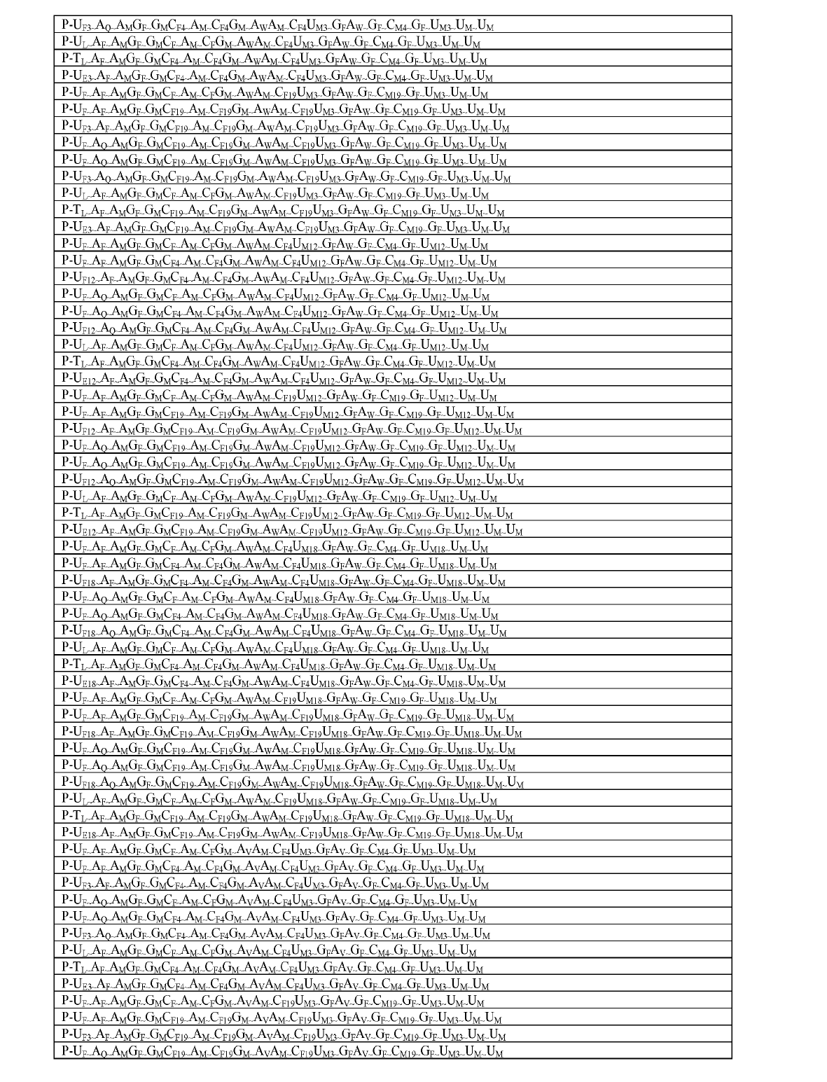

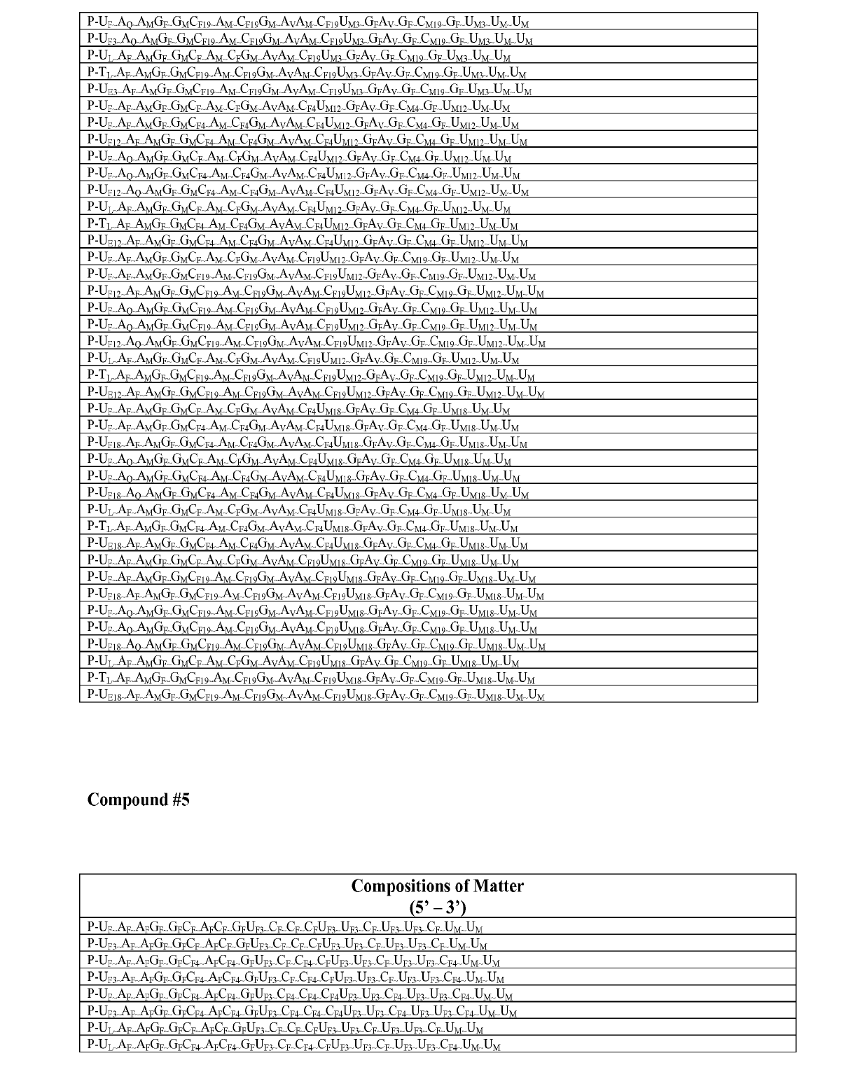

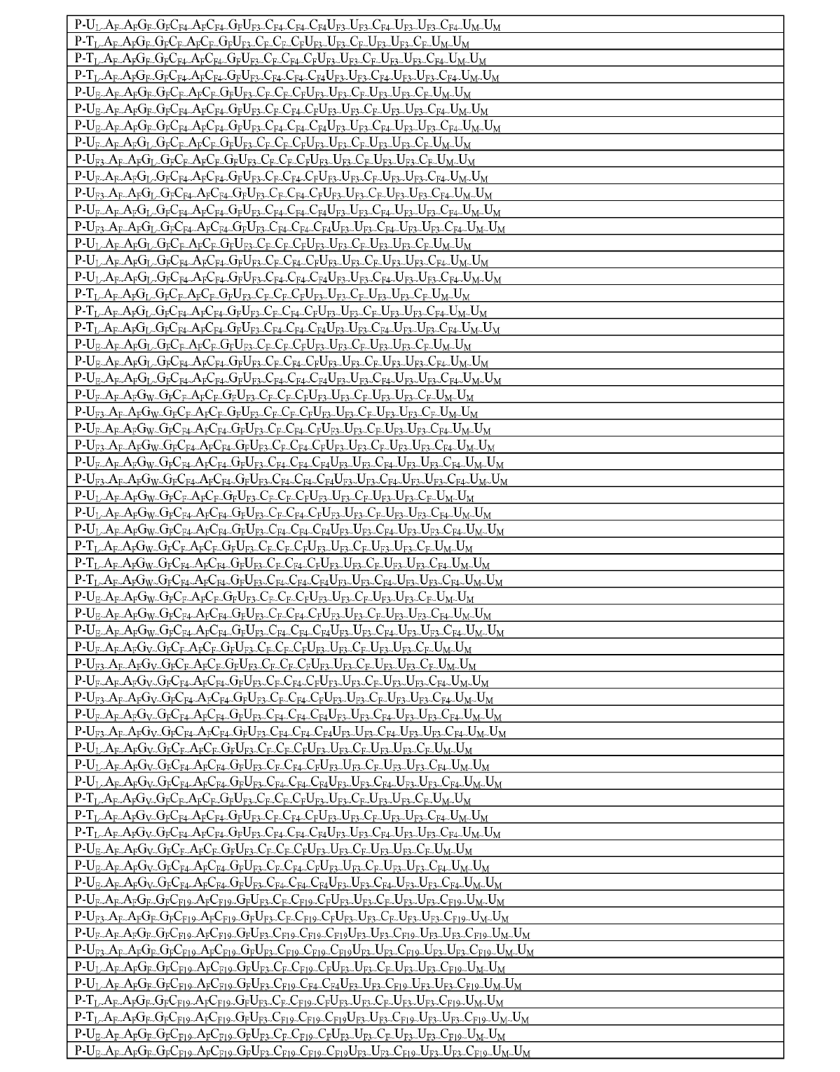

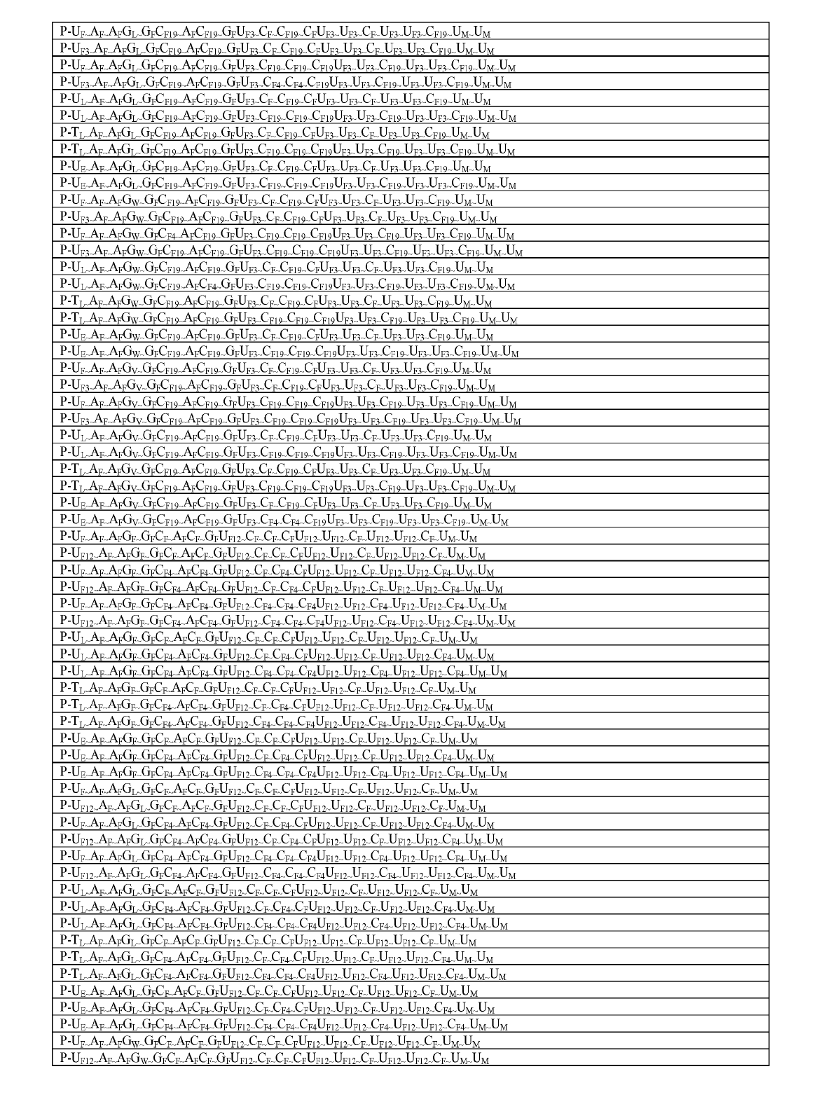

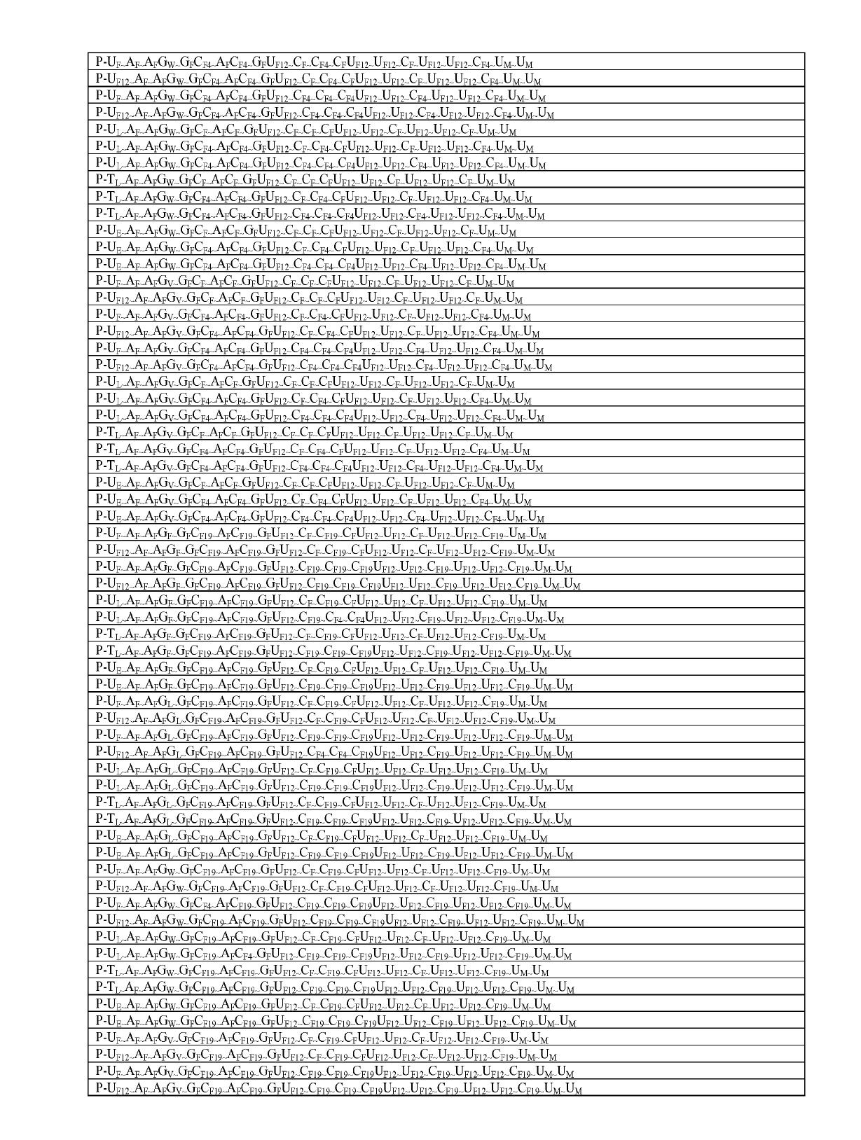

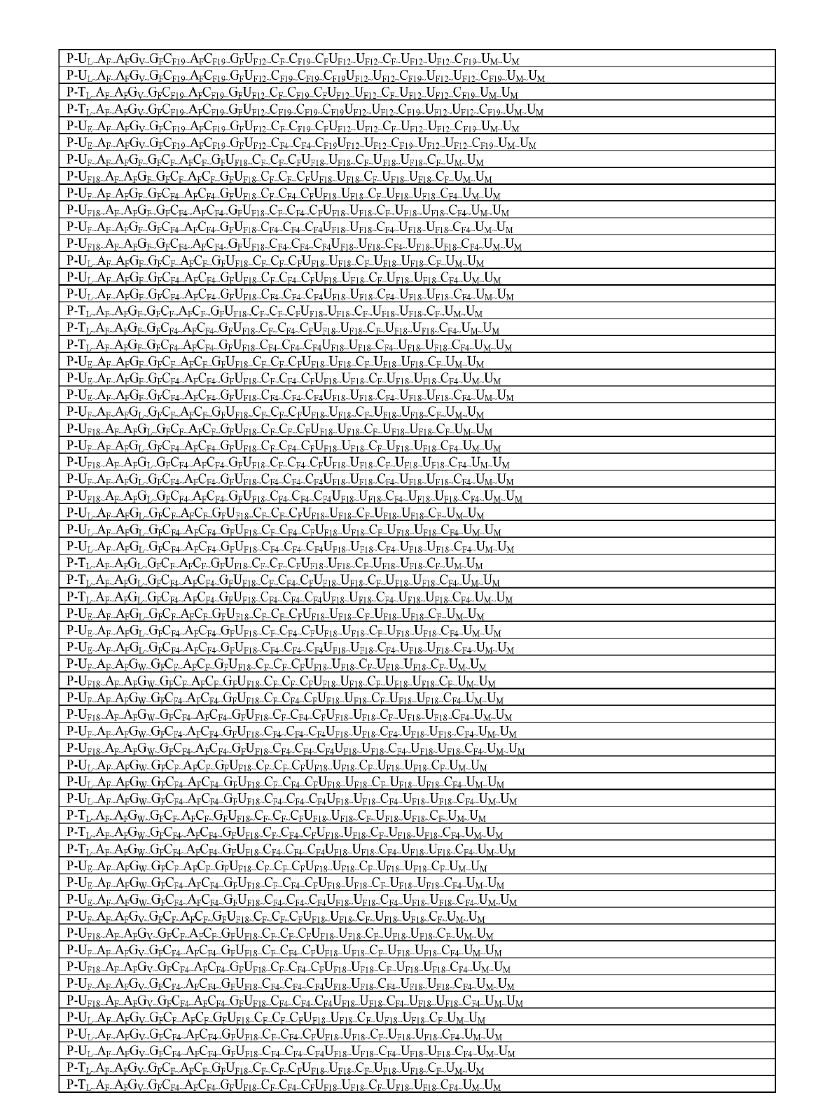

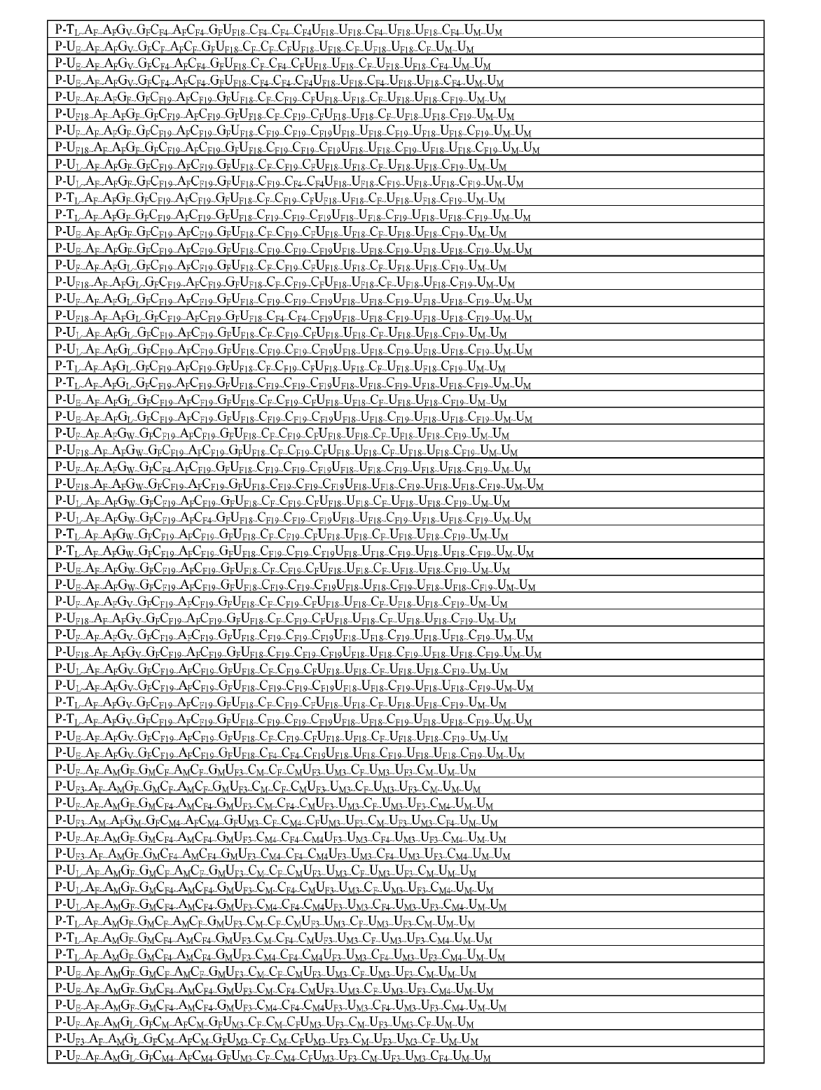

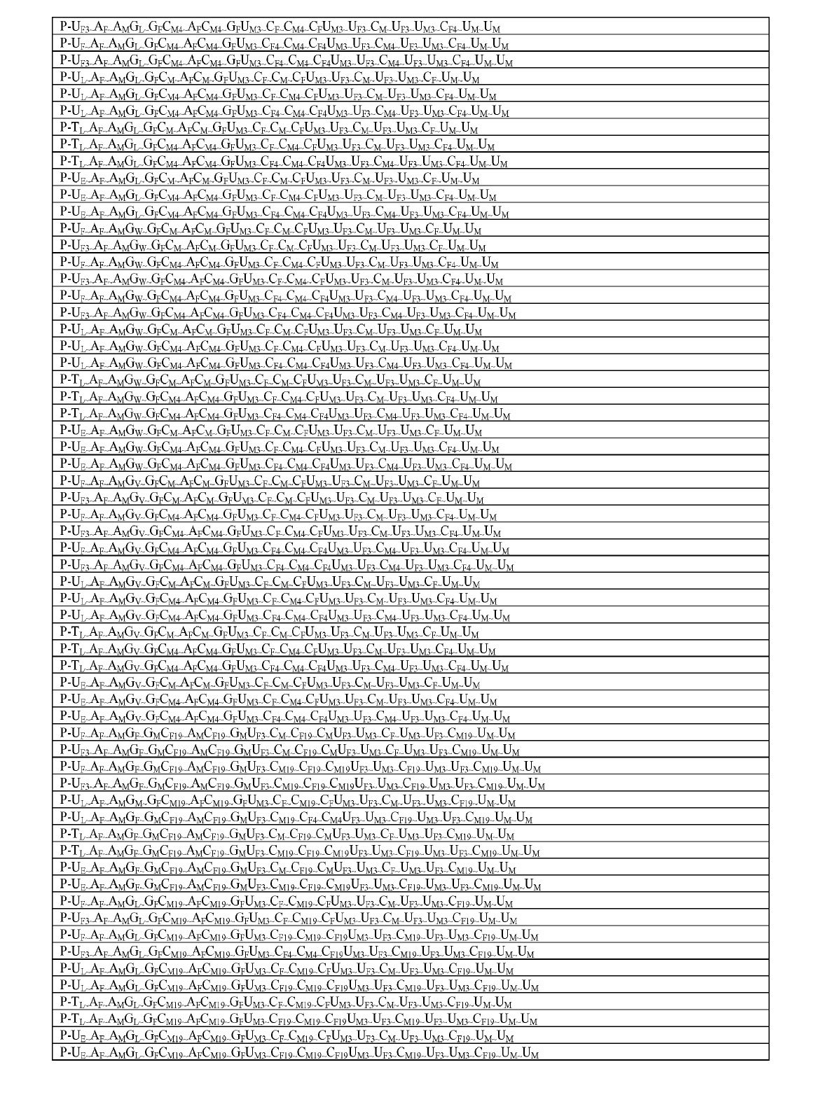

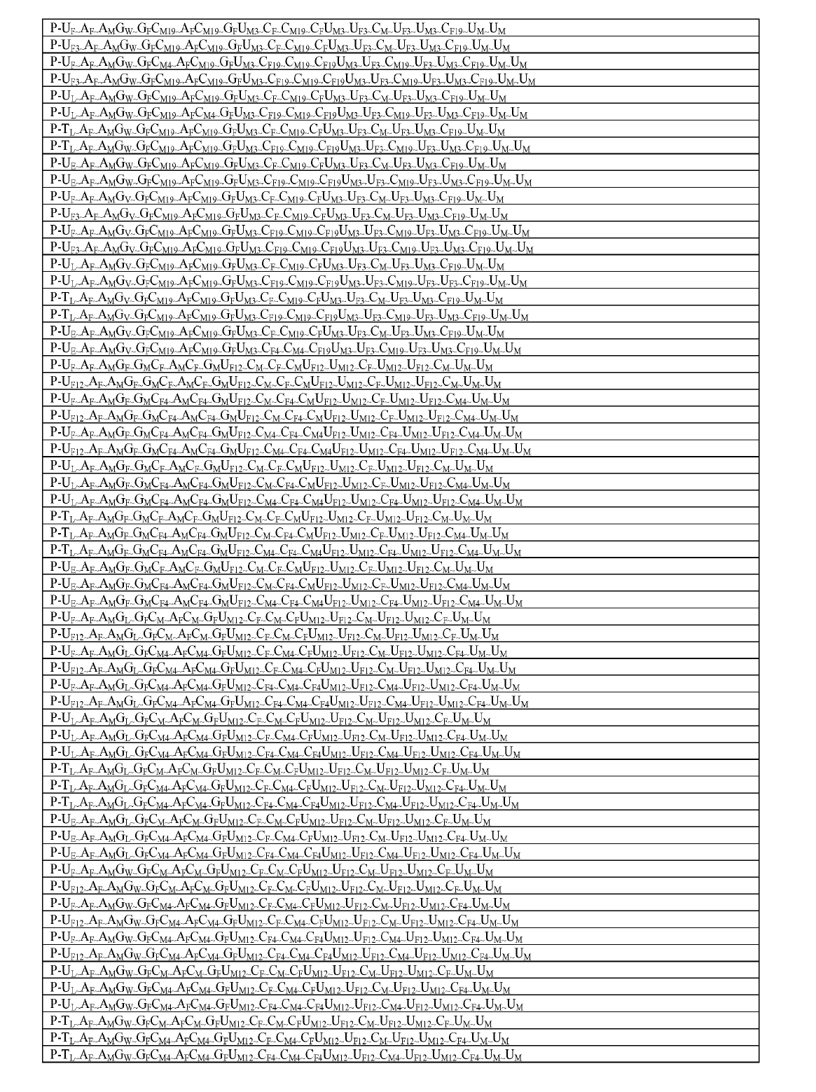

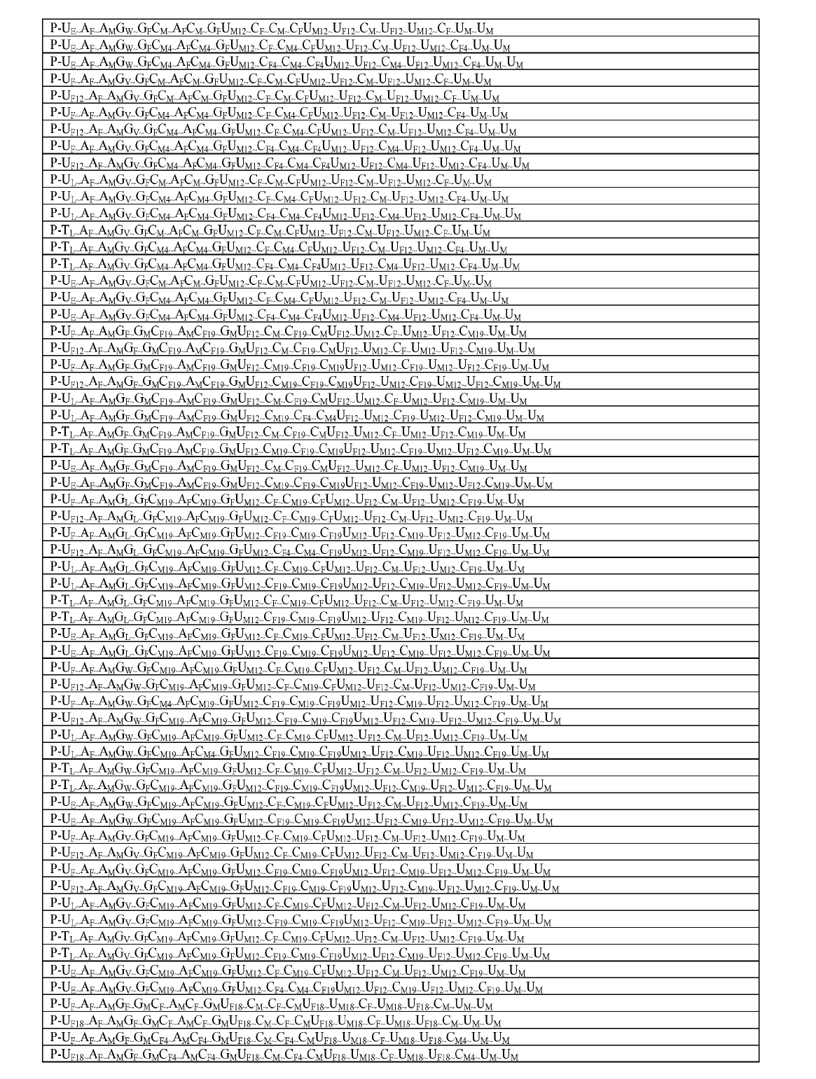

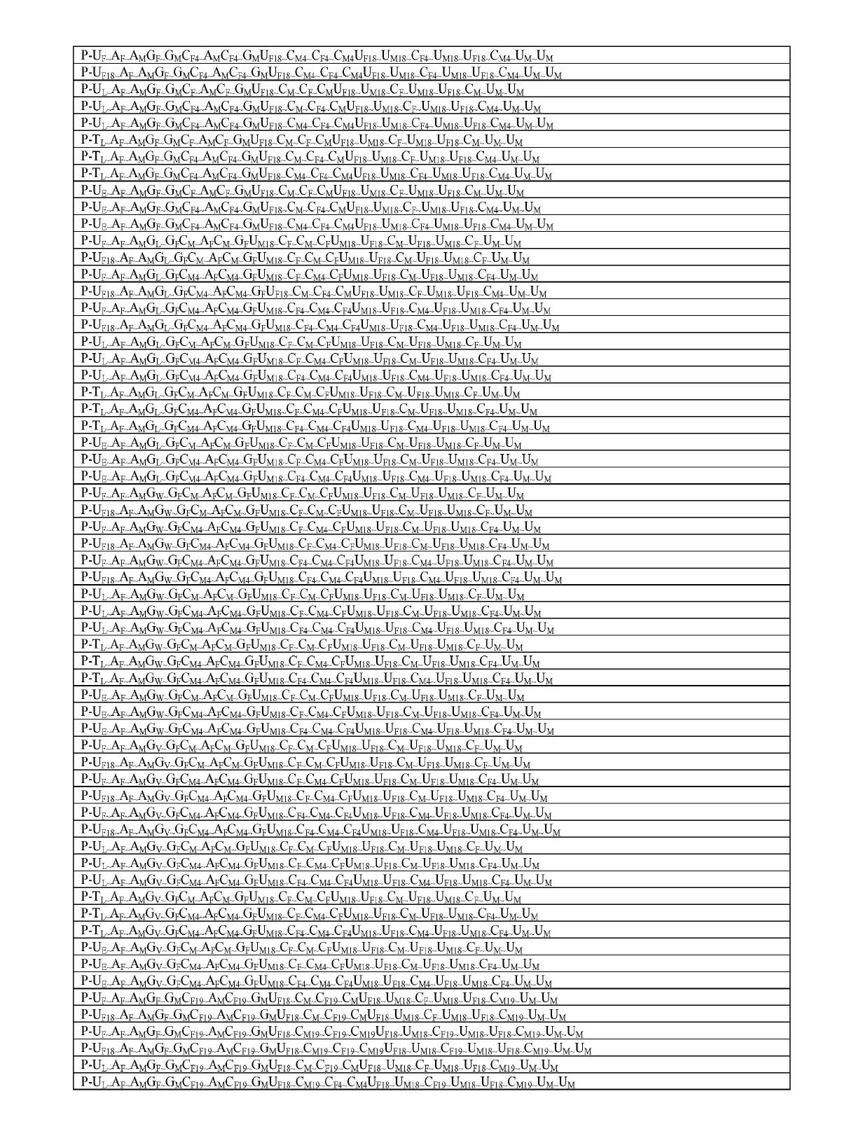

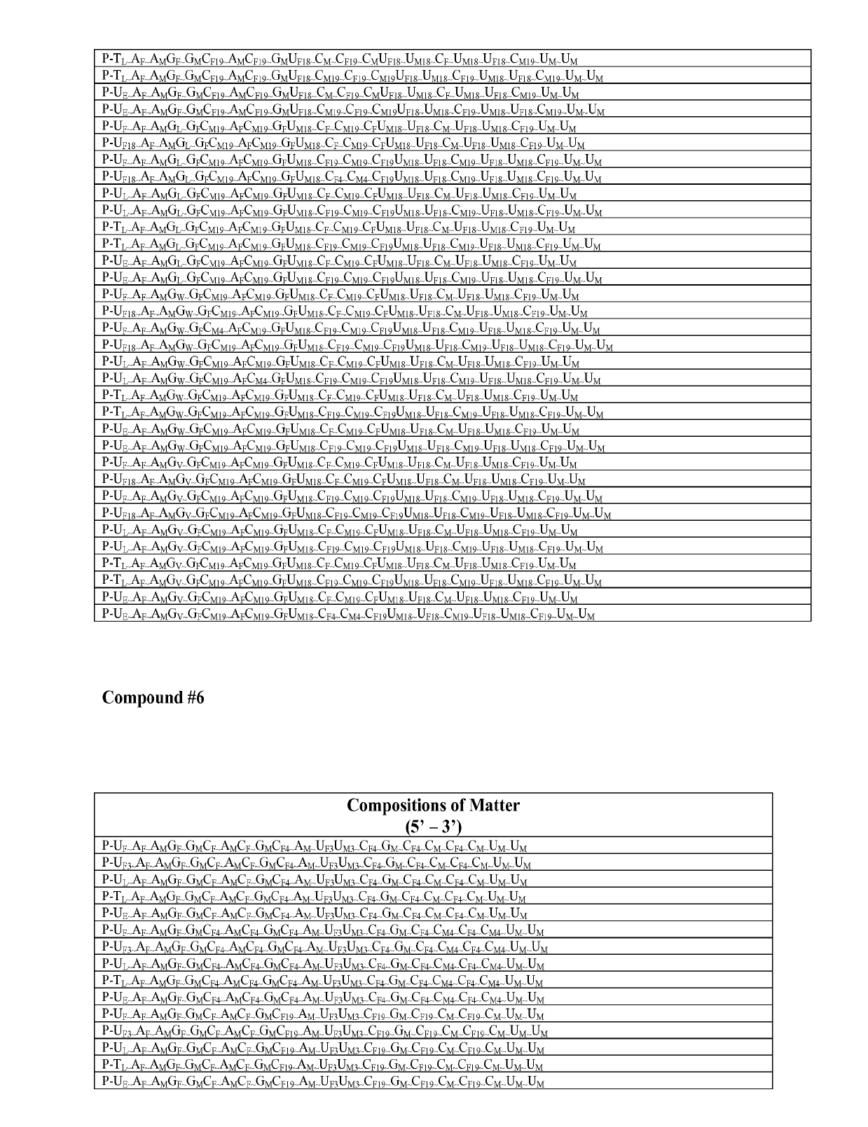

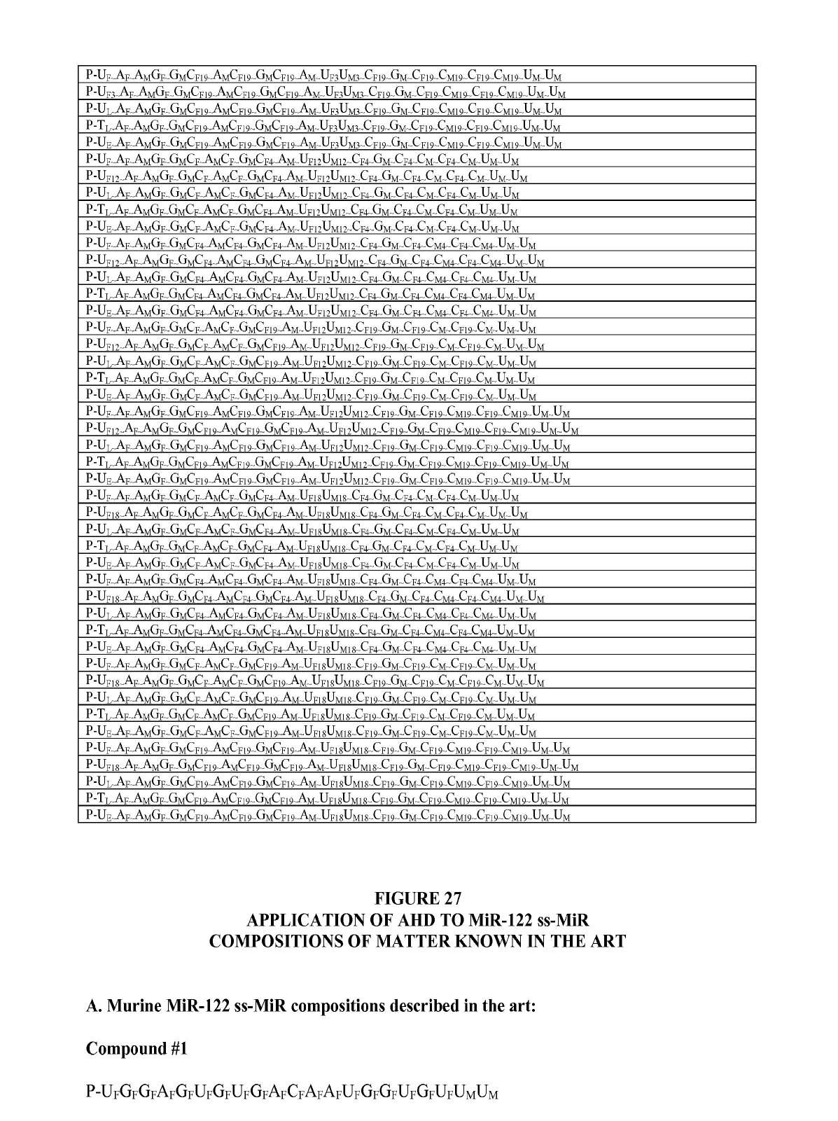

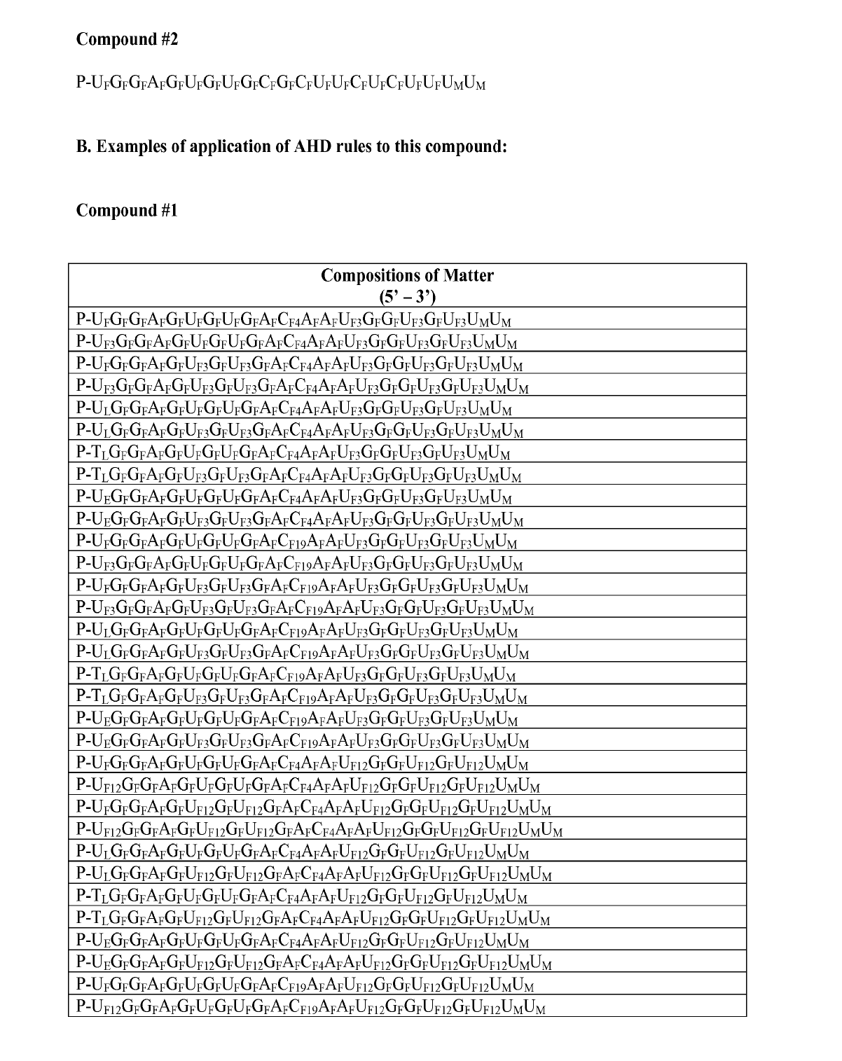

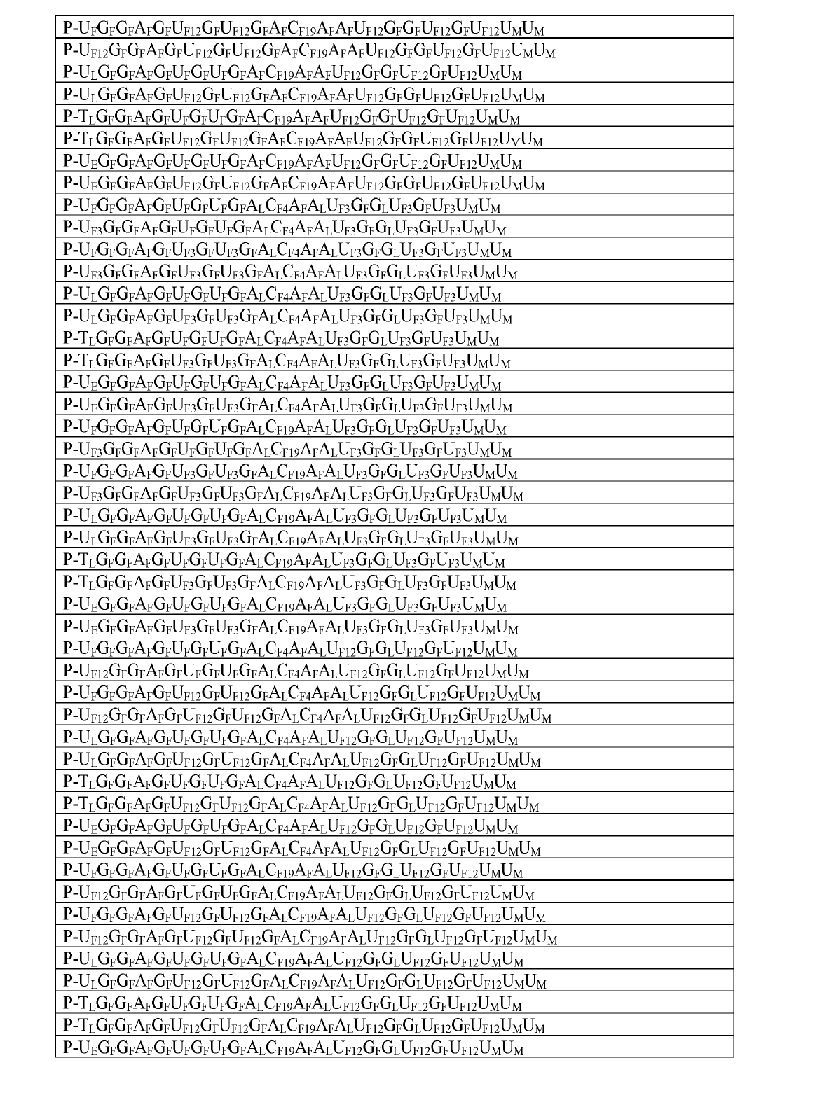

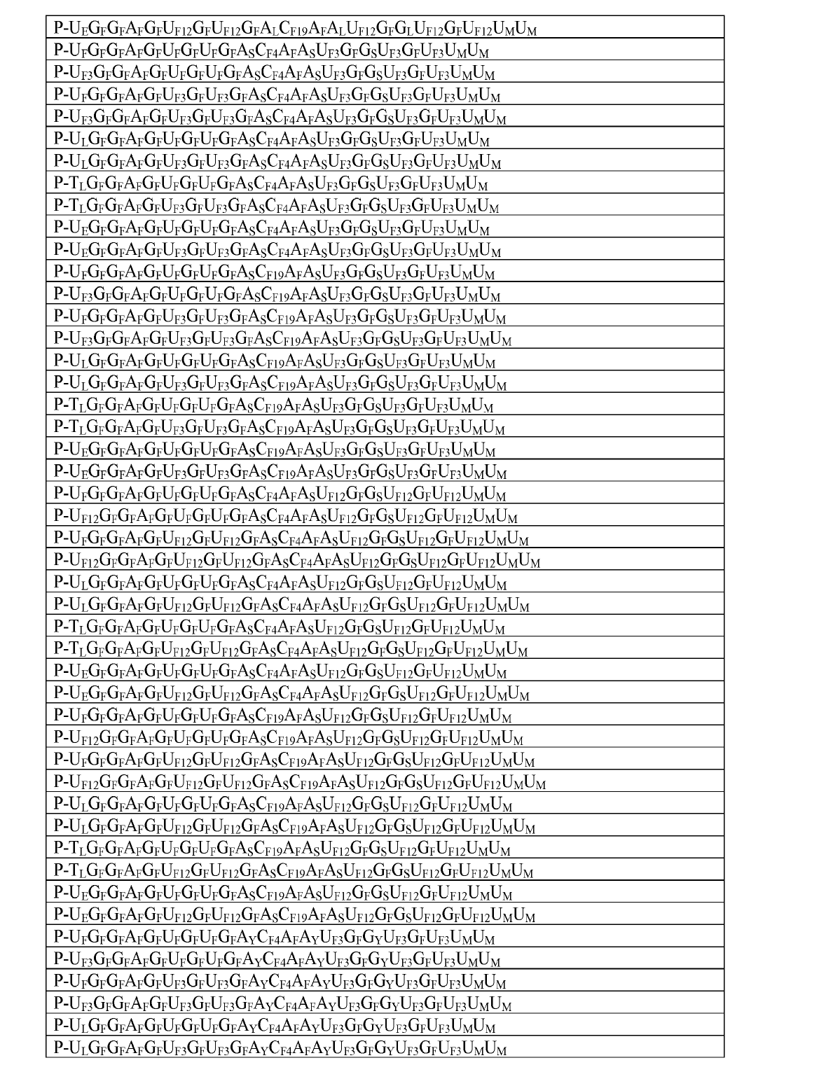

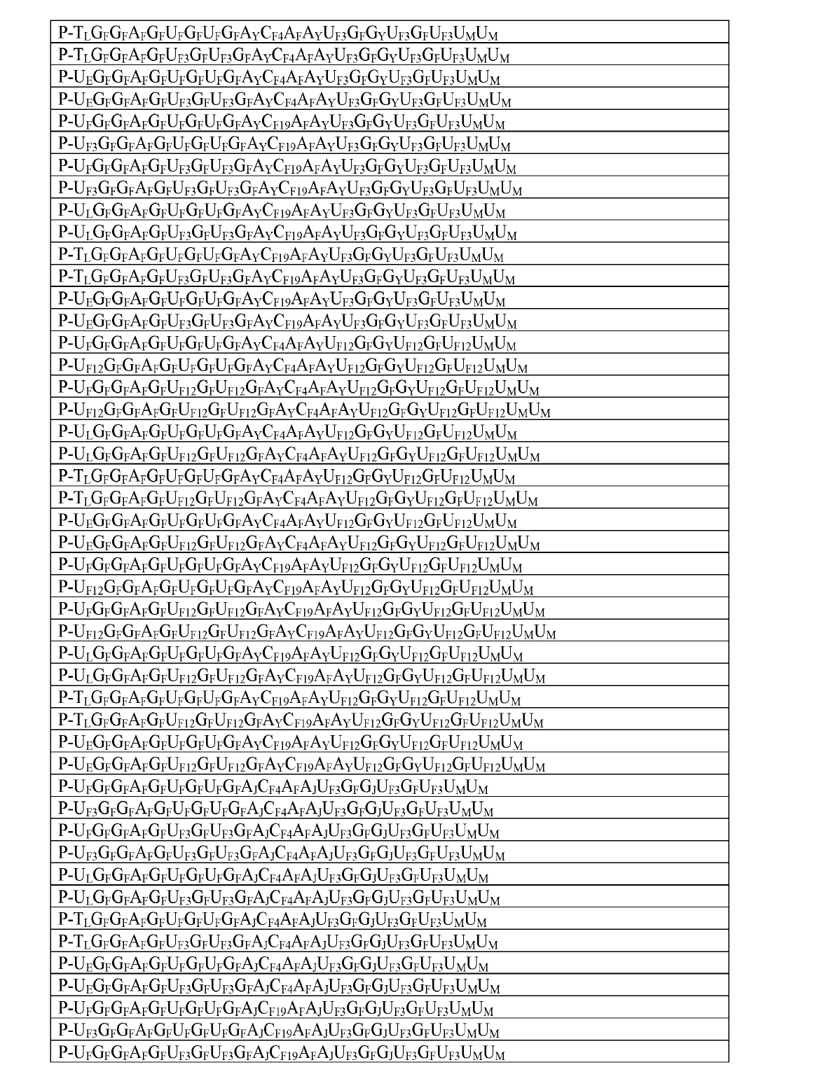

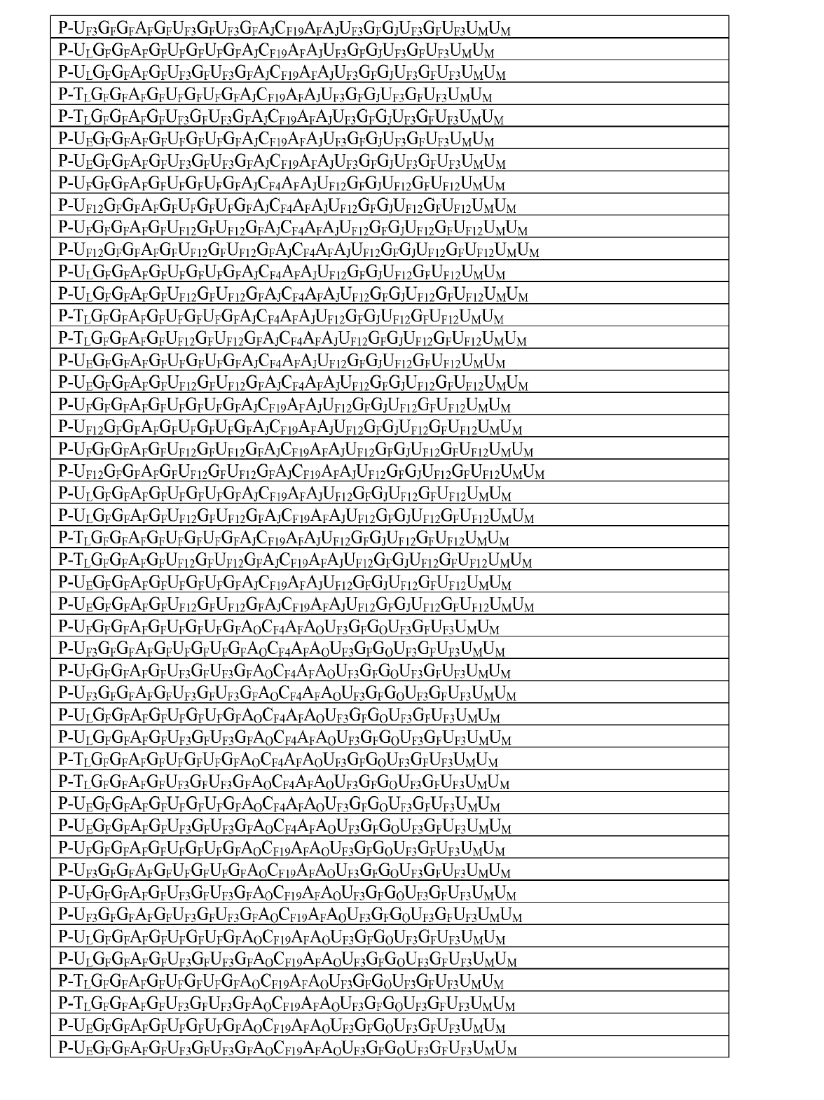

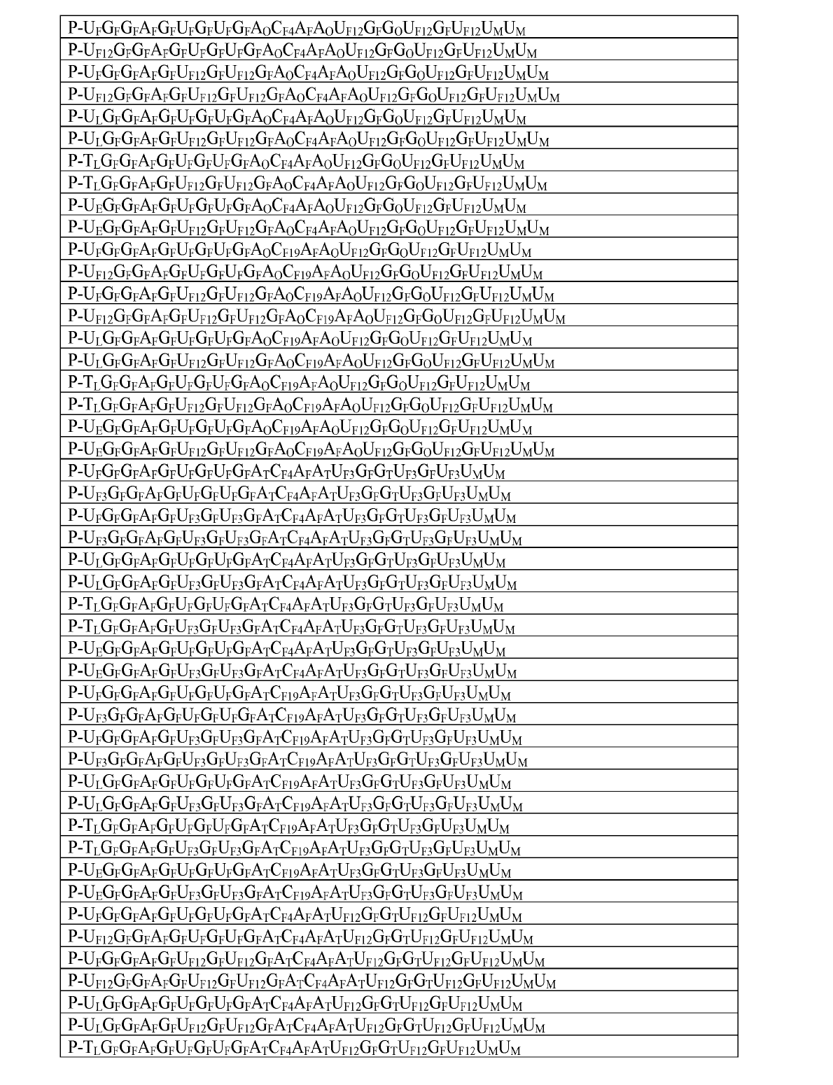

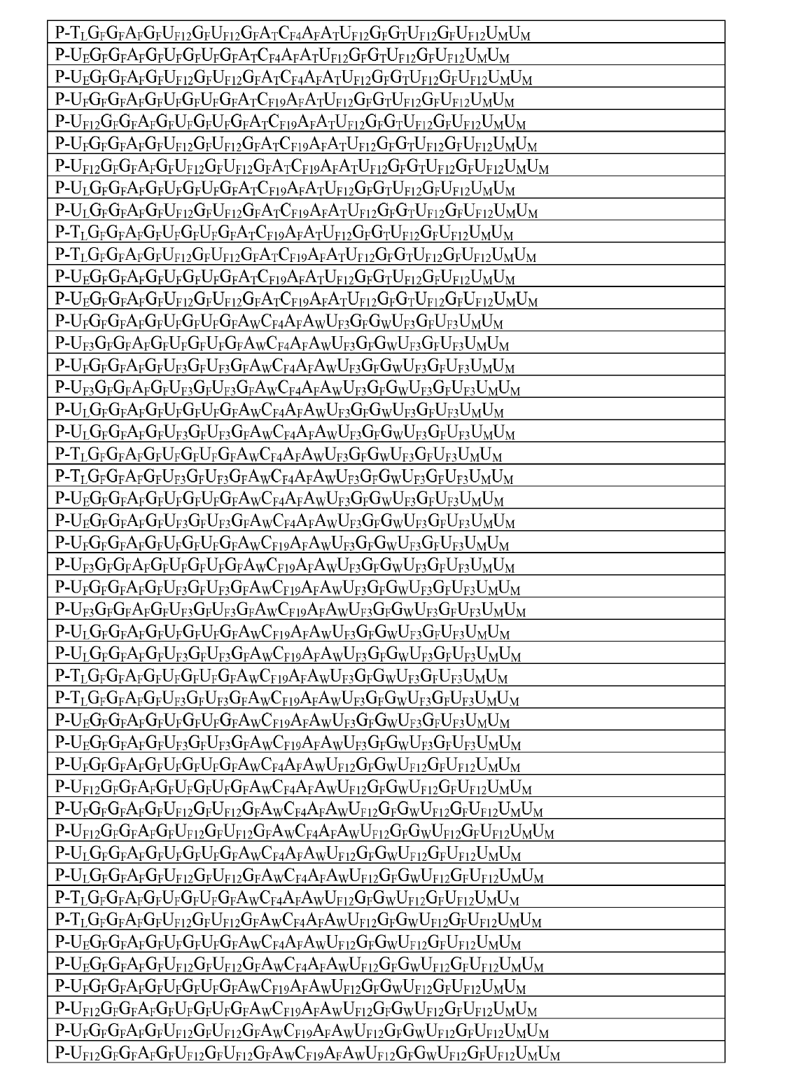

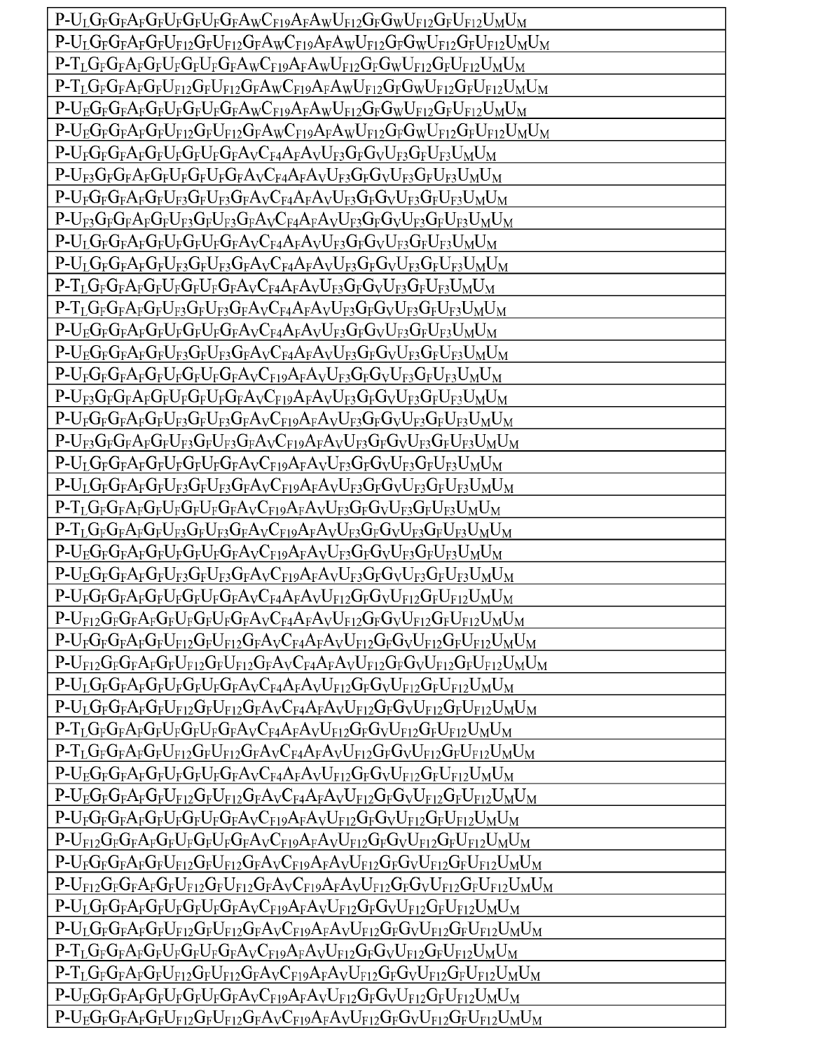

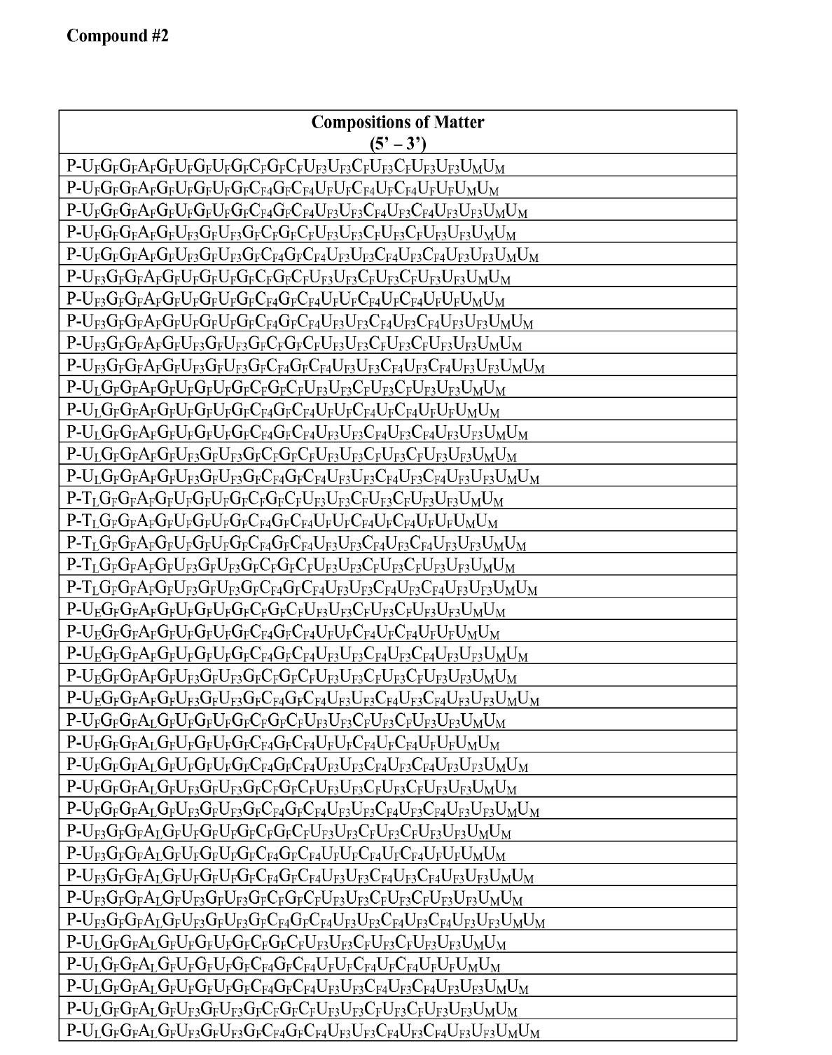

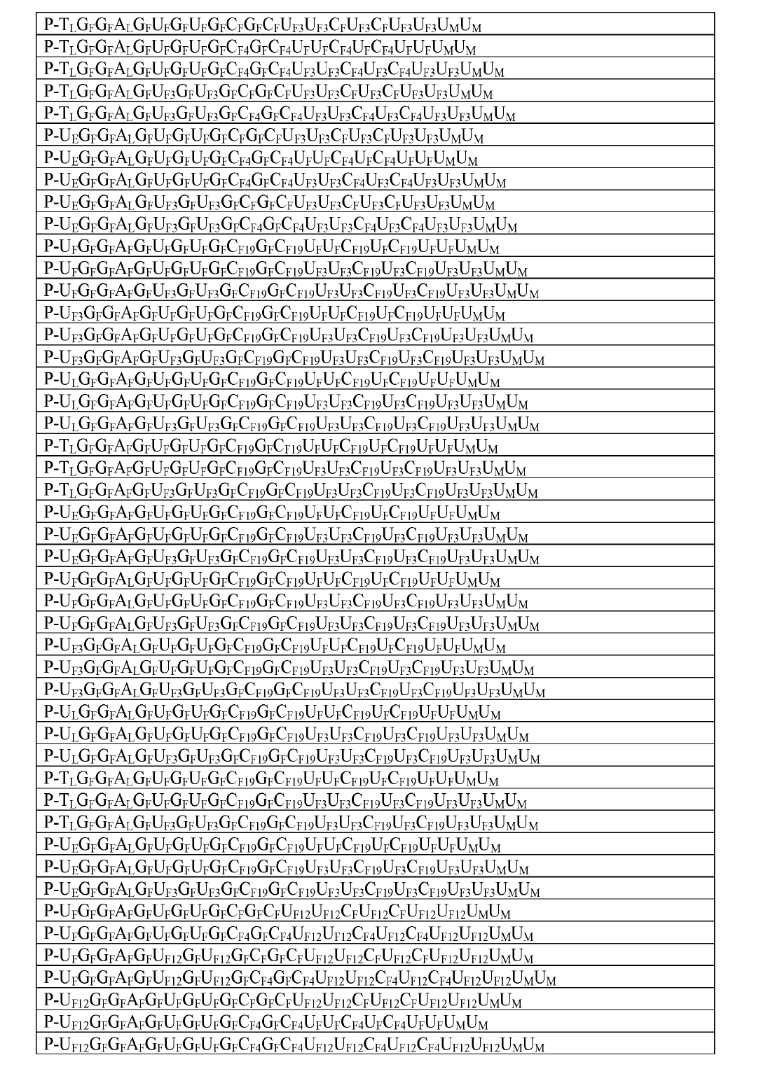

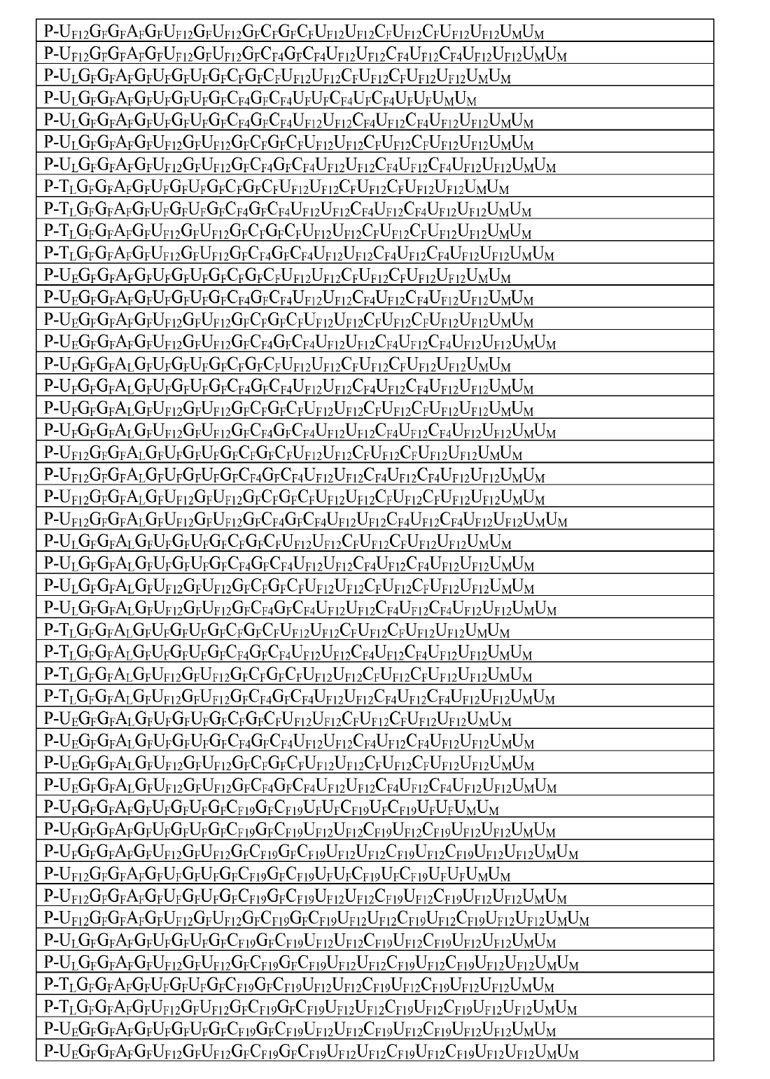

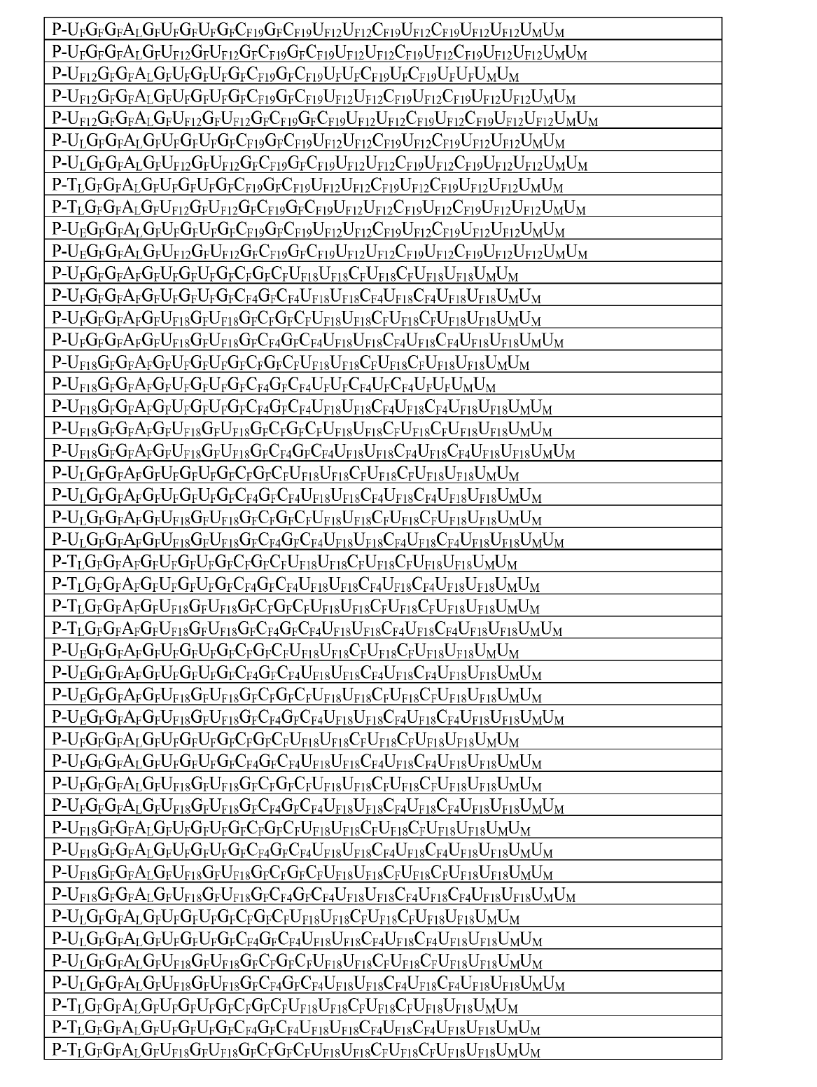

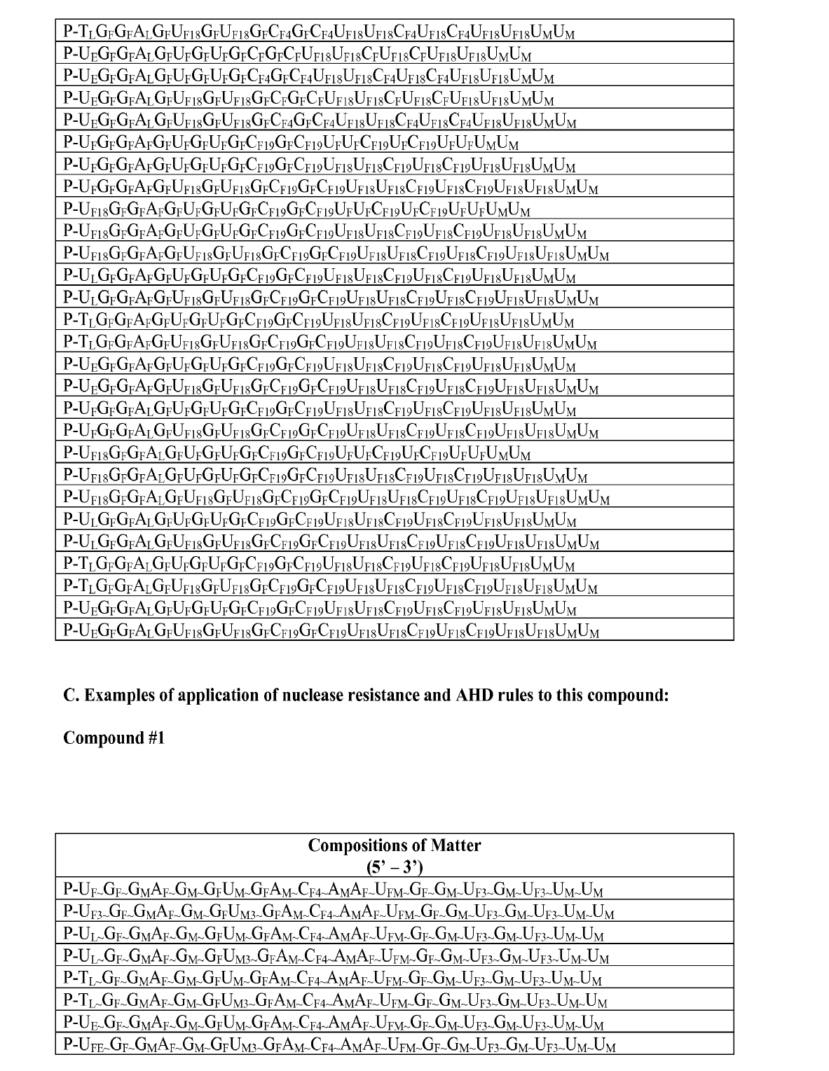

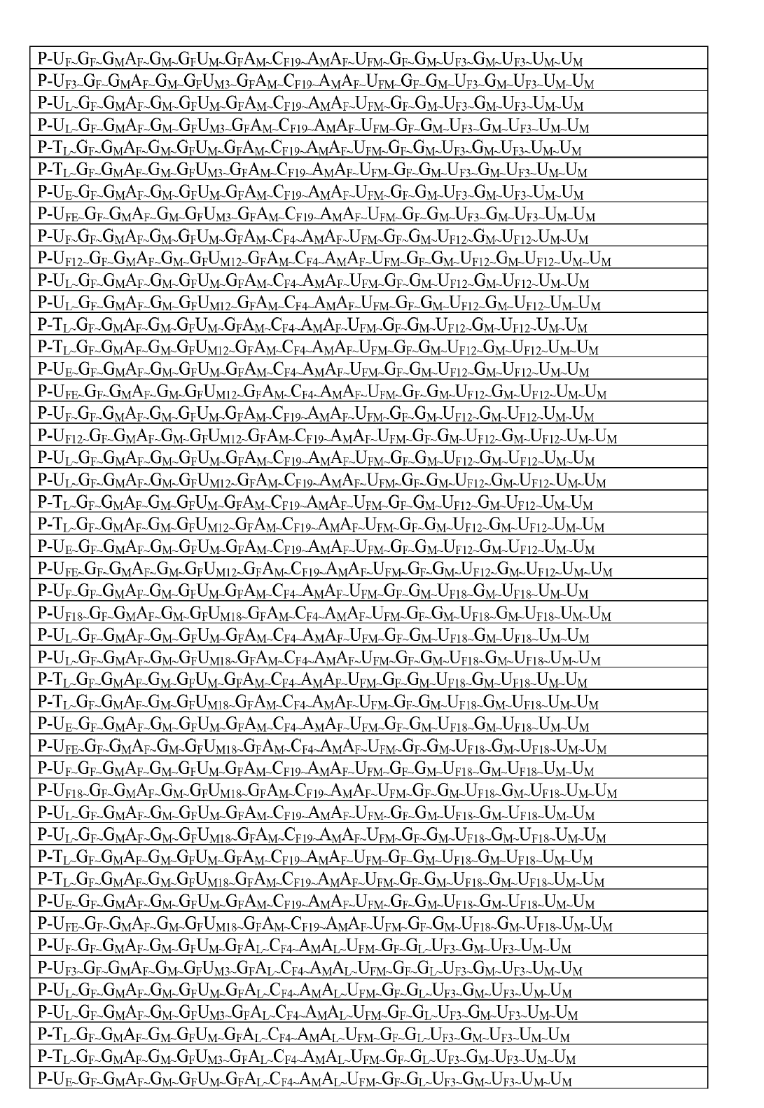

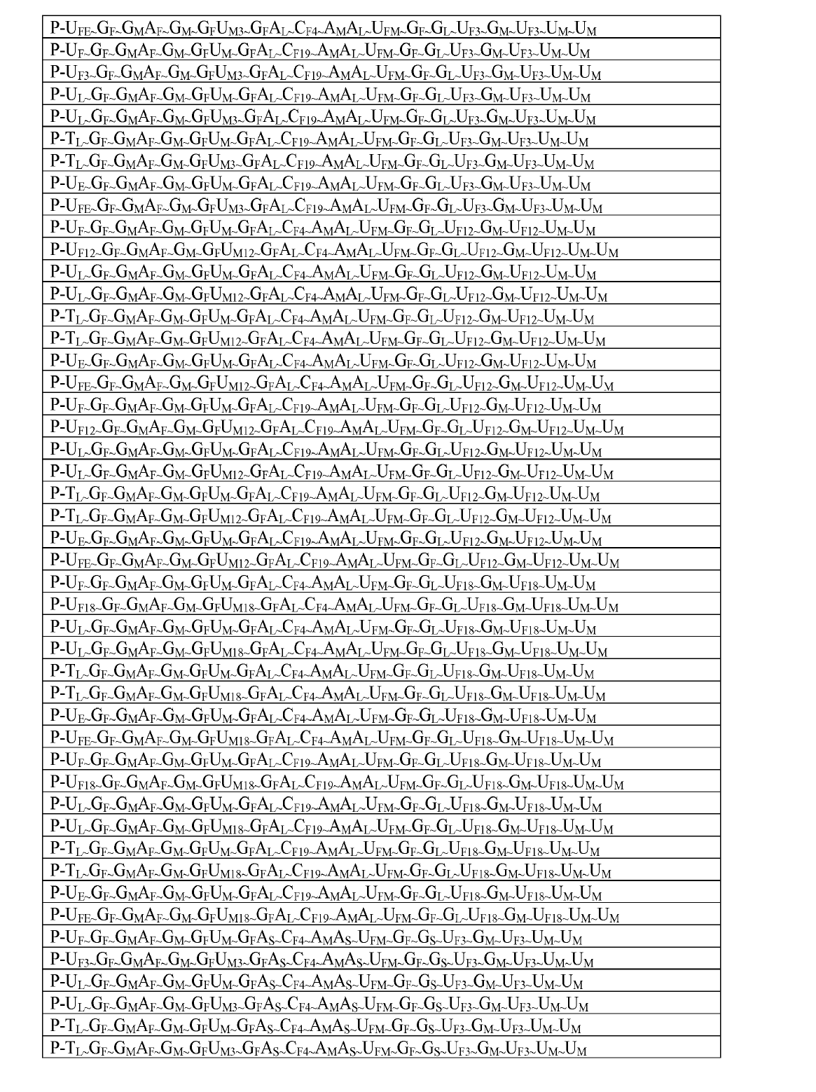

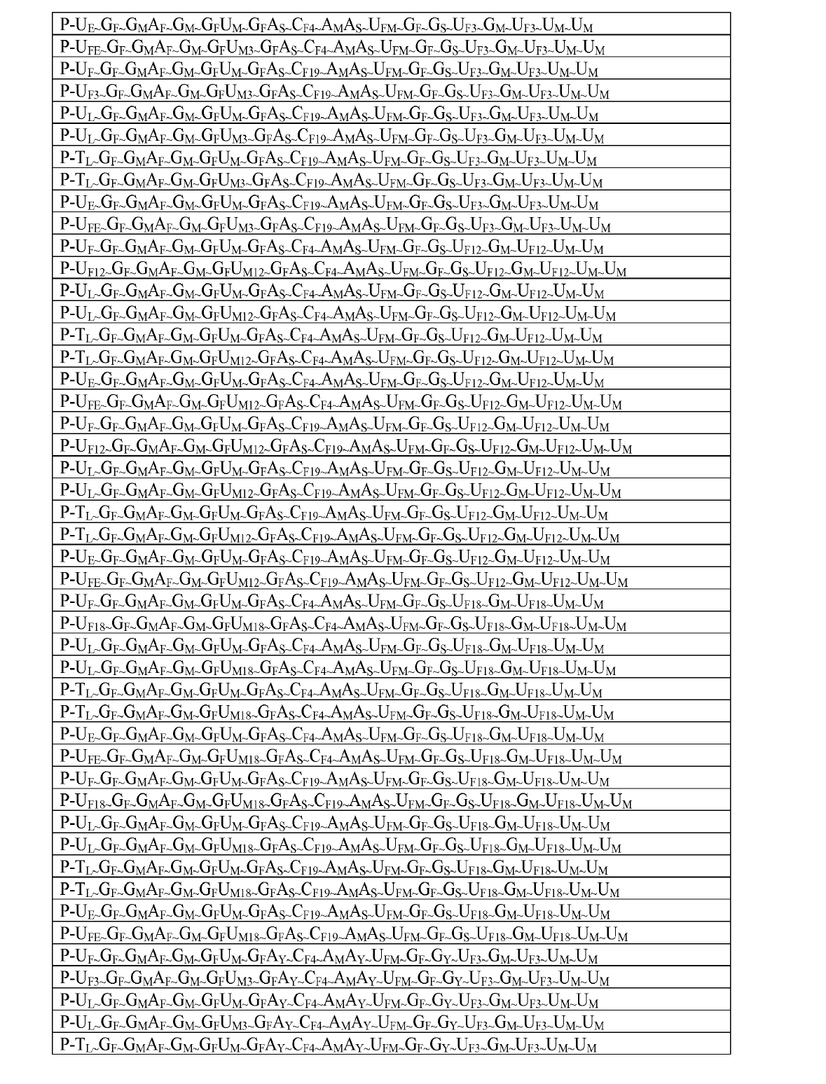

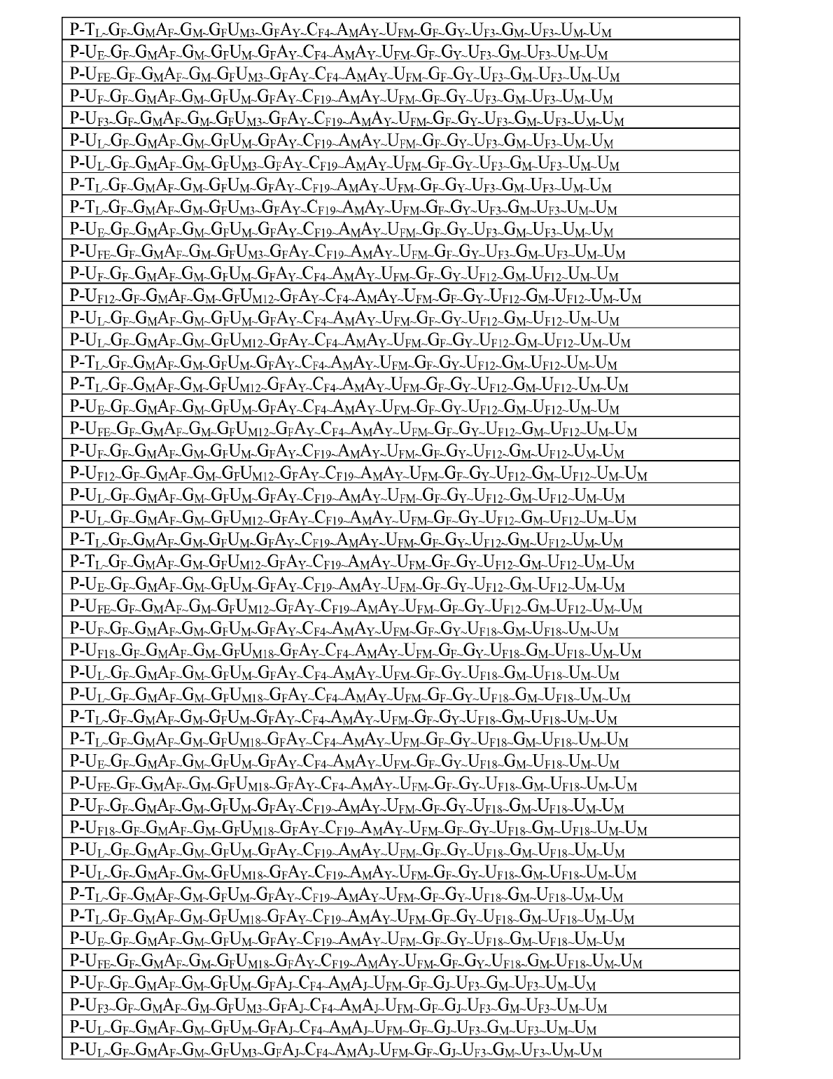

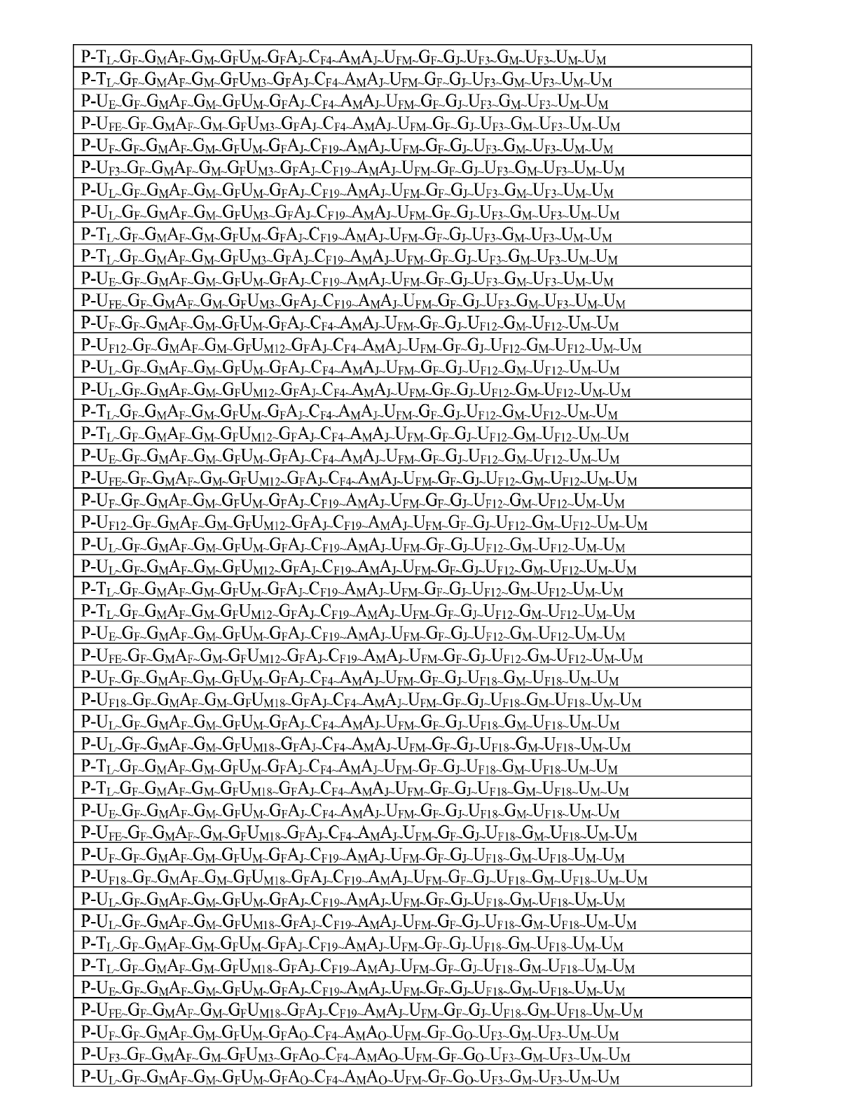

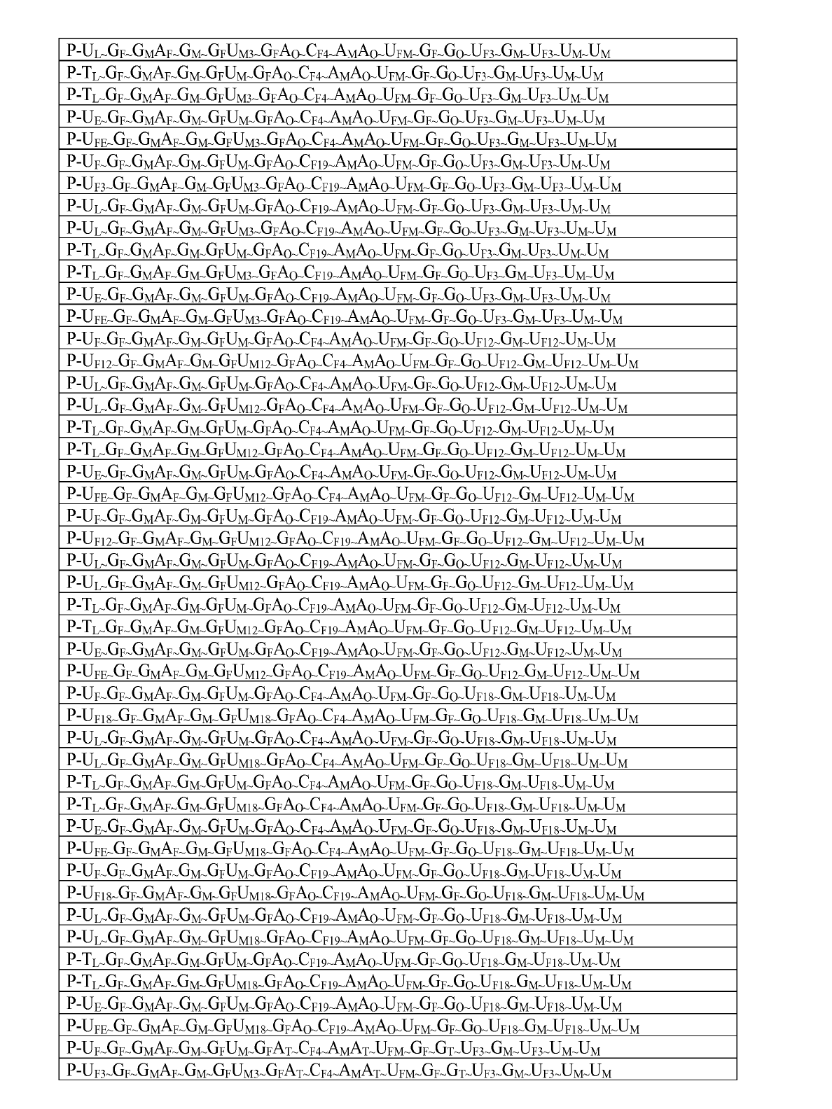

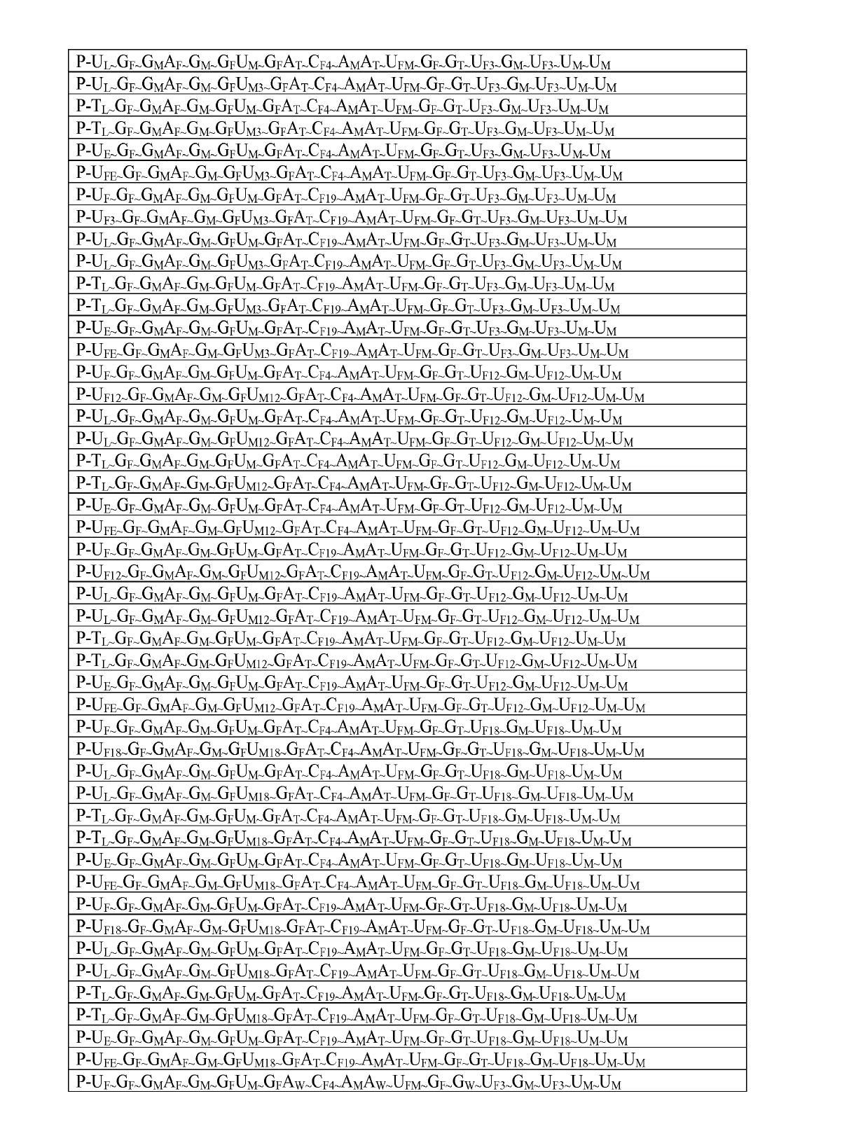

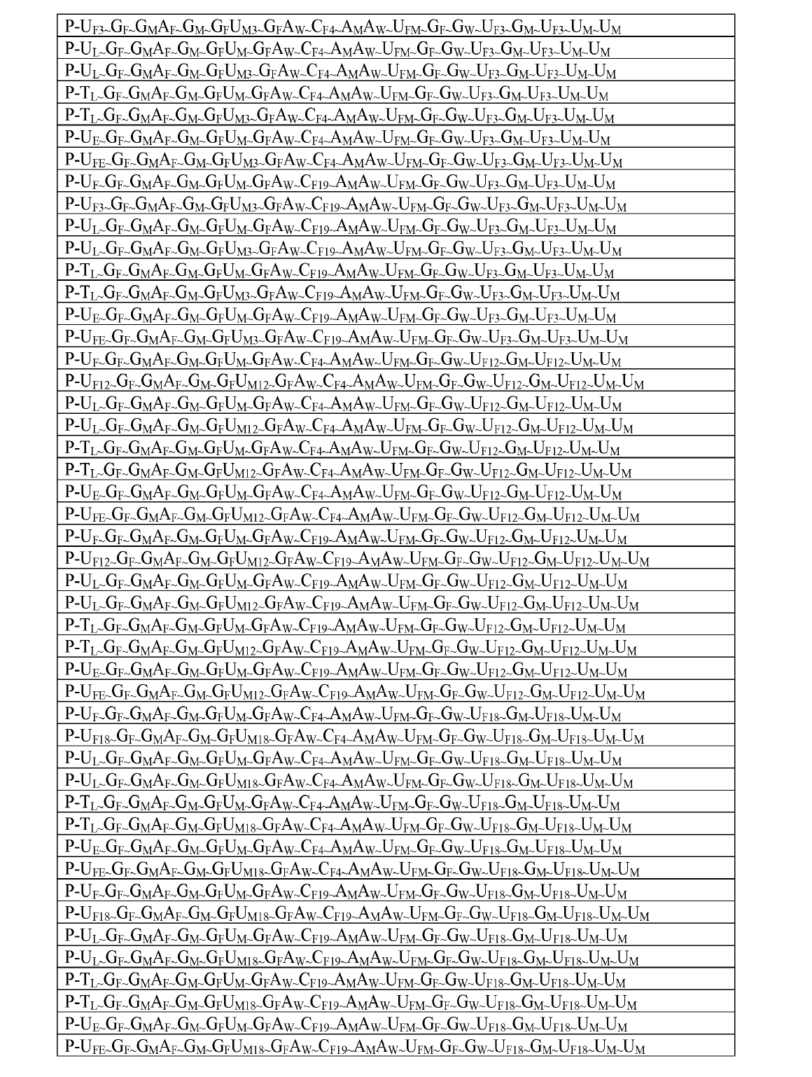

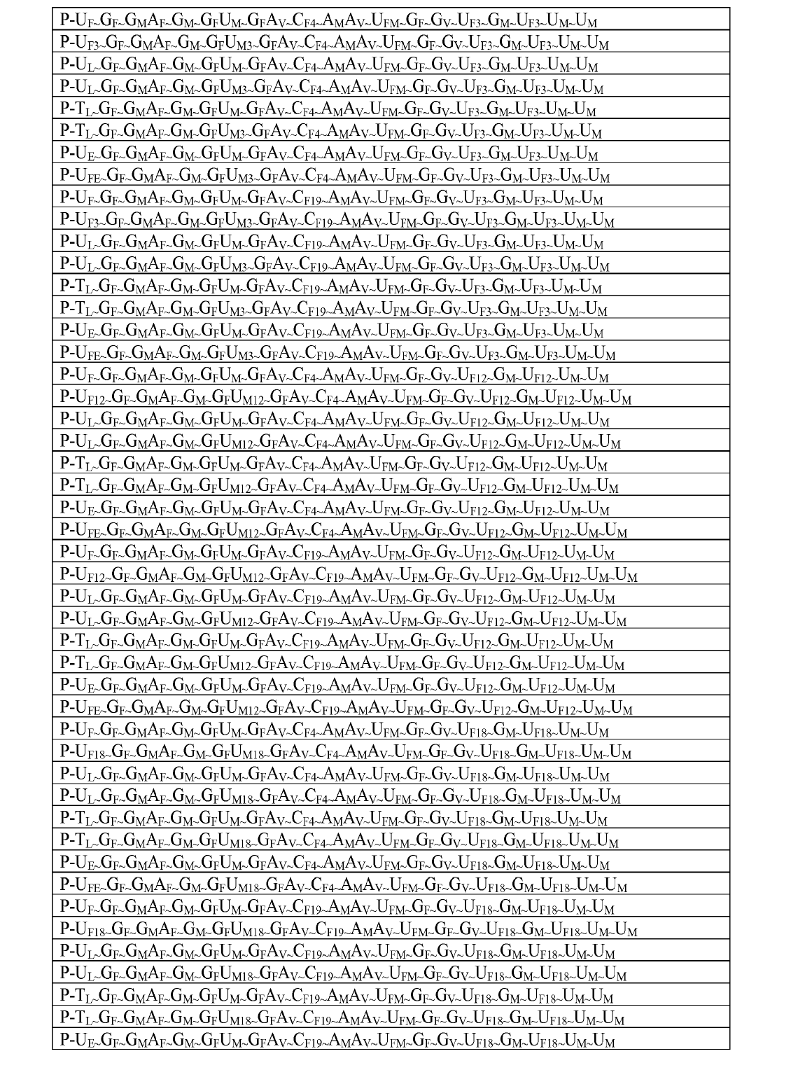

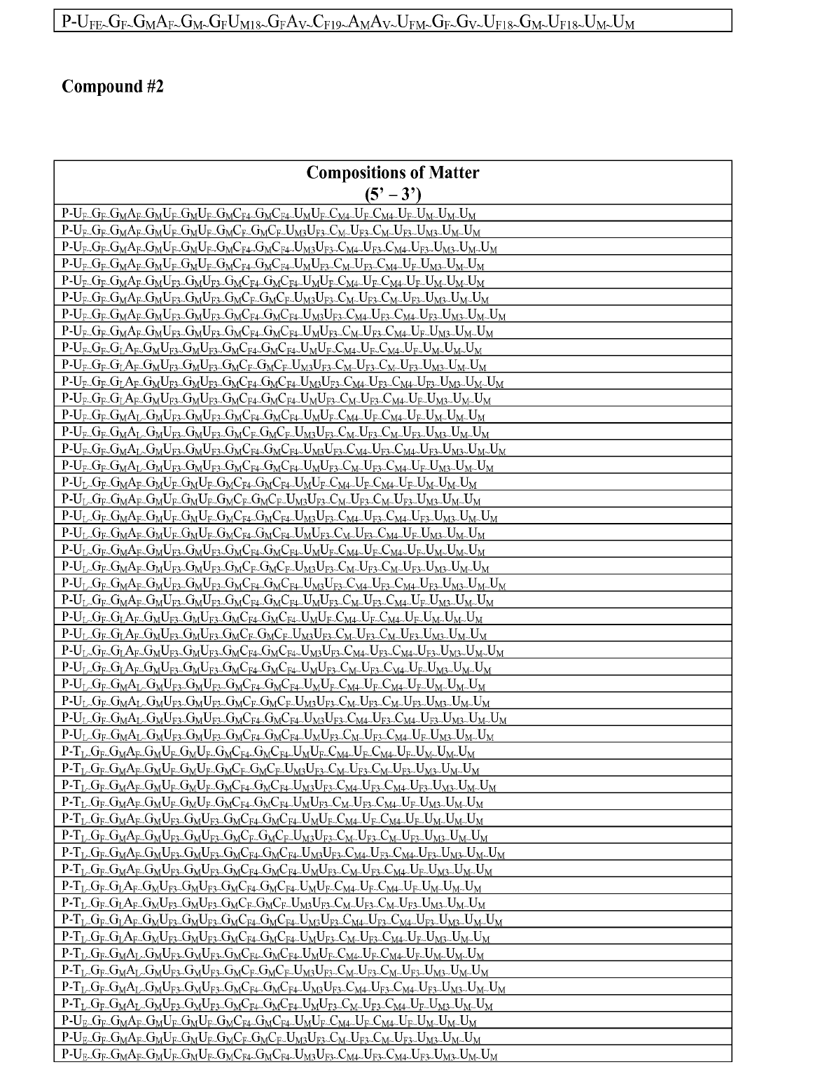

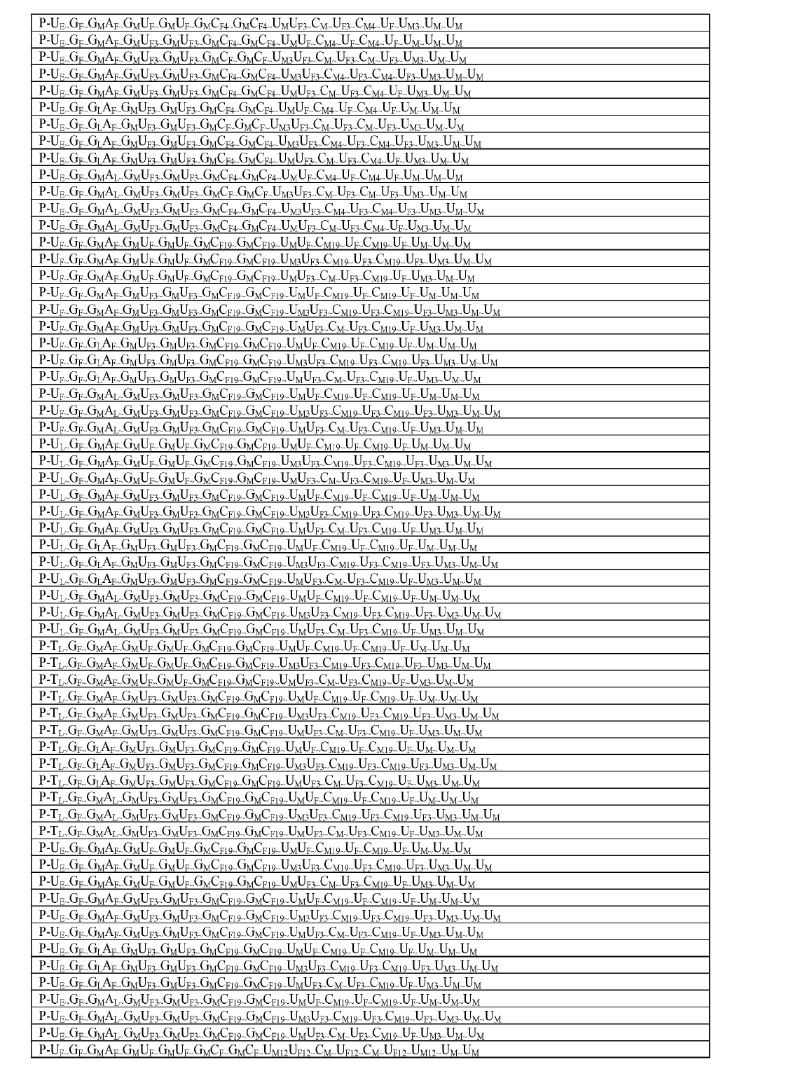

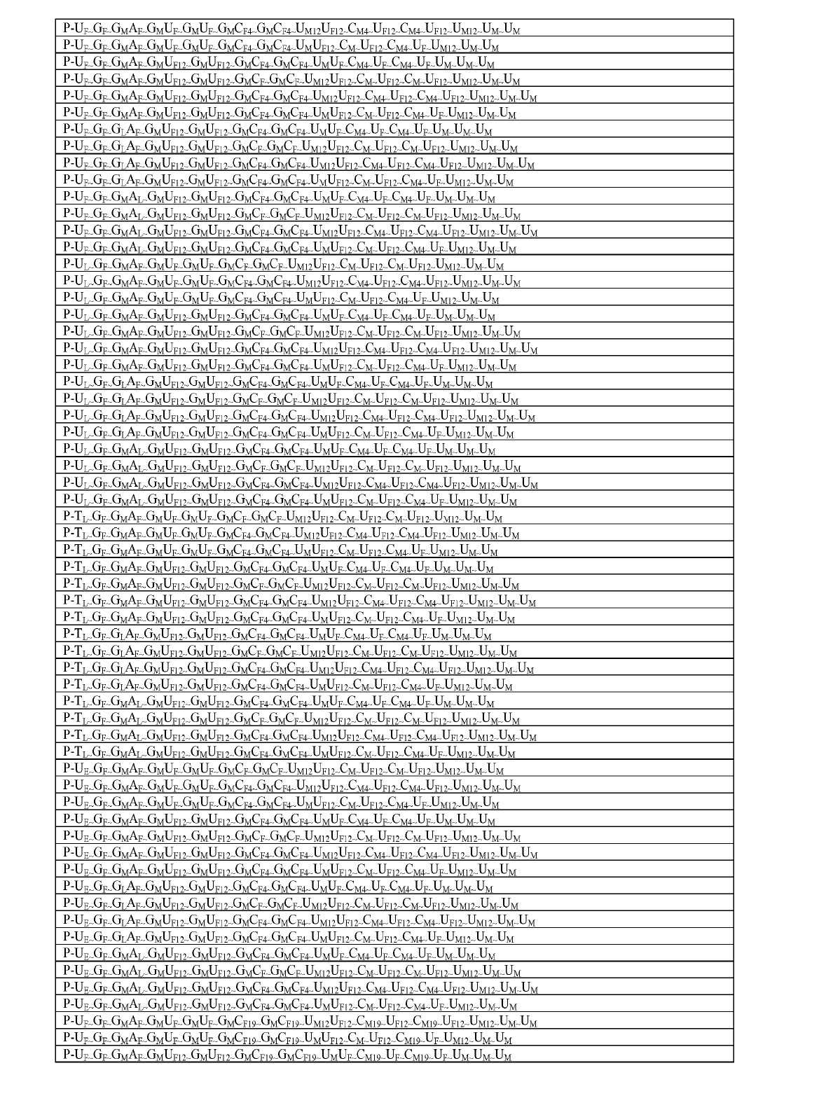

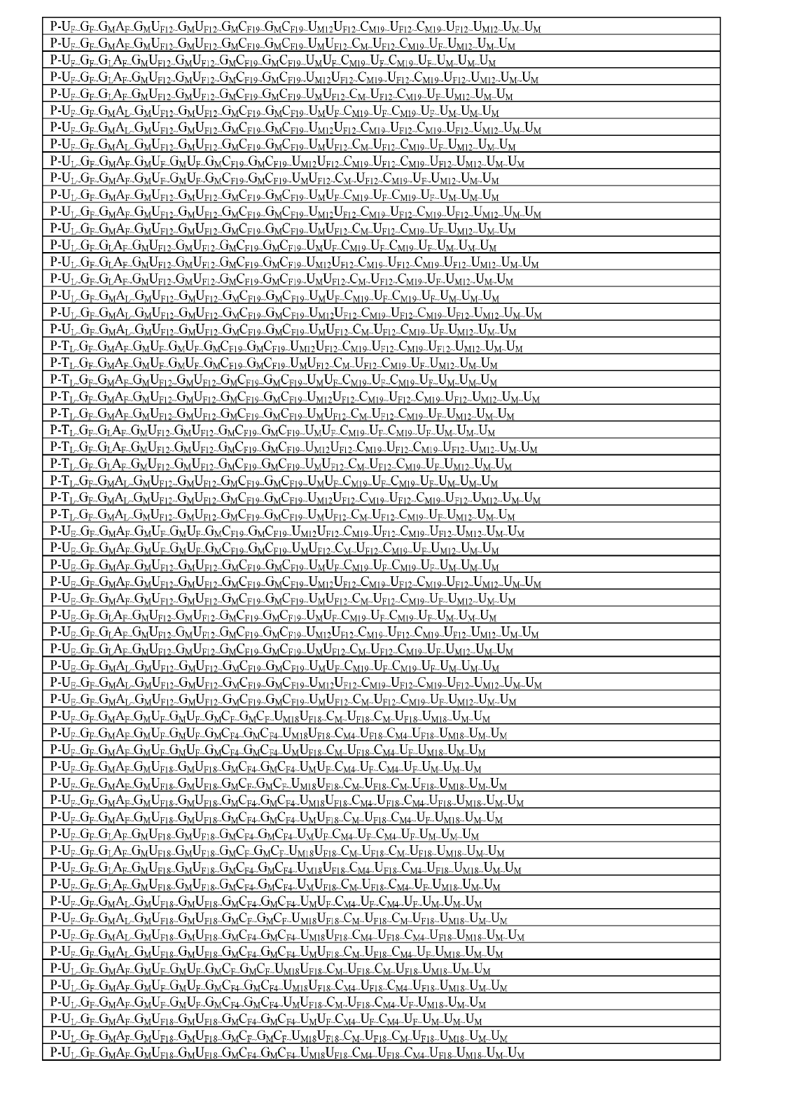

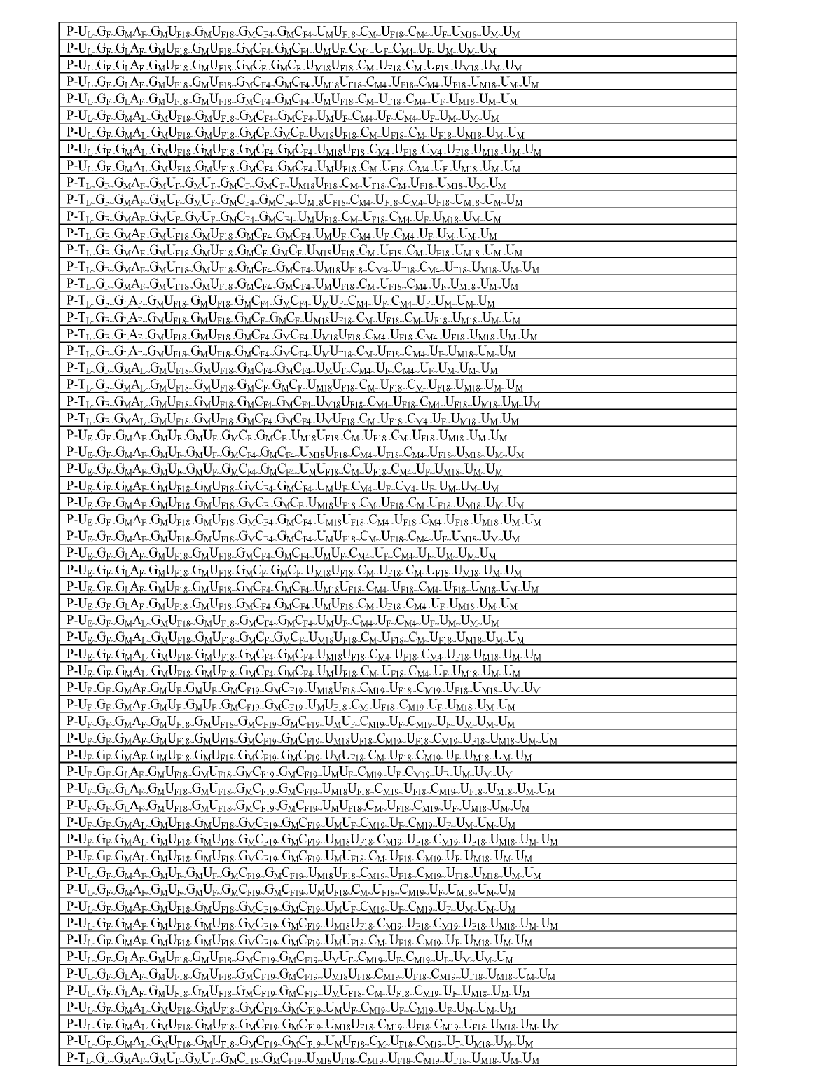

Ss-RNAi miRNA mimic compounds known in the art are shown in FIGS. 26A and 27A. They are based on miR-124 and miR-122 respectively (Chorn et al., RNA 18: 1-9, 2012). The first compound listed in section A of each figure is completely based on the naturally occurring miRNA guide strand sequence. The authors tested for activity a large series of compounds with either the miR-124 or miR-122 seed sequence where the sequence downstream of the seed sequence was replaced by sequences that had been randomly generated. Some of those with the highest level of activity against select miR-124 or miR-122 targets are included in FIGS. 26A and 27A and are modified in sections B and C of these figures using either the AHD modifications or AHD plus nuclease stabilizing modifications of the present invention.

FIG. 28 provides examples of 5 seed vehicles that can be used with the terminal 5′-end 9 nucleosides of the guide strand any miRNA found in MiRBase and with any of the overhang precursors provided herein mimic any known miRNA using the modular approach described herein. FIG. 17 provides examples of the use of seed vehicle #1 found in FIG. 28 to generate miR-34a mimics.

### Methods of Administration of the Single Strand Oligo Compounds of the Present Invention

A major advantage of the present invention in effecting RNAi is that many of the modifications described employ chemistries commonly used in conventional antisense oligos where the pharmacology and toxicology of the compounds is already largely understood and is described in the literature. References that summarize much of pharmacology for a range of different types of oligo therapeutics includes the following: Antisense Drug Technology: Principles, Strategies, and Applications, 2^{nd} ed., Stanley T. Crooke (ed.) CRC Press July 2007; Encyclopedia of Pharmaceutical Technology,—6 Volume Set, J Swarbrick (Editor) 3rd edition, 2006, Informa HealthCare; Pharmaceutical Perspectives of Nucleic Acid-Based Therapy, RI Mahato and SW Kim (Editora) 1 edition, 2002, CRC press; Pharmaceutical Aspects of Oligonucleotides, P Couvreur and C Malvy (Editors) 1st edition, 1999, CRC press; Therapeutic Oligonucleotides (RSC Biomolecular Sciences) (RSC Biomolecular Sciences) (Hardcover) by Jens Kurreck (Editor) Royal Society of Chemistry; 1 edition, 2008, CRC press; Clinical Trials of Genetic Therapy with Antisense DNA and DNA Vectors, E Wickstrom (Editor) 1st edition, 1998, CRC press.

The ss-RNAi compounds of the present invention will be given to subjects in the dose range established for conventional antisense oligos. For most systemic in vivo purposes administration of a strand to humans or non-human primates into the circulation at an infusion rate of up to 10 mg/kg/hr is appropriate. Single injections into tissues for systemic treatments generally are on the order of 20 mg/kg or less to reduce the likelihood of an injection site reaction.

The timing of strand administration i.v. or i.a. can also serve for a number of other administrative routes where the compounds are juxtaposed to the target tissue such as i.p., intrathecal, intraocular and intravesical.

In certain embodiments, (e.g., for the treatment of lung disorders, such as pulmonary fibrosis or asthma or to allow for self administration for local or systemic purposes) it may desirable to deliver the oligos described herein in aerosolized form. A pharmaceutical composition comprising at least one oligo can be administered as an aerosol formulation that contains the oligos in dissolved, suspended or emulsified form in a propellant or a mixture of solvent and propellant. The aerosolized formulation is then administered through the respiratory system or nasal passages.

An aerosol formulation used for nasal administration is generally an aqueous solution designed to be administered to the nasal passages as drops or sprays. Nasal solutions are generally prepared to be similar to nasal secretions and are generally isotonic and slightly buffered to maintain a pH of about 5.5 to about 6.5, although pH values outside of this range can also be used. Antimicrobial agents or preservatives can also be included in the formulation.

An aerosol formulation for use in inhalations and inhalants is designed so that the oligos are carried into the respiratory tree of the patient. See (WO 01/82868; WO 01/82873; WO 01/82980; WO 02/05730; WO 02/05785. Inhalation solutions can be administered, for example, by a nebulizer. Inhalations or insufflations, comprising finely powdered or liquid drugs, are delivered to the respiratory system as a pharmaceutical aerosol of a solution or suspension of the drug in a propellant.

An aerosol formulation generally contains a propellant to aid in disbursement of the oligos. Propellants can be liquefied gases, including halocarbons, for example, fluorocarbons such as fluorinated chlorinated hydrocarbons, hydrochlorofluorocarbons, and hydrochlorocarbons as well as hydrocarbons and hydrocarbon ethers (Remington's Pharmaceutical Sciences 18th ed., Gennaro, A. R., ed., Mack Publishing Company, Easton, Pa. (1990)).

Halocarbon propellants useful in the invention include fluorocarbon propellants in which all hydrogens are replaced with fluorine, hydrogen-containing fluorocarbon propellants, and hydrogen-containing chlorofluorocarbon propellants. Halocarbon propellants are described in Johnson, U.S. Pat. No. 5,376,359, and Purewal et al., U.S. Pat. No. 5,776,434.

Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as numerous other ethers.

The oligos can also be dispensed with a compressed gas. The compressed gas is generally an inert gas such as carbon dioxide, nitrous oxide or nitrogen.

An aerosol formulation of the invention can also contain more than one propellant. For example, the aerosol formulation can contain more than one propellant from the same class such as two or more fluorocarbons. An aerosol formulation can also contain more than one propellant from different classes. An aerosol formulation can contain any combination of two or more propellants from different classes, for example, a fluorohydrocarbon and a hydrocarbon.

Effective aerosol formulations can also include other components, for example, ethanol, isopropanol, propylene glycol, as well as surfactants or other components such as oils and detergents (Remington's Pharmaceutical Sciences, 1990; Purewal et al., U.S. Pat. No. 5,776,434). These aerosol components can serve to stabilize the formulation and lubricate valve components.

The aerosol formulation can be packaged under pressure and can be formulated as an aerosol using solutions, suspensions, emulsions, powders and semisolid preparations. A solution aerosol consists of a solution of an active ingredient such as oligos in pure propellant or as a mixture of propellant and solvent. The solvent is used to dissolve the active ingredient and/or retard the evaporation of the propellant. Solvents useful in the invention include, for example, water, ethanol and glycols. A solution aerosol contains the active ingredient peptide and a propellant and can include any combination of solvents and preservatives or antioxidants.

An aerosol formulation can also be a dispersion or suspension. A suspension aerosol formulation will generally contain a suspension of an effective amount of the oligos and a dispersing agent. Dispersing agents useful in the invention include, for example, sorbitan trioleate, oleyl alcohol, oleic acid, lecithin and corn oil. A suspension aerosol formulation can also include lubricants and other aerosol components.

An aerosol formulation can similarly be formulated as an emulsion. An emulsion can include, for example, an alcohol such as ethanol, a surfactant, water and propellant, as well as the active ingredient, the oligos. The surfactant can be nonionic, anionic or cationic. One example of an emulsion can include, for example, ethanol, surfactant, water and propellant. Another example of an emulsion can include, for example, vegetable oil, glyceryl monostearate and propane.

Oligos may be formulated for oral delivery (Tillman et al., J Pharm Sci 97: 225, 2008; Raoof et al., J Pharm Sci 93: 1431, 2004; Raoof et al., Eur J Pharm Sci 17: 131, 2002; U.S. Pat. No. 6,747,014; US 2003/0040497; US 2003/0083286; US 2003/0124196; US 2003/0176379; US 2004/0229831; US 2005/0196443; US 2007/0004668; US 2007/0249551; WO 02/092616; WO 03/017940; WO 03/018134; WO 99/60012). Such formulations may incorporate one or more permeability enhancers such as sodium caprate that may be incorporated into an enteric-coated dosage form with the oligo.

There are also delivery mechanisms applicable to oligos with or without carriers that can be applied to particular parts of the body such as the CNS. These include the use of convection-enhanced delivery methods such as but not limited to intracerebral clysis (convection-enhanced microinfusion into the brain—Jeffrey et al., Neurosurgery 46: 683, 2000) to help deliver the cell-permeable carrier/oligo complex to the target cells in the CNS as described in WO 2008/033285.

Drug delivery mechanisms based on the exploitation of so-called leverage-mediated uptake mechanisms are also suitable for the practice of this invention (Schmidt and Theopold, Bioessays 26: 1344, 2004). These mechanisms involve targeting by means of soluble adhesion molecules (SAMs) such as tetrameric lectins, cross-linked membrane-anchored molecules (MARMs) around lipoproteins or bulky hinge molecules leveraging MARMs to cause a local inversion of the cell membrane curvature and formation of an internal endosome, lysosome or phagosome. More specifically leverage-mediated uptake involves lateral clustering of MARMs by SAMs thus generating the configurational energy that can drive the reaction towards internalization of the oligo carrying complex by the cell. These compositions, methods, uses and means of production are provided in WO 2005/074966.

As for many drugs, dose schedules for treating patients with oligos can be readily extrapolated from animal studies. The extracellular concentration that must be generally achieved with highly active conventional antisense or complementary sense and antisense oligos for use in the two-step method is in the 1-200 nanomolar (nM) range. Higher extracellular levels, up to 1.5 micromolar, may be more appropriate for some applications as this can result in an increase in the speed and the amount of the oligos driven into the tissues. Such levels can readily be achieved in the plasma.

For in vivo applications, the concentration of the oligos to be used is readily calculated based on the volume of physiologic balanced-salt solution or other medium in which the tissue to be treated is being bathed. With fresh tissue, 1-1000 nM represents the concentration extremes needed for oligos with moderate to excellent activity. Two hundred nanomolar (200 nM) is a generally serviceable level for most applications. With most cell lines a carrier will typically be needed for in vitro administration. Incubation of the tissue with the oligos at 5% rather than atmospheric (ambient) oxygen levels may improve the results significantly.

Pharmacologic/toxicologic studies of phosphorothioate oligos, for example, have shown that they are adequately stable under in vivo conditions, and that they are readily taken up by all the tissues in the body following systemic administration with a few exceptions such as the central nervous system (Iversen, Anticancer Drug Design 6:531, 1991; Iversen, Antisense Res. Develop. 4:43, 1994; Crooke, Ann. Rev. Pharm. Toxicol. 32: 329, 1992; Cornish et al., Pharmacol. Comm. 3: 239, 1993; Agrawal et al., Proc. Natl. Acad. Sci. USA 88: 7595, 1991; Cossum et al., J. Pharm. Exp. Therapeutics 269: 89, 1994). These compounds readily gain access to the tissue in the central nervous system in large amounts following injection into the cerebral spinal fluid (Osen-Sand et al., Nature 364: 445, 1993; Suzuki et al., Amer J. Physiol. 266: R1418, 1994; Draguno et al., Neuroreport 5: 305, 1993; Sommer et al., Neuroreport 5: 277, 1993; Heilig et al., Eur. J. Pharm. 236: 339, 1993; Chiasson et al., Eur J. Pharm. 227: 451, 1992). Phosphorothioates per se have been found to be relatively non-toxic, and the class specific adverse effects that are seen occur at higher doses and at faster infusion rates than is needed to obtain a therapeutic effect with a well-chosen sequence. In addition to providing for nuclease resistance, one potential advantage of phosphorothioate and boranophosphate linkages over the phosphodiester linkage is the promotion of binding to plasma proteins and albumin in particular with the resulting effect of an increased plasma half-life. By retaining the oligo for a longer period of time in plasma the oligo has more time to enter tissues as opposed to being excreted by the kidney. Oligos with primarily or exclusively phosphodiester linkages have a plasma half-life of only a few minutes. Thus, they are of little use for in vivo applications when used without a carrier. In the case of oligos with a preponderance of or exclusively phosphodiester linkages, plasma protein binding can be improved by covalently attaching the oligo a molecule that binds a plasma protein such as serum albumin. Such molecules include, but are not limited to, an arylpropionic acid, for example, ibuprofen, suprofen, ketoprofen, pranoprofen, tiaprofenic acid, naproxen, flurpibrofen and carprofen (U.S. Pat. No. 6,656,730). As for other moieties that might be linked to the oligos suitable for use with the present invention the preferred site is the 3′-end of the oligo. Intravenous administrations of oligos can be continuous for days or be administered over a period of minutes depending on the particular oligos and the medical indication.

The most prominent toxicities associated with intravenous administration of phosphorothioates have been related to the chemical class and generally independent of the mRNA target sequence and, therefore, independent of hybridization. The observed and measured toxicities have been consistent from drug to drug pre-clinically and clinically, with non-human primates being most similar to humans for certain key toxicities.

The class-related toxicities that have been most compelling in choosing dose and schedule for pre-clinical and clinical evaluation occur as a function of binding to specific plasma proteins and include transient inhibition of the clotting cascade and activation of the complement cascade. Both of these toxicities may be related to the polyanionic nature of the molecules.

The effect of phosphorothioates on the clotting cascade results in plasma concentration-related prolongation of the activated partial thromboplastin (aPPT) time. Maximum prolongation of the aPTT correlates closely with the maximum plasma concentration so doses and schedules that avoid high peak concentrations can be selected to avoid significant effects on the aPTT. Because the plasma half-life of these drugs is short (30 to 60 minutes), the effect on clotting is transient. Several of these drugs have been evaluated in the clinic with prolonged intravenous infusions lasting up to 3 weeks. Shorter IV infusions (e.g., 2 hours) have also been studied. For example, aprinocarsen (ISIS 3521) and ISIS 5132 were studied with both 2 hour and 3-week continuous infusion schedules. At a dose of 3 mg/kg/dose over 2 hours, transient prolongation of the aPTT was observed. When 3 mg/kg was given daily by continuous infusion for 21 days, there was no effect on aPTT. The effect of antisense molecules of this chemical class on the clotting cascade is consistent.

Similarly, the activation of complement is a consistent observation; however, the relationship between plasma concentration of oligonucleotides and complement activation is more complex than the effect on clotting. Also, while the effect on clotting is found in rats as well as monkeys, the effect on the complement cascade has only been observed in monkeys and humans.

When these drugs are given to cynomolgus monkeys by 2-hour infusion, increases in complement split products (i.e., C3a, C5a, and Bb) occur only when plasma concentrations exceed a threshold value of 40-50 μg/mL. In monkeys, there is a low incidence of cardiovascular collapse associated with increases in these proteins. For the most part, clinical investigations of phosphorothioates have been designed to avoid these high plasma concentrations.

When ISIS 3521 was given as a weekly 24 hour infusion at doses as high as 24 mg/kg (1 mg/kg/hour×24 hours), the steady state plasma concentrations reached approximately 12 μg/mL at the high dose. On this schedule, however, there were substantial increases in C3a and Bb even though these plasma levels were much lower than those seen with the shorter infusions. Thus, activation of complement may be associated with both dose and schedule where plasma concentrations that are well tolerated over shorter periods of time (e.g. 2 hours), are associated with toxicity when the plasma concentrations are maintained for longer. This likely provides the explanation for the findings with cenersen in rhesus monkeys where complement activation was observed at concentrations of 14-19 μg/mL.

When ISIS 3521 was given at 1.0 and 1.25 mg/kg/hour×2 hours, the mean peak plasma concentrations were 11.1±0.98 and 6.82±1.33 μg/mL, respectively. There was no complement activation at these or other higher doses and no other safety issues. It is expected that the maximum peak plasma concentrations for similarly sized phosphorothioate given at 1.2 mg/kg/hour×1 hour would be similar to that observed with ISIS 3521.

### EXAMPLES OF COMMERCIAL USES BASED ON PARTICULAR TARGET(S)

The following examples are provided to illustrate certain embodiments of the present invention. They are not intended to limit the invention in any way.

### Example I

### Applications for si-RNA

The genes targeted for silencing are shown in Table 6 and in the examples. They are not meant to provide an exhaustive set of illustrations of how the designs presented herein can be applied in general or in particular. One skilled in the art can readily use the design principles and the examples provided herein to arrive at a very limited set of compounds that can be generated in accordance with the present invention using any given gene target in a subject.

TABLE 6
EXAMPLES OF COMMERCIAL APPLICATIONS FOR ss-RNAi
INHIBITORS FOR ILLUSTRATIVE GENE TARGETS

| GENE TARGET | MEDICAL CONDITIONS TO BE TREATED OR OTHER COMMERCIAL OBJECTIVES |
|---|---|
| Apoliprotein B (Apo B) | Atherosclerosis; Congestive heart failure; Familial hypercholesterolemia; Statin |
| | resistant hypercholesterolemia; HDL/LDL cholesterol imbalance; dyslipidemias; |
| | Acquired hyperlipidemia; Coronary artery disease; Thrombosis |
| FAS/APO-1 | Myocardial infarction; Fatty liver disease; Fulminant hepatitis; Cirrhosis of the liver; |
| (CD-95; Tnfrsf6) | Alcoholic hepatitis; Cholestatic liver injury; Acute liver failure; Cystic fibrosis; |
| | Systemic lupus erythematosus; Arthritis; Parkinson's Disease; Autoimmune diabetes; |
| | Central nervous system injuries, Demyelinating diseases; Stroke; Chemotherapy- |
| | induced neuropathy; Neurodegenerative diseases; Spinal cord injury; Ischemia - |
| | reperfusion injury |
| p53 | Sensitize cancers with wild type p53 to cytotoxic therapies; Cancers with mutant p53; |
| | Sensitize cancers with mutant p53 to the induction of apoptosis by any apoptosis |
| | inducer; Stem cell quiescence including malignant stem cells (expand normal stem |
| | cells and progeny or put malignant stem cells in cycle so they can be attacked by cell |
| | cycle dependent anti-cancer agents; Heart failure; Medical conditions where apoptosis |
| | is promoted; Inhibiting apoptosis in non-malignant stem cells; Huntington's disease; |
| | Diamond-Blackfan syndrome; Shwachman Diamond Syndrome and other disorders |
| | involving defective ribosomes and/or imbalances in ribosomal components |
| | (ribosomopathies); Fatty liver disease; Stress induced immunosuppression; Sequellae |
| | associated with subarachnoid hemorrhage; Pathologic hyperpigmentation; |
| | Hyperkeratosis; Toxic effects of cancer chemotherapy and radiation including but not |
| | limited to the following: hair loss, mucositis, myelosupression, hearing loss, |
| | peripheral nerve damage, impaired brain function and kidney damage; Inflammatory |
| | bowel disease; Crohn's disease; ARDS; Multiple organ failure; Sensitize cancers to |
| | cytotoxic treatments dependent on cell proliferation and/or DNA replication; Amyloid |
| | deposition; Neurodegenerative diseases; Ischemia-reperfusion injury; Avoidance of |
| | effects of cytotoxic therapy due to quiescence of malignant stem cells; Reduced |
| | expansion of non-malignant tissue due to stem cell quiescence; Prevent |
| | demyelination; Multiple sclerosis; Alzheimer's Disease; Parkinson's disease; Prevent |
| | cell death associated with diabetic ischemia; Spontaneous apoptosis, cell cycle arrest, |
| | senescence and differentiation in stem cells including embryonic stem cells and iPS |
| | such as reduces the efficiency of preparing such cells for transplantation organ |
| | generation, the generation of animals or for use in scientific research; Prevent cell |
| | death associated with cerebral ischemia; Prevent cell death associated with |
| | myocardial infarction including consequent heart wall rupture; Schizophrenia; |
| | Psoriasis; AIDS; Prevent rupture of atherosclerotic plaques; Prevent aneurysm |
| | rupture; Graft vs host disease; Systemic lupus erythematosus; Promote healing of |
| | hard to heal wounds; Capillary leak syndrome; Emphysema; Reduce enodosomal, |
| | lysosomal or phagosomal sequestration of oligo therapeutics with the effect of |
| | increasing their biologic activity; Promote proliferation of stem cells such as |
| | hematopoietic or neural; Diabetes mellitus including insulin resistant diabetes; 5q- |
| | syndrome; Porokeratosis; Ferritin induced cell death such as occurs in iron overload; |
| | Anemia; Dyskeratosis congentia including that form with telomerase insufficiency; |
| | Prevent emphysema; Prevent COPD; Insulin resistance in heart failure |
| PCSK9 | Atherosclerosis; Hypercholesterolemia; Statin resistant hypercholesterolemia; |
| (NARC-1) | HDL/LDL cholesterol imbalance; dyslipidemias; Acquired hyperlipidemia; Coronary |
| | artery disease |
| PTEN | Cancers with mutated p53; Activate cell proliferation including hematopoietic stem |
| (MMAC1; TEP1) | and progenitor cells; Increase efficiency of gene transfer including into hematopoietic |
| | stem and progenitor cells; Nerve cell regeneration |
| PTP-1B | Insulin resistance; Type II Diabetes |
| Stat3 | Cancer, Autoimmune disease |

### A. Compounds for Down-Regulating p53 Expression

p53 is involved in the regulation of a variety of cellular programs including those involving stem cell self-renewal, cellular proliferation and viability such as proliferation, differentiation, apoptosis, senescence, mitotic catastrophe and autophagy.

The pathological expression or failure of expression of such programs, and the death programs in particular, underlie many of the morbidities associated with a wide variety of medical conditions where blocking p53 function can prevent much if not all of such morbidity.

In cancer, for example, both wild type and mutant p53 play key roles in tumor maintenance that include increasing the threshold for the induction of programs that can lead to the death of the cancer cells. Typically the use of a p53 inhibitor, such as a siRNA directed to the p53 gene target, in combination with an inducer of a cell death program, such as a DNA damaging agent, can be used to promote the death of cancer cells. At the same time inhibition of p53 protects many normal tissues from the toxic effects of many such second agents including chemotherapy and radiation.

Further, the present inventor has found that Boron Neutron Capture Therapy (BNCT) can be used in combination with ss-siRNA, double stranded siRNA or conventional antisense oligos that inhibit p53 (such as but not limited to those described in PCT/US09/02365) as a method for treating cancer (Brownell et al., “Boron Neutron Capture Therapy” In; “Therapy of Nuclear Medicine,” RP Spencer (ed), Grune and Stratton, NY, 1978; Barth et al. Cancer Res 50: 1061, 1990; Summers and Shaw, Curr Med Chem 8: 1147, 2001). Specifically, the ¹⁰B atom undergoes fission to generate ⁷Li and energetic alpha (helium) particles following capturing a thermal neutron. Within their 10-14 mm path, such particles cause DNA and other types of damage that enhance apoptosis and other inactivating effects on cancer cells when wild type or mutated p53 is inhibited.

The use of conventional antisense oligos which function using an RNAse H mechanism of action and directed to the p53 gene target have been studied in vitro and in patients. These oligos have been shown to promote the anti-cancer effects of certain conventional treatments and to protect normal tissues from genome damaging agents. Few cell types, with the exception of stem cells, possess sufficient levels of RNase H to support conventional antisense oligos dependent on this enzyme for their activity. Consequently, RNAi directed to the p53 gene target which are not dependent on RNAse H activity for function offer the potential advantage of being active in vivo in a broader range of cell types while still being catalytic. As for RNAi, generally this potential is severely limited by the well known problems associated with the poor uptake of conventional siRNA uptake in vivo and the lack of carriers that can broadly address this problem.

Molitoris et al. (J Am Soc Nephrol 20: 1754, 2009) presents data showing that conventional siRNA directed to the p53 gene target can attenuate cisplatin induced kidney damage in rats. The siRNA described was a blunt ended 19-mer with alternating 2′-0-methy/native ribose nucleosides. A carrier was not needed because the proximal tubule cells in the kidney are both a major site of kidney injury associated with ischemia or nephrotoxicity such as that caused by cisplatin and is the site of oligo reabsorption by the kidney. Thus, this carrier free approach with conventional siRNA is of very limited use for preventing the pathologic effects of p53-dependent programs that kill cells or otherwise incapacitate them, but it does illustrate the potential usefulness of inhibiting p53 for this medical indication.

Zhao et al. (Cell Stem Cell 3: 475, 2008) demonstrated that inhibiting p53 expression with siRNA can be used to enhance the production of iPSC. Human fibroblasts, for example, were converted to iPSC by using expression vectors for several genes to gain their expression in the cells. The efficiency of iPSC production was very low but was increased approximately two logs when shRNA directed to the p53 gene target was installed in the cells using a lentiviral vector. The approach described herein provides the means to transiently suppress p53 compared to the long term suppression provided by shRNA. This is important when the iPSC are to be induced to differentiate into particular cell type such as would be needed in tissue repair applications. As described herein the two-step administration approach combined with the linkage of a short cell penetrating peptide (CPP) to each strand provides an efficient way to obtain RNAi activity in stem cells in vitro with minimal carrier related toxicity.

RNAi compounds directed to the human p53 gene target that can be reconfigured for use in the two-step method provided by the present invention are found in WO 2006/035434, US 2009/0105173 and US 2004/0014956.

Table 6 lists a variety of disorders that would benefit with treatment of the p53 directed compounds described herein. For example, heart failure is a serious condition that results from various cardiovascular diseases. p53 plays a significant role in the development of heart failure. Cardiac angiogenesis directly related to the maintenance of cardiac function as well as the development of cardiac hypertrophy induced by pressure-overload. Upregulated p53 induced the transition from cardiac hypertrophy to heart failure through the suppression of hypoxia inducible factor-1 (HIF-1), which regulates angiogenesis in the hypertrophied heart. In addition, p53 is known to promote apoptosis, and apoptosis is thought to be involved in heart failure. Thus, p53 is a key molecule that triggers the development of heart failure via multiple mechanisms.

Accordingly, the p53 directed compounds of the invention can be employed to diminish or alleviate the pathological symptoms associated with cardiac cell death due to apoptosis of heart cells. Initially the compound(s) will be incubated with a cardiac cell and the ability of the oligo to modulate p53 gene function (e.g., reduction in production p53, apoptosis, improved cardiac cell signaling, Ca++ transport, or morphology etc.) can be assessed. For example, the H9C2 cardiac muscle cell line can be obtained from American Type Culture Collection (Manassas, Va., USA) at passage 14 and cultured in DMEM complete culture medium (DMEM/F12 supplemented with 10% fetal calf serum (FCS), 2 mM α-glutamine, 0.5 mg/1 Fungizone and 50 mg/1 gentamicin). This cell line is suitable for characterizing the inhibitory functions of the p53 directed compounds of the invention and for characterization of modified versions thereof. HL-1 cells, described by Clayton et al. (1998) PNAS 95:2979-2984, can be repeatedly passaged and yet maintain a cardiac-specific phenotype. These cells can also be used to further characterize the effects of the oligos described herein.

It appears that expression of the apoptosis regulator p53 is governed, in part, by a molecule that in mice is termed murine double minute 2 (MDM2), or in man, human double minute 2 (HDM2), an E3 enzyme that targets p53 for ubiquitination and proteasomal processing, and by the deubiquitinating enzyme, herpesvirus-associated ubiquitin-specific protease (HAUSP), which rescues p53 by removing ubiquitin chains from it. Birks et al. (Cardiovasc Res. 2008 Aug. 1; 79 (3):472-80) examined whether elevated expression of p53 was associated with dysregulation of ubiquitin-proteasome system (UPS) components and activation of downstream effectors of apoptosis in human dilated cardiomyopathy (DCM). In these studies, left ventricular myocardial samples were obtained from patients with DCM (n=12) or from non-failing (donor) hearts (n=17). Western blotting and immunohistochemistry revealed that DCM tissues contained elevated levels of p53 and its regulators HDM2, MDM2 or the homologs thereof found in other species, and HAUSP (all P<0.01) compared with non-failing hearts. DCM tissues also contained elevated levels of polyubiquitinated proteins and possessed enhanced 20S-proteasome chymotrypsin-like activities (P<0.04) as measured in vitro using a fluorogenic substrate. DCM tissues contained activated caspases 9 and 3 (P<0.001) and reduced expression of the caspase substrate PARP-1 (P<0.05). Western blotting and immunohistochemistry revealed that DCM tissues contained elevated expression levels of caspase-3-activated DNase (CAD; P<0.001), which is a key effector of DNA fragmentation in apoptosis and also contained elevated expression of a potent inhibitor of CAD (ICAD-S; P<0.01). These investigators concluded that expression of p53 in human DCM is associated with dysregulation of UPS components, which are known to regulate p53 stability. Elevated p53 expression and caspase activation in DCM was not associated with activation of both CAD and its inhibitor, ICAD-S. These findings are consistent with the concept that apoptosis may be interrupted and therefore potentially reversible in human HF.

In view of the foregoing, it is clear that the p53 directed compounds provided herein should exhibit efficacy for the treatment of heart failure. Accordingly, in one embodiment of the invention, p53 directed compounds are administered to patients to inhibit cardiac cell apoptosis, thereby reducing the incidence of heart failure.

Cellular transformation during the development of cancer involves multiple alterations in the normal pattern of cell growth regulation and dysregulated transcriptional control. Primary events in the process of carcinogenesis can involve the activation of oncogene function by some means (e.g., amplification, mutation, chromosomal rearrangement) or altered or aberrant expression of transcriptional regulator functions, and in many cases the removal of anti-oncogene function. One reason for the enhanced growth and invasive properties of some tumors may be the acquisition of increasing numbers of mutations in oncogenes and anti-oncogenes, with cumulative effect (Bear et al., Proc. Natl. Acad. Sci. USA 86:7495-7499, 1989). Alternatively, insofar as oncogenes function through the normal cellular signaling pathways required for organismal growth and cellular function (reviewed in McCormick, Nature 363:15-16, 1993), additional events corresponding to mutations or deregulation in the oncogenic signaling pathways may also contribute to tumor malignancy (Gilks et al., Mol. Cell Biol. 13:1759-1768, 1993), even though mutations in the signaling pathways alone may not cause cancer.

p53 provides a powerful target for efficacious anti-cancer agents. Combination of the p53 directed compounds with one or more therapeutic agents that promote apoptosis effectively induces cell death in cancer cells. Such agents include but are not limited to conventional chemotherapy, radiation and biological agents such as monoclonal antibodies and agents that manipulate hormone pathways.

p53 protein is an important transcription factor which plays a central role in cell cycle regulation mechanisms and cell proliferation control. Baran et al. performed studies to identify the expression and localization of p53 protein in lesional and non-lesional skin samples taken from psoriatic patients in comparison with healthy controls (Acta Dermatovenerol Alp Panonica Adriat. (2005) 14:79-83). Sections of psoriatic lesional and non-lesional skin (n=18) were examined. A control group (n=10) of healthy volunteers with no personal and family history of psoriasis was also examined. The expression of p53 was demonstrated using the avidin-biotin complex immunoperoxidase method and the monoclonal antibody DO7. The count and localization of cells with stained nuclei was evaluated using a light microscope in 10 fields for every skin biopsy. In lesional psoriatic skin, the count of p53 positive cells was significantly higher than in the skin samples taken from healthy individuals (p<0.01) and non-lesional skin taken from psoriatic patients (p=0.02). No significant difference between non-lesional psoriatic skin and normal skin was observed (p=0.1). A strong positive correlation between mean count and mean per cent of p53 positive cells was found (p<0.0001). p53 positive cells were located most commonly in the basal layer of the epidermis of both healthy skin and non-lesional psoriatic skin. In lesional psoriatic skin p53 positive cells were present in all layers of the epidermis. In view of these data, it is clear that p53 protein appears to be an important factor in the pathogenesis of psoriasis. Accordingly, compounds which effectively down regulate p53 expression in the skin used alone or in combination with other agents used to treat psoriasis should alleviate the symptoms of this painful and unsightly disorder.

### B. Compounds for Down-Regulating Fas (Apo-1 or CD95) Expression

Fas (APO-1 or CD95) is a cell surface receptor that controls a pathway leading to cell death via apoptosis. This pathway is involved in a number of medical conditions where blocking fas function can provide a clinical benefit. See Table 6. Fas-mediated apoptosis, for example, is a key contributor to the pathology seen in a broad spectrum of liver diseases where inhibiting hepatocyte death can be life saving.

Lieberman and her associates have studied the effects of siRNA directed to the murine fas receptor gene target in murine models of fulminant hepatitis and renal ischemia-reperfusion injury (Song et al., Nature Med 9: 347, 2003; Hamar et al., Proc Natl Acad Sci USA 101: 14883, 2004). siRNA delivered by a hydrodynamic transfection method showed that such siRNA protects mice from concanavalin A generated hepatocyte apoptosis as evidenced by a reduction in liver fibrosis or from death associated with injections of a more hepatotoxic fas specific antibody. In the second study, siRNA was shown to protect mice from acute renal failure after clamping of the renal artery.

RNAi compounds directed to the human fas (apo-1 or CD95) receptor or ligand gene target are provided in WO 2009/0354343, US 2005/0119212,WO 2005/042719 and US 2008/0227733.

Recently, Feng et al. reported that during myocardial ischemia, cardiomyocytes can undergo apoptosis or compensatory hypertrophy (Coron Artery Dis. 2008 November; 19(7):527-34). Fas expression is upregulated in the myocardial ischemia and is coupled to both apoptosis and hypertrophy of cardiomyocytes. Some reports suggested that Fas might induce myocardial hypertrophy. Apoptosis of ischemic cardiomyocytes and Fas expression in the nonischemic cardiomyocytes occurs during the early stage of ischemic heart failure. Hypertrophic cardiomyocytes easily undergo apoptosis in response to ischemia, after which apoptotic cardiomyocytes are replaced by fibrous tissue. In the late stage of ischemic heart failure, hypertrophy, apoptosis, and fibrosis are thought to accelerate each other and might thus form a vicious circle that eventually results in heart failure. Based on these observations, it is clear that Fas directed compounds provide useful therapeutic agents for ameliorating the pathological effects associated with myocardial ischemia and hypertrophy. Accordingly, fas directed oligos will be administered cardiac cells and their effects on apoptosis assessed. As discussed above, certain modifications of the fas directed compounds will also be assessed. These include conjugation to heart homing peptides, inclusion of CPPs, as well as encapsulation in liposomes or nanoparticles as appropriate.

In their article entitled, “Fas Pulls the Trigger on Psoriasis”, Gilhar et al. describe an animal model of psoriasis and the role played by Fas mediated signal transduction (2006) Am. J. Pathology 168:170-175). Fas/FasL signaling is best known for induction of apoptosis. However, there is an alternate pathway of Fas signaling that induces inflammatory cytokines, particularly tumor necrosis factor alpha (TNF-α) and interleukin-8 (IL-8). This pathway is prominent in cells that express high levels of anti-apoptotic molecules such as Bcl-xL. Because TNF-α is central to the pathogenesis of psoriasis and psoriatic epidermis has a low apoptotic index with high expression of Bcl-xL, these authors hypothesized that inflammatory Fas signaling mediates induction of psoriasis by activated lymphocytes. Noninvolved skin from psoriasis patients was grafted to beige-severe combined immunodeficiency mice, and psoriasis was induced by injection of FasL-positive autologous natural killer cells that were activated by IL-2. Induction of psoriasis was inhibited by injection of a blocking anti-Fas (ZB4) or anti-FasL (4A5) antibody on days 3 and 10 after natural killer cell injection. Anti-Fas monoclonal antibody significantly reduced cell proliferation (Ki-67) and epidermal thickness, with inhibition of epidermal expression of TNF-α, IL-15, HLA-DR, and ICAM-1. Fas/FasL signaling is an essential early event in the induction of psoriasis by activated lymphocytes and is necessary for induction of key inflammatory cytokines including TNF-α and IL-15.

Such data provide the rationale for therapeutic regimens entailing topical administration of Fas directed compounds and/or BCL-xL directed compounds for the treatment and alleviation of symptoms associated with psoriasis.

### C. Compounds for Down-Regulating Apo-B Expression

Apolipoprotein B (apoB) is an essential protein for the formation of low-density lipoproteins (LDL) and is the ligand for LDL receptor. LDL is responsible for carrying cholesterol to tissues. High levels of apoB can lead to plaques that cause atherosclerosis. Accordingly, blocking apo B expression is a useful treatment modality for a variety of medical disorders including those listed in Table 6

Soutschek et al. (Nature 432: 173, 2004) have described two siRNA compounds simultaneously directed to both the murine and human apoB gene targets suitable for use in the present invention. These compounds have 21-mer passenger and 23-mer guide strands with cholesterol conjugated to the 3′-ends of the passenger strand. The cholesterol promoted both nuclease resistance and cellular uptake into the target tissues. The reductions in apoB expression in liver and jejunum were associated with reductions in plasma levels of apoB-100 protein and LDL. The authors indicated that the unconjugated compounds (lacking cholesterol) were inactive and concluded that the conjugated compounds need further optimization to achieve improved in vivo potency at doses and dose regimens that are clinically acceptable.

The same group of investigators filed US20060105976, WO06036916 and U.S. Pat. No. 7,528,118 that also provide siRNA compounds suitable for down modulating both human and mouse apoB gene expression. Eighty-one distinct RNAi compounds with demonstrated activity in the human HepG2 and/or the murine liver cell line NmuLi that expresses apoB were described. Twenty-seven of these double stranded siRNA compounds were found to reduce apoB protein expression in HepG2 cells to less than 35% of control. One of these siRNA was tested in human apoB-100 transgenic mice where following three daily tail vein injections, the siRNA reduced mouse apoB mRNA levels 43+/−10% in liver and 58+/−12% in jejunum and also reduced human apoB mRNA in livers to 40+/−10%. Other siRNA compounds directed to apoB suitable for use in the present invention have been disclosed in US 2006/0134189. These have been described for use in combination with the SNALP (stable nucleic acid lipid particles) delivery technology.

Conventional antisense oligos directed to gene targets such as the apoB can be converted to RNAi compounds in accordance with the present invention and can be used as described herein. A series of conventional antisense oligos directed to apoB and suitable for use with the present invention have been described in Merki et al., Circulation 118: 743, 2008; Crooke et al., J Lipid Res 46: 872, 2005; Kastelein et al., Circulation 114: 1729, 2006; U.S. Pat. No. 7,407,943, US 2006/0035858 and WO 2007/143315.

The conventional antisense oligos described in filing WO 2007/143315 are 8-16-mers. It is known that guide strands shorter than 15-mers are not active. Further 16-mer guide strands are the shortest suggested for use with the present invention. Thus, the compounds listed in this filing that are suitable for use in the present example are limited to 16-mers or to 15-12-mers that are extended to 16-mers using the human ApoB sequence. Such 16-mers can be further lengthened by the use of overhangs which as described herein do not necessarily need to base pair with the gene target.

A number of treatment regimens suitable for use with such conventional antisense oligos or for use with the two-step administration described by the present invention are provided in WO 2008/118883.The sequence used for human ApoB is provided in GenBank, Accession No. X04714.1.

Atherosclerosis is a condition in which vascular smooth muscle cells are pathologically reprogrammed. Fatty material collects in the walls of arteries and there is typically chronic inflammation. This leads to a situation where the vascular wall thickens, hardens, forms plaques, which may eventually block the arteries or promote the blockage of arteries by promoting clotting. The latter becomes much more prevalent when there is a plaque rupture.

If the coronary arteries become narrow due to the effects of the plaque formation, blood flow to the heart can slow down or stop, causing chest pain (stable angina), shortness of breath, heart attack, and other symptoms. Pieces of plaque can break apart and move through the bloodstream. This is a common cause of heart attack and stroke. If the clot moves into the heart, lungs, or brain, it can cause a stroke, heart attack, or pulmonary embolism.

Risk factors for atherosclerosis include: diabetes, high blood pressure, high cholesterol, high-fat diet, obesity, personal or family history of heart disease and smoking The following conditions have also been linked to atherosclerosis: cerebrovascular disease, kidney disease involving dialysis and peripheral vascular disease. Down modulation of apoB s can have a beneficial therapeutic effect for the treatment of atherosclerosis and associated pathologies. WO/2007/030556 provides an animal model for assessing the effects of apoB directed compounds on the formation of atherosclerotic lesions.

### D. Compounds for Down-Regulating PCSK9 Expression

Protein convertase subtilisin-like kexin type 9 (PCSK9) is a serine protease that destroys LDL receptors in liver and consequently the level of LDL in plasma. PCSK9 mutants can have gain-of-function attributes that promote certain medical disorders associated with alterations in the proportions of plasma lipids. Agents that inhibit PCSK9 function have a role to play in the treatment of such medical disorders including those listed in Table 6.

Frank-Kamenetsky et al. (Proc Natl Acad Sci USA 105: 11915, 2008) have described four siRNA compounds suitable for use in the present invention with three different sequences directed to the PCSK9 gene targets of human, mouse, rats, and nonhuman primates (and have characterized their activity in model systems. These siRNA were selected from a group of 150 by screening for activity using HepG2 cells. These compounds were formulated in lipidoid nanoparticles for in vivo testing. These compounds reduced PCSK9 expression in the livers of rats and mice by 50-70% and this was associated with up to a 60% reduction in plasma cholesterol levels. In transgenic mice carrying the human PCSK9 gene siRNA compounds were shown to reduce the levels of the transcripts of this gene in livers by >70%. In nonhuman primates after a single bolus injection of PCSK9 siRNA the negative effect on PCSK9 expression lasted 3 weeks. During this time apoB and LDL cholesterol (LDLc) levels were reduced. There were no detectable effects on HDL cholesterol or triglycerides. US2008/0113930 and WO 2007/134161 disclose additional PCSK9 RNAi compounds which can be modified as disclosed herein.

Conventional antisense oligos directed to the PCSK9 gene target provide another example showing how conventional antisense oligos can be reconfigured to provide novel compositions of matter suitable for use in the present invention. Such a reconfiguration is useful in situations where siRNA has advantages over conventional antisense oligos as described herein. A series of conventional antisense oligos directed to human PCSK9 and suitable for use with the present invention have been described in WO 2007/143315. These sequences were among the most active of those that were screened for PCSK9 inhibiting activity in vitro using the Hep3B cell line. The conventional antisense oligos described in this filing are 8-16-mers. It is known that guide strands shorter than 15-mers are not active. Further 16-mer guide strands are the shortest suggested for use with the present invention. Such 16-mers can be further lengthened by the use of overhangs which as described herein do not necessarily need to base pair with the gene target in the case of the guide strand.

A number of treatment regimens suitable for use with such conventional antisense oligos or for use with the two-step administration of strands capable of forming siRNA in cells and where the guide strand is directed to PCSK9 are described in WO 2008/118883. The conventional antisense oligos in this filing are targeted to apoB but the tissues involved and the therapeutic purposes involving PCSK9 are the same and thus essentially the same treatment regimens can be used.

This protein plays a major regulatory role in cholesterol homeostasis. PCSK9 binds to the epidermal growth factor-like repeat A (EGF-A) domain of the low-density lipoprotein receptor (LDLR), inducing LDLR degradation. Reduced LDLR levels result in decreased metabolism of low-density lipoproteins, which could lead to hypercholesterolemia. Inhibition of PSCK9 function provides a means of lowering cholesterol levels. PCSK9 may also have a role in the differentiation of cortical neurons.

Further, the usefulness of conventional antisense oligos directed to the murine PCSK9 gene target for the treatment of hypercholesterolemia has been demonstrated by Graham et al. (J lipid Res 48: 763, 2007). A series of antisense oligos were screened for activity and the most active (ISIS 394814) selected for in vivo studies. Administration of ISIS 394814 to high fat fed mice for 6 weeks resulted in a 53% reduction in total plasma cholesterol and a 38% reduction in plasma LDL. This was accompanied by a 92% reduction in liver PCSK9 expression.

### E. Compounds for Down-Regulating Phosphatase and Tensin Homolog (PTEN) Expression

PTEN is a phosphatase (phosphatidylinositol-3,4,5-trisphosphate 3-phosphatase) that is frequently mutated in cancers with wild type p53 where the effect or the mutation is to inhibit its enzymatic activity. In this context, PTEN is thought to function as a tumor suppressor. In cancers with mutated p53, however, PTEN supports the viability and growth of the tumor in part by increasing the levels of gain-of-function p53 mutants (Li et al., Cancer Res 68: 1723, 2008). PTEN also modulates cell cycle regulatory proteins with the effect of inhibiting cell proliferation. Thus, PTEN inhibitors have a role in the treatment of some cancers and in promoting cell proliferation such as expanding cell populations for purposes such as transplantation.

In vivo regeneration of peripheral neurons is constrained and rarely complete, and unfortunately patients with major nerve trunk transections experience only limited recovery. Intracellular inhibition of neuronal growth signals may be among these constraints. Christie et al. investigated the role of PTEN (phosphatase and tensin homolog deleted on chromosome 10) during regeneration of peripheral neurons in adult Sprague Dawley rats (J. Neuroscience 30:9306-9315 (2010). PTEN inhibits phosphoinositide 3-kinase (PI3-K)/Akt signaling, a common and central outgrowth and survival pathway downstream of neuronal growth factors. While PI3-K and Akt outgrowth signals were expressed and activated within adult peripheral neurons during regeneration, PTEN was similarly expressed and poised to inhibit their support. PTEN was expressed in neuron perikaryal cytoplasm, nuclei, regenerating axons, and Schwann cells. Adult sensory neurons in vitro responded to both graded pharmacological inhibition of PTEN and its mRNA knockdown using siRNA. Both approaches were associated with robust rises in the plasticity of neurite outgrowth that were independent of the mTOR (mammalian target of rapamycin) pathway. Importantly, this accelerated outgrowth was in addition to the increased outgrowth generated in neurons that had undergone a preconditioning lesion. Moreover, following severe nerve transection injuries, local pharmacological inhibition of PTEN or siRNA knockdown of PTEN at the injury site accelerated axon outgrowth in vivo. The findings indicated a remarkable impact on peripheral neuron plasticity through PTEN inhibition, even within a complex regenerative milieu. Overall, these findings identify a novel route to propagate intrinsic regeneration pathways within axons to benefit nerve repair. In view of these findings, it is clear that the PTEN directed compounds of the invention can be useful for the treatment of nerve injury and damage. In a preferred embodiment, such agents would be administered intrathecally as described for insulin in Toth et al., Neuroscience(2006) 139:429-49. Czauderna et al. (Nuc Acids Res 31: 2705, 2003) have described an active siRNA compound that is directed to the human PTEN gene target which is suitable for use in accordance with the present invention as described herein. Allerson et al. (J Med Chem 48: 901, 2005) have described two siRNA compounds suitable for use in the present invention that are targeted to human PTEN.

### F. Compounds for Down-Regulating PTP1B Expression

PTP1B, a non-transmembrane protein tyrosine phosphatase that has long been studied as a negative regulator of insulin and leptin signaling, has received renewed attention as an unexpected positive factor in tumorigenesis. These dual characteristics make PTP1B a particularly attractive therapeutic target for diabetes, obesity, and perhaps breast cancer.

In the case of insulin signaling, PTP1B dephosphorylates the insulin receptor (IR) as well as its primary substrates, the IRS proteins; by contrast, in leptin signaling a downstream element, the tyrosine kinase JAK2(Janus kinase 2), is the primary target for dephosphorylation. However, hints that PTP1B might also play a positive signaling role in cell proliferation began to emerge a few years ago, with the finding by a number of groups that PTP1B dephosphorylates the inhibitory Y529 site in Src, thereby activating this kinase. Other PTP1B substrates might also contribute to pro-growth effects. Indeed, the idea that PTP1B can serve as a signaling stimulant in some cases received key confirmation in previous work that showed PTP plays a positive role in a mouse model of ErbB2-induced breast cancer. See Yip et al. Trends in Biochemical Sciences 35:442-449 (2010). For these reasons, PTP1B has attracted particular attention as a potential therapeutic target in obesity, diabetes, and now, cancer. Accordingly, the compounds directed at PTP1B can be used to advantage for the treatment of such disorders.

### Example II

### Applications for ss-IMiRs

MiRNAs have been shown to have wide ranging effects on gene expression. In certain instances, these effects are detrimental and related to certain pathologies. Accordingly, specific miRNA inhibitors which target such miRNAs for degradation are highly desirable. The present inventor has devised strategies for the synthesis of miRNA inhibitors suitable for in vivo delivery which exhibit enhanced stability, the ability to form active duplexes in cells, which act in turn to inhibit the activity of endogenous miRNAs associated with disease. These design paradigms and the resulting miRNA inhibitors are described herein below.

Table 7 provides a listing of some of the medical uses of the ss-IMiRs directed to the indicated miRNAs. The methods of the present invention, however, can be used to generate ss-IMiRs against any miRNA. Methods for administration of the oligos of the invention are provided in detail above.

TABLE 7
MICRORNA TARGETS FOR INHIBITION BY ss-
ImiRs AND COMMERCIAL APPLICATIONS

| MicroRNA | Medical Conditions to be Treated using the ss-IMiR |
|---|---|
| Targets | Compounds of the Invention |
| miR-24 | Treat cancer including hormone resistant prostate |
| miR-29a | Inhibit pathologic apoptosis including that due |
| | to ischemia reperfusion injury such as occurs |
| | after the removal of a clot |
| miR-29b | Inhibit pathologic apoptosis |
| miR-29c | Inhibit pathologic apoptosis including that due |
| | to ischemia reperfusion injury such as occurs |
| | after the removal of a clot |
| miR-33 | Raise good cholesterol (HDL) levels |
| miR-34a | Myocardial infarction |
| miR-122 | Hepatitis C |
| miR-155 | Arthritis; Autoimmune inflammation including that |
| | associated with cystic fibrosis; Atopic dermatitis |
| miR-208a | Chronic heart failure |

Conventional antisense oligos of different types are under development for potential use as competitive inhibitors of particular endogenous miRNAs for research, development and therapeutic purposes. Such oligos are designed to bind particularly tightly one strand of the miRNA whose actions are to be inhibited. These oligos work by a steric hindrance mechanism.

Elevated levels of miR-21, for example, occur in numerous cancers where it promotes oncogenesis at least in part by preventing the translation and accumulation of PDCD4. Another example is miR-122 a liver specific miRNA that promotes replication of the hepatitis C virus. Conventional antisense oligos that inhibit these miRNAs are in development as potential therapeutic agents.

Compared to antisense oligos that engender catalytic activity against their targets, such as those that are RNase H dependent, the antisense oligos that function as competitive inhibitors must be used at substantially higher concentrations. In vivo various tissues take up oligos in widely ranging amounts. For example, liver and kidney take up relatively large amounts while resting lymphocytes, testis, skeletal muscle the CNS and other tissues take up much smaller amounts. Further, antisense oligos that have a competitive inhibitor function have been shown to perform poorly in tissues that do not avidly take up oligos. Therefore, it would be highly desirable to have oligonucleotide based miRNA inhibitors that have a catalytic activity against them so that a wider range of tissues types can be subject to efficient miRNA inhibition. The present invention provides a solution to this pressing need.

### Example III

### Examples of Applications for ss-MiRs

Table 8 below provides a listing of miRNAs for which examples of specific ss-MiR compounds have been provided herein. The methods of the present invention can be used to mimic any endogenous miRNA, to improve on the mRNA type silencing pattern of an endogenous miRNA for commercial purposes and can be used to generate designer novel miRNA-like compounds.

TABLE 8
MICRORNAS MIMICKED BY ss-MiRsAND
COMMERCIAL APPLICATIONS

| MicroRNA | |
|---|---|
| Mimicked | Medical Conditions to be Treated using the ss-MiR |
| by ss-MiR | Compounds of the Invention |
| Let-7i and | Cancer |
| Let-7 family | |
| generally | |
| miR-24-1 | Ischemia reperfusion injury including that associated |
| | with myocardial infarction; Cardiac fibrosis; Diabetes |
| miR-24-2 | Ischemia reperfusion injury including that associated |
| | with myocardial infarction; Cardiac fibrosis; Diabetes |
| miR-26a-1 | Cancer including liver, head and neck, breast |
| miR-26a-2 | Cancer including liver, head and neck, breast |
| miR-29a | Fibrosis including liver, lung, kidney and heart; |
| | Systemic sclerosis; Cancers including lung, liver, |
| | chronic lymphocytic leukemia; Osteoporosis; Systemic |
| | sclerosis; |
| miR-29b-1 | Fibrosis including liver, lung, kidney and heart; |
| | Systemic sclerosis; Cancers including lung, liver, colon |
| | breast, chronic lymphocytic leukemia, acute myeloid |
| | leukemia |
| miR-29b-2 | Fibrosis including liver, lung, kidney and heart; Systemic |
| | sclerosis; Cancers including lung, liver, colon, breast, |
| | rhabdomyosarcoma, chronic lymphocytic leukemia, acute |
| | myeloid leukemia; |
| miR-29c | Fibrosis including liver, lung, kidney and heart; Systemic |
| | sclerosis; Cancers including lung, liver, rhabdomyosar- |
| | coma, chronic lymphocytic leukemia; |
| miR-34a | Cancer including prostate, ovarian, non-small cell lung |
| | cancer, pancreatic cancer, stomach cancer, retinoblastoma |
| | and chronic lymphocytic leukemia; |
| miR-34b | Cancer including prostate, ovarian, non-small cell lung |
| | cancer, pancreatic cancer, stomach cancer, retinoblastoma |
| | and chronic lymphocytic leukemia; |
| miR-34c | Cancer including prostate, ovarian, non-small cell lung |
| | cancer, pancreatic cancer, stomach cancer, retinoblastoma |
| | and chronic lymphocytic leukemia; |
| miR-122 | Cancer including liver, lung and cervical; |
| miR-146a | Atherosclerosis |
| miR-203 | Sensitize cancers with mutant p53 including colon cancer |
| | to chemotherapy including taxanes |
| miR-214 | Nerve regeneration; Diabetes including type 2; |
| miR-499 | Myocardial infarction including the ischemia-reperfusion |
| | injury related to reversing vessel occlusion; |

It is now well established that post-transcriptional gene silencing (PTGS) by miRNA and other RNAi-associated pathways represents an essential layer of complexity to gene regulation. Gene silencing using RNAi additionally demonstrates huge potential as a therapeutic strategy for eliminating gene expression associated with the pathology underlying a number of different disorders.

A number of conventional miRNA compounds closely based on their endogenous miRNA counterparts are in development as possible therapeutic agents. Cancer is one area of focus since it has been found that several different miRNAs are expressed at very low levels in cancer cells compared to their normal counterparts. Further, it has been shown that replacing these miRNAs can have profound anticancer effects. Several specific examples are provided in the Table.

The miRNA mimics provided should also be effective in cell culture in vitro. In this approach, the first strand can be transfected into the target cells following by subsequent transfection of the second strand after a certain time frame has elapsed. This method should facilitate drug discovery efforts, target validation and also provide the means to reduce or eliminate any undesirable off target effects.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A composition for inhibiting expression of at least one target ribonucleic acid sequence of interest in a cell, comprising;
a modified single stranded oligoribonucleic acid in a pharmaceutically acceptable vehicle, said strand comprising one or more accommodating helical design (AHD) modifications and other chemical modifications effective to alter at least one parameter selected from the group consisting of
a) enhanced resistance to 5′and 3′ exonucleases and endonucleases in vivo;
b) enhanced C3′-endo conformation in one or more flexible sugar moieties in said oligoribonucleotide strand comprising said AHD modifications;
c) increased potency in vivo and in vitro;
d) reduced steric hinderance of strand interaction with RISC machinery via omission of moieties which project into major or minor grooves of duplexed RNAi triggers while maintaining RNAi activity;
e) reduced off-target effects; and
f) enhanced activity of the RNAi mechanism within cells relative to RNA strands lacking said AHD modifications; and
wherein
i) said composition does not comprise a pro-drug;
ii) said strand is between 16 and 22 nucleotides in length, exclusive of any 3′-end overhang precursor and has a region of complementarity to the target ribonucleic acid that is at least 6 contiguous nucleosides in length, comprises at least one of ribose, 2′-fluoro modification, 2′-O-methyl modification, an AHD modified sugar or sugar substitute, and an AHD modified base;
iii) said strand optionally comprises a 5′ end modification which inhibits 5′ exonuclease activity and/or promotes RISC loading;
iv) said strand optionally comprises a 3′ end modification which inhibits 3′ exonuclease activity and/or promotes RISC loading;
v) said strand optionally comprises at least one modification that inhibits endonuclease activity;
v) said strand optionally comprises a 3′-end overhang precursor between 1 and 4 units in length; and
wherein said modified oligoribonucleotide strand exhibits increased inhibition of expression of said target ribonucleic acid within said cell relative to identical oligoribonucleotide strands lacking said AHD modifications.

2. The composition as claimed in claim 1, wherein nucleosides in said 3′ overhang precursor in said modified oligoribonucleotide are operably linked via nuclease resistant linkages selected from the group consisting of phosphorothioate, phosphonoacetate, thiophosphonoacetate, methylborane phosphine, amide, carbamate, urea and remaining linkages in the strand are selected from the group consisting of phosphodiester, phosphorothioate and boranophosphate.

3. The composition as claimed in claim 1, wherein said modified oligoribonucleotide comprises a modified base selected from the group consisting of modified adenine, modified cytosine, modified guanine, modified uracil, thymine, 2,6-Diaminopurine, 2-thiouracil, 4-thiouracil, Pseudouracil, 3-methyluracil , 5-methyluracil, and 5-methylcytosine.

4. The composition as claimed in claim 1, wherein said ribose, AHD modified sugar or sugar substitute in said modified oligoribonucleotide is selected from the group consisting of 2′-fluoro, 2′-O-methyl, 2′-O-methyoxyethyl (2′MOE), AENA, ALN, ANA, CENA, CRN, EA, FANA, Arabinonucleoside, HM, HNA, FHNA, LNA, UNA, CeNA, and F—CeNA.

5. The composition as claimed in claim 1, wherein a 5′ carbon of said 5′-end ribose or AHD modified sugar or sugar substitute is selected from the group consisting of a hydroxyl group, a phosphate group, 5′-monophosphate[(HO)₂(O)P—O-5′], 5′-diphosphate[(HO)₂(O)P—O—P(HO)(O)—O-5′], 5′-triphosphate[(HO)₂(O)P—O—(HO)(O)P—O—P(HO)(O)—O—5′], 5′3′ diphosphate, 5′-guanosine cap (7-methylated or not methylated) [7m-G-O-5′-(HO)(O)P—O—(HO)(O)P—O—P(HO)(O)—O-5′], 5′-adenosine cap (Appp), [N—O-5′-(HO)(O)P—O—(HO)(O)P—O—P(HO)(O)—O—5′], 5′-monothiophosphate (phosphorothioate) (HO)₂(S)P—O-5′, 5′-monodithiophosphate (phosphorodithioate) (HO)(HS)(S)P—O-5′, 5′-phosphorothiolate[(HO)₂(O)P—S-5′]; 5′-α-thiotriphosphate, 5′-γ-thiotriphosphate, 5′-phosphoramidates[(HO)₂(O)P—NH-5′, (HO)(NH₂)(O)P—O-5′], 5′alkylphosphonates; isopropyl, propyl, [RP(OH)(O)—O-5′-, (OH)₂(O)P-5′-CH₂—], 5′alkyletherphosphonates; RP(OH)(O)—O-5′, 5′methylenephosphonate (5′-MP) and 5′-(E)-vinylphosphonate (5′-VP).

6. The composition of claim 1 comprising an overhang precursor selected from the group of precursors set forth in Table 4.

7. The composition of claim 1, wherein said target nucleic acid is selected from the group consisting of mRNA, non-coding regulatory RNA, miRNA, endogenous anti-sense RNA, and long non-coding RNA.

8. The composition as claimed in claim 1, which is a ss-RNAi directed to a target selected from the group consisting of PTEN, p53, Factor VII, apo-CIII, SSB, apo-B.

9. The composition as claimed in claim 8 wherein said ss-RNAi is directed to PTEN and is modified as shown in FIGS. 19 and 21.

10. The composition as claimed in claim 8 wherein said ss-RNAi is directed to p53 and is modified as shown in FIG. 20.

11. The composition as claimed in claim 8 wherein said ss-RNAi is directed to Factor VII and is modified as shown in FIG. 22.

12. The composition as claimed in claim 8 wherein said ss-RNAi is directed to apo-CIII and is modified as shown in FIG. 23.

13. The composition as claimed in claim 8 wherein said ss-RNAi is directed to SSB and is modified as shown in FIG. 24.

14. The composition as claimed in claim 8 wherein said ss-RNAi is directed to apo-B and is modified as shown in FIG. 25.

15. The composition of claim 1, wherein said modified oligoribonucleotide is a ss-MiR about 16-22 nucleosides in length exclusive of any overhang precursor, comprising a 9 nucleoside 5′ end module, a seed vehicle portion of about 8-14 nucleosides and optionally a 3′ overhang precursor.

16. The composition of claim 15, comprising a 3′ overhang precursor and a phosphate or phosphate isostere structure conjugated to the 5′ carbon of the 5′-end nucleoside sugar or sugar substitute.

17. The composition of claim 16, wherein said seed vehicle portion is a 10-mer selected from the group consisting of those listed in FIG. 28.

18. The composition of claim 15 wherein said ss-MiR mimics a naturally occurring miRNA selected from the group consisting of MiR-34a, MiR-124, and MiR-122.

19. The composition of claim 18, wherein said ss-MiR mimics MiR-34-a and is modified as shown in FIG. 17.

20. The composition of claim 18, wherein said ss-MiR mimics MiR-124 and is modified as shown in FIG. 26.

21. The composition of claim 18, wherein said ss-MiR mimics Mir-122 and is modified as shown in FIG. 27.

22. The composition of claim 1 further comprising a protective carrier.

23. The composition of claim 1, wherein said oligoribonucleotide is conjugated to a carrier which is effective to promote cellular uptake and/or cellular targeting.

24. A method of inhibiting expression of a target nucleic acid comprising contacting a cell expressing said target nucleic acid with an effective amount of the composition of any of the previous claims, said composition being effective to degrade target RNA or inhibit translation of mRNA encoding a protein produced by said target nucleic acid.

25. The method of claim 24, wherein said target encodes a protein which modulates a disease selected from the group consisting of Cancer, AIDS, Alzheimer's disease, Amyotrophic lateral sclerosis, Atherosclerosis, Autoimmune Diseases, Cerebellar degeneration, Cancer, Diabetes Mellitus, Glomerulonephritis, Heart Failure, Macular Degeneration, Multiple sclerosis, Myelodysplastic syndromes, Parkinson's disease, Prostatic hyperplasia, Psoriasis, Asthma, Retinal Degeneration, Retinitis pigmentosa, Rheumatoid arthritis, Rupture of atherosclerotic plaques, Systemic lupus erythematosis, Ulcerative colitis, viral infection, ischemia reperfusion injury, spinal cord injury, nerve damage, cardiohypertrophy, and Diamond Black Fan anemia.

26. An in vitro method of improving an RNAi effect in vitro or in vivo against a target nucleic acid, said method comprising;
(i) obtaining an oligoribonucleotide sequence which specifically hybridizes to said target nucleic acid;
(ii) introducing one or more accommodating helical design (AHD) modifications and other chemical modifications into said oligoribonucleotide, thereby producing a modified oligoribonucleotide, wherein said modifications are effective to modulate at least one parameter selected from the group consisting of
a) enhanced resistance to 5′and 3′ exonucleases and endonucleases in vivo;
b) enhanced C3′-endo conformation in one or more flexible sugar moieties in said oligoribonucleotide strand comprising said AHD modifications;
c) increased potency in vivo and in vitro;
d) reduced steric hinderance of strand interaction with RISC machinery via omission of moieties which project into major or minor grooves of duplexed RNAi triggers while maintaining RNAi activity;
e) reduced off-target effects; and
f) enhanced activity of the RNAi mechanism within target tissue in vivo relative to RNA strands lacking said AHD modifications; and
(iii) contacting a first population of cells expressing said target nucleic acid with the modified oligoribonucleotide of step ii) and a second population of identical cells expressing said target nucleic acid with an identical oligoribonucleotide strand lacking said modifications; and;
(iv) determining the effect of said contact of step iii) on said parameter, parameters being affected by those strands comprising said AHD modifications being identified as AHD modifications which improve RNAi effects in vitro and in vivo.

27. The method of claim 26, wherein said target nucleic acid is selected from the group consisting of mRNA, non-coding regulatory RNA, miRNA, endogenous anti-sense RNA, and long non-coding RNA.

28. The method of claim 26, wherein said modified oligoribonucleotide strand comprises a 3′ overhang precursor operably linked via nuclease resistant linkages selected from the group consisting of phosphorothioate, phosphonoacetate, thiophosphonoacetate, methylborane phosphine, amide, carbamate, urea while remaining linkages in said modified oligoribonucleotide strand are selected from the group consisting of phosphodiester, phosphorothioate and boranophosphate.

29. The method of claim 26, wherein said modified oligoribonucleotide comprises a modified base selected from the group consisting of adenine, cytosine, guanine, 2-thiouracil, 4-thiouracil, Pseudouracil, 3-methyluracil , 5-methyluracil, and 5-methylcytosine.

29. The method of claim 26, wherein said modified oligoribonucleotide comprises a modification selected from the group consisting of 2′-fluoro, 2′-O-methyl, 2′-O-methyoxyethyl (2′MOE), AENA, ALN, ANA, CENA, CRN, EA, FANA, Arabinonucleoside, HM, HNA, FHNA, LNA, UNA, CeNA, and F—CeNA.

30. The method of claim 26, wherein a 5′ carbon of a 5′-end ribose or a AHD modified sugar or sugar substitute is selected from the group consisting of a hydroxyl group, a phosphate group, 5′-monophosphate[(HO)₂(O)P—O-5′], 5′-diphosphate[(HO)₂(O)P—O—P(HO)(O)—O-5′], 5′-triphosphate[(HO)₂(O)P—O—(HO)(O)P—O—P(HO)(O)—O-5], 5′3′ diphosphate, 5′-guanosine cap (7-methylated or not methylated) [7m-G-O-5′-(HO)(O)P—O—(HO)(O)P—O—P(HO)(O)—O-5], 5′-adenosine cap (Appp), [N—O-5′-(HO)(O)P—O—(HO)(O)P—O—P(HO)(O)—O-5], 5′-monothiophosphate (phosphorothioate) (HO)₂(S)P—O-5′, 5′-monodithiophosphate (phosphorodithioate) (HO)(HS)(S)P—O-5′, 5′-phosphorothiolate[(HO)₂(O)P—S-5′]; 5′-α-thiotriphosphate, 5′-γ-thiotriphosphate, 5′-phosphoramidates[(HO)₂(O)P—NH-5′, (HO)(NH₂)(O)P—O-5′], 5′alkylphosphonates; isopropyl, propyl, [RP(OH)(O)—O-5′-, (OH)₂(O)P-5′-CH₂—], 5′alkyletherphosphonates; RP(OH)(O)—O-5′, 5′methylenephosphonate (5′-MP) and 5′-(E)-vinylphosphonate (5′-VP).

31. The method of claim 28, wherein said 3′ overhang precursor is selected from the group of precursors set forth in Table 7.

32. An RNAi molecule identified by the method of claim 26.
